SEQUENCE LISTING (8 sequences)

SEQ ID NO: 1 - DNA, 59 bp, Artificial Sequence
  lacZ alpha-RI primer for PCR amplification
gacgaattcgctagcaagaggagaagtcgacatgtccaattcactggccgtcgttttac

SEQ ID NO: 2 - DNA, 47 bp, Artificial Sequence
  lacZ alpha-3-prime BB primer for PCR amplification
gaccctaggaagctttctagagtcgacctatgcggcatcagagcaga

SEQ ID NO: 3 - protein, 474 aa, Artificial Sequence
  Mutated LpdA from E. coli K-12 MG1655
MSTEIKTQVVVLGAGPAGYSAAFRCADLGLETVIVERYNTLGGVCLNVGCIPSKALLHVAKVIEEAKALAEHGIVFGEPKTDIDKIRTWKEKVINQLTGGLAGMAKGRKVKVVNGLGKFTGANTLEVEGENGKTVINFDNAIIAAGSRPIQLPFIPHEDPRIWDSTDALELKEVPERLLVMGGGIIGLEMGTVYHALGSQIDVVVRKHQVIRAADKDIVKVFTKRISKKFNLMLETKVTAVEAKEDGIYVTMEGKKAPAEPQRYDAVLVAIGRVPNGKNLDAGKAGVEVDDRGFIRVDKQLRTNVPHIFAIGDIVGQPMLAHKGVHEGHVAAEVIAGKKHYFDPKVIPSIAYTEPEVAWVGLTEKEAKEKGISYETATFPWAASGRAIASDCADGMTKLIFDKESHRVIGGAIVGTNGGELLGEIGLAIEMGCDAEDIALTIHAHPTLHESVGLAAEVFEGSITDLPNPKAKKK

SEQ ID NO: 4 - protein, 474 aa, Artificial Sequence
  Mutated LpdA from E. coli K-12 MG1655
MSTEIKTQVVVLGAGPAGYSAAFRCADLGLETVIVERYNTLGGVCLNVGCIPSKALLHVAKVIEEAKALAEHGIVFGEPKTDIDKIRTWKEKVINQLTGGLAGMAKGRKVKVVNGLGKFTGANTLEVEGENGKTVINFDNAIIAAGSRPIQLPFIPHEDPRIWDSTDALELKEVPERLLVMGGGIIALEMATVYHALGSQIDVVVRKHQVIRAADKDIVKVFTKRISKKFNLMLETKVTAVEAKEDGIYVTMEGKKAPAEPQRYDAVLVAIGRVPNGKNLDAGKAGVEVDDRGFIRVDKQLRTNVPHIFAIGDIVGQPMLAHKGVHEGHVAAEVIAGKKHYFDPKVIPSIAYTEPEVAWVGLTEKEAKEKGISYETATFPWAASGRAIASDCADGMTKLIFDKESHRVIGGAIVGTNGGELLGEIGLAIEMGCDAEDIALTIHAHPTLHESVGLAAEVFEGSITDLPNPKAKKK

SEQ ID NO: 5 - protein, 364 aa, Artificial Sequence
  Mutant Candida bodinii enzyme
MKIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYQRQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 6 - protein, 364 aa, Artificial Sequence
  Mutant Candida bodinii enzyme
MKIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYSPQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 7 - protein, 376 aa, Artificial Sequence
  Mutant Saccharomyces cerevisiae enzyme
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYARQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 8 - protein, 20 aa, Euglena gracilis
  first 20 amino acids starting from the N-terminus of a KDC from Euglena gracilis
MTYKAPVKDVKFLLDKVFKV

(19) United States
(12) Patent Application Publication
(10) Pub. No.: US 2017/0247709 A1
(43) Pub. Date: Aug. 31, 2017

(54) EUKARYOTIC ORGANISMS AND METHODS FOR INCREASING THE AVAILABILITY OF CYTOSOLIC ACETYL-COA, AND FOR PRODUCING 1,3-BUTANEDIOL

(71) Applicant: Genomatica, Inc., San Diego, CA (US)

(72) Inventors: Anthony P. BURGARD, Bellefonte, PA (US); Mark J. BURK, San Diego, CA (US); Robin E. OSTERHOUT, San Diego, CA (US); Priti PHARKYA, San Diego, CA (US); Jingyi LI, Carlsbad, CA (US)

(21) Appl. No.: 15/290,895

(22) Filed: Oct. 11, 2016

Related U.S. Application Data
(63) Continuation of application No. 13/607,527, filed on Sep. 7, 2012, now abandoned.
(60) Provisional application No. 61/655,355, filed on Jun. 4, 2012.
(60) Provisional application No. 61/649,039, filed on May 18, 2012.
(60) Provisional application No. 61/558,959, filed on Nov. 11, 2011.
(60) Provisional application No. 61/541,951, filed on Sep. 30, 2011.
(60) Provisional application No. 61/532,492, filed on Sep. 8, 2011.

(51) Int. Cl. C12N 15/52; C12P 7/18; C12N 1/20; C12N 1/16; C12N 1/14

(52) U.S. Cl. CPC C12N 15/52; C12N 1/16; C12N 1/14; C12N 1/20; C12P 7/18

(57) ABSTRACT

Provided herein are non-naturally occurring eukaryotic organisms that can be engineered to produce and increase the availability of cytosolic acetyl-CoA. Also provided herein are non-naturally occurring eukaryotic organisms having a 1,3-butanediol (1,3-BDO) pathway. and methods of using such organisms to produce 1,3-BDO.

This application is a continuation of U.S. Ser. No. 13/607,527 filed Sep. 7, 2012, which claims the benefit of U.S. Ser. Nos. 61/532,492 filed Sep. 8, 2011; 61/541,951 filed Sep. 30, 2011; 61/558,959 filed Nov. 11, 2011; 61/649,039 filed May 18, 2012; and 61/655,355 filed Jun. 4, 2012, each hereby incorporated by reference in its entirety.

### 1. BACKGROUND

Provided herein are methods generally relating to biosynthetic processes and eukaryotic organisms capable of producing organic compounds. More specifically, in certain embodiments, provided herein are non-naturally occurring eukaryotic organisms that can be engineered to produce and increase the availability of cytosolic acetyl-CoA. In many eukaryotic organisms, acetyl-CoA is mainly synthesized by pyruvate dehydrogenase in the mitochondrion (FIG. 1). Thus, there exists a need to develop eukaryotic organisms that can produce and increase the availability of cytosolic acetyl-CoA. A mechanism for exporting acetyl-CoA from the mitochondrion to the cytosol enables deployment of a cytosolic production pathway that originates from acetyl-CoA. Such cytosolic production pathways include, for example, the production of commodity chemicals, such as 1,3-butanediol (1,3-BDO) and/or other compounds of interest.

Also provided herein are non-naturally occurring eukaryotic organisms that can be engineered to produce 1,3-BDO. The reliance on petroleum based feedstocks for production of 1,3-BDO warrants the development of alternative routes to producing 1,3-BDO and butadiene using renewable feedstocks. Thus, there exists a need to develop eukaryotic organisms and methods of their use to produce 1,3-BDO.

The organisms and methods provided herein satisfy these needs and provides related advantages as well.

### 2. SUMMARY

Provided herein are non-naturally occurring eukaryotic organisms that can be engineered to produce and increase the availability of cytosolic acetyl-CoA. Such organisms would advantageously allow for the production of cytosolic acetyl-CoA, which can then be used by the organism to produce compounds of interest, such as 1,3-BDO, using a cytosolic production pathway. Also provided herein are non-naturally occurring eukaryotic organisms having a 1,3-BDO pathway. and methods of using such organisms to produce 1,3-BDO.

In a first aspect, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a phosphoenolpyruvate (PEP) carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

In another aspect, provided herein is a method for transporting acetyl-CoA from a mitochondrion and/or peroxisome to a cytosol of a non-naturally occurring eukaryotic organism, comprising culturing a non-naturally occurring eukaryotic organism comprising an acetyl-CoA pathway under conditions and for a sufficient period of time to transport the acetyl-CoA from a mitochondrion and/or peroxisome to a cytosol of the non-naturally occurring eukaryotic organism. In some embodiments, provided herein is a method for transporting acetyl-CoA from a mitochondrion to a cytosol of said non-naturally occurring eukaryotic organism. In other embodiments, provided herein is a method for transporting acetyl-CoA from a peroxisome to a cytosol of said non-naturally occurring eukaryotic organism. In some embodiments culturing a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a mitochondrion and/or peroxisome of said organism to the cytosol of said organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; and a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

In another aspect, provided herein is a method for producing cytosolic acetyl-CoA, comprising culturing a non-naturally occurring eukaryotic organism comprising an acetyl-CoA pathway under conditions and for a sufficient period of time to produce cytosolic acetyl-CoA. In one embodiment, provided herein is a method for producing cytosolic acetyl-CoA, comprising culturing a non-naturally occurring eukaryotic organism comprising an acetyl-CoA pathway, wherein said organism comprises at least one exogenous nucleic acid encoding an acetyl-CoA pathway enzyme expressed in a sufficient amount to produce cytosolic acetyl-CoA in said organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; and a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

In another aspect, provided herein is a method for increasing acetyl-CoA in the cytosol of a non-naturally occurring eukaryotic organism, comprising culturing a non-naturally occurring eukaryotic organism comprising an acetyl-CoA pathway under conditions and for a sufficient period of time to increase the acetyl-CoA in the cytosol of the organism. In some embodiments, provided herein is a method for increasing acetyl-CoA in the cytosol of a non-naturally occurring eukaryotic organism, comprising culturing a non-naturally occurring eukaryotic organism comprising an acetyl-CoA pathway, wherein said organism comprises at least one exogenous nucleic acid encoding an acetyl-CoA pathway enzyme expressed in a sufficient amount to increase acetyl-CoA in the cytosol of said non-naturally occurring eukaryotic organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; and a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

Provided herein are non-naturally occurring eukaryotic organisms and methods thereof to produce and increase the availability of cytosolic acetyl-CoA in the eukaryotic organisms thereof. Also provided herein are non-naturally occurring eukaryotic organisms and methods thereof to produce optimal yields of certain commodity chemicals, such as 1,3-BDO, or other compounds of interest.

In another aspect, provided herein is a non-naturally occurring eukaryotic organism, comprising (1) an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism, and (2) a 1,3-BDO pathway, comprising at least one exogenous nucleic acid encoding a 1,3-BDO pathway enzyme expressed in a sufficient amount to produce 1,3-BDO. In certain embodiments, (1) the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase; and/or (2) the 1,3-BDO pathway comprises one or more enzymes selected from the group consisting of an acetoacetyl-CoA thiolase; an acetyl-CoA carboxylase; an acetoacetyl-CoA synthase; an acetoacetyl-CoA reductase (CoA-dependent, alcohol forming); 3-oxobutyraldehyde reductase (aldehyde reducing); 4-hydroxy,2-butanone reductase; an acetoacetyl-CoA reductase (CoA-dependent, aldehyde forming); a 3-oxobutyraldehyde reductase (ketone reducing); 3-hydroxybutyraldehyde reductase; an acetoacetyl-CoA reductase (ketone reducing); a 3-hydroxybutyryl-CoA reductase (aldehyde forming); a 3-hydroxybutyryl-CoA reductase (alcohol forming); an acetoacetyl-CoA transferase, an acetoacetyl-CoA hydrolase, an acetoacetyl-CoA synthetase, or a phosphotransacetoacetylase and acetoacetate kinase; an acetoacetate reductase; a 3-hydroxybutyryl-CoA transferase, hydrolase, or synthetase; a 3-hydroxybutyrate reductase; and a 3-hydroxybutyrate dehydrogenase.

In another aspect, provided herein is a method for producing 1,3-BDO, comprising culturing a non-naturally occurring eukaryotic organism under conditions and for a sufficient period of time to produce the 1,3-BDO, wherein the non-naturally occurring eukaryotic organism comprises (1) an acetyl-CoA pathway, and (2) a 1,3-BDO pathway. In certain embodiments, provided herein is a method for producing 1,3-BDO, comprising culturing a non-naturally occurring eukaryotic organism, comprising an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism; and/or (2) a 1,3-BDO pathway, comprising at least one exogenous nucleic acid encoding a 1,3-BDO pathway enzyme expressed in a sufficient amount to produce 1,3-BDO. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase; and (2) the 1,3-BDO pathway comprises one or more enzymes selected from the group consisting of an acetoacetyl-CoA thiolase; an acetyl-CoA carboxylase; an acetoacetyl-CoA synthase; an acetoacetyl-CoA reductase (CoA-dependent, alcohol forming); 3-oxobutyraldehyde reductase (aldehyde reducing); 4-hydroxy,2-butanone reductase; an acetoacetyl-CoA reductase (CoA-dependent, aldehyde forming); a 3-oxobutyraldehyde reductase (ketone reducing); 3-hydroxybutyraldehyde reductase; an acetoacetyl-CoA reductase (ketone reducing); a 3-hydroxybutyryl-CoA reductase (aldehyde forming); a 3-hydroxybutyryl-CoA reductase (alcohol forming); an acetoacetyl-CoA transferase, an acetoacetyl-CoA hydrolase, an acetoacetyl-CoA synthetase, or a phosphotransacetoacetylase and acetoacetate kinase; an acetoacetate reductase; a 3-hydroxybutyryl-CoA transferase, hydrolase, or synthetase; a 3-hydroxybutyrate reductase; and a 3-hydroxybutyrate dehydrogenase.

In another aspect, provided herein is a non-naturally occurring eukaryotic organism comprising (1) a 1,3-BDO pathway, wherein said organism comprises at least one exogenous nucleic acid encoding a 1,3-BDO pathway enzyme expressed in a sufficient amount to produce 1,3-BDO and (2) a deletion or attenuation of one or more enzymes or pathways that utilize one or more precursors and/or intermediates of a 1,3-BDO pathway. In a specific embodiment, the non-naturally occurring eukaryotic organism comprises a deletion or attenuation of a competing pathway that utilizes acetyl-CoA. In a specific embodiment, the non-naturally occurring eukaryotic organism comprises a deletion or attenuation of a 1,3-BDO intermediate byproduct pathway.

In another aspect, provided herein is a non-naturally occurring eukaryotic organism comprising (1) a 1,3-BDO pathway, wherein said organism comprises at least one exogenous nucleic acid encoding a 1,3-BDO pathway enzyme expressed in a sufficient amount to produce 1,3-BDO and (2) a deletion or attenuation of one or more enzymes or pathways that utilize one or more cofactors of a 1,3-BDO pathway.

In another aspect, provided herein is a non-naturally occurring eukaryotic organism comprising a 1,3-BDO pathway, wherein said organism comprises one or more endogenous and/or exogenous nucleic acids encoding an attenuated 1,3-BDO pathway enzyme selected from the group consisting of an acetoacetyl-CoA reductase (CoA-dependent, alcohol forming), a 3-oxobutyraldehyde reductase (aldehyde reducing), a 4-hydroxy-2-butanone reductase, an acetoacetyl-CoA reductase (CoA-dependent, aldehyde forming), a 3-oxobutyraldehyde reductase (ketone reducing), a 3-hydroxybutyraldehyde reductase, an acetoacetyl-CoA reductase (ketone reducing), a 3-hydroxybutyryl-CoA reductase (aldehyde forming), a 3-hydroxybutyryl-CoA reductase (alcohol forming), an acetoacetate reductase, 3-hydroxybutyrate reductase, a 3-hydroxybutyrate dehydrogenase and a 3-hydroxybutyraldehyde reductase; and wherein the attenuated 1,3-BDO pathway enzyme is NAPDH-dependent and has lower enzymatic activity as compared to the 1,3-BDO pathway enzyme encoded by an unaltered or wild-type nucleic acid.

In another aspect, provided herein is a non-naturally occurring eukaryotic organism comprising a 1,3-BDO pathway, wherein said organism one or more endogenous and/or exogenous nucleic acids encoding a 1,3-BDO pathway enzyme selected from the group consisting of an acetoacetyl-CoA reductase (CoA-dependent, alcohol forming), a 3-oxobutyraldehyde reductase (aldehyde reducing), a 4-hydroxy-2-butanone reductase, an acetoacetyl-CoA reductase (CoA-dependent, aldehyde forming), a 3-oxobutyraldehyde reductase (ketone reducing), a 3-hydroxybutyraldehyde reductase, an acetoacetyl-CoA reductase (ketone reducing), a 3-hydroxybutyryl-CoA reductase (aldehyde forming), a 3-hydroxybutyryl-CoA reductase (alcohol forming), an acetoacetate reductase, 3-hydroxybutyrate reductase, a 3-hydroxybutyrate dehydrogenase and a 3-hydroxybutyraldehyde reductase; wherein at least one nucleic acid has been altered such that the 1,3-BDO pathway enzyme encoded by the nucleic acid has a greater affinity for NADH than the 1,3-BDO pathway enzyme encoded by an unaltered or wild-type nucleic acid.

In another aspect, provided herein is a non-naturally occurring eukaryotic organism comprising a 1,3-BDO pathway, wherein said organism comprises one or more endogenous and/or exogenous nucleic acids encoding a 1,3-BDO pathway enzyme selected from the group consisting of an acetoacetyl-CoA reductase (CoA-dependent, alcohol forming), a 3-oxobutyraldehyde reductase (aldehyde reducing), a 4-hydroxy-2-butanone reductase, an acetoacetyl-CoA reductase (CoA-dependent, aldehyde forming), a 3-oxobutyraldehyde reductase (ketone reducing), a 3-hydroxybutyraldehyde reductase, an acetoacetyl-CoA reductase (ketone reducing), a 3-hydroxybutyryl-CoA reductase (aldehyde forming), a 3-hydroxybutyryl-CoA reductase (alcohol forming), an acetoacetate reductase, 3-hydroxybutyrate reductase, a 3-hydroxybutyrate dehydrogenase and a 3-hydroxybutyraldehyde reductase, wherein at least one nucleic acid has been altered such that the 1,3-BDO pathway enzyme encoded by the nucleic acid has a lesser affinity for NADPH than the 1,3-BDO pathway enzyme encoded by an unaltered or wild-type nucleic acid.

In another aspect, provided herein is a non-naturally occurring eukaryotic organism comprising: (1) a 1,3-BDO pathway, wherein said organism comprises at least one endogenous and/or exogenous nucleic acid encoding a 1,3-BDO pathway enzyme expressed in a sufficient amount to produce 1,3-BDO; and (2) an acetyl-CoA pathway, wherein said organism comprises at least one endogenous and/or exogenous nucleic acid encoding an acetyl-CoA pathway enzyme expressed in a sufficient amount to increase NADH in the organism; wherein the acetyl-CoA pathway comprises (i.) an NAD-dependent pyruvate dehydrogenase; (ii.) a pyruvate formate lyase and an NAD-dependent formate dehydrogenase; (iii.) a pyruvate:ferredoxin oxidoreductase and an NADH:ferredoxin oxidoreductase; (iv.) a pyruvate decarboxylase and an NAD-dependent acylating acetylaldehyde dehydrogenase; (v.) a pyruvate decarboxylase, a NAD-dependent acylating acetaldehyde dehydrogenase, an acetate kinase, and a phosphotransacetylase; or (vi.) a pyruvate decarboxylase, an NAD-dependent acylating acetaldehyde dehydrogenase, and an acetyl-CoA synthetase.

In another aspect, provided herein is a non-naturally occurring eukaryotic organism comprising: (1) a 1,3-BDO pathway, wherein said organism comprises at least one endogenous and/or exogenous nucleic acid encoding an NADPH-dependent 1,3-BDO pathway enzyme expressed in a sufficient amount to produce 1,3-BDO; and (2) a pentose phosphate pathway, wherein said organism comprises at least one endogenous and/or exogenous nucleic acid encoding a pentose phosphate pathway enzyme selected from the group consisting of glucose-6-phosphate dehydrogenase, 6-phosphogluconolactonase, and 6-phosphogluconate dehydrogenase (decarboxylating).

In another aspect, provided herein is a non-naturally occurring eukaryotic organism comprising: (1) a 1,3-BDO pathway, wherein said organism comprises at least one endogenous and/or exogenous nucleic acid encoding an NADPH-dependent 1,3-BDO pathway enzyme expressed in a sufficient amount to produce 1,3-BDO; and (2) an Entner Doudoroff pathway, wherein said organism comprises at least one endogenous and/or exogenous nucleic acid encoding an Entner Doudoroff pathway enzyme selected from the group consisting of glucose-6-phosphate dehydrogenase, 6-phosphogluconolactonase, phosphogluconate dehydratase, and 2-keto-3-deoxygluconate 6-phosphate aldolase.

In another aspect, provided herein is a non-naturally occurring eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) an endogenous and/or exogenous nucleic acid encoding a soluble or membrane-bound transhydrogenase, wherein the transhydrogenase is expressed in a sufficient amount to convert NADH to NADPH.

In another aspect, provided herein is a non-naturally occurring eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) an endogenous and/or exogenous nucleic acid encoding an NADP-dependent phosphorylating or non-phosphorylating glyceraldehyde-3-phosphate dehydrogenase.

In another aspect, provided herein is a non-naturally occurring eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) an acetyl-CoA pathway, wherein said organism comprises at least one endogenous and/or exogenous nucleic acid encoding an acetyl-CoA pathway enzyme expressed in a sufficient amount to increase NADPH in the organism; wherein the acetyl-CoA pathway comprises (i) an NADP-dependent pyruvate dehydrogenase; (ii) a pyruvate formate lyase and an NADP-dependent formate dehydrogenase; (iii) a pyruvate:ferredoxin oxidoreductase and an NADPH:ferredoxin oxidoreductase; (iv) a pyruvate decarboxylase and an NADP-dependent acylating acetylaldehyde dehydrogenase; (v) a pyruvate decarboxylase, a NADP-dependent acylating acetaldehyde dehydrogenase, an acetate kinase, and a phosphotransacetylase; or (vi) a pyruvate decarboxylase, an NADP-dependent acylating acetaldehyde dehydrogenase, and an acetyl-CoA synthetase.

In another aspect, provided herein is a non-naturally occurring eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) one or more endogenous and/or exogenous nucleic acids encoding a NAD(P)H cofactor enzyme selected from the group consisting of phosphorylating or non-phosphorylating glyceraldehyde-3-phosphate dehydrogenase; pyruvate dehydrogenase; formate dehydrogenase; and acylating acetylaldehyde dehydrogenase; wherein the one or more nucleic acids encoding a NAD(P)H cofactor enzyme has been altered such that the NAD(P)H cofactor enzyme encoded by the nucleic acid has a greater affinity for NADPH than the NAD(P)H cofactor enzyme encoded by an unaltered or wild-type nucleic acid.

In another aspect, provided herein is a non-naturally occurring eukaryotic organism comprising: (1) a 1,3-BDO pathway, comprising at least one endogenous and/or exogenous nucleic acid encoding a NADPH dependent 1,3-BDO pathway enzyme expressed in a sufficient amount to produce 1,3-BDO; and (2) one or more endogenous and/or exogenous nucleic acids encoding a NAD(P)H cofactor enzyme selected from the group consisting of a phosphorylating or non-phosphorylating glyceraldehyde-3-phosphate dehydrogenase; a pyruvate dehydrogenase; a formate dehydrogenase; and an acylating acetylaldehyde dehydrogenase; wherein the one or more nucleic acids encoding NAD(P)H cofactor enzyme nucleic acid has been altered such that the NAD(P)H cofactor enzyme that it encodes for has a lesser affinity for NADH than the NAD(P)H cofactor enzyme encoded by an unaltered or wild-type nucleic acid.

In another aspect, provided herein is a non-naturally occurring eukaryotic organism comprising a 1,3-BDO pathway, wherein said organism, and wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in a endogenous and/or exogenous nucleic acid encoding a NADH dehydrogenase; (ii) expresses an attenuated NADH dehydrogenase; and/or (iii) has lower or no NADH dehydrogenase enzymatic activity as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a cytochrome oxidase; (ii) expresses an attenuated cytochrome oxidase; and/or (iii) has lower or no cytochrome oxidase enzymatic activity as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a glycerol-3-phosphate (G3P) dehydrogenase; (ii) expresses an attenuated G3P dehydrogenase; (iii) has lower or no G3P dehydrogenase enzymatic activity as compared to a wild-type version of the eukaryotic organism; and/or (iv) produces lower levels of glycerol as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a G3P phosphatase; (ii) expresses an attenuated G3P phosphatase; (iii) has lower or no G3P phosphatase enzymatic activity as compared to a wild-type version of the eukaryotic organism; and/or (iv) produces lower levels of glycerol as compared to a wild-type version of the eukaryotic organism.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a pyruvate decarboxylase; (ii) expresses an attenuated pyruvate decarboxylase; (iii) has lower or no pyruvate decarboxylase enzymatic activity as compared to a wild-type version of the eukaryotic organism; and/or (iv) produces lower levels of ethanol from pyruvate as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an ethanol dehydrogenase; (ii) expresses an attenuated ethanol dehydrogenase; (iii) has lower or no ethanol dehydrogenase enzymatic activity as compared to a wild-type version of the eukaryotic organism; and/or (iv) produces lower levels of ethanol as compared to a wild-type version of the eukaryotic organism.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a malate dehydrogenase; (ii) expresses an attenuated malate dehydrogenase; (iii) has lower or no malate dehydrogenase enzymatic activity as compared to a wild-type version of the eukaryotic organism; and/or (iv) has an attenuation or blocking of a malate-asparate shuttle, a malate oxaloacetate shuttle, and/or a malate-pyruvate shuttle.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an acetoacetyl-CoA hydrolase or transferase; (ii) expresses an attenuated acetoacetyl-CoA hydrolase or transferase; and/or (iii) has lower or no acetoacetyl-CoA hydrolase or transferase enzymatic activity as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 3-hydroxybutyryl-CoA hydrolase or transferase; (ii) expresses an attenuated 3-hydroxybutyryl-CoA hydrolase or transferase; and/or (iii) has lower or no 3-hydroxybutyryl-CoA hydrolase or transferase enzymatic activity as compared to a wild-type version of the eukaryotic organism

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO; and wherein the organism: (i) comprises a disruption in an endogenous and/or exogenous nucleic acid encoding an acetaldehyde dehydrogenase (acylating); (ii) expresses an attenuated acetaldehyde dehydrogenase (acylating); and/or (iii) has lower or no acetaldehyde dehydrogenase (acylating) enzymatic activity as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 3-hydroxybutyraldehyde dehydrogenase; (ii) expresses an attenuated 3-hydroxybutyraldehyde dehydrogenase; and/or (iii) has lower or no 3-hydroxybutyraldehyde dehydrogenase enzymatic activity as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 3-oxobutyraldehyde dehydrogenase; (ii) expresses an attenuated 3-oxobutyraldehyde dehydrogenase; and/or (iii) has lower or no 3-oxobutyraldehyde dehydrogenase enzymatic activity as compared to a wild-type version of the eukaryotic organism.

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 1,3-butanediol dehydrogenase; (ii) expresses an attenuated 1,3-butanediol dehydrogenase; and/or (iii) has lower or no 1,3-butanediol dehydrogenase enzymatic activity as compared to a wild-type version of the eukaryotic organism

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an acetoacetyl-CoA thiolase; (ii) expresses an attenuated acetoacetyl-CoA thiolase; and/or (iii) has lower or no acetoacetyl-CoA thiolase enzymatic activity as compared to a wild-type version of the eukaryotic organism

In another aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO; and wherein said organism further comprises an endogenous and/or exogenous nucleic acid encoding a 1,3-BDO transporter, wherein the nucleic acid encoding the 1,3-BDO transporter is expressed in a sufficient amount for the exportation of 1,3-BDO from the eukaryotic organism.

In another aspect, provided herein is a non-naturally occurring eukaryotic organism comprising a combined mitochondrial/cytosolic 1,3-BDO pathway, wherein said organism comprises at least endogenous and/or exogenous nucleic acid encoding a combined mitochondrial/cytosolic 1,3-BDO pathway enzyme expressed in a sufficient amount to produce 1,3-BDO. In certain embodiments, the combined mitochondrial/cytosolic 1,3-BDO pathway comprises one or more enzymes selected from the group consisting of a mitochondrial acetoacetyl-CoA thiolase; an acetyl-CoA carboxylase; an acetoacetyl-CoA synthase; a mitochondrial acetoacetyl-CoA reductase; a mitochondrial acetoacetyl-CoA hydrolase, transferase or synthetase; a mitochondrial 3-hydroxybutyryl-CoA hydrolase, transferase or synthetase; a mitochondrial. 3-hydroxybutyrate dehydrogenase; an acetoacetate transporter; a 3-hydroxybutyrate transporter; a 3-hydroxybutyryl-CoA transferase or synthetase, a cytosolic acetoacetyl-CoA transferase or synthetase; an acetoacetyl-CoA reductase (CoA-dependent, alcohol forming); a 3-oxobutyraldehyde reductase (aldehyde reducing); a 4-hydroxy-2-butanone reductase; an acetoacetyl-CoA reductase (CoA-dependent, aldehyde forming); a 3-oxobutyraldehyde reductase (ketone reducing); a 3-hydroxybutyraldehyde reductase; an acetoacetyl-CoA reductase (ketone reducing); a 3-hydroxybutyryl-CoA reductase (aldehyde forming); a 3-hydroxybutyryl-CoA reductase (alcohol forming); an acetoacetate reductase; a 3-hydroxybutyryl-CoA transferase, hydrolase, or synthetase; a 3-hydroxybutyrate reductase; and a 3-hydroxybutyrate dehydrogenase.

In another aspect, provided herein is a method for producing 1,3-BDO, comprising culturing any one of the non-naturally occurring eukaryotic organisms comprising a 1,3-BDO pathway provided herein under conditions and for a sufficient period of time to produce 1,3-BDO. In certain embodiments, the eukaryotic organism is cultured in a substantially anaerobic culture medium. In other embodiments, the eukaryotic organism is a Crabtree positive organism.

In another aspect, provided herein is a method for selecting an exogenous 1,3-BDO pathway enzyme to be introduced into a non-naturally occurring eukaryotic organism, wherein the exogenous 1,3-BDO pathway enzyme is expressed in a sufficient amount in the organism to produce 1,3-BDO, said method comprising (i.) measuring the activity of at least one 1,3-BDO pathway enzyme that uses NADH as a cofactor; (ii.) measuring the activity of at least 1,3-BDO pathway enzyme that uses NADPH as a cofactor; and (iii.) introducing into the organism at least one 1,3-BDO pathway enzyme that has a greater preference for NADH than NADPH as a cofactor as determined in steps 1 and 2.

### 3. BRIEF DESCRIPTIONS OF THE DRAWINGS

FIG. 1 shows an exemplary pathway for the production of acetyl-CoA in the cytosol of a eukaryotic organism.

FIG. 2 shows pathways for the production of cytosolic acetyl-CoA from mitochondrial acetyl-CoA using citrate and oxaloacetate transporters. Enzymes are: A) citrate synthase; B) citrate transporter; C) citrate/oxaloacetate transporter; D) ATP citrate lyase; E) citrate lyase; F) acetyl-CoA synthetase or transferase, or acetate kinase and phosphotransacetylase; G) oxaloacetate transporter; K) acetate kinase; and L) phosphotransacetylase.

FIG. 3 shows pathways for the production of cytosolic acetyl-CoA from mitochondrial acetyl-CoA using citrate and malate transporters. Enzymes are A) citrate synthase; B) citrate transporter; C) citrate/malate transporter; D) ATP citrate lyase; E) citrate lyase; F) acetyl-CoA synthetase or transferase, or acetate kinase and phosphotransacetylase; H) cytosolic malate dehydrogenase; I) malate transporter; J) mitochondrial malate dehydrogenase; K) acetate kinase; and L) phosphotransacetylase.

FIG. 4 shows pathways for the biosynthesis of 1,3-BDO from acetyl-CoA. The enzymatic transformations shown are carried out by the following enzymes: A) Acetoacetyl-CoA thiolase, B) Acetoacetyl-CoA reductase (CoA-dependent, alcohol forming), C) 3-oxobutyraldehyde reductase (aldehyde reducing), D) 4-hydroxy-2-butanone reductase, E) Acetoacetyl-CoA reductase (CoA-dependent, aldehyde forming), F) 3-oxobutyraldehyde reductase (ketone reducing), G) 3-hydroxybutyraldehyde reductase, H) Acetoacetyl-CoA reductase (ketone reducing), I) 3-hydroxybutyryl-CoA reductase (aldehyde forming), J) 3-hydroxybutyryl-CoA reductase (alcohol forming), K) an acetoacetyl-CoA transferase, an acetoacetyl-CoA hydrolase, an acetoacetyl-CoA synthetase, or a phosphotransacetoacetylase and acetoacetate kinase, L) acetoacetate reductase, M) 3-hydroxybutyryl-CoA transferase, hydrolase, or synthetase, N) 3-hydroxybutyrate reductase, and O) 3-hydroxybutyrate dehydrogenase. An alternative to the conversion of acetyl-CoA to acetoacetyl-CoA by acetoacetyl-CoA thiolase (step A) in the 1,3-BDO pathways depicted in FIG. 4 involves the conversion of acetyl-CoA to malonyl-CoA by acetyl-CoA carboxylase, and the conversion of an acetyl-CoA and the malonyl-CoA to acetoacetyl-CoA by acetoacetyl-CoA synthetase (not shown; refer to FIG. 7, steps E and F, or FIG. 9).

FIG. 5 shows pathways for the production of cytosolic acetyl-CoA from cytosolic pyruvate. Enzymes are A) pyruvate oxidase (acetate-forming), B) acetyl-CoA synthetase, ligase or transferase, C) acetate kinase, D) phosphotransacetylase, E) pyruvate decarboxylase, F) acetaldehyde dehydrogenase, G) pyruvate oxidase (acetyl-phosphate forming), H) pyruvate dehydrogenase, pyruvate:ferredoxin oxidoreductase or pyruvate formate lyase, I) acetaldehyde dehydrogenase (acylating), and J) threonine aldolase.

FIG. 6 shows pathways for the production of cytosolic acetyl-CoA from mitochondrial or peroxisomal acetyl-CoA. Enzymes are A) mitochondrial acetylcarnitine transferase, B) peroxisomal acetylcarnitine transferase, C) cytosolic acetylcarnitine transferase, D) mitochondrial acetylcarnitine translocase, E) peroxisomal acetylcarnitine translocase.

FIG. 7 depicts an exemplary 1,3-BDO pathway. A) acetoacetyl-CoA thiolase, B) acetoacetyl-CoA reductase, C) 3-hydroxybutyryl-CoA reductase (aldehyde forming), D) 3-hydroxybutyraldehyde reductase, E) acetyl-CoA carboxylase, F) acetoacetyl-CoA synthase. G3P is glycerol-3-phosphate. In this pathway, two equivalents of acetyl-CoA are converted to acetoacetyl-CoA by an acetoacetyl-CoA thiolase. Alternatively, acetyl-CoA is converted to malonyl-CoA by acetyl-CoA carboxylase, and acetoacetyl-CoA is synthesized from acetyl-CoA and malonyl-CoA by acetoacetyl-CoA synthetase. Acetoacetyl-CoA is then reduced to 3-hydroxybutyryl-CoA by 3-hydroxybutyryl-CoA reductase. The 3-hydroxybutyryl-CoA intermediate is further reduced to 3-hydroxybutyraldehyde, and further to 1,3-BDO by 3-hydroxybutyryl-CoA reductase and 3-hydroxybutyraldehyde reductase. The organism can optionally be further engineered to delete one or more of the exemplary byproduct pathways (“X”).

FIG. 8 depicts exemplary combined mitochondrial/cytosolic 1,3-BDO pathways. Pathway enzymes include: A) acetoacetyl-CoA thiolase, B) acetoacetyl-CoA reductase, C) acetoacetyl-CoA hydrolase, transferase or synthetase, D) 3-hydroxybutyryl-CoA hydrolase, transferase or synthetase, E) 3-hydroxybutyrate dehydrogenase, F) acetoacetate transporter, G) 3-hydroxybutyrate transporter, H) 3-hydroxybutyryl-CoA transferase or synthetase, I) acetoacetyl-CoA transferase or synthetase, J) acetyl-CoA carboxylase, and K). acetoacetyl-CoA synthase.

FIG. 9 depicts an exemplary pathway for the conversion of acetyl CoA and malonyl-CoA to acetoacetyl-CoA by acetoacetyl-CoA synthase.

FIG. 10 depicts exemplary pathways from phosphoenolpyruvate (PEP) and pyruvate to acetyl-CoA and acetoacetyl-CoA. A) PEP carboxylase or PEP carboxykinase, B) oxaloacetate decarboxylase, C) malonate semialdehyde dehydrogenase (acetylating), D) acetyl-CoA carboxylase or malonyl-CoA decarboxylase, E) acetoacetyl-CoA synthase, F) oxaloacetate dehydrogenase or oxaloacetate oxidoreductase, G) malonyl-CoA reductase, H) pyruvate carboxylase, I) acetoacetyl-CoA thiolase, J) malonate semialdehyde dehydrogenase, K) malonyl-CoA synthetase or transferase, L) malic enzyme, M) malate dehydrogenase or oxidoreductase, N) pyruvate kinase or PEP phosphatase.

### 4. DETAILED DESCRIPTION

Provided herein are non-naturally occurring eukaryotic organisms and methods thereof to produce and increase the availability of cytosolic acetyl-CoA in the eukaryotic organisms thereof. Also provided herein are non-naturally occurring eukaryotic organisms and methods thereof to produce commodity chemicals, such as 1.3-BDO, and/or other compounds of interest.

### 4.1 Definitions

As used herein, the term “non-naturally occurring” when used in reference to a eukaryotic organism provided herein is intended to mean that the eukaryotic organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the eukaryotic organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within an acetyl-CoA pathway.

A metabolic modification refers to a biochemical reaction that is altered from its naturally occurring state. Therefore, non-naturally occurring eukaryotic organisms can have genetic modifications to nucleic acids encoding metabolic polypeptides, or functional fragments thereof. Exemplary metabolic modifications are disclosed herein.

As used herein, the term “isolated” when used in reference to a eukaryotic organism is intended to mean an organism that is substantially free of at least one component as the referenced eukaryotic organism is found in nature. The term includes a eukaryotic organism that is removed from some or all components as it is found in its natural environment. The term also includes a eukaryotic organism that is removed from some or all components as the eukaryotic organism is found in non-naturally occurring environments. Therefore, an isolated eukaryotic organism is partly or completely separated from other substances as it is found in nature or as it is grown, stored or subsisted in non-naturally occurring environments. Specific examples of isolated eukaryotic organisms include partially pure microbes, substantially pure microbes and microbes cultured in a medium that is non-naturally occurring.

As used herein, the terms “eukaryotic,” “eukaryotic organism,” or “eukaryote” are intended to refer to any single celled or multi-cellular organism of the taxon Eukarya or Eukaryota. In particular, the terms encompass those organisms whose cells comprise a mitochondrion. The term also includes cell cultures of any species that can be cultured for the increased levels of cytosolic acetyl-CoA. In certain embodiments of the compositions and methods provided herein, the eukaryotic organism is a yeast.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host eukaryotic organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the eukaryotic organism. When used in reference to a biosynthetic activity, the term refers to an activity that is introduced into the host reference organism. The source can be, for example, a homologous or heterologous encoding nucleic acid that expresses the referenced activity following introduction into the host eukaryotic organism. Therefore, the term “endogenous” refers to a referenced molecule or activity that is present in the host. Similarly, the term when used in reference to expression of an encoding nucleic acid refers to expression of an encoding nucleic acid contained within the eukaryotic organism. The term “heterologous” refers to a molecule or activity derived from a source other than the referenced species whereas “homologous” refers to a molecule or activity derived from the host eukaryotic organism. Accordingly, exogenous expression of an encoding nucleic acid provided herein can utilize either or both a heterologous or homologous encoding nucleic acid.

It is understood that when more than one exogenous nucleic acid is included in a eukaryotic organism that the more than one exogenous nucleic acids refers to the referenced encoding nucleic acid or biochemical activity, as discussed above. It is further understood, as disclosed herein, that such more than one exogenous nucleic acids can be introduced into the host eukaryotic organism on separate nucleic acid molecules, on polycistronic nucleic acid molecules, or a combination thereof, and still be considered as more than one exogenous nucleic acid. For example, as disclosed herein a eukaryotic organism can be engineered to express two or more exogenous nucleic acids encoding a desired pathway enzyme or protein. In the case where two exogenous nucleic acids encoding a desired activity are introduced into a host eukaryotic organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biochemical activities, not the number of separate nucleic acids introduced into the host organism.

The non-naturally occurring eukaryotic organisms provided herein can contain stable genetic alterations, which refers to eukaryotic organisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. With respect to the metabolic pathways described herein, those skilled in the art will understand that the orthologous gene harboring the metabolic activity to be introduced or disrupted is to be chosen for construction of the non-naturally occurring eukaryotic organism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other, suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring eukaryotic organisms provided herein having cytosolic acetyl-CoA biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced eukaryotic organism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

### 4.2 Eukaryotic Organisms that Utilize Cytosolic Acetyl-CoA

In a first aspect, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase. Such organisms would advantageously allow for the production of cytosolic acetyl-CoA, which can then be used by the organism to produce compounds of interest, for example, 1,3-BDO, using a cytosolic production pathway.

In one embodiment, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a mitochondrion of said organism to the cytosol of said organism. In another embodiment, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a peroxisome of said organism to the cytosol of said organism. In one embodiment, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to produce acetyl-CoA in the cytoplasm of said organism. In another embodiment, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to increase acetyl-CoA in the cytosol of said organism. In other embodiments, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a mitochondrion and produce acetyl-CoA in the cytoplasm of said organism. In another embodiment, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a peroxisome of said organism to the cytosol of said organism and produce acetyl-CoA in the cytoplasm of said organism. In other embodiments, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a mitochondrion and increase acetyl-CoA in the cytoplasm of said organism. In another embodiment, provided herein is a non-naturally occurring eukaryotic organism comprising an acetyl-CoA pathway, wherein said organism comprises at least one exogenous nucleic acid encoding an acetyl-CoA pathway enzyme expressed in a sufficient amount to increase acetyl-CoA from a peroxisome and increase acetyl-CoA in the cytosol of said organism.

In a second aspect, provided herein is a method for transporting acetyl-CoA from a mitochondrion and/or peroxisome to a cytosol of a non-naturally occurring eukaryotic organism, comprising culturing a non-naturally occurring eukaryotic organism comprising an acetyl-CoA pathway under conditions and for a sufficient period of time to transport the acetyl-CoA from a mitochondrion and/or peroxisome to a cytosol of the non-naturally occurring eukaryotic organism. In one embodiment, provided herein is a method for transporting acetyl-CoA from a mitochondrion to a cytosol of a non-naturally occurring eukaryotic organism, comprising culturing a non-naturally occurring eukaryotic organism comprising an acetyl-CoA pathway under conditions and for a sufficient period of time to transport the acetyl-CoA from a mitochondrion to a cytosol of the non-naturally occurring eukaryotic organism. In another embodiment, provided herein is a method for transporting acetyl-CoA from a peroxisome to a cytosol of a non-naturally occurring eukaryotic organism, comprising culturing a non-naturally occurring eukaryotic organism comprising an acetyl-CoA pathway under conditions and for a sufficient period of time to transport the acetyl-CoA from a peroxisome to a cytosol of the non-naturally occurring eukaryotic organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

In another embodiment, provided herein is a method for transporting acetyl-CoA from a mitochondrion to a cytosol of a non-naturally occurring eukaryotic organism, comprising culturing a non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a mitochondrion of said organism to the cytosol of said organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a cytosolic acetylcarnitine transferase; and a mitochondrial acetylcarnitine translocase.

In some embodiments, provided herein is a method for transporting acetyl-CoA from a peroxisome to a cytosol of a non-naturally occurring eukaryotic organism, comprising culturing said non-naturally occurring eukaryotic organism comprising an acetyl-CoA pathway, wherein said organism comprises at least one exogenous nucleic acid encoding an acetyl-CoA pathway enzyme expressed in a sufficient amount to transport acetyl-CoA from a peroxisome of said organism to the cytosol of said organism. In certain embodiments, the acetyl-CoA pathway comprises one or more enzymes selected from the group consisting of a peroxisomal acetylcarnitine transferase and a peroxisomal acetylcarnitine translocase.

In a third aspect, provided herein is a method for producing cytosolic acetyl-CoA, comprising culturing a non-naturally occurring eukaryotic organism comprising an acetyl-CoA pathway under conditions and for a sufficient period of time to produce cytosolic acetyl-CoA. In one embodiment, said organism comprises at least one exogenous nucleic acid encoding an acetyl-CoA pathway enzyme expressed in a sufficient amount to produce cytosolic acetyl-CoA in said organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

In a fourth aspect, provided herein is a method for increasing acetyl-CoA in the cytosol of a non-naturally occurring eukaryotic organism, comprising culturing a non-naturally occurring eukaryotic organism comprising an acetyl-CoA pathway under conditions and for a sufficient period of time to increase the acetyl-CoA in the cytosol of the organism. In some embodiments, the organism comprises at least one exogenous nucleic acid encoding an acetyl-CoA pathway enzyme expressed in a sufficient amount to increase acetyl-CoA in the cytosol of said non-naturally occurring eukaryotic organism. In certain embodiments, the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase.

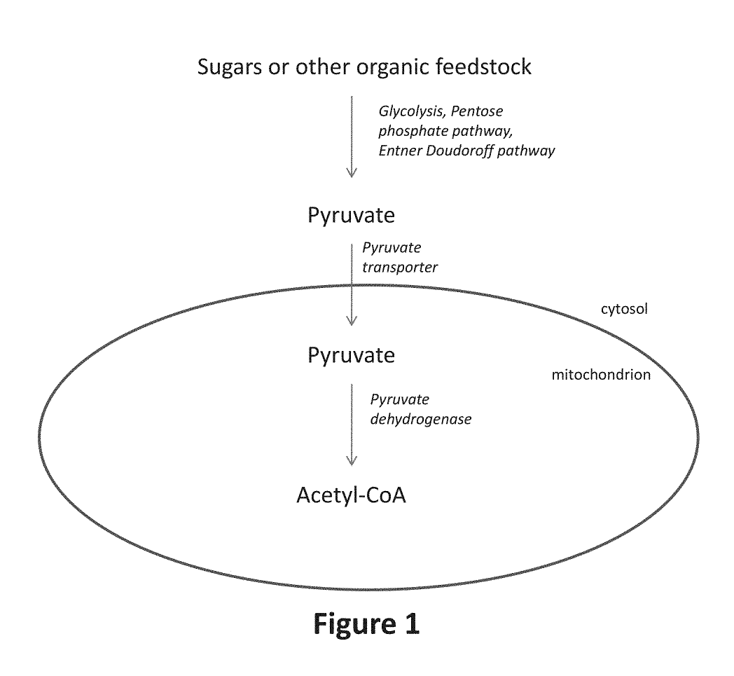

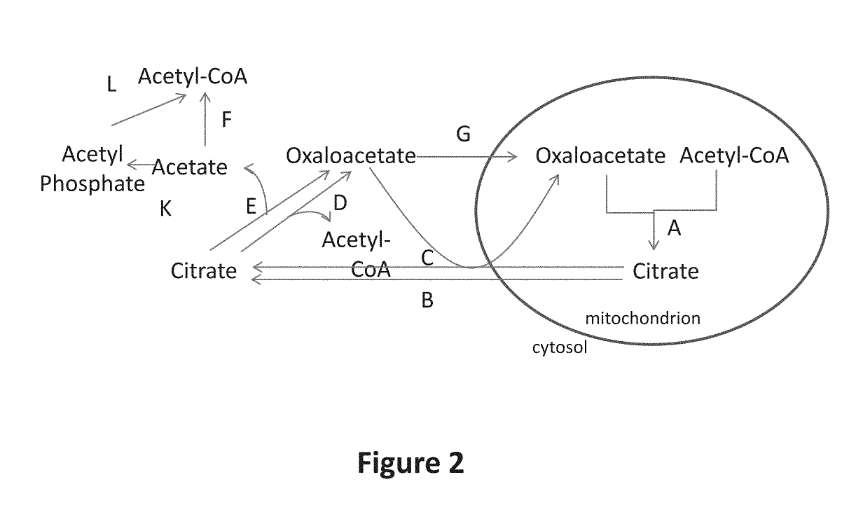

In many eukaryotic organisms, acetyl-CoA is mainly synthesized by pyruvate dehydrogenase in the mitochondrion (FIG. 1). A mechanism for exporting acetyl-CoA from the mitochondrion to the cytosol can enable deployment of, for example, a cytosolic 1,3-BDO production pathway that originates from acetyl-CoA. Exemplary mechanisms for exporting acetyl-CoA include those depicted in FIGS. 2, 3 and 8, which can involve forming citrate from acetyl-CoA and oxaloacetate in the mitochondrion, exporting the citrate from the mitochondrion to the cytosol, and converting the citrate to oxaloacetate and either acetate or acetyl-CoA. In certain embodiments, provided herein are methods for engineering a eukaryotic organism to increase its availability of cytosolic acetyl-CoA by introducing enzymes capable of carrying out the transformations depicted in any one of FIGS. 2, 3 and 8. Exemplary enzymes capable of carrying out the required transformations are also disclosed herein.

Acetyl-CoA localized in cellular organelles, such as peroxisomes and mitochondria, can also be exported into the cytosol by the aid of a carrier protein, such as carnitine or other acetyl carriers. In some embodiments of the composition and methods provided herein, the translocation of acetyl units across organellar membranes, such as a mitochondrial or peroxisomal membrane, utilizes a carrier molecule or acyl-CoA transporter. An exemplary acetyl carrier molecule is carnitine. Other exemplary acetyl carrier molecules or transporters include glutamate, pyruvate, imidazole and glucosamine.

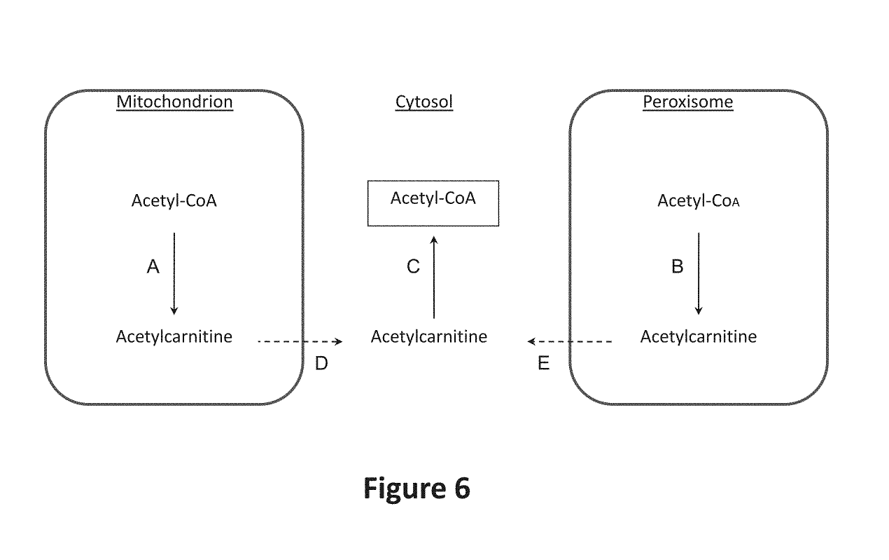

A mechanism for exporting acetyl-CoA localized in cellular organelles such as peroxisomes and mitochondria to the cytosol using a carrier protein could enable deployment of, for example, a cytosolic 1,3-BDO production pathway that originates from acetyl-CoA. Exemplary acetylcarnitine translocation pathways are depicted in FIG. 6. In one pathway, mitochondrial acetyl-CoA is converted to acetylcarnitine by a mitochondrial acetylcarnitine transferase. Mitochondrial acetylcarnitine can then be translocated across the mitochondrial membrane into the cytosol by a mitochondrial acetylcarnitine translocase, and then converted to cytosolic acetyl-CoA by a cytosolic acetylcarnitine transferase. In another pathway, peroxisomal acetyl-CoA is converted to acetylcarnitine by a peroxisomal acetylcarnitine transferase. Peroxisomal acetylcarnitine can then be translocated across the peroxisomal membrane into the cytosol by a peroxisomal acetylcarnitine translocase, and then converted to cytosolic acetyl-CoA by a cytosolic acetylcarnitine transferase.

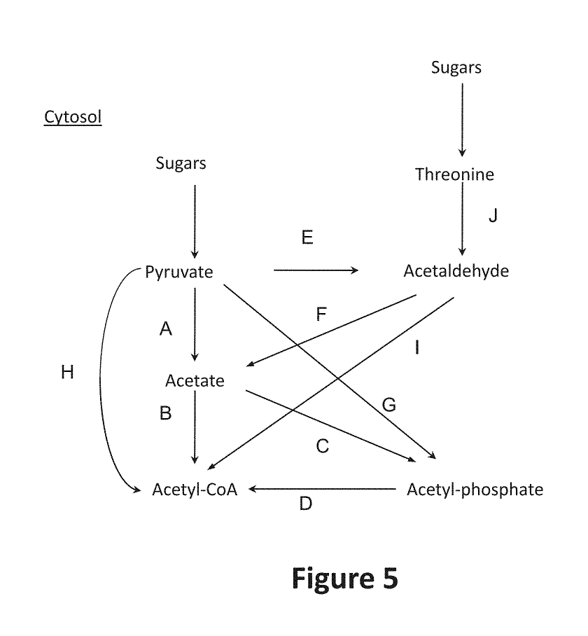

Pathways for the conversion of cytosolic pyruvate and threonine to cytosolic acetyl-CoA could enable deployment of, for example, a cytosolic 1,3-BDO production pathway that originates from acetyl-CoA. In addition to several known pathways, FIG. 5 depicts four novel exemplary pathways for converting cytosolic pyruvate to cytosolic acetyl-CoA. In one pathway, pyruvate is converted to acetate by pyruvate oxidase (acetate forming). Acetate is subsequently converted to acetyl-CoA either directly, by acetyl-CoA synthetase, ligase or transferase, or indirectly via an acetyl-phosphate intermediate. In an alternate route, pyruvate is decarboxylated to acetaldehyde by pyruvate decarboxylase. An acetaldehyde dehydrogenase oxidizes acetaldehyde to acetate. Acetate is then converted to acetyl-CoA by acetate kinase and phosphotransacetylase. In yet another route, pyruvate is oxidized to acetylphosphate by pyruvate oxidase (acetyl-phosphate forming). Phosphotransacetylase then converts acetylphopshate to acetyl-CoA. Exemplary enzymes capable of carrying out the required transformations are also disclosed herein.

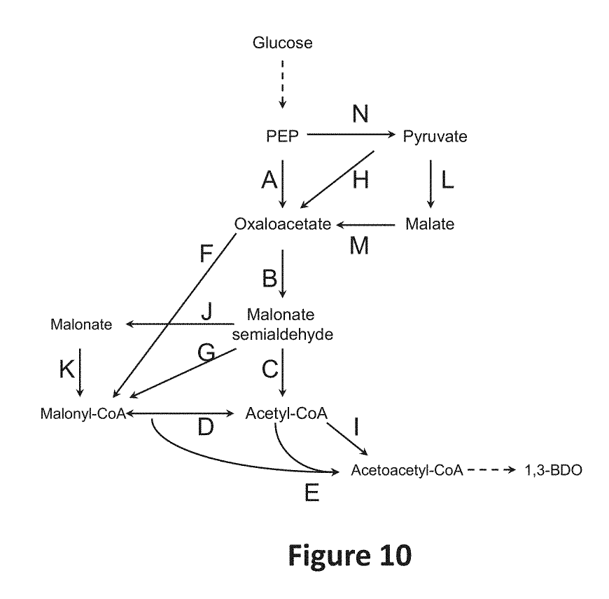

Pathways for the conversion of cytosolic phosphoenolpyruvate (PEP) and pyruvate to cytosolic acetyl-CoA could also enable deployment of, for example, a cytosolic 1,3-BDO production pathway from acetyl-CoA. FIG. 10 depicts twelve exemplary pathways for converting cytosolic PEP and pyruvate to cytosolic acetyl-CoA. In one pathway, PEP carboxylase or PEP carboxykinase converts PEP to oxaloacetate (step A); oxaloacetate decarboxylase converts the oxaloacetate to malonate (step B); and malonate semialdehyde dehydrogenase (acetylating) converts the malonate semialdehyde to acetyl-CoA (step C). In another pathway pyruvate kinase or PEP phosphatase converts PEP to pyruvate (step N); pyruvate carboxylase converts the pyruvate to (step H); oxaloacetate decarboxylase converts the oxaloacetate to malonate (step B); and malonate semialdehyde dehydrogenase (acetylating) converts the malonate semialdehyde to acetyl-CoA (step C). In another pathway pyruvate kinase or PEP phosphatase converts PEP to pyruvate (step N); malic enzyme converts the pyruvate to malate (step L); malate dehydrogenase or oxidoreductase converts the malate to oxaloacetate (step M); oxaloacetate decarboxylase converts the oxaloacetate to malonate (step B); and malonate semialdehyde dehydrogenase (acetylating) converts the malonate semialdehyde to acetyl-CoA (step C). In another pathway, PEP carboxylase or PEP carboxykinase converts PEP to oxaloacetate (step A); oxaloacetate decarboxylase converts the oxaloacetate to malonate semialdehyde (step B); malonyl-CoA reductase converts the malonate semialdehyde to malonyl-CoA (step G); and malonyl-CoA decarboxylase converts the malonyl-CoA to acetyl-CoA (step (D). In another pathway, pyruvate kinase or PEP phosphatase converts PEP to pyruvate (step N); pyruvate carboxylase converts the pyruvate to oxaloacetate (step H); (oxaloacetate decarboxylase converts the oxaloacetate to malonate semialdehyde (step B); malonyl-CoA reductase converts the malonate semialdehyde to malonyl-CoA (step G); and malonyl-CoA decarboxylase converts the malonyl-CoA to acetyl-CoA (step (D). In another pathway, pyruvate kinase or PEP phosphatase converts PEP to pyruvate (step N); malic enzyme converts the pyruvate to malate (step L); malate dehydrogenase or oxidoreductase converts the malate to oxaloacetate (step M); oxaloacetate decarboxylase converts the oxaloacetate to malonate semialdehyde (step B); malonyl-CoA reductase converts the malonate semialdehyde to malonyl-CoA (step G); and malonyl-CoA decarboxylase converts the malonyl-CoA to acetyl-CoA (step (D). In another pathway, PEP carboxylase or PEP carboxykinase converts PEP to oxaloacetate (step A); oxaloacetate decarboxylase converts the oxaloacetate to malonate semialdehyde (step B); malonate semialdehyde dehydrogenase converts the malonate semialdehyde to malonate (step J); malonyl-CoA synthetase or transferase converts the malonate to malonyl-CoA (step K); and malonyl-CoA decarboxylase converts the malonyl-CoA to acetyl-CoA (step D). In another pathway, pyruvate kinase or PEP phosphatase converts PEP to pyruvate (step N); pyruvate carboxylase converts the pyruvate to oxaloacetate (step H); oxaloacetate decarboxylase converts the oxaloacetate to malonate semialdehyde (step B); malonate semialdehyde dehydrogenase converts the malonate semialdehyde to malonate (step J); malonyl-CoA synthetase or transferase converts the malonate to malonyl-CoA (step K); and malonyl-CoA decarboxylase converts the malonyl-CoA to acetyl-CoA (step D). In another pathway, pyruvate kinase or PEP phosphatase converts PEP to pyruvate (step N); malic enzyme converts the pyruvate to malate (step L); malate dehydrogenase or oxidoreductase converts the malate to oxaloacetate (step M); oxaloacetate decarboxylase converts the oxaloacetate to malonate semialdehyde (step B); malonate semialdehyde dehydrogenase converts the malonate semialdehyde to malonate (step J); malonyl-CoA synthetase or transferase converts the malonate to malonyl-CoA (step K); and malonyl-CoA decarboxylase converts the malonyl-CoA to acetyl-CoA (step D). In another pathway, PEP carboxylase or PEP carboxykinase converts PEP to oxaloacetate (step A); oxaloacetate dehydrogenase or oxaloacetate oxidoreductase converts the oxaloacetate to malonyl-CoA (step F); and malonyl-CoA decarboxylase converts the malonyl-CoA to acetyl-CoA (step D). In another pathway, pyruvate kinase or PEP phosphatase converts PEP to pyruvate (step N); pyruvate carboxylase converts the pyruvate to oxaloacetate (step H); oxaloacetate dehydrogenase or oxaloacetate oxidoreductase converts the oxaloacetate to malonyl-CoA (step F); and malonyl-CoA decarboxylase converts the malonyl-CoA to acetyl-CoA (step D). In another pathway, pyruvate kinase or PEP phosphatase converts PEP to pyruvate (step N); malic enzyme converts the pyruvate to malate (step L); malate dehydrogenase or oxidoreductase converts the malate to oxaloacetate (step M); oxaloacetate dehydrogenase or oxaloacetate oxidoreductase converts the oxaloacetate to malonyl-CoA (step F); and malonyl-CoA decarboxylase converts the malonyl-CoA to acetyl-CoA (step D).

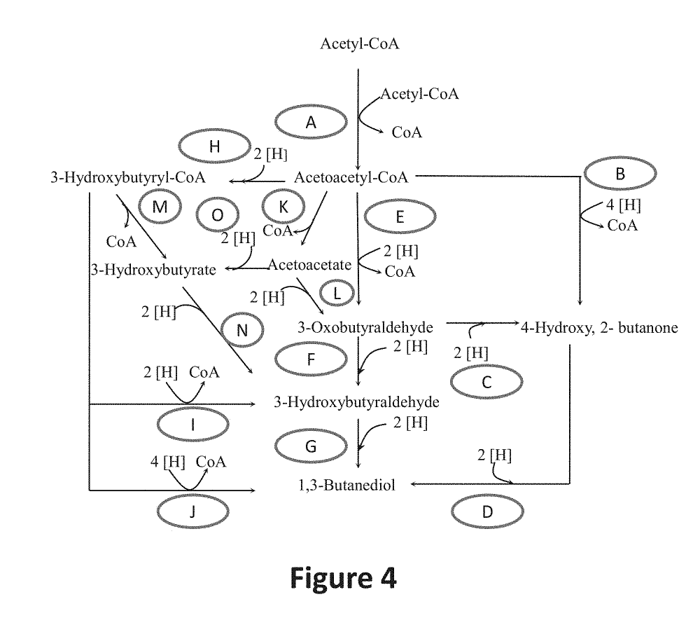

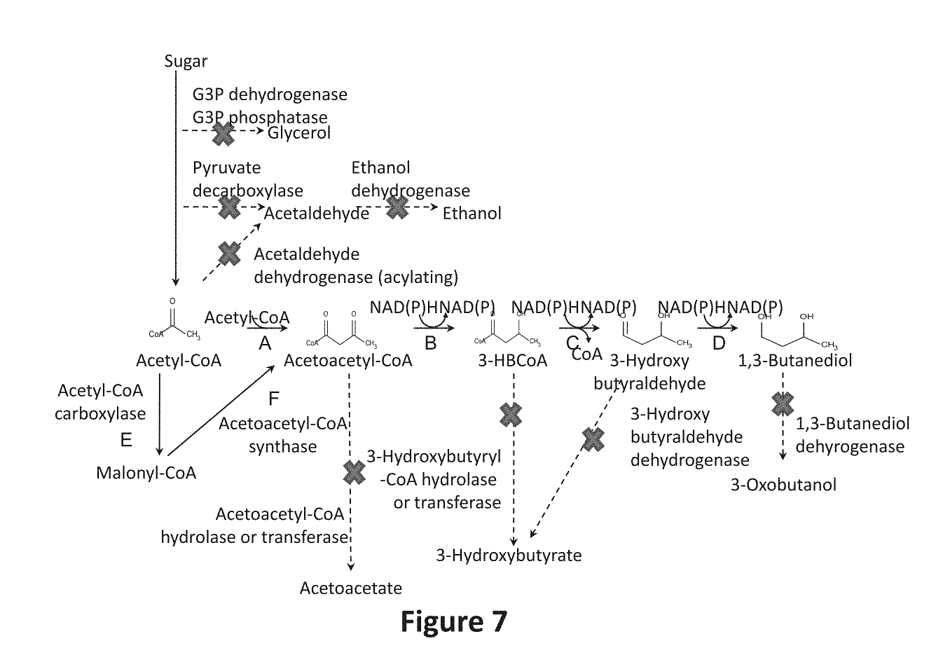

In certain embodiments, any pathway (e.g., an acetyl-CoA and/or 1,3-BDO pathway) provided herein further comprises the conversion of acetyl-CoA to acetoacetyl-CoA, e.g., as exemplified in FIG. 4, 7 or 10. In some embodiments, the pathway comprises acetoacetyl-CoA thiolase, which converts acetyl-CoA to acetoacetyl-CoA (FIG. 4, step A; FIG. 7, step A; FIG. 10, step I). In another embodiment, the pathway comprises acetyl-CoA carboxylase, which converts acetyl-CoA to malonyl-CoA (FIG. 7, step E; FIG. 10, step D); acetoacetyl-CoA synthase, which converts malonyl-CoA and acetyl-CoA to acetoacetyl-CoA (FIG. 7, step F; FIG. 10, step E).

In certain embodiments, non-naturally occurring eukaryotic organisms provided herein express genes encoding an acetyl-CoA pathway for the production of cytosolic acetyl-CoA. In some embodiments, successful engineering of an acetyl CoA pathway entails identifying an appropriate set of enzymes with sufficient activity and specificity, cloning their corresponding genes into a production host, optimizing culture conditions for the conversion of mitochondrial acetyl-CoA to cytosolic acetyl-CoA, and assaying for the production or increase in levels of cytosolic acetyl-CoA following exportation.

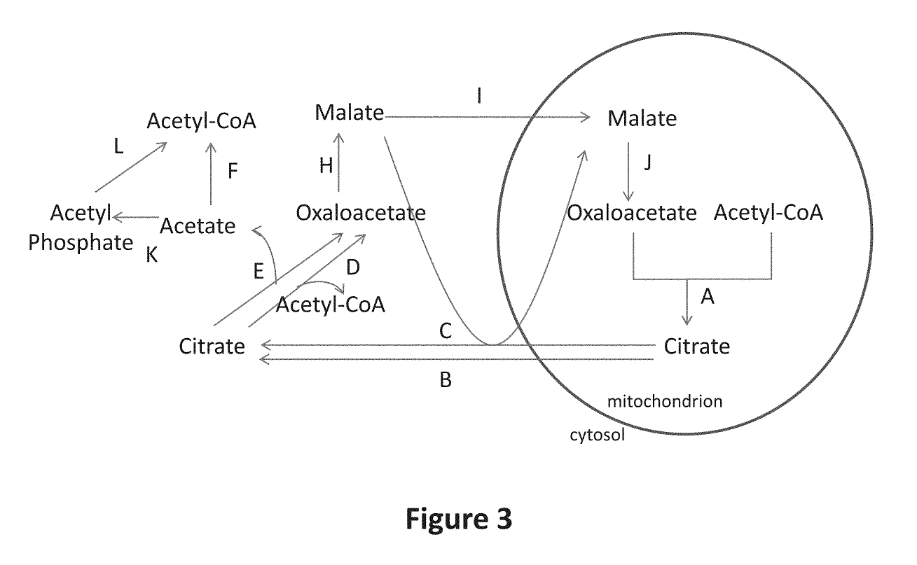

The production of cytosolic acetyl-CoA from mitochondrial or peroxisomal acetyl-CoA can be accomplished by a number of pathways, for example, in about two to five enzymatic steps. In one exemplary pathway, mitochondrial acetyl-CoA and oxaloacetate are combined into citrate by a citrate synthase and exported out of the mitochondrion by a citrate or citrate/oxaloacetate transporter (see, e.g., FIG. 2). Enzymatic conversion of the citrate in the cytosol results in cytosolic acetyl-CoA and oxaloacetate. The cytosolic oxaloacetate can then optionally be transported back into the mitochondrion by an oxaloacetate transporter and/or a citrate/oxaloacetate transporter. In another exemplary pathway, the cytosolic oxaloacetate is first enzymatically converted into malate in the cytosol and then optionally transferred into the mitochondrion by a malate transporter and/or a malate/citrate transporter (see, e.g., FIG. 3). Mitochondrial malate can then be converted into oxaloacetate with a mitochondrial malate dehydrogenase. In another exemplary pathway, mitochondrial acetyl-CoA is converted to acetylcarnitine by a mitochondrial acetylcarnitine transferase. Mitochondrial acetylcarnitine can then be translocated across the mitochondrial membrane into the cytosol by a mitochondrial acetylcarnitine translocase, and then converted to cytosolic acetyl-CoA by a cytosolic acetylcarnitine transferase. In yet another exemplary pathway, peroxisomal acetyl-CoA is converted to acetylcarnitine by a peroxisomal acetylcarnitine transferase. Peroxisomal acetylcarnitine can then be translocated across the peroxisomal membrane into the cytosol by a peroxisomal acetylcarnitine translocase, and then converted to cytosolic acetyl-CoA by a cytosolic acetylcarnitine transferase.

The production of cytosolic acetyl-CoA from cytosolic pyruvate can be accomplished by a number of pathways, for example, in about two to four enzymatic steps, and exemplary pathways are depicted in FIG. 5. In one pathway, pyruvate is converted to acetate by pyruvate oxidase (acetate forming). Acetate is subsequently converted to acetyl-CoA either directly, by acetyl-CoA synthetase, ligase or transferase, or indirectly via an acetyl-phosphate intermediate. In an alternate pathway, pyruvate is decarboxylated to acetaldehyde by pyruvate decarboxylase. An acetaldehyde dehydrogenase oxidizes acetaldehyde to acetate. Acetate is then converted to acetyl-CoA by acetate kinase and phosphotransacetylase. In yet another route, pyruvate is oxidized to acetylphosphate by pyruvate oxidase (acetyl-phosphate forming). Phosphotransacetylase then converts acetylphopshate to acetyl-CoA. Other exemplary pathways for the conversion of cytosolic pyruvate to acetyl-CoA are depicted in FIG. 10.

As discussed above, methods for the conversion of mitochondrial acetyl-CoA to cytosolic acetyl-CoA and increasing the levels of cytosolic acetyl-CoA within a eukaryotic organism would allow for the cytosolic production of several compounds of industrial interest, including 1,3-BDO, via a cytosolic production pathway that uses cytosolic acetyl-CoA as a starting material. In certain embodiments, the organisms provided herein further comprise a biosynthetic pathway for the production of a compound using cytosolic acetyl-CoA as a starting material. In certain embodiments, the compound is 1,3-BDO.

Microorganisms can be engineered to produce several compounds of industrial interest using acetyl-CoA, including 1,3-BDO. Thus, provided herein are non-naturally occurring eukaryotic organisms that can be engineered to produce the commodity chemicals, such as 1,3-butanediol. 1,3-BDO is a four carbon diol traditionally produced from acetylene via its hydration. The resulting acetaldehyde is then converted to 3-hydroxybutyraldehdye which is subsequently reduced to form 1,3-BDO. In more recent years, acetylene has been replaced by the less expensive ethylene as a source of acetaldehyde. 1,3-BDO is commonly used as an organic solvent for food flavoring agents. It is also used as a co-monomer for polyurethane and polyester resins and is widely employed as a hypoglycaemic agent. Optically active 1,3-BDO is a useful starting material for the synthesis of biologically active compounds and liquid crystals. A substantial commercial use of 1,3-BDO is subsequent dehydration to afford 1,3-butadiene (Ichikawa et al., J. of Molecular Catalysis A—Chemical, 256:106-112 (2006); Ichikawa et al., J. of Molecular Catalysis A—Chemical, 231:181-189 (2005)), a 25 billion lb/yr petrochemical used to manufacture synthetic rubbers (e.g., tires), latex, and resins. The reliance on petroleum based feedstocks for production of 1,3-BDO warrants the development of alternative routes to producing 1,3-BDO and butadiene using renewable feedstocks.

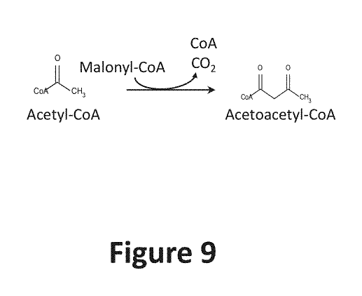

FIG. 4 depicts various exemplary pathways using acetyl-CoA as the starting material that can be used to produce 1,3-BDO from acetyl-CoA. In certain embodiments, the acetoacetyl-CoA depicted in the 1.3-BDO pathway(s) of FIG. 4 is synthesized from acetyl-CoA and malonyl-CoA by acetoacetyl-CoA synthetase, for example, as depicted in FIG. 7 (steps E and F) or FIG. 9, wherein acetyl-CoA is converted to malonyl-CoA by acetyl-CoA carboxylase, and acetoacetyl-CoA is synthesized from acetyl-CoA and malonyl-CoA by acetoacetyl-CoA synthetase.

1,3-BDO production in the cytosol relies on the native cell machinery to provide the necessary precursors. As shown in FIG. 4, acetyl CoA can provide a carbon precursor for the production of 1,3-BDO. Thus, acetyl-CoA pathways that are capable of producing high concentrations of cytosolic acetyl-CoA are desirable for enabling deployment of a cytosolic 1,3-BDO production pathway that originates from acetyl-CoA.

In certain acetyl-CoA pathways provided herein, acetyl-CoA is synthesized in the cytosol from a pyruvate or threonine precursor (FIG. 5). In other acetyl-CoA pathways provided herein, acetyl-CoA is synthesized in the cytosol from phosphoenolpyruvate (PEP) or pyruvate (FIG. 10). In other acetyl-CoA pathways provided herein, acetyl-CoA is synthesized in cellular compartments and transported to the cytosol, either directly or indirectly. One exemplary mechanism for transporting acetyl units from mitochondria or peroxisomes to the cytosol is the carnitine shuttle (FIG. 6). Another exemplary mechanism involves converting mitochondrial acetyl-CoA to a metabolic intermediate such as citrate or citramalate, transporting that intermediate to the cytosol, and then regenerating the acetyl-CoA (see FIGS. 2, 3 and 8). Exemplary acetyl-CoA pathways and corresponding enzymes are describe in further detail below and in Examples I-III.

Thus, in another aspect, provided herein is a non-naturally occurring eukaryotic organism, comprising (1) an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism, and (2) a 1,3-BDO pathway, comprising at least one exogenous nucleic acid encoding a 1,3-BDO pathway enzyme expressed in a sufficient amount to produce 1,3-BDO. In certain embodiments, (1) the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase; and/or (2) the 1,3-BDO pathway comprises one or more enzymes selected from the group consisting of an acetoacetyl-CoA thiolase; an acetyl-CoA carboxylase; an acetoacetyl-CoA synthase; an acetoacetyl-CoA reductase (CoA-dependent, alcohol forming); 3-oxobutyraldehyde reductase (aldehyde reducing); 4-hydroxy,2-butanone reductase; an acetoacetyl-CoA reductase (CoA-dependent, aldehyde forming); a 3-oxobutyraldehyde reductase (ketone reducing); 3-hydroxybutyraldehyde reductase; an acetoacetyl-CoA reductase (ketone reducing); a 3-hydroxybutyryl-CoA reductase (aldehyde forming); a 3-hydroxybutyryl-CoA reductase (alcohol forming); an acetoacetyl-CoA transferase, an acetoacetyl-CoA hydrolase, an acetoacetyl-CoA synthetase, or a phosphotransacetoacetylase and acetoacetate kinase; an acetoacetate reductase; a 3-hydroxybutyryl-CoA transferase, hydrolase, or synthetase; a 3-hydroxybutyrate reductase; and a 3-hydroxybutyrate dehydrogenase.

Any non-naturally occurring eukaryotic organism comprising an acetyl-CoA pathway and engineered to comprise an acetyl-CoA pathway enzyme, such as those provided herein, can be engineered to further comprise one or more 1,3-BDO pathway enzymes, such as those provided herein.

Also provided herein is a method for producing 1,3-BDO, comprising culturing any one of the organisms provided herein comprising a 1,3-BDO pathway under conditions and for a sufficient period of time to produce 1,3-BDO. Dehydration of 1,3-BDO produced by the organisms and methods described herein, provides an opportunity to produce renewable butadiene in small end-use facilities, obviating the need to transport this flammable and reactive chemical.

In a sixth aspect, provided herein is a method for producing 1,3-BDO, comprising culturing a non-naturally occurring eukaryotic organism under conditions and for a sufficient period of time to produce the 1,3-BDO, wherein the non-naturally occurring eukaryotic organism comprises (1) an acetyl-CoA pathway; and (2) a 1,3-BDO pathway. In certain embodiments, provided herein is a method for producing 1,3-BDO, comprising culturing a non-naturally occurring eukaryotic organism, comprising (1) an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism; and (2) a 1,3-BDO pathway, wherein said organism comprises at least one exogenous nucleic acid encoding a 1,3-BDO pathway enzyme expressed in a sufficient amount to produce 1,3-BDO. In certain embodiments, (1) the acetyl-CoA pathway comprises one or more enzymes selected from the group consisting of a citrate synthase; a citrate transporter; a citrate/oxaloacetate transporter; a citrate/malate transporter; an ATP citrate lyase; a citrate lyase; an acetyl-CoA synthetase; an oxaloacetate transporter; a cytosolic malate dehydrogenase; a malate transporter; a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; and a PEP phosphatase; and (2) the 1,3-BDO pathway comprises one or more enzymes selected from the group consisting of an acetoacetyl-CoA thiolase; an acetyl-CoA carboxylase; an acetoacetyl-CoA synthase; an acetoacetyl-CoA reductase (CoA-dependent, alcohol forming); 3-oxobutyraldehyde reductase (aldehyde reducing); 4-hydroxy,2-butanone reductase; an acetoacetyl-CoA reductase (CoA-dependent, aldehyde forming); a 3-oxobutyraldehyde reductase (ketone reducing); 3-hydroxybutyraldehyde reductase; an acetoacetyl-CoA reductase (ketone reducing); a 3-hydroxybutyryl-CoA reductase (aldehyde forming); a 3-hydroxybutyryl-CoA reductase (alcohol forming); an acetoacetyl-CoA transferase, an acetoacetyl-CoA hydrolase, an acetoacetyl-CoA synthetase, or a phosphotransacetoacetylase and acetoacetate kinase; an acetoacetate reductase; a 3-hydroxybutyryl-CoA transferase, hydrolase, or synthetase; and a 3-hydroxybutyrate reductase; and a 3-hydroxybutyrate dehydrogenase.

Any non-naturally occurring eukaryotic organism comprising an acetyl-CoA pathway and engineered to comprise an acetyl-CoA pathway enzyme, such as those provided herein, can be engineered to further comprise one or more 1,3-BDO pathway enzymes. In some embodiments, successful engineering of an acetyl CoA pathway in combination with a 1,3-BDO pathway entails identifying an appropriate set of enzymes with sufficient activity and specificity, cloning their corresponding genes into a production host, optimizing culture conditions for the production of cytosolic acetyl-CoA and the production of 1,3-BDO, and assaying for the production or increase in levels of 1,3-BDO product formation.

The conversion of acetyl-CoA to 1,3-BDO, for example, can be accomplished by a number of pathways in about three to six enzymatic steps as shown in FIG. 4. FIG. 4 outlines multiple routes for producing 1,3-BDO from acetyl-CoA. Each of these pathways from acetyl-CoA to 1,3-BDO utilizes three reducing equivalents and provides a theoretical yield of 1 mole of 1,3-BDO per mole of glucose consumed. Other carbon substrates such as syngas can also be used for the production of acetoacetyl-CoA. Gasification of glucose to form syngas will result in the maximum theoretical yield of 1.09 moles of 1,3-BDO per mole of glucose consumed, assuming that 6 moles of CO and 6 moles of H₂ are obtained from glucose

6CO+6H_{2→1.091}C₄H₁₀O₂+1.636 CO₂+0.545H₂

The methods provided herein are directed, in part, to methods for producing 1,3-BDO through culturing of these non-naturally occurring eukaryotic organisms. Dehydration of 1,3-BDO produced by the organisms and methods described herein, provides an opportunity to produce renewable butadiene in small end-use facilities obviating the need to transport this flammable and reactive chemical.

In some embodiments, the non-naturally occurring eukaryotic organism comprises an acetyl-CoA pathway, wherein said organism comprises at least one exogenous nucleic acid encoding at least one acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism. In one embodiment, the at least one acetyl-CoA pathway enzyme expressed in a sufficient amount to transport acetyl-CoA from a mitochondrion and/or peroxisome of said organism to the cytosol of the organism. In one embodiment, the at least one acetyl-CoA pathway enzyme expressed in a sufficient amount to produce cytosolic acetyl CoA in said organism. In another embodiment, the at least one acetyl-CoA pathway enzyme is expressed in a sufficient amount to increase acetyl-CoA in the cytosol of said organism.

In certain embodiments, the acetyl-CoA pathway comprises: 2A, 2B, 2C, 2D, 2E, 2F, 2G, 2K, 2L, 3H, 3I or 3J, or any combination of 2A, 2B, 2C, 2D, 2E, 2F, 2G, 2K, 2L, 3H, 3I and 3J, thereof; wherein 2A is a citrate synthase; 2B is a citrate transporter; 2C is a citrate/oxaloacetate transporter or a citrate/malate transporter; 2D is an ATP citrate lyase; 2E is a citrate lyase; 2F is an acetyl-CoA synthetase; 2G is an oxaloacetate transporter; 2K is an acetate kinase; 2L is a phosphotransacetylase; 3H is a cytosolic malate dehydrogenase; 3I is a malate transporter; and 3J is a mitochondrial malate dehydrogenase. In some embodiments, 2C is a citrate/oxaloacetate transporter. In other embodiments, 2C is a citrate/malate transporter.

In some embodiments, the acetyl-CoA pathway is an acetyl-CoA pathway depicted in FIG. 2. In other embodiments, the acetyl-CoA pathway is an acetyl-CoA pathway depicted in FIG. 3. In one embodiment, the acetyl-CoA pathway comprises 2A, 2B and 2D. In another embodiment, the acetyl-CoA pathway comprises 2A, 2C and 2D. In yet another embodiment, the acetyl-CoA pathway comprises 2A, 2B, 2C and 2D. In an embodiment, the acetyl-CoA pathway comprises 2A, 2B, 2E and 2F. In another embodiment, the acetyl-CoA pathway comprises 2A, 2C, 2E and 2F. In other embodiments, the acetyl-CoA pathway comprises 2A, 2B, 2C, 2E and 2F. In some embodiments, the acetyl CoA pathway comprises 2A, 2B, 2E, 2K and 2L. In another embodiment, the acetyl CoA pathway comprises 2A, 2C, 2E, 2K and 2L. In other embodiments, the acetyl CoA pathway comprises 2A, 2B, 2C, 2E, 2K and 2L. In some embodiments, the acetyl-CoA pathway further comprises 2G, 3H, 3I, 3J, or any combination thereof. In certain embodiments, the acetyl-CoA pathway further comprises 2G. In some embodiments, the acetyl-CoA pathway further comprises 3H. In other embodiments, the acetyl-CoA pathway further comprises 3I. In yet other embodiments, the acetyl-CoA pathway further comprises 3J. In some embodiments, the acetyl-CoA pathway further comprises 2G and 3H. In an embodiment, the acetyl-CoA pathway further comprises 2G and 3I. In one embodiment, the acetyl-CoA pathway further comprises 2G and 3J. In some embodiments, the acetyl-CoA pathway further comprises 3H and 3I. In other embodiments, the acetyl-CoA pathway further comprises 3H and 3J. In certain embodiments, the acetyl-CoA pathway further comprises 3I and 3J. In another embodiment, the acetyl-CoA pathway further comprises 2G, 3H and 3I. In yet another embodiment, the acetyl-CoA pathway further comprises 2G, 3H and 3J. In some embodiments, the acetyl-CoA pathway further comprises 2G, 3I and 3J. In other embodiments, the acetyl-CoA pathway further comprises 3H, 3I and 3J.

In one embodiment, the acetyl-CoA pathway comprises 2A. In another embodiment, the acetyl-CoA pathway comprises 2B. In an embodiment, the acetyl-CoA pathway comprises 2C. In another embodiment, the acetyl-CoA pathway comprises 2D. In one embodiment, the acetyl-CoA pathway comprises 2E. In yet another embodiment, the acetyl-CoA pathway comprises 2F. In some embodiments, the acetyl-CoA pathway comprises 2G. In some embodiments, the acetyl-CoA pathway comprises 2K. In another embodiment, the acetyl-coA pathway comprises 2L. In other embodiments, the acetyl-CoA pathway comprises 3H. In another embodiment, the acetyl-CoA pathway comprises 3I. In one embodiment, the acetyl-CoA pathway comprises 3J.

In some embodiments, the acetyl-CoA pathway comprises: 2A and 2B; 2A and 2C; 2A and 2D; 2A and 2E; 2A and 2F; 2A and 2G; 2A and 2K; 2A and 2L; 2A and 3H; 2A and 3I; 2A and 3J; 2B and 2C; 2B and 2D; 2B and 2E; 2B and 2F; 2B and 2G; 2B and 2K; 2B and 2L; 2B and 3H; 2B and 3I; 2B and 3J; 2C and 2D; 2C and 2E; 2C and 2F; 2C and 2G; 2C and 2K; 2C and 2L; 2C and 3H; 2C and 3I; 2C and 3J; 2D and 2E; 2D and 2F; 2D and 2G; 2D and 2E; 2D and 2F; 2D and 2G; 2D and 2K; 2D and 2L; 2D and 3H; 2D and 3I; 2D and 3J; 2E and 2F; 2E and 2G; 2E and 2K; 2E and 2L; 2E and 3H; 2E and 3I; 2E and 3J; 2F and 2G; 2F and 2K; 2F and 2L; 2F and 3H; 2F and 3I; 2F and 3J; 2G and 2K; 2G and 2L; 2G and 3H; 2G and 3I; 2G and 3J; 2K and 2L; 2K and 3H; 2K and 3I; 2K and 3J; 2L and 3H; 2L and 3I; 2L and 3J; 3H and 3I; 3H and 3J; or 3I and 3J. In some embodiments, the non-naturally occurring eukaryotic organism comprises two or more exogenous nucleic acids, wherein each of the two or more exogenous nucleic acids encodes a different acetyl-CoA pathway enzyme.

In other embodiments, the acetyl-CoA pathway comprises: 2A, 2B and 2C; 2A, 2B and 2D; 2A, 2B and 2E; 2A, 2B and 2F; 2A, 2B and 2G; 2A, 2B and 2K; 2A, 2B and 2L; 2A, 2B and 3H; 2A, 2B and 3I; 2A, 2B and 3J; 2A, 2C and 2D; 2A, 2C and 2E; 2A, 2C and 2F; 2A, 2C and 2G; 2A, 2C and 2K; 2A, 2C and 2L; 2A, 2C and 3H; 2A, 2C and 3I; 2A, 2C and 3J; 2A, 2D and 2E; 2A, 2D and 2F; 2A, 2D and 2G; 2A, 2D and 2K; 2A, 2D and 2L; 2A, 2D and 3H; 2A, 2D and 3I; 2A, 2D and 3J; 2A, 2E and 2F; 2A, 2E and 2G; 2A, 2E and 2K; 2A, 2E and 2L; 2A, 2E and 3H; 2A, 2E and 3I; 2A, 2E and 3J; 2A, 2F and 2G; 2A, 2F and 2K; 2A, 2F and 2L; 2A, 2F and 3H; 2A, 2F and 3I; 2A, 2F and 3J; 2B, 2C and 2D; 2B, 2C and 2E; 2B, 2C and 2F; 2B, 2C and 2G; 2B, 2C and 2K; 2B, 2C and 2L; 2B, 2C and 3H; 2B, 2C and 3I; 2B, 2C and 3J; 2B, 2D and 2E; 2B, 2D and 2F; 2B, 2D and 2G; 2B, 2D and 2K; 2B, 2D and 2L; 2B, 2D and 3H; 2B, 2D and 3I; 2B, 2D and 3J; 2B, 2E and 2F; 2B, 2E and 2G; 2B, 2E and 2K; 2B, 2E and 2L; 2B, 2E and 3H; 2B, 2E and 3I; 2B, 2E and 3J; 2B, 2F and 2G; 2B, 2F and 2K; 2B, 2F and 2L; 2B, 2F and 3H; 2B, 2F and 3I; 2B, 2F and 3J; 2B, 2G and 2K; 2B, 2G and 2L; 2B, 2G and 3H; 2B, 2G and 3I; 2B, 2G and 3J; 2B, 2K and 2L; 2B, 2K and 3H; 2B, 2K and 3I; 2B, 2K and 3J; 2B, 2L and 3H; 2B, 2L and 3I; 2B, 2L and 3J; 2C, 2D and 2E; 2C, 2D and 2F; 2C, 2D and 2G; 2C, 2D and 2K; 2C, 2D and 2L; 2C, 2D and 3H; 2C, 2D and 3I; 2C, 2D and 3J; 2C, 2E and 2F; 2C, 2E and 2G; 2C, 2E and 2K; 2C, 2E and 2L; 2C, 2E and 3H; 2C, 2E and 3I; 2C, 2E and 3J; 2C, 2F and 2G; 2C, 2F and 2K; 2C, 2F and 2L; 2C, 2F and 2G; 2C, 2F and 2K; 2C, 2F and 2L; 2C, 2F and 3H; 2C, 2F and 3I; 2C, 2F and 3J; 2D, 2E and 2F; 2D, 2E and 2G; 2D, 2E and 2K; 2D, 2E and 2L; 2D, 2E and 3H; 2D, 2E and 3I; 2D, 2E and 3J; 2D, 2F and 2G; 2D, 2F and 2K; 2D, 2F and 2L; 2D, 2F and 3H; 2D, 2F and 3I; 2D, 2F and 3J; 2D, 2G and 2K; 2D, 2G and 2L; 2D, 2G and 3H; 2D, 2G and 3I; 2D, 2G and 3J; 2D, 2K and 2L; 2D, 2K and 3H; 2D, 2K and 3I; 2D, 2K and 3J; 2D, 2L and 3H; 2D, 2L and 3I; 2D, 2L and 3J; 2D, 3H and 3I; 2D, 3H and 3J; 2D, 3I and 3J; 2E, 2F and 2G; 2E, 2F and 2K; 2E, 2F and 2L; 2E, 2F and 3H; 2E, 2F and 3I; 2E, 2F and 3J; 2E, 2G and 2K; 2E, 2G and 2L; 2E, 2G and 3H; 2E, 2G and 3I; 2E, 2G and 3J; 2K, 2L and 3H; 2K, 2L and 3I; 2K, 2L and 3J; 2K, 3H and 3I; 2K, 3H and 3J; 2K, 3I and 3J; 2L, 3H and 3I; 2L, 3H and 3J; 2L, 3I and 3J; or 3H, 3I and 3J. In some embodiments, the non-naturally occurring eukaryotic organism comprises three or more exogenous nucleic acids, wherein each of the three or more exogenous nucleic acids encodes a different acetyl-CoA pathway enzyme.

In certain embodiments, the acetyl CoA pathway comprises: 2A, 2B, 2C and 2D; 2A, 2B, 2C and 2E; 2A, 2B, 2C and 2F; 2A, 2B, 2C and 2G; 2A, 2B, 2C and 2K; 2A, 2B, 2C and 2L; 2A, 2B, 2C and 3H; 2A, 2B, 2C and 3I; 2A, 2B, 2C and 3J; 2A, 2B, 2D and 2E; 2A, 2B, 2D and 2F; 2A, 2B, 2D and 2G; 2A, 2B, 2D and 2K; 2A, 2B, 2D and 2L; 2A, 2B, 2D and 3H; 2A, 2B, 2D and 3I; 2A, 2B, 2D and 3J; 2A, 2B, 2E and 2F; 2A, 2B, 2E and 2G; 2A, 2B, 2E and 2K; 2A, 2B, 2E and 2L; 2A, 2B, 2E and 3H; 2A, 2B, 2E and 3I; 2A, 2B, 2E and 3J; 2A, 2B, 2F and 2G; 2A, 2B, 2F and 2H; 2A, 2B, 2F and 21; 2A, 2B, 2F and 3H; 2A, 2B, 2F and 3I; 2A, 2B, 2F and 3J; 2A, 2B, 2G and 2K; 2A, 2B, 2G and 2L; 2A, 2B, 2G and 3H; 2A, 2B, 2G and 3I; 2A, 2B, 2G and 3J; 2A, 2B, 2K and 2L; 2A, 2B, 2K and 3H; 2A, 2B, 2K and 3I; 2A, 2B, 2K and 3J; 2A, 2B, 2L and 3H; 2A, 2B, 2L and 3I; 2A, 2B, 2L and 3J; 2A, 2B, 3H and 3I; 2A, 2B, 3H and 3J; 2A, 2B, 3I and 3J; 2A, 2C, 2D and 2E; 2A, 2C, 2D and 2F; 2A, 2C, 2D and 2G; 2A, 2C, 2D and 2K; 2A, 2C, 2D and 2L; 2A, 2C, 2D and 3H; 2A, 2C, 2D and 3I; 2A, 2C, 2D and 3J; 2A, 2C, 2E and 2F; 2A, 2C, 2E and 2G; 2A, 2C, 2E and 2K; 2A, 2C, 2E and 2L; 2A, 2C, 2E and 3H; 2A, 2C, 2E and 3I; 2A, 2C, 2E and 3J; 2A, 2C, 2F and 2G; 2A, 2C, 2F and 2K; 2A, 2C, 2F and 2L; 2A, 2C, 2F and 3H; 2A, 2C, 2F and 3I; 2A, 2C, 2F and 3J; 2A, 2C, 2G and 2K; 2A, 2C, 2G and 2L; 2A, 2C, 2G and 3H; 2A, 2C, 2G and 3I; 2A, 2C, 2G and 3J; 2A, 2C, 2K and 2L; 2A, 2C, 2K and 3H; 2A, 2C, 2K and 3I; 2A, 2C, 2K and 3J; 2A, 2C, 2L and 3H; 2A, 2C, 2L and 3I; 2A, 2C, 2L and 3J; 2A, 2C, 3H and 3I; 2A, 2C, 3H and 3J; 2A, 2C, 3I and 3J; 2A, 2D, 2E and 2F; 2A, 2D, 2E and 2G; 2A, 2D, 2E and 2K; 2A, 2D, 2E and 2L; 2A, 2D, 2E and 3H; 2A, 2D, 2E and 3I; 2A, 2D, 2E and 3J; 2A, 2D, 2F and 2G; 2A, 2D, 2F and 2K; 2A, 2D, 2F and 2L; 2A, 2D, 2F and 3H; 2A, 2D, 2F and 3I; 2A, 2D, 2F and 3J; 2A, 2D, 2G and 2K; 2A, 2D, 2G and 2L; 2A, 2D, 2G and 3H; 2A, 2D, 2G and 3I; 2A, 2D, 2G and 3J; 2A, 2D, 2K and 2L; 2A, 2D, 2K and 3H; 2A, 2D, 2K and 3I; 2A, 2D, 2K and 3J; 2A, 2D, 2L and 3H; 2A, 2D, 2L and 3I; 2A, 2D, 2L and 3J; 2A, 2D, 3H and 3I; 2A, 2D, 3H and 3J; 2A, 2D, 3I and 3J; 2A, 2E, 2F and 2G; 2A, 2E, 2F and 2K; 2A, 2E, 2F and 2L; 2A, 2E, 2F and 3H; 2A, 2E, 2F and 3I; 2A, 2E, 2F and 3J; 2A, 2E, 2G and 2K; 2A, 2E, 2G and 2L; 2A, 2E, 2G and 3H; 2A, 2E, 2G and 3I; 2A, 2E, 2G and 3J; 2A, 2E, 2K and 2L; 2A, 2E, 2K and 3H; 2A, 2E, 2K and 3I; 2A, 2E, 2K and 3J; 2A, 2E, 2L and 3H; 2A, 2E, 2L and 3I; 2A, 2E, 2L and 3J; 2A, 2E, 3H and 3I; 2A, 2E, 3H and 3J; 2A, 2E, 3I and 3J; 2A, 2F, 2G and 2K; 2A, 2F, 2G and 2L; 2A, 2F, 2G and 3H; 2A, 2F, 2G and 3I; 2A, 2F, 2G and 3J; 2A, 2F, 2K and 2L; 2A, 2F, 2K and 3H; 2A, 2F, 2K and 3I; 2A, 2F, 2K and 3J; 2A, 2F, 2L and 3H; 2A, 2F, 2L and 3I; 2A, 2F, 2L and 3J; 2A, 2F, 3H and 3I; 2A, 2F, 3H and 3J; 2A, 2F, 3I and 3J; 2A, 2G, 2K and 2L; 2A, 2G, 2K and 3H; 2A, 2G, 2K and 3I; 2A, 2G, 2K and 3J; 2A, 2G, 2L and 3H; 2A, 2G, 2L and 3I; 2A, 2G, 2L and 3J; 2A, 2G, 3H and 3I; 2A, 2G, 3H and 3J; 2A, 2G, 3I and 3J; 2A, 3H, 3I and 3J; 2B, 2C, 2D and 2E; 2B, 2C, 2D and 2F; 2B, 2C, 2D and 2G; 2B, 2C, 2D and 2K; 2B, 2C, 2D and 2L; 2B, 2C, 2D and 3H; 2B, 2C, 2D and 3I; 2B, 2C, 2D and 3J; 2B, 2C, 2E and 2F; 2B, 2C, 2E and 2G; 2B, 2C, 2E and 2K; 2B, 2C, 2E and 2L; 2B, 2C, 2E and 3H; 2B, 2C, 2E and 3I; 2B, 2C, 2E and 3J; 2B, 2C, 2F and 2G; 2B, 2C, 2F and 2K; 2B, 2C, 2F and 2L; 2B, 2C, 2F and 3H; 2B, 2C, 2F and 3I; 2B, 2C, 2F and 3J; 2B, 2C, 2G and 2K; 2B, 2C, 2G and 2L; 2B, 2C, 2G and 3H; 2B, 2C, 2G and 3I; 2B, 2C, 2G and 3J; 2B, 2C, 2K and 2L; 2B, 2C, 2K and 3H; 2B, 2C, 2K and 3I; 2B, 2C, 2K and 3J; 2B, 2C, 2L and 3H; 2B, 2C, 2L and 3I; 2B, 2C, 2L and 3J; 2B, 2C, 3H and 3I; 2B, 2C, 3H and 3J; 2B, 2C, 3I and 3J; 2B, 2D, 2E and 2F; 2B, 2D, 2E and 2G; 2B, 2D, 2E and 2K; 2B, 2D, 2E and 2L; 2B, 2D, 2E and 3H; 2B, 2D, 2E and 3I; 2B, 2D, 2E and 3J; 2B, 2D, 2F and 2G; 2B, 2D, 2F and 2K; 2B, 2D, 2F and 2L; 2B, 2D, 2F and 3H; 2B, 2D, 2F and 3I; 2B, 2D, 2F and 3J; 2B, 2D, 2G and 2K; 2B, 2D, 2G and 2L; 2B, 2D, 2G and 3H; 2B, 2D, 2G and 3I; 2B, 2D, 2G and 3J; 2B, 2D, 2K and 2L; 2B, 2D, 2K and 3H; 2B, 2D, 2K and 3I; 2B, 2D, 2K and 3J; 2B, 2D, 2L and 3H; 2B, 2D, 2L and 3I; 2B, 2D, 2L and 3J; 2B, 2D, 3H and 3I; 2B, 2D, 3H and 3J; 2B, 2D, 3I and 3J; 2B, 2E, 2F and 2G; 2B, 2E, 2F and 2K; 2B, 2E, 2F and 2L; 2B, 2E, 2F and 3H; 2B, 2E, 2F and 3I; 2B, 2E, 2F and 3J; 2B, 2E, 2G and 2K; 2B, 2E, 2G and 2L; 2B, 2E, 2G and 3H; 2B, 2E, 2G and 3I; 2B, 2E, 2G and 3J; 2B, 2E, 2K and 2L; 2B, 2E, 2K and 3H; 2B, 2E, 2K and 3I; 2B, 2E, 2K and 3J; 2B, 2E, 2L and 3H; 2B, 2E, 2L and 3I; 2B, 2E, 2L and 3J; 2B, 2E, 3H and 3I; 2B, 2E, 3H and 3J; 2B, 2E, 3I and 3J; 2B, 2F, 2G and 2K; 2B, 2F, 2G and 2L; 2B, 2F, 2G and 3H; 2B, 2F, 2G and 3I; 2B, 2F, 2G and 3J; 2B, 2F, 2K and 2L; 2B, 2F, 2K and 3H; 2B, 2F, 2K and 3I; 2B, 2F, 2K and 3J; 2B, 2F, 2L and 3H; 2B, 2F, 2L and 3I; 2B, 2F, 2L and 3J; 2B, 2F, 3H and 3I; 2B, 2F, 3H and 3J; 2B, 2F, 3I and 3J; 2B, 2G, 2K and 2L; 2B, 2G, 2K and 3H; 2B, 2G, 2K and 3I; 2B, 2G, 2K and 3J; 2B, 2G, 2L and 3H; 2B, 2G, 2L and 3I; 2B, 2G, 2L and 3J; 2B, 2G, 3H and 3I; 2B, 2G, 3H and 3J; 2B, 3H, 3I and 3J; 2B, 2K, 2L and 3H; 2B, 2K, 2L and 3I; 2B, 2K, 2L and 3J; 2B, 2K, 3H and 3I; 2B, 2K, 3H and 3J; 2B, 2K, 3I and 3J; 2B, 2L, 3H and 3I; 2B, 2L, 3H and 3J; 2B, 2L, 3I and 3J; 2B, 3H, 3I and 3J; 2C, 2D, 2E and 2F; 2C, 2D, 2E and 2G; 2C, 2D, 2E and 2K; 2C, 2D, 2E and 2L; 2C, 2D, 2E and 3H; 2C, 2D, 2E and 3I; 2C, 2D, 2E and 3J; 2C, 2D, 2F and 2G; 2C, 2D, 2F and 2K; 2C, 2D, 2F and 2L; 2C, 2D, 2F and 3H; 2C, 2D, 2F and 3I; 2C, 2D, 2F and 3J; 2C, 2D, 2G and 2K; 2C, 2D, 2G and 2L; 2C, 2D, 2G and 3H; 2C, 2D, 2G and 3I; 2C, 2D, 2G and 3J; 2C, 2D, 3H and 3I; 2C, 2D, 2K and 2L; 2C, 2D, 2K and 3H; 2C, 2D, 2K and 3I; 2C, 2D, 2K and 3J; 2C, 2D, 2L and 3H; 2C, 2D, 2L and 3I; 2C, 2D, 2L and 3J; 2C, 2D, 3H and 3I; 2C, 2D, 3H and 3J; 2C, 2D, 3I and 3J; 2C, 2E, 2F and 2G; 2C, 2E, 2F and 2K; 2C, 2E, 2F and 2L; 2C, 2E, 2F and 3H; 2C, 2E, 2F and 3I; 2C, 2E, 2F and 3J; 2C, 2E, 2G and 2K; 2C, 2E, 2G and 2L; 2C, 2E, 2G and 3H; 2C, 2E, 2G and 3I; 2C, 2E, 2G and 3J; 2C, 2E, 2K and 2L; 2C, 2E, 2K and 3H; 2C, 2E, 2K and 3I; 2C, 2E, 2K and 3J; 2C, 2E, 2L and 3H; 2C, 2E, 2L and 3I; 2C, 2E, 2L and 3J; 2C, 2E, 3H and 3I; 2C, 2E, 3H and 3J; 2C, 2E, 3I and 3J; 2C, 2F, 2G and 2K; 2C, 2F, 2G and 2L; 2C, 2F, 2G and 3H; 2C, 2F, 2G and 3I; 2C, 2F, 2G and 3J; 2C, 2F, 2K and 2L; 2C, 2F, 2K and 3H; 2C, 2F, 2K and 3I; 2C, 2F, 2K and 3J; 2C, 2F, 2L and 3H; 2C, 2F, 2L and 3I; 2C, 2F, 2L and 3J; 2C, 2F, 3H and 3I; 2C, 2F, 3H and 3J; 2C, 2F, 3I and 3J; 2C, 2G, 2K and 2L; 2C, 2G, 2K and 3H; 2C, 2G, 2K and 3I; 2C, 2G, 2K and 3J; 2C, 2G, 2L and 3H; 2C, 2G, 2L and 3I; 2C, 2G, 2L and 3J; 2C, 2G, 3H and 3I; 2C, 2G, 3H and 3J; 2C, 2G, 3I and 3J; 2C, 2K, 2L and 3H; 2C, 2K, 2L and 3I; 2C, 2K, 2L and 3J; 2C, 2K, 3H and 3I; 2C, 2K, 3H and 3J; 2C, 2K, 3I and 3J; 2C, 2L, 3H and 3I; 2C, 2L, 3H and 3J; 2C, 2L, 3I and 3J; 2C, 3H, 3I and 3J; 2D, 2E, 2F and 2G; 2D, 2E, 2F and 2K; 2D, 2E, 2F and 2L; 2D, 2E, 2F and 3H; 2D, 2E, 2F and 3I; 2D, 2E, 2F and 3J; 2D, 2E, 2G and 2K; 2D, 2E, 2G and 2L; 2D, 2E, 2G and 3H; 2D, 2E. 2G and 3I; 2D, 2E, 2G and 3J; 2D, 2E, 2K and 2L; 2D, 2E, 2K and 3H; 2D, 2E. 2K and 3I; 2D, 2E, 2K and 3J; 2D, 2E, 2L and 3H; 2D, 2E. 2L and 3I; 2D, 2E, 2L and 3J; 2D, 2E, 3H and 3I; 2D, 2E, 3H and 3J; 2D, 2E, 3I and 3J; 2D, 2F, 2G and 2K; 2D, 2F, 2G and 2L; 2D, 2F, 2G and 3H; 2D, 2F, 2G and 3I; 2D, 2F, 2G and 3J; 2D, 2F, 2K and 2L; 2D, 2F, 2K and 3H; 2D, 2F, 2K and 3I; 2D, 2F, 2K and 3J; 2D, 2F, 2L and 3H; 2D, 2F, 2L and 3I; 2D, 2F, 2L and 3J; 2D, 2F, 3H and 3I; 2D, 2F, 3H and 3J; 2D, 2F, 3I and 3J; 2E, 2F, 2G and 3H; 2E, 2F, 2G and 3I; 2E, 2F, 2G and 3J; 2E, 2F, 3H and 3I; 2E, 2F, 3H and 3J; 2E, 2F, 3I and 3J; 2F, 2G, 3H and 3I; 2F, 2G, 3H and 3J; 2F, 2G, 3I and 3J; or 2G, 3H, 3I and 3J. 2D, 2G, 2K and 2L; 2D, 2G, 2K and 3H; 2D, 2G, 2K and 3I; 2D, 2G, 2K and 3J; 2D, 2G, 2L and 3H; 2D, 2G, 2L and 3I; 2D, 2G, 2L and 3J; 2D, 2G, 2H and 3I; 2D, 2G, 2H and 3J; 2D, 2G, 3I and 3J; 2D, 2K, 2L and 3H; 2D, 2K, 2L and 3I; 2D, 2K, 2L and 3J; 2D, 2K, 3H and 3I; 2D, 2K, 3H and 3J; 2D, 2K, 3I and 3J; 2D, 2L, 3H and 3I; 2D, 2L, 3H and 3J; 2D, 3H, 3I and 3J; 2D, 3H, 3I and 3J; 2E, 2F, 2G and 2K; 2E, 2F, 2G and 2L; 2E, 2F, 2G and 3H; 2E, 2F, 2G and 3I; 2E, 2F, 2G and 3J; 2E, 2F, 2K and 2L; 2E, 2F, 2K and 3H; 2E, 2F, 2K and 3I; 2E, 2F, 2K and 3J; 2E, 2F, 2L and 3H; 2E, 2F, 2L and 3I; 2E, 2F, 2L and 3J; 2E, 2F, 3H and 3I; 2E, 2F, 3H and 3J; 2E, 2F, 3I and 3J; 2E, 2G, 2K and 2L; 2E, 2G, 2K and 3H; 2E, 2G, 2K and 3I; 2E, 2G, 2K and 3J; 2E, 2G, 2L and 3H; 2E, 2G, 2L and 3I; 2E, 2G, 2L and 3J; 2E, 2G, 3H and 3I; 2E, 2G, 3H and 3J; 2E, 2G, 3I and 3J; 2E, 2K, 2L and 3H; 2E, 2K, 2L and 3I; 2E, 2K, 2L and 3J; 2E, 2K, 3H and 3I; 2E, 2K, 3H and 3J; 2E, 2K, 3I and 3J; 2E, 2L, 3H and 3I; 2E, 2L, 3H and 3J; 2E, 2L, 3I and 3J; 2E, 3H, 3I and 3J. 2F, 2G, 2K and 2L; 2F, 2G, 2K and 3H; 2F, 2G, 2K and 3I; 2F, 2G, 2K and 3J; 2F, 2G, 2L and 3H; 2F, 2G, 2L and 3I; 2F, 2G, 2L and 3J; 2F, 2G, 3H and 3I; 2F, 2G, 3H and 3J; 2F, 2G, 3I and 3J; 2F, 2K, 2L and 3H; 2F, 2K, 2L and 3I; 2F, 2K, 2L and 3J; 2F, 2K, 3H and 3I; 2F, 2K, 3H and 3J; 2F, 2K, 3I and 3J; 2F, 3H, 3I and 3J; 2G, 2K, 2L and 3H; 2G, 2K, 2L and 3I; 2G, 2K, 2L and 3J; 2G, 2K, 3H and 3I; 2G, 2K, 3H and 3J; 2G, 2K, 3I and 3J; 2G, 2L, 3H and 3I; 2G, 2L, 3H and 3J; 2G, 2L, 3I and 3J; 2G, 3H, 3I and 3J; 2K, 2L, 3H and 3I; 2K, 2L, 3H and 3J; 2K, 2L, 3I and 3J; or 2L, 3H, 3I and 3J. In some embodiments, the non-naturally occurring eukaryotic organism comprises four or more exogenous nucleic acids, wherein each of the four or more exogenous nucleic acids encodes a different acetyl-CoA pathway enzyme.

In other embodiments, the acetyl CoA pathway comprises: 2A, 2B, 2C, 2D and 2E; 2A, 2B, 2C, 2D and 2F; 2A, 2B, 2C, 2D and 2G; 2A, 2B, 2C, 2D and 3H; 2A, 2B, 2C, 2D and 3I; 2A, 2B, 2C, 2D and 3J; 2A, 2B, 2C, 2E and 2F; 2A, 2B, 2C, 2E and 2G; 2A, 2B, 2C, 2E and 3H; 2A, 2B, 2C, 2E and 3I; 2A, 2B, 2C, 2E and 3J; 2A, 2B, 2C, 2F and 2G; 2A, 2B, 2C, 2F and 3H; 2A, 2B, 2C, 2F and 3I; 2A, 2B, 2C, 2F and 3J; 2A, 2B, 2C, 2G and 3H; 2A, 2B, 2C, 2G and 3I; 2A, 2B, 2C, 2G and 3J; 2A, 2B, 2C, 3H and 3I; 2A, 2B, 2C, 3H and 3J; 2A, 2B, 2C, 3I and 3J; 2A, 2B, 2D, 2E and 3H; 2A, 2B, 2D, 2E and 3I; 2A, 2B, 2D, 2E and 3J; 2A, 2B, 2D, 2F and 2G; 2A, 2B, 2D, 2F and 3H; 2A, 2B, 2D, 2F and 3I; 2A, 2B, 2D, 2F and 3J; 2A, 2B, 2D, 2G and 3H; 2A, 2B, 2D, 2G and 3I; 2A, 2B, 2D, 2G and 3J; 2A, 2B, 2D, 3H and 3I; 2A, 2B, 2D, 3H and 3J; 2A, 2B, 2D, 3I and 3J; 2A, 2B, 2E, 2F and 2G; 2A, 2B, 2E, 2F and 3H; 2A, 2B, 2E, 2F and 3I; 2A, 2B, 2E, 2F and 3J; 2A, 2B, 2E, 2G and 3H; 2A, 2B, 2E, 2G and 3I; 2A, 2B, 2E, 2G and 3J; 2A, 2B, 2E, 3H and 3I; 2A, 2B, 2E, 3H and 3J; 2A, 2B, 2E, 3I and 3J; 2A, 2B, 2F, 2G and 3H; 2A, 2B, 2F, 2G and 3I; 2A, 2B, 2F, 2G and 3J; 2A, 2B, 2F, 3H and 3I; 2A, 2B, 2F, 3H and 3J; 2A, 2B, 2F, 3I and 3J; 2A, 2B, 2G, 3H and 3I; 2A, 2B, 2G, 3H and 3J; 2A, 2B, 2G, 3I and 3J; 2A, 2B, 3H, 3I and 3J; 2A, 2C, 2D, 2E and 2F; 2A, 2C, 2D, 2E and 2G; 2A, 2C, 2D, 2E and 3H; 2A, 2C, 2D, 2E and 3I; 2A, 2C, 2D, 2E and 3J; 2A, 2C, 2D, 2F and 2G; 2A, 2C, 2D, 2F and 3H; 2A, 2C, 2D, 2F and 3I; 2A, 2C, 2D, 2F and 3J; 2A, 2C, 2D, 2G and 3H; 2A, 2C, 2D, 2G and 3I; 2A, 2C, 2D, 2G and 3J; 2A, 2C, 2D, 3H and 3I; 2A, 2C, 2D, 3H and 3J; 2A, 2C, 2D, 3I and 3J; 2A, 2C, 2E, 2F and 2G; 2A, 2C, 2E, 2F and 3H; 2A, 2C, 2E, 2F and 3I; 2A, 2C, 2E, 2F and 3J; 2A, 2C, 2E, 2G and 3H; 2A, 2C, 2E, 2G and 3I; 2A, 2C, 2E, 2G and 3J; 2A, 2C, 2E, 3H and 3I; 2A, 2C, 2E, 3H and 3J; 2A, 2C, 2E, 3I and 3J; 2A, 2C, 2F, 2G and 3H; 2A, 2C, 2F, 2G and 3I; 2A, 2C, 2F, 2G and 3J; 2A, 2C, 2F, 3H and 3I; 2A, 2C, 2F, 3H and 3J; 2A, 2C, 2F, 3I and 3J; 2A, 2C, 2G, 3H and 3I; 2A, 2C, 2G, 3H and 3J; 2A, 2C, 2G, 3I and 3J; 2A, 2C, 3H, 3I and 3J; 2A, 2D, 2E, 2F and 2G; 2A, 2D, 2E, 2F and 3H; 2A, 2D, 2E, 2F and 3I; 2A, 2D, 2E, 2F and 3J; 2A, 2D, 2E, 2G and 3H; 2A, 2D, 2E, 2G and 3I; 2A, 2D, 2E, 2G and 3J; 2A, 2D, 2E, 3H and 3I; 2A, 2D, 2E, 3H and 3J; 2A, 2D, 2E, 3I and 3J; 2A, 2D, 2F, 2G and 3H; 2A, 2D, 2F, 2G and 3I; 2A, 2D, 2F, 2G and 3J; 2A, 2D, 2F, 3H and 3I; 2A, 2D, 2F, 3H and 3J; 2A, 2D, 2F, 3I and 3J; 2A, 2D, 2G, 3H and 3I; 2A, 2D, 2G, 3H and 3J; 2A, 2D, 2G, 3I and 3J; 2A, 2D, 3H, 3I and 3J; 2A, 2E, 2F, 2G and 3H; 2A, 2E, 2F, 2G and 3I; 2A, 2E, 2F, 2G and 3J; 2A, 2E, 2F, 3H and 3I; 2A, 2E, 2F, 3H and 3J; 2A, 2E, 2F, 3I and 3J; 2A, 2E, 2G, 3H and 3I; 2A, 2E, 2G, 3H and 3J; 2A, 2E, 2G, 3I and 3J; 2A, 2E, 3H, 3I and 3J; 2A, 2F, 2G, 3H and 3I; 2A, 2F, 2G, 3H and 3J; 2A, 2F, 2G, 3I and 3J; 2A, 2F, 3H, 3I and 3J; 2A, 2G, 3H, 3I and 3J; 2B, 2C, 2D, 2E and 2F; 2B, 2C, 2D, 2E and 2G; 2B, 2C, 2D, 2E and 3H; 2B, 2C, 2D, 2E and 3I; 2B, 2C, 2D, 2E and 3J; 2B, 2C, 2D, 2F and 2G; 2B, 2C, 2D, 2F and 3H; 2B, 2C, 2D, 2F and 3I; 2B, 2C, 2D, 2F and 3J; 2B, 2C, 2D, 2G and 3H; 2B, 2C, 2D, 2G and 3I; 2B, 2C, 2D, 2G and 3J; 2B, 2C, 2D, 3H and 3I; 2B, 2C, 2D, 3H and 3J; 2B, 2C, 2D, 3I and 3J; 2B, 2C, 2E, 2F and 2G; 2B, 2C, 2E, 2F and 3H; 2B, 2C, 2E, 2F and 3I; 2B, 2C, 2E, 2F and 3J; 2B, 2C, 2E, 2G and 3H; 2B, 2C, 2E, 2G and 3I; 2B, 2C, 2E, 2G and 3J; 2B, 2C, 2E, 3H and 3I; 2B, 2C, 2E, 3H and 3J; 2B, 2C, 2E, 3I and 3J; 2B, 2C, 2F, 2G and 3H; 2B, 2C, 2F, 2G and 3I; 2B, 2C, 2F, 2G and 3J; 2B, 2C, 2F, 3H and 3I; 2B, 2C, 2F, 3H and 3J; 2B, 2C, 2F, 3I and 3J; 2B, 2C, 2G, 3H and 3I; 2B, 2C, 2G, 3H and 3J; 2B, 2C, 2G, 3I and 3J; 2B, 2C, 3H, 3I and 3J; 2B, 2D, 2E, 2F and 2G; 2B, 2D, 2E, 2F and 3H; 2B, 2D, 2E, 2F and 3I; 2B, 2D, 2E, 2F and 3J; 2B, 2D, 2E, 2G and 3H; 2B, 2D, 2E, 2G and 3I; 2B, 2D, 2E, 2G and 3J; 2B, 2D, 2E, 3H and 3I; 2B, 2D, 2E, 3H and 3J; 2B, 2D, 2E, 3I and 3J; 2B, 2D, 2F, 2G and 3H; 2B, 2D, 2F, 2G and 3I; 2B, 2D, 2F, 2G and 3J; 2B, 2D, 2F, 3H and 3I; 2B, 2D, 2F, 3H and 3J; 2B, 2D, 2F, 3I and 3J; 2B, 2E, 2F, 2G and 3H; 2B, 2E, 2F, 2G and 3I; 2B, 2E, 2F, 2G and 3J; 2B, 2E, 2F, 3H and 3I; 2B, 2E, 2F, 3H and 3J; 2B, 2E, 2F, 3I and 3J; 2B, 2E, 2G, 3H and 3I; 2B, 2E, 2G, 3H and 3J; 2B, 2E, 2G, 3I and 3J; 2B, 2E, 3H, 3I and 3J; 2B, 2F, 2G, 3H and 3I; 2B, 2F, 2G, 3H and 3J; 2B, 2F, 2G, 3I and 3J; 2B, 2G, 3H, 3I and 3J; 2C, 2D, 2E, 2F and 3H; 2C, 2D, 2E, 2F and 3I; 2C, 2D, 2E, 2F and 3J; 2C, 2D, 2E, 2G and 3H; 2C, 2D, 2E, 2G and 3I; 2C, 2D, 2E, 2G and 3J; 2C, 2D, 2E, 3H and 3I; 2C, 2D, 2E, 3H and 3J; 2C, 2D, 2E, 3I and 3J; 2C, 2D, 2F, 2G and 3H; 2C, 2D, 2F, 2G and 3I; 2C, 2D, 2F, 2G and 3J; 2C, 2D, 2F, 3H and 3I; 2C, 2D, 2F, 3H and 3J; 2C, 2D, 2F, 3I and 3J; 2C, 2D, 2G, 3H and 3I; 2C, 2D, 2G, 3H and 3J; 2C, 2D, 2G, 3I and 3J; 2C, 2D, 3H, 3I and 3J; 2D, 2E, 2F, 2G and 3H; 2D, 2E, 2F, 2G and 3I; 2D, 2E, 2F, 2G and 3J; 2D, 2E, 2F, 3H and 3I; 2D, 2E, 2F, 3H and 3J; 2D, 2E, 2F, 3I and 3J; 2D, 2E, 2G, 3H and 3I; 2D, 2E, 2G, 3H and 3J; 2D, 2E. 2G, 3I and 3J; 2D, 2E, 3H, 3I and 3J; 2E, 2F, 2G, 3H and 3I; 2E, 2F, 2G, 3H and 3J; 2E, 2F, 2G, 3I and 3J; 2E, 2F, 3H, 3I and 3J; or 2F, 2G, 3H, 3I and 3J. In some embodiments, the non-naturally occurring eukaryotic organism, comprises five or more exogenous nucleic acids, wherein each of the five or more exogenous nucleic acids encodes a different acetyl-CoA pathway enzyme.

In yet other embodiments, the acetyl-CoA pathway comprises: 2A, 2B, 2C, 2D, 2E and 2F; 2A, 2B, 2C, 2D, 2E and 2G; 2A, 2B, 2C, 2D, 2E and 3H; 2A, 2B, 2C, 2D, 2E and 3I; 2A, 2B, 2C, 2D, 2E and 3J; 2A, 2B, 2C, 2D, 2F and 2G; 2A, 2B, 2C, 2D, 2F and 3H; 2A, 2B, 2C, 2D, 2F and 3I; 2A, 2B, 2C, 2D, 2F and 3H; 2A, 2B, 2C, 2D, 2G and 3H; 2A, 2B, 2C, 2D, 2G and 3I; 2A, 2B, 2C, 2D, 2G and 3J; 2A, 2B, 2C, 2D, 3H and 3I; 2A, 2B, 2C, 2D, 3H and 3J; 2A, 2B, 2C, 2D, 3I and 3J; 2A, 2B, 2C, 2E, 2F and 2G; 2A, 2B, 2C, 2E, 2F and 3H; 2A, 2B, 2C, 2E, 2F and 3I; 2A, 2B, 2C, 2E, 2F and 3J; 2A, 2B, 2C, 2E, 2G and 3H; 2A, 2B, 2C, 2E, 2G and 3I; 2A, 2B, 2C, 2E, 2G and 3J; 2A, 2B, 2C, 2E, 3H and 3I; 2A, 2B, 2C, 2E, 3H and 3J; 2A, 2B, 2C, 2E, 3I and 3J; 2A, 2B, 2C, 2F, 2G and 3H; 2A, 2B, 2C, 2F, 2G and 3I; 2A, 2B, 2C, 2F, 2G and 3J; 2A, 2B, 2C, 2F, 3H and 3I; 2A, 2B, 2C, 2F, 3H and 3J; 2A, 2B, 2C, 2F, 3I and 3J; 2A, 2B, 2C, 2G, 3H and 3I; 2A, 2B, 2C, 2G, 3H and 3J; 2A, 2B, 2C, 2G, 3I and 3J; 2A, 2B, 2C, 3H, 3I and 3J; 2A, 2B, 2D, 2E, 3H and 3I; 2A, 2B, 2D, 2E, 3H and 3J; 2A, 2B, 2D, 2E, 3I and 3J; 2A, 2B, 2D, 2F, 2G and 3H; 2A, 2B, 2D, 2F, 2G and 3I; 2A, 2B, 2D, 2F, 2G and 3J; 2A, 2B, 2D, 2F, 3H and 3I; 2A, 2B, 2D, 2F, 3H and 3J; 2A, 2B, 2D, 2F, 3I and 3J; 2A, 2B, 2D, 2G, 3H and 3I; 2A, 2B, 2D, 2G, 3H and 3J; 2A, 2B, 2D, 2G, 3I and 3J; 2A, 2B, 2D, 3H, 3I and 3J; 2A, 2B, 2E, 2F, 2G and 3H; 2A, 2B, 2E, 2F, 2G and 3I; 2A, 2B, 2E, 2F, 2G and 3J; 2A, 2B, 2E, 2F, 3H and 3I; 2A, 2B, 2E, 2F, 3H and 3J; 2A, 2B, 2E, 2F, 3I and 3J; 2A, 2B, 2E, 2G, 3H and 3I; 2A, 2B, 2E, 2G, 3H and 3J; 2A, 2B, 2E, 2G, 3I and 3J; 2A, 2B, 2E, 3H, 3I and 3J; 2A, 2B, 2F, 2G, 3H and 3I; 2A, 2B, 2F, 2G, 3H and 3J; 2A, 2B, 2F, 2G, 3I and 3J; 2A, 2B, 2F, 3H, 3I and 3J; 2A, 2B, 2G, 3H, 3I and 3J; 2A, 2C, 2D, 2E, 2F and 2G; 2A, 2C, 2D, 2E, 2F and 3H; 2A, 2C, 2D, 2E, 2F and 3I; 2A, 2C, 2D, 2E, 2F and 3J; 2A, 2C, 2D, 2E, 2G and 3H; 2A, 2C, 2D, 2E, 2G and 3I; 2A, 2C, 2D, 2E, 2G and 3J; 2A, 2C, 2D, 2E, 3H and 3I; 2A, 2C, 2D, 2E, 3H and 3J; 2A, 2C, 2D, 2E, 3I and 3J; 2A, 2C, 2D, 2F, 2G and 3H; 2A, 2C, 2D, 2F, 2G and 3I; 2A, 2C, 2D, 2F, 2G and 3J; 2A, 2C, 2D, 2F, 3H and 3I; 2A, 2C, 2D, 2F, 3H and 3J; 2A, 2C, 2D, 2F, 3I and 3J; 2A, 2C, 2D, 2G, 3H and 3I; 2A, 2C, 2D, 2G, 3H and 3J; 2A, 2C, 2D, 2G, 3I and 3J; 2A, 2C, 2D, 3H, 3I and 3J; 2A, 2C, 2E, 2F, 2G and 3H; 2A, 2C, 2E, 2F, 2G and 3I; 2A, 2C, 2E, 2F, 2G and 3J; 2A, 2C, 2E, 2F, 3H and 3I; 2A, 2C, 2E, 2F, 3H and 3J; 2A, 2C, 2E, 2F, 3I and 3J; 2A, 2C, 2E, 2G, 3H and 3I; 2A, 2C, 2E, 2G, 3H and 3J; 2A, 2C, 2E, 2G, 3I and 3J; 2A, 2C, 2E, 3H, 3I and 3J; 2A, 2C, 2F, 2G, 3H and 3I; 2A, 2C, 2F, 2G, 3H and 3J; 2A, 2C, 2F, 2G, 3I and 3J; 2A, 2C, 2F, 3H, 3I and 3J; 2A, 2C, 2G, 3H, 3I and 3J; 2A, 2D, 2E, 2F, 2G and 3H; 2A, 2D, 2E, 2F, 2G and 3I; 2A, 2D, 2E, 2F, 2G and 3J; 2A, 2D, 2E, 2F, 3H and 3I; 2A, 2D, 2E, 2F, 3H and 3J; 2A, 2D, 2E, 2F, 3I and 3J; 2A, 2D, 2E, 2G, 3H and 3I; 2A, 2D, 2E, 2G, 3H and 3J; 2A, 2D, 2E, 2G, 3I and 3J; 2A, 2D, 2E, 3H, 3I and 3J; 2A, 2D, 2F, 2G, 3H and 3I; 2A, 2D, 2F, 2G, 3H and 3J; 2A, 2D, 2F, 2G, 3I and 3J; 2A, 2D, 2F, 3H, 3I and 3J; 2A, 2D, 2G, 3H, 3I and 3J; 2A, 2E, 2F, 2G, 3H and 3I; 2A, 2E, 2F, 2G, 3H and 3J; 2A, 2E, 2F, 2G, 3I and 3J; 2A, 2E, 2F, 3H, 3I and 3J; 2A, 2E, 2G, 3H, 3I and 3J; 2A, 2F, 2G, 3H, 3I and 3J; 2B, 2C, 2D, 2E, 2F and 2G; 2B, 2C, 2D, 2E, 2F and 3H; 2B, 2C, 2D, 2E, 2F and 3I; 2B, 2C, 2D, 2E, 2F and 3J; 2B, 2C, 2D, 2E, 2G and 3H; 2B, 2C, 2D, 2E, 2G and 3I; 2B, 2C, 2D, 2E, 2G and 3J; 2B, 2C, 2D, 2E, 3H and 3I; 2B, 2C, 2D, 2E, 3H and 3I; 2B, 2C, 2D, 2E, 3I and 3J; 2B, 2C, 2D, 2F, 2G and 3H; 2B, 2C, 2D, 2F, 2G and 3I; 2B, 2C, 2D, 2F, 2G and 3J; 2B, 2C, 2D, 2F, 3H and 3I; 2B, 2C, 2D, 2F, 3H and 3J; 2B, 2C, 2D, 2F, 3I and 3J; 2B, 2C, 2D, 2G, 3H and 3I; 2B, 2C, 2D, 2G, 3H and 3J; 2B, 2C, 2D, 2G, 3I and 3J; 2B, 2C, 2D, 3H, 3I and 3J; 2B, 2C, 2E, 2F, 2G and 3H; 2B, 2C, 2E, 2F, 2G and 3I; 2B, 2C, 2E, 2F, 2G and 3J; 2B, 2C, 2E, 2F, 3H and 3I; 2B, 2C, 2E, 2F, 3H and 3J; 2B, 2C, 2E, 2F, 3I and 3J; 2B, 2C, 2E, 2G, 3H and 3I; 2B, 2C, 2E, 2G, 3H and 3J; 2B, 2C, 2E, 2G, 3I and 3J; 2B, 2C, 2E, 3H, 3I and 3J; 2B, 2C, 2F, 2G, 3H and 3I; 2B, 2C, 2F, 2G, 3H and 3J; 2B, 2C, 2F, 2G, 3I and 3J; 2B, 2C, 2F, 3H, 3I and 3J; 2B, 2C, 2G, 3H, 3I and 3J; 2B, 2D, 2E, 2F, 2G and 3H; 2B, 2D, 2E, 2F, 2G and 3I; 2B, 2D, 2E, 2F, 2G and 3J; 2B, 2D, 2E, 2F, 3H and 3I; 2B, 2D, 2E, 2F, 3H and 3J; 2B, 2D, 2E, 2F, 3I and 3J; 2B, 2D, 2E, 2G, 3H and 3I; 2B, 2D, 2E, 2G, 3H and 3J; 2B, 2D, 2E, 2G, 3I and 3J; 2B, 2D, 2E, 3H, 3I and 3J; 2B, 2D, 2F, 2G, 3H and 3I; 2B, 2D, 2F, 2G, 3H and 3J; 2B, 2D, 2F, 2G, 3I and 3J; 2B, 2D, 2F, 3H, 3I and 3J; 2B, 2E, 2F, 2G, 3H and 3I; 2B, 2E, 2F, 2G, 3H and 3J; 2B, 2E, 2F, 2G, 3I and 3J; 2B, 2E, 2F, 3H, 3I and 3J; 2B, 2E, 2G, 3H, 3I and 3J; 2B, 2F, 2G, 3H, 3I and 3J; 2C, 2D, 2E, 2F, 3H and 3I; 2C, 2D, 2E, 2F, 3H and 3J; 2C, 2D, 2E, 2F, 3I and 3J; 2C, 2D, 2E, 2G, 3H and 3I; 2C, 2D, 2E, 2G, 3H and 3J; 2C, 2D, 2E, 2G, 3I and 3J; 2C, 2D, 2E, 3H, 3I and 3J; 2C, 2D, 2F, 2G, 3H and 3I; 2C, 2D, 2F, 2G, 3H and 3J; 2C, 2D, 2F, 2G, 3I and 3J; 2C, 2D, 2F, 3H, 3I and 3J; 2C, 2D, 2G, 3H, 3I and 3J; 2D, 2E, 2F, 2G, 3H and 3I; 2D, 2E, 2F, 2G, 3H and 3J; 2D, 2E, 2F, 2G, 3I and 3J; 2D, 2E, 2F, 3H, 3I and 3J; 2D, 2E, 2G, 3H, 3I and 3J; or 2E, 2F, 2G, 3H, 3I and 3J. In some embodiments, the non-naturally occurring eukaryotic organism, comprises six or more exogenous nucleic acids, wherein each of the six or more exogenous nucleic acids encodes a different acetyl-CoA pathway enzyme.

In some embodiments, the acetyl-CoA pathway comprises: 2A, 2B, 2C, 2D, 2E, 2F and 2G; 2A, 2B, 2C, 2D, 2E, 2F and 3H; 2A, 2B, 2C, 2D, 2E, 2F and 3I; 2A, 2B, 2C, 2D, 2E, 2F and 3J; 2A, 2B, 2C, 2D, 2E, 2G and 3H; 2A, 2B, 2C, 2D, 2E, 2G and 3I; 2A, 2B, 2C, 2D, 2E, 2G and 3J; 2A, 2B, 2C, 2D, 2E, 3H and 3I; 2A, 2B, 2C, 2D, 2E, 3H and 3J; 2A, 2B, 2C, 2D, 2E, 3I and 3J; 2A, 2B, 2C, 2D, 2F, 2G and 3H; 2A, 2B, 2C, 2D, 2F, 2G and 3I; 2A, 2B, 2C, 2D, 2F, 2G and 3J; 2A, 2B, 2C, 2D, 2F, 3H and 3I; 2A, 2B, 2C, 2D, 2F, 3H and 3J; 2A, 2B, 2C, 2D, 2F, 3I and 3J; 2A, 2B, 2C, 2D, 2F, 3H and 3I; 2A, 2B, 2C, 2D, 2F, 3H and 3J; 2A, 2B, 2C, 2D, 2G, 3H and 3I; 2A, 2B, 2C, 2D, 2G, 3H and 3J; 2A, 2B, 2C, 2D, 2G, 3I and 3J; 2A, 2B, 2C, 2D, 3H, 3I and 3J; 2A, 2B, 2C, 2E, 2F, 2G and 3H; 2A, 2B, 2C, 2E, 2F, 2G and 3I; 2A, 2B, 2C, 2E, 2F, 2G and 3J; 2A, 2B, 2C, 2E, 2F, 3H and 3I; 2A, 2B, 2C, 2E, 2F, 3H and 3J; 2A, 2B, 2C, 2E, 2F, 3I and 3J; 2A, 2B, 2C, 2E, 2G, 3H and 3I; 2A, 2B, 2C, 2E, 2G, 3H and 3J; 2A, 2B, 2C, 2E, 2G, 3I and 3J; 2A, 2B, 2C, 2E, 3H, 3I and 3J; 2A, 2B, 2C, 2F, 2G, 3H and 3I; 2A, 2B, 2C, 2F, 2G, 3H and 3J; 2A, 2B, 2C, 2F, 2G, 3I and 3J; 2A, 2B, 2C, 2F, 3H, 3I and 3J; 2A, 2B, 2C, 2G, 3H, 3I and 3J; 2A, 2B, 2D, 2E, 3H, 3I and 3J; 2A, 2B, 2D, 2F, 2G, 3H and 3I; 2A, 2B, 2D, 2F, 2G, 3H and 3J; 2A, 2B, 2D, 2F, 2G, 3I and 3J; 2A, 2B, 2D, 2F, 3H, 3I and 3J; 2A, 2B, 2D, 2G, 3H, 3I and 3J; 2A, 2B, 2E, 2F, 2G, 3H and 3I; 2A, 2B, 2E, 2F, 2G, 3H and 3J; 2A, 2B, 2E, 2F, 2G, 3I and 3J; 2A, 2B, 2E, 2F, 3H, 3I and 3J; 2A, 2B, 2E, 2G, 3H, 3I and 3J; 2A, 2B, 2F, 2G, 3H, 3I and 3J; 2A, 2C, 2D, 2E, 2F, 2G and 3H; 2A, 2C, 2D, 2E, 2F, 2G and 3I; 2A, 2C, 2D, 2E, 2F, 2G and 3J; 2A, 2C, 2D, 2E, 2F, 3H and 3I; 2A, 2C, 2D, 2E, 2F, 3H and 3J; 2A, 2C, 2D, 2E, 2F, 3I and 3J; 2A, 2C, 2D, 2E, 2G, 3H and 3I; 2A, 2C, 2D, 2E, 2G, 3H and 3J; 2A, 2C, 2D, 2E, 2G, 3I and 3J; 2A, 2C, 2D, 2E, 3H, 3I and 3J; 2A, 2C, 2D, 2F, 2G, 3H and 3I; 2A, 2C, 2D, 2F, 2G, 3H and 3J; 2A, 2C, 2D, 2F, 2G, 3I and 3J; 2A, 2C, 2D, 2F, 3H, 3I and 3J; 2A, 2C, 2D, 2G, 3H, 3I and 3J; 2A, 2C, 2E, 2F, 2G, 3H and 3I; 2A, 2C, 2E, 2F, 2G, 3H and 3J; 2A, 2C, 2E, 2F, 2G, 3I and 3J; 2A, 2C, 2E, 2F, 3H, 3I and 3J; 2A, 2C, 2E, 2G, 3H, 3I and 3J; 2A, 2C, 2F, 2G, 3H, 3I and 3J; 2A, 2D, 2E, 2F, 2G, 3H and 3I; 2A, 2D, 2E, 2F, 2G, 3H and 3J; 2A, 2D, 2E, 2F, 2G, 3I and 3J; 2A, 2D, 2E, 2F, 3H, 3I and 3J; 2A, 2D, 2E, 2G, 3H, 3I and 3J; 2A, 2D, 2F, 2G, 3H, 3I and 3J; 2A, 2E, 2F, 2G, 3H, 3I and 3J; 2B, 2C, 2D, 2E, 2F, 2G and 3H; 2B, 2C, 2D, 2E, 2F, 2G and 3I; 2B, 2C, 2D, 2E, 2F, 2G and 3J; 2B, 2C, 2D, 2E, 2F, 3H and 3I; 2B, 2C, 2D, 2E, 2F, 3H and 3J; 2B, 2C, 2D, 2E, 2F, 3I and 3J; 2B, 2C, 2D, 2E, 2G, 3H and 3I; 2B, 2C, 2D, 2E, 2G, 3H and 3J; 2B, 2C, 2D, 2E, 2G, 3I and 3J; 2B, 2C, 2D, 2E, 3H, 3I and 3J; 2B, 2C, 2D, 2F, 2G, 3H and 3I; 2B, 2C, 2D, 2F, 2G, 3H and 3J; 2B, 2C, 2D, 2F, 2G, 3I and 3J; 2B, 2C, 2D, 2F, 3H, 3I and 3J; 2B, 2C, 2D, 2G, 3H, 3I and 3J; 2B, 2C, 2E, 2F, 2G, 3H and 3I; 2B, 2C, 2E, 2F, 2G, 3H and 3J; 2B, 2C, 2E, 2F, 2G, 3I and 3J; 2B, 2C, 2E, 2F, 3H, 3I and 3J; 2B, 2C, 2E, 2G, 3H, 3I and 3J; 2B, 2C, 2F, 2G, 3H, 3I and 3J; 2B, 2D, 2E, 2F, 2G, 3H and 3I; 2B, 2D, 2E, 2F, 2G, 3H and 3J; 2B, 2D, 2E, 2F, 2G, 3I and 3J; 2B, 2D, 2E, 2F, 3H, 3I and 3J; 2B, 2D, 2E, 2G, 3H, 3I and 3J; 2B, 2D, 2F, 2G, 3H, 3I and 3J; 2B, 2E, 2F, 2G, 3H, 3I and 3J; 2C, 2D, 2E, 2F, 3H, 3I and 3J; 2C, 2D, 2E, 2G, 3H, 3I and 3J; 2C, 2D, 2F, 2G, 3H, 3I and 3J; or 2D, 2E, 2F, 2G, 3H, 3I and 3J. In some embodiments, the non-naturally occurring eukaryotic organism, comprises seven or more exogenous nucleic acids, wherein each of the seven or more exogenous nucleic acids encodes a different acetyl-CoA pathway enzyme.

In certain embodiments, the acetyl-CoA pathway comprises: 2A, 2B, 2C, 2D, 2E, 2F, 2G and 3H; 2A, 2B, 2C, 2D, 2E, 2F, 2G and 3I; 2A, 2B, 2C, 2D, 2E, 2F, 2G and 3J; 2A, 2B, 2C, 2D, 2E, 2F, 3H and 3I; 2A, 2B, 2C, 2D, 2E, 2F, 3H and 3J; 2A, 2B, 2C, 2D, 2E, 2F, 3I and 3J; 2A, 2B, 2C, 2D, 2E, 2G, 3H and 3I; 2A, 2B, 2C, 2D, 2E, 2G, 3H and 3J; 2A, 2B, 2C, 2D, 2E, 2G, 3I and 3J; 2A, 2B, 2C, 2D, 2E, 3H, 3I and 3J; 2A, 2B, 2C, 2D, 2F, 2G, 3H and 3I; 2A, 2B, 2C, 2D, 2F, 2G, 3H and 3J; 2A, 2B, 2C, 2D, 2F, 2G. 3I and 3J; 2A, 2B, 2C, 2D, 2F, 3H, 3I and 3J; 2A, 2B, 2C, 2D, 2F, 3H, 3I and 3J; 2A, 2B, 2C, 2D, 2G, 3H, 3I and 3J; 2A, 2B, 2C, 2E, 2F, 2G, 3H and 3I; 2A, 2B, 2C, 2E, 2F, 2G, 3H and 3J; 2A, 2B, 2C, 2E, 2F, 2G, 3I and 3J; 2A, 2B, 2C, 2E, 2F, 3H, 3I and 3J; 2A, 2B, 2C, 2E, 2G, 3H, 3I and 3J; 2A, 2B, 2C, 2F, 2G, 3H, 3I and 3J; 2A, 2B, 2D, 2F, 2G, 3H, 3I and 3J; 2A, 2B, 2E, 2F, 2G, 3H, 3I and 3J; 2A, 2C, 2D, 2E, 2F, 2G, 3H and 3I; 2A, 2C, 2D, 2E, 2F, 2G, 3H and 3J; 2A, 2C, 2D, 2E, 2F, 2G, 3I and 3J; 2A, 2C, 2D, 2E, 2F, 3H, 3I and 3J; 2A, 2C, 2D, 2E, 2G, 3H, 3I and 3J; 2A, 2C, 2D, 2F, 2G, 3H, 3I and 3J; 2A, 2C, 2E, 2F, 2G, 3H, 3I and 3J; 2A, 2D, 2E, 2F, 2G, 3H, 3I and 3J; 2B, 2C, 2D, 2E, 2F, 2G, 3H and 3I; 2B, 2C, 2D, 2E, 2F, 2G, 3H and 3J; 2B, 2C, 2D, 2E, 2F, 2G, 3I and 3J; 2B, 2C, 2D, 2E, 2F, 3H, 3I and 3J; 2B, 2C, 2D, 2E, 2G, 3H, 3I and 3J; 2B, 2C, 2D, 2F, 2G, 3H, 3I and 3J; 2B, 2C, 2E, 2F, 2G, 3H, 3I and 3J; or 2B, 2D, 2E, 2F, 2G, 3H, 3I and 3J. In some embodiments, the non-naturally occurring eukaryotic organism, comprises eight or more exogenous nucleic acids, wherein each of the eight or more exogenous nucleic acids encodes a different acetyl-CoA pathway enzyme.

In some embodiments, the acetyl-CoA pathway comprises 2A, 2B, 2C, 2D, 2E, 2F, 2G, 3H and 3I; 2A, 2B, 2C, 2D, 2E, 2F, 2G, 3H and 3J; 2A, 2B, 2C, 2D, 2E, 2F, 2G, 3I and 3J; 2A, 2B, 2C, 2D, 2E, 2F, 3H, 3I and 3J; 2A, 2B, 2C, 2D, 2E, 2G, 3H, 3I and 3J; 2A, 2B, 2C, 2D, 2F, 2G, 3H, 3I and 3J; 2A, 2B, 2C, 2E, 2F, 2G, 3H, 3I and 3J; 2A, 2C, 2D, 2E, 2F, 2G, 3H, 3I and 3J; or 2B, 2C, 2D, 2E, 2F, 2G, 3H, 3I and 3J. In some embodiments, the non-naturally occurring eukaryotic organism, comprises nine or more exogenous nucleic acids, wherein each of the nine or more exogenous nucleic acids encodes a different acetyl-CoA pathway enzyme.

In other embodiments, the acetyl-CoA pathway comprises 2A, 2B, 2C, 2D, 2E, 2F, 2G, 3H, 3I and 3J. In some embodiments, the non-naturally occurring eukaryotic organism, comprises ten or more exogenous nucleic acids, wherein each of the ten or more exogenous nucleic acids encodes a different acetyl-CoA pathway enzyme.

In certain embodiments, the acetyl-CoA pathway comprises 5A, 5B, 5C, 5D, 5E, 5F, 5G, 5H, 5I, 5J or any combination of 5A, 5B, 5C, 5D, 5E, 5F, 5G, 5H, 5I, or 5J thereof, wherein 5A is a pyruvate oxidase (acetate forming); 5B is an acetyl-CoA synthetase, ligase or transferase; 5C is an acetate kinase; 5D is a phosphotransacetylase; 5E is a pyruvate decarboxylase; 5F is an acetaldehyde dehydrogenase; 5G is a pyruvate oxidase (acetyl-phosphate forming); 5H is a pyruvate dehydrogenase, pyruvate:ferredoxin oxidoreductase or pyruvate formate lyase; 5I acetaldehyde dehydrogenase (acylating); and 5J is a threonine aldolase. In certain embodiments, 5B is an acetyl-CoA synthetase. In another embodiment, 5B is an acetyl-CoA ligase. In other embodiments, 5B is an acetyl-CoA transferase. In some embodiments, 5H is a pyruvate dehydrogenase. In other embodiments, 5H is a pyruvate:ferredoxin oxidoreductase. In yet other embodiments, 5H is a pyruvate formate lyase.

In some embodiments, the acetyl-CoA pathway is an acetyl-CoA pathway depicted in FIG. 5. In a specific embodiment, the acetyl-CoA pathway comprises 5A and 5B. In another embodiment, the acetyl-CoA pathway comprises 5A, 5C and 5D. In another embodiment, the acetyl-CoA pathway comprises 5G and 5D. In yet another specific embodiment, the acetyl-CoA pathway comprises 5E, 5F, 5C and 5D. In other embodiments, the acetyl-CoA pathway comprises 5J and 5I. In some embodiments, the acetyl-CoA pathway comprises 5J, 5F and 5B. In yet other specific embodiments, the acetyl-CoA pathway comprises 5H.

In one embodiment, the acetyl-CoA pathway comprises 5A. In another embodiment, the acetyl-CoA pathway comprises 5B. In some embodiments, the acetyl-CoA pathway comprises 5C. In some embodiments, the acetyl-CoA pathway comprises 5D. In some embodiments, the acetyl-CoA pathway comprises 5E. In other embodiments, the acetyl-CoA pathway comprises 5F. In yet other embodiments, the acetyl-CoA pathway comprises 5G. In some embodiments, the acetyl-CoA pathway comprises 5G. In another embodiment, the acetyl-CoA pathway comprises 5H. In some embodiments, the acetyl-CoA pathway comprises 5I. In some embodiments, the acetyl-CoA pathway comprises 5J. In some embodiments, the non-naturally occurring eukaryotic organism, comprises one or more exogenous nucleic acids, wherein each of the one or more exogenous nucleic acids encodes a different acetyl-CoA pathway enzyme.

In some embodiments, the acetyl-CoA pathway comprises: 5A and 5B; 5A and 5C; 5A and 5D; 5A and 5E; 5A and 5F; 5A and 5G; 5A and 5H; 5A and 5I; 5A and 5J; 5B and 5C; 5B and 5D; 5B and 5E; 5B and 5F; 5B and 5G; 5B and 5H; 5B and 5I; 5B and 5J; 5C and 5D; 5C and 5E; 5C and 5F; 5C and 5G; 5C and 5H; 5C and 5I; 5C and 5J; 5D and 5E; 5D and 5F; 5D and 5G; 5D and 5E; 5D and 5F; 5D and 5G; 5D and 5H; 5D and 5I; 5D and 5J; 5E and 5F; 5E and 5G; 5E and 5H; 5E and 5I; 5E and 5J; 5F and 5G; 5F and 5H; 5F and 5I; 5F and 5J; 5G and 5H; 5G and 5I; 5G and 5J; 5H and 5I; 5H and 5I; or 5I and 5J. In some embodiments, the non-naturally occurring eukaryotic organism comprises two or more exogenous nucleic acids, wherein each of the two or more exogenous nucleic acids encodes a different acetyl-CoA pathway enzyme.

In other embodiments, the acetyl-CoA pathway comprises: 5A, 5B and 5C; 5A, 5B and 5D; 5A, 5B and 5E; 5A, 5B and 5F; 5A, 5B and 5G; 5A, 5B and 5H; 5A, 5B and 5I; 5A, 5B and 5J; 5A, 5C and 5D; 5A, 5C and 5E; 5A, 5C and 5F; 5A, 5C and 5G; 5A, 5C and 5H; 5A, 5C and 5I; 5A, 5C and 5J; 5A, 5D and 5E; 5A, 5D and 5F; 5A, 5D and 5G; 5A, 5D and 5H; 5A, 5D and 5I; 5A, 5D and 5J; 5A, 5E and 5F; 5A, 5E and 5G; 5A, 5E and 5H; 5A, 5E and 5I; 5A, 5E and 5J; 5A, 5F and 5G; 5A, 5F and 5H; 5A, 5F and 5I; 5A, 5F and 5J; 5B, 5C and 5D; 5B, 5C and 5E; 5B, 5C and 5F; 5B, 5C and 5G; 5B, 5C and 5H; 5B, 5C and 5I; 5B, 5C and 5J; 5B, 5D and 5E; 5B, 5D and 5F; 5B, 5D and 5G; 5B, 5D and 5H; 5B, 5D and 5I; 5B, 5D and 5J; 5B, 5E and 5F; 5B, 5E and 5G; 5B, 5E and 5H; 5B, 5E and 5I; 5B, 5E and 5J; 5B, 5F and 5G; 5B, 5F and 5H; 5B, 5F and 5I; 5B, 5F and 5J; 5C, 5D and 5E; 5C, 5D and 5F; 5C, 5D and 5G; 5C, 5D and 5H; 5C, 5D and 5I; 5C, 5D and 5J; 5C, 5E and 5F; 5C, 5E and 5G; 5C, 5E and 5H; 5C, 5E and 5I; 5C, 5E and 5J; 5C, 5F and 5G; 5C, 5F and 5H; 5C, 5F and 5I; 5C, 5F and 5J; 5D, 5E and 5F; 5D, 5E and 5G; 5D, 5E and 5H; 5D, 5E and 5I; 5D, 5E and 5J; 5D, 5F and 5G; 5D, 5F and 5H; 5D, 5F and 5I; 5D, 5F and 5J; 5D, 5G and 5H; 5D, 5G and 5I; 5D, 5G and 5J; 5D, 5E and 5F; 5D, 5E and 5G; 5D, 5E and 5H; 5D, 5E and 5I; 5D, 5E and 5J; 5D, 5F and 5G; 5D, 5F and 5H; 5D, 5F and 5I; 5D, 5F and 5J; 5D, 5G and 5H; 5D, 5G and 5I; 5D, 5G and 5J; 5D, 5H and 5I; 5D, 5H and 5J; 5D, 5I and 5J; 5E, 5F and 5G; 5E, 5F and 5H; 5E, 5F and 5I; 5E, 5F and 5J; 5F, 5G and 5H; 5F, 5G and 5I; 5F, 5G and 5J; 5G, 5H and 5I; 5G, 5H and 5J; or 5H, 5I and 5J. In some embodiments, the non-naturally occurring eukaryotic organism comprises three or more exogenous nucleic acids, wherein each of the three or more exogenous nucleic acids encodes a different acetyl-CoA pathway enzyme.

In certain embodiments, the acetyl CoA pathway comprises: 5A, 5B, 5C and 5D; 5A, 5B, 5C and 5E; 5A, 5B, 5C and 5F; 5A, 5B, 5C and 5G; 5A, 5B, 5C and 5H; 5A, 5B, 5C and 5I; 5A, 5B, 5C and 5J; 5A, 5B, 5D and 5E; 5A, 5B, 5D and 5F; 5A, 5B, 5D and 5G; 5A, 5B, 5D and 5H; 5A, 5B, 5D and 5I; 5A, 5B, 5D and 5J; 5A, 5B, 5E and 5F; 5A, 5B, 5E and 5G; 5A, 5B, 5E and 5H; 5A, 5B, 5E and 5I; 5A, 5B, 5E and 5J; 5A, 5B, 5F and 5G; 5A, 5B, 5F and 5H; 5A, 5B, 5F and 5I; 5A, 5B, 5F and 5J; 5A, 5B, 5G and 5H; 5A, 5B, 5G and 5I; 5A, 5B, 5G and 5J; 5A, 5B, 5H and 5I; 5A, 5B, 5H and 5J; 5A, 5B, 5I and 5J; 5A, 5C, 5D and 5E; 5A, 5C, 5D and 5F; 5A, 5C, 5D and 5G; 5A, 5C, 5D and 5H; 5A, 5C, 5D and 5I; 5A, 5C, 5D and 5J; 5A, 5C, 5E and 5F; 5A, 5C, 5E and 5G; 5A, 5C, 5E and 5H; 5A, 5C, 5E and 5I; 5A, 5C, 5E and 5J; 5A, 5C, 5F and 5G; 5A, 5C, 5F and 5H; 5A, 5C, 5F and 5I; 5A, 5C, 5F and 5J; 5A, 5C, 5G and 5H; 5A, 5C, 5G and 5I; 5A, 5C, 5G and 5J; 5A, 5C, 5H and 5I; 5A, 5C, 5H and 5J; 5A, 5C, 5I and 5J; 5A, 5D, 5E and 5F; 5A, 5D, 5E and 5G; 5A, 5D, 5E and 5H; 5A, 5D, 5E and 5I; 5A, 5D, 5E and 5J; 5A, 5D, 5F and 5G; 5A, 5D, 5F and 5H; 5A, 5D, 5F and 5I; 5A, 5D, 5F and 5J; 5A, 5D, 5G and 5H; 5A, 5D, 5G and 5I; 5A, 5D, 5G and 5J; 5A, 5D, 5H and 5I; 5A, 5D, 5H and 5J; 5A, 5D, 5I and 5J; 5A, 5E, 5F and 5G; 5A, 5E, 5F and 5H; 5A, 5E, 5F and 5I; 5A, 5E, 5F and 5J; 5A, 5E, 5G and 5H; 5A, 5E, 5G and 5I; 5A, 5E, 5G and 5J; 5A, 5E, 5H and 5I; 5A, 5E, 5H and 5J; 5A, 5E, 5I and 5J; 5A, 5F, 5G and 5H; 5A, 5F, 5G and 5I; 5A, 5F, 5G and 5J; 5A, 5F, 5H and 5I; 5A, 5F, 5H and 5J; 5A, 5F, 5I and 5J; 5A, 5G, 5H and 5I; 5A, 5G, 5H and 5J; 5A, 5G, 5I and 5J; 5A, 5H, 5I and 5J; 5B, 5C, 5D and 5E; 5B, 5C, 5D and 5F; 5B, 5C, 5D and 5G; 5B, 5C, 5D and 5H; 5B, 5C, 5D and 5I; 5B, 5C, 5D and 5J; 5B, 5C, 5E and 5F; 5B, 5C, 5E and 5G; 5B, 5C, 5E and 5H; 5B, 5C, 5E and 5I; 5B, 5C, 5E and 5J; 5B, 5C, 5F and 5G; 5B, 5C, 5F and 5H; 5B, 5C, 5F and 5I; 5B, 5C, 5F and 5J; 5B, 5C, 5G and 5H; 5B, 5C, 5G and 5I; 5B, 5C, 5G and 5J; 5B, 5C, 5H and 5I; 5B, 5C, 5H and 5J; 5B, 5C, 5I and 5J; 5B, 5D, 5E and 5F; 5B, 5D, 5E and 5G; 5B, 5D, 5E and 5H; 5B, 5D, 5E and 5I; 5B, 5D, 5E and 5J; 5B, 5D, 5F and 5G; 5B, 5D, 5F and 5H; 5B, 5D, 5F and 5I; 5B, 5D, 5F and 5J; 5B, 5E, 5F and 5G; 5B, 5E, 5F and 5H; 5B, 5E, 5F and 5I; 5B, 5E, 5F and 5J; 5B, 5E, 5G and 5H; 5B, 5E, 5G and 5I; 5B, 5E, 5G and 5J; 5B, 5E, 5H and 5I; 5B, 5E, 5H and 5J; 5B, 5E, 5I and 5J; 5B, 5F, 5G and 5H; 5B, 5F, 5G and 5I; 5B, 5F, 5G and 5J; 5B, 5G, 5H and 5I; 5B, 5G, 5H and 5J; 5B, 5H, 5I and 5J; 5C, 5D, 5E and 5F; 5C, 5D, 5E and 5G; 5C, 5D, 5E and 5H; 5C, 5D, 5E and 5I; 5C, 5D, 5E and 5J; 5C, 5D, 5F and 5G; 5C, 5D, 5F and 5H; 5C, 5D, 5F and 5I; 5C, 5D, 5F and 5J; 5C, 5D, 5G and 5H; 5C, 5D, 5G and 5I; 5C, 5D, 5G and 5J; 5C, 5D, 5H and 5I; 5C, 5D, 5H and 5J; 5C, 5D, 5I and 5J; 5D, 5E, 5F and 5G; 5D, 5E, 5F and 5H; 5D, 5E, 5F and 5I; 5D, 5E, 5F and 5J; 5D, 5E, 5G and 5H; 5D, 5E. 5G and 5I; 5D, 5E, 5G and 5J; 5D, 5E, 5H and 5I; 5D, 5E, 5H and 5J; 5D, 5E, 5I and 5J; 5E, 5F, 5G and 5H; 5E, 5F, 5G and 5I; 5E, 5F, 5G and 5J; 5E, 5F, 5H and 5I; 5E, 5F, 5H and 5J; 5E, 5F, 5I and 5J; 5F, 5G, 5H and 5I; 5F, 5G, 5H and 5J; 5F, 5G, 5I and 5J; or 5G, 5H, 5I and 5J. In some embodiments, the non-naturally occurring eukaryotic organism comprises four or more exogenous nucleic acids, wherein each of the four or more exogenous nucleic acids encodes a different acetyl-CoA pathway enzyme.

In other embodiments, the acetyl CoA pathway comprises: 5A, 5B, 5C, 5D and 5E; 5A, 5B, 5C, 5D and 5F; 5A, 5B, 5C, 5D and 5G; 5A, 5B, 5C, 5D and 5H; 5A, 5B, 5C, 5D and 5I; 5A, 5B, 5C, 5D and 5J; 5A, 5B, 5C, 5E and 5F; 5A, 5B, 5C, 5E and 5G; 5A, 5B, 5C, 5E and 5H; 5A, 5B, 5C, 5E and 5I; 5A, 5B, 5C, 5E and 5J; 5A, 5B, 5C, 5F and 5G; 5A, 5B, 5C, 5F and 5H; 5A, 5B, 5C, 5F and 5I; 5A, 5B, 5C, 5F and 5J; 5A, 5B, 5C, 5G and 5H; 5A, 5B, 5C, 5G and 5I; 5A, 5B, 5C, 5G and 5J; 5A, 5B, 5C, 5H and 5I; 5A, 5B, 5C, 5H and 5J; 5A, 5B, 5C, 5I and 5J; 5A, 5B, 5D, 5E and 5H; 5A, 5B, 5D, 5E and 5I; 5A, 5B, 5D, 5E and 5J; 5A, 5B, 5D, 5F and 5G; 5A, 5B, 5D, 5F and 5H; 5A, 5B, 5D, 5F and 5I; 5A, 5B, 5D, 5F and 5J; 5A, 5B, 5D, 5G and 5H; 5A, 5B, 5D, 5G and 5I; 5A, 5B, 5D, 5G and 5J; 5A, 5B, 5D, 5H and 5I; 5A, 5B, 5D, 5H and 5J; 5A, 5B, 5D, 5I and 5J; 5A, 5B, 5E, 5F and 5G; 5A, 5B, 5E, 5F and 5H; 5A, 5B, 5E, 5F and 5I; 5A, 5B, 5E, 5F and 5J; 5A, 5B, 5E, 5G and 5H; 5A, 5B, 5E, 5G and 5I; 5A, 5B, 5E, 5G and 5J; 5A, 5B, 5E, 5H and 5I; 5A, 5B, 5E, 5H and 5J; 5A, 5B, 5E, 5I and 5J; 5A, 5B, 5F, 5G and 5H; 5A, 5B, 5F, 5G and 5I; 5A, 5B, 5F, 5G and 5J; 5A, 5B, 5F, 5H and 5I; 5A, 5B, 5F, 5H and 5J; 5A, 5B, 5F, 5I and 5J; 5A, 5B, 5G, 5H and 5I; 5A, 5B, 5G, 5H and 5J; 5A, 5B, 5G, 5I and 5J; 5A, 5B, 5H, 5I and 5J; 5A, 5C, 5D, 5E and 5F; 5A, 5C, 5D, 5E and 5G; 5A, 5C, 5D, 5E and 5H; 5A, 5C, 5D, 5E and 5I; 5A, 5C, 5D, 5E and 5J; 5A, 5C, 5D, 5F and 5G; 5A, 5C, 5D, 5F and 5H; 5A, 5C, 5D, 5F and 5I; 5A, 5C, 5D, 5F and 5J; 5A, 5C, 5D, 5G and 5H; 5A, 5C, 5D, 5G and 5I; 5A, 5C, 5D, 5G and 5J; 5A, 5C, 5D, 5H and 5I; 5A, 5C, 5D, 5H and 5J; 5A, 5C, 5D, 5I and 5J; 5A, 5C, 5E, 5F and 5G; 5A, 5C, 5E, 5F and 5H; 5A, 5C, 5E, 5F and 5I; 5A, 5C, 5E, 5F and 5J; 5A, 5C, 5E, 5G and 5H; 5A, 5C, 5E, 5G and 5I; 5A, 5C, 5E, 5G and 5J; 5A, 5C, 5E, 5H and 5I; 5A, 5C, 5E, 5H and 5J; 5A, 5C, 5E, 5I and 5J; 5A, 5C, 5F, 5G and 5H; 5A, 5C, 5F, 5G and 5I; 5A, 5C, 5F, 5G and 5J; 5A, 5C, 5F, 5H and 5I; 5A, 5C, 5F, 5H and 5J; 5A, 5C, 5F, 5I and 5J; 5A, 5C, 5G, 5H and 5I; 5A, 5C, 5G, 5H and 5J; 5A, 5C, 5G, 5I and 5J; 5A, 5C, 5H, 5I and 5J; 5A, 5D, 5E, 5F and 5G; 5A, 5D, 5E, 5F and 5H; 5A, 5D, 5E, 5F and 5I; 5A, 5D, 5E, 5F and 5J; 5A, 5D, 5E, 5G and 5H; 5A, 5D, 5E, 5G and 5I; 5A, 5D, 5E, 5G and 5J; 5A, 5D, 5E, 5H and 5I; 5A, 5D, 5E, 5H and 5J; 5A, 5D, 5E, 5I and 5J; 5A, 5D, 5F, 5G and 5H; 5A, 5D, 5F, 5G and 5I; 5A, 5D, 5F, 5G and 5J; 5A, 5D, 5F, 5H and 5I; 5A, 5D, 5F, 5H and 5J; 5A, 5D, 5F, 5I and 5J; 5A, 5D, 5G, 5H and 5I; 5A, 5D, 5G, 5H and 5J; 5A, 5D, 5G, 5I and 5J; 5A, 5D, 5H, 5I and 5J; 5A, 5E, 5F, 5G and 5H; 5A, 5E, 5F, 5G and 5I; 5A, 5E, 5F, 5G and 5J; 5A, 5E, 5F, 5H and 5I; 5A, 5E, 5F, 5H and 5J; 5A, 5E, 5F, 5I and 5J; 5A, 5E, 5G, 5H and 5I; 5A, 5E, 5G, 5H and 5J; 5A, 5E, 5G, 5I and 5J; 5A, 5E, 5H, 5I and 5J; 5A, 5F, 5G, 5H and 5I; 5A, 5F, 5G, 5H and 5J; 5A, 5F, 5G, 5I and 5J; 5A, 5F, 5H, 5I and 5J; 5A, 5G, 5H, 5I and 5J; 5B, 5C, 5D, 5E and 5F; 5B, 5C, 5D, 5E and 5G; 5B, 5C, 5D, 5E and 5H; 5B, 5C, 5D, 5E and 5I; 5B, 5C, 5D, 5E and 5J; 5B, 5C, 5D, 5F and 5G; 5B, 5C, 5D, 5F and 5H; 5B, 5C, 5D, 5F and 5I; 5B, 5C, 5D, 5F and 5J; 5B, 5C, 5D, 5G and 5H; 5B, 5C, 5D, 5G and 5I; 5B, 5C, 5D, 5G and 5J; 5B, 5C, 5D, 5H and 5I; 5B, 5C, 5D, 5H and 5J; 5B, 5C, 5D, 5I and 5J; 5B, 5C, 5E, 5F and 5G; 5B, 5C, 5E, 5F and 5H; 5B, 5C, 5E, 5F and 5I; 5B, 5C, 5E, 5F and 5J; 5B, 5C, 5E, 5G and 5H; 5B, 5C, 5E, 5G and 5I; 5B, 5C, 5E, 5G and 5J; 5B, 5C, 5E, 5H and 5I; 5B, 5C, 5E, 5H and 5J; 5B, 5C, 5E, 5I and 5J; 5B, 5C, 5F, 5G and 5H; 5B, 5C, 5F, 5G and 5I; 5B, 5C, 5F, 5G and 5J; 5B, 5C, 5F, 5H and 5I; 5B, 5C, 5F, 5H and 5J; 5B, 5C, 5F, 5I and 5J; 5B, 5C, 5G, 5H and 5I; 5B, 5C, 5G, 5H and 5J; 5B, 5C, 5G, 5I and 5J; 5B, 5C, 5H, 5I and 5J; 5B, 5D, 5E, 5F and 5G; 5B, 5D, 5E, 5F and 5H; 5B, 5D, 5E, 5F and 5I; 5B, 5D, 5E, 5F and 5J; 5B, 5D, 5E, 5G and 5H; 5B, 5D, 5E, 5G and 5I; 5B, 5D, 5E, 5G and 5J; 5B, 5D, 5E, 5H and 5I; 5B, 5D, 5E, 5H and 5J; 5B, 5D, 5E, 5I and 5J; 5B, 5D, 5F, 5G and 5H; 5B, 5D, 5F, 5G and 5I; 5B, 5D, 5F, 5G and 5J; 5B, 5D, 5F, 5H and 5I; 5B, 5D, 5F, 5H and 5J; 5B, 5D, 5F, 5I and 5J; 5B, 5E, 5F, 5G and 5H; 5B, 5E, 5F, 5G and 5I; 5B, 5E, 5F, 5G and 5J; 5B, 5E, 5F, 5H and 5I; 5B, 5E, 5F, 5H and 5J; 5B, 5E, 5F, 5I and 5J; 5B, 5E, 5G, 5H and 5I; 5B, 5E, 5G, 5H and 5J; 5B, 5E, 5G, 5I and 5J; 5B, 5E, 5H, 5I and 5J; 5B, 5F, 5G, 5H and 5I; 5B, 5F, 5G, 5H and 5J; 5B, 5F, 5G, 5I and 5J; 5B, 5G, 5H, 5I and 5J; 5C, 5D, 5E, 5F and 5H; 5C, 5D, 5E, 5F and 5I; 5C, 5D, 5E, 5F and 5J; 5C, 5D, 5E, 5G and 5H; 5C, 5D, 5E, 5G and 5I; 5C, 5D, 5E, 5G and 5J; 5C, 5D, 5E, 5H and 5I; 5C, 5D, 5E, 5H and 5J; 5C, 5D, 5E, 5I and 5J; 5C, 5D, 5F, 5G and 5H; 5C, 5D, 5F, 5G and 5I; 5C, 5D, 5F, 5G and 5J; 5C, 5D, 5F, 5H and 5I; 5C, 5D, 5F, 5H and 5J; 5C, 5D, 5F, 5I and 5J; 5C, 5D, 5G, 5H and 5I; 5C, 5D, 5G, 5H and 5J; 5C, 5D, 5G, 5I and 5J; 5C, 5D, 5H, 5I and 5J; 5D, 5E, 5F, 5G and 5H; 5D, 5E, 5F, 5G and 5I; 5D, 5E, 5F, 5G and 5J; 5D, 5E, 5F, 5H and 5I; 5D, 5E, 5F, 5H and 5J; 5D, 5E, 5F, 5I and 5J; 5D, 5E, 5G, 5H and 5I; 5D, 5E, 5G, 5H and 5J; 5D, 5E. 5G, 5I and 5J; 5D, 5E, 5H, 5I and 5J; 5E, 5F, 5G, 5H and 5I; 5E, 5F, 5G, 5H and 5J; 5E, 5F, 5G, 5I and 5J; 5E, 5F, 5H, 5I and 5J; or 5F, 5G, 5H, 5I and 5J. In some embodiments, the non-naturally occurring eukaryotic organism, comprises five or more exogenous nucleic acids, wherein each of the five or more exogenous nucleic acids encodes a different acetyl-CoA pathway enzyme.

In yet other embodiments, the acetyl-CoA pathway comprises: 5A, 5B, 5C, 5D, 5E and 5F; 5A, 5B, 5C, 5D, 5E and 5G; 5A, 5B, 5C, 5D, 5E and 5H; 5A, 5B, 5C, 5D, 5E and 5I; 5A, 5B, 5C, 5D, 5E and 5J; 5A, 5B, 5C, 5D, 5F and 5G; 5A, 5B, 5C, 5D, 5F and 5H; 5A, 5B, 5C, 5D, 5F and 5I; 5A, 5B, 5C, 5D, 5F and 5H; 5A, 5B, 5C, 5D, 5G and 5H; 5A, 5B, 5C, 5D, 5G and 5I; 5A, 5B, 5C, 5D, 5G and 5J; 5A, 5B, 5C, 5D, 5H and 5I; 5A, 5B, 5C, 5D, 5H and 5J; 5A, 5B, 5C, 5D, 5I and 5J; 5A, 5B, 5C, 5E, 5F and 5G; 5A, 5B, 5C, 5E, 5F and 5H; 5A, 5B, 5C, 5E, 5F and 5I; 5A, 5B, 5C, 5E, 5F and 5J; 5A, 5B, 5C, 5E, 5G and 5H; 5A, 5B, 5C, 5E, 5G and 5I; 5A, 5B, 5C, 5E, 5G and 5J; 5A, 5B, 5C, 5E, 5H and 5I; 5A, 5B, 5C, 5E, 5H and 5J; 5A, 5B, 5C, 5E, 5I and 5J; 5A, 5B, 5C, 5F, 5G and 5H; 5A, 5B, 5C, 5F, 5G and 5I; 5A, 5B, 5C, 5F, 5G and 5J; 5A, 5B, 5C, 5F, 5H and 5I; 5A, 5B, 5C, 5F, 5H and 5J; 5A, 5B, 5C, 5F, 5I and 5J; 5A, 5B, 5C, 5G, 5H and 5I; 5A, 5B, 5C, 5G, 5H and 5J; 5A, 5B, 5C, 5G, 5I and 5J; 5A, 5B, 5C, 5H, 5I and 5J; 5A, 5B, 5D, 5E, 5H and 5I; 5A, 5B, 5D, 5E, 5H and 5J; 5A, 5B, 5D, 5E, 5I and 5J; 5A, 5B, 5D, 5F, 5G and 5H; 5A, 5B, 5D, 5F, 5G and 5I; 5A, 5B, 5D, 5F, 5G and 5J; 5A, 5B, 5D, 5F, 5H and 5I; 5A, 5B, 5D, 5F, 5H and 5J; 5A, 5B, 5D, 5F, 5I and 5J; 5A, 5B, 5D, 5G, 5H and 5I; 5A, 5B, 5D, 5G, 5H and 5J; 5A, 5B, 5D, 5G, 5I and 5J; 5A, 5B, 5D, 5H, 5I and 5J; 5A, 5B, 5E, 5F, 5G and 5H; 5A, 5B, 5E, 5F, 5G and 5I; 5A, 5B, 5E, 5F, 5G and 5J; 5A, 5B, 5E, 5F, 5H and 5I; 5A, 5B, 5E, 5F, 5H and 5J; 5A, 5B, 5E, 5F, 5I and 5J; 5A, 5B, 5E, 5G, 5H and 5I; 5A, 5B, 5E, 5G, 5H and 5J; 5A, 5B, 5E, 5G, 5I and 5J; 5A, 5B, 5E, 5H, 5I and 5J; 5A, 5B, 5F, 5G, 5H and 5I; 5A, 5B, 5F, 5G, 5H and 5J; 5A, 5B, 5F, 5G, 5I and 5J; 5A, 5B, 5F, 5H, 5I and 5J; 5A, 5B, 5G, 5H, 5I and 5J; 5A, 5C, 5D, 5E, 5F and 5G; 5A, 5C, 5D, 5E, 5F and 5H; 5A, 5C, 5D, 5E, 5F and 5I; 5A, 5C, 5D, 5E, 5F and 5J; 5A, 5C, 5D, 5E, 5G and 5H; 5A, 5C, 5D, 5E, 5G and 5I; 5A, 5C, 5D, 5E, 5G and 5J; 5A, 5C, 5D, 5E, 5H and 5I; 5A, 5C, 5D, 5E, 5H and 5J; 5A, 5C, 5D, 5E, 5I and 5J; 5A, 5C, 5D, 5F, 5G and 5H; 5A, 5C, 5D, 5F, 5G and 5I; 5A, 5C, 5D, 5F, 5G and 5J; 5A, 5C, 5D, 5F, 5H and 5I; 5A, 5C, 5D, 5F, 5H and 5J; 5A, 5C, 5D, 5F, 5I and 5J; 5A, 5C, 5D, 5G, 5H and 5I; 5A, 5C, 5D, 5G, 5H and 5J; 5A, 5C, 5D, 5G, 5I and 5J; 5A, 5C, 5D, 5H, 5I and 5J; 5A, 5C, 5E, 5F, 5G and 5H; 5A, 5C, 5E, 5F, 5G and 5I; 5A, 5C, 5E, 5F, 5G and 5J; 5A, 5C, 5E, 5F, 5H and 5I; 5A, 5C, 5E, 5F, 5H and 5J; 5A, 5C, 5E, 5F, 5I and 5J; 5A, 5C, 5E, 5G, 5H and 5I; 5A, 5C, 5E, 5G, 5H and 5J; 5A, 5C, 5E, 5G, 5I and 5J; 5A, 5C, 5E, 5H, 5I and 5J; 5A, 5C, 5F, 5G, 5H and 5I; 5A, 5C, 5F, 5G, 5H and 5J; 5A, 5C, 5F, 5G, 5I and 5J; 5A, 5C, 5F, 5H, 5I and 5J; 5A, 5C, 5G, 5H, 5I and 5J; 5A, 5D, 5E, 5F, 5G and 5H; 5A, 5D, 5E, 5F, 5G and 5I; 5A, 5D, 5E, 5F, 5G and 5J; 5A, 5D, 5E, 5F, 5H and 5I; 5A, 5D, 5E, 5F, 5H and 5J; 5A, 5D, 5E, 5F, 5I and 5J; 5A, 5D, 5E, 5G, 5H and 5I; 5A, 5D, 5E, 5G, 5H and 5J; 5A, 5D, 5E, 5G, 5I and 5J; 5A, 5D, 5E, 5H, 5I and 5J; 5A, 5D, 5F, 5G, 5H and 5I; 5A, 5D, 5F, 5G, 5H and 5J; 5A, 5D, 5F, 5G, 5I and 5J; 5A, 5D, 5F, 5H, 5I and 5J; 5A, 5D, 5G, 5H, 5I and 5J; 5A, 5E, 5F, 5G, 5H and 5I; 5A, 5E, 5F, 5G, 5H and 5J; 5A, 5E, 5F, 5G, 5I and 5J; 5A, 5E, 5F, 5H, 5I and 5J; 5A, 5E, 5G, 5H, 5I and 5J; 5A, 5F, 5G, 5H, 5I and 5J; 5B, 5C, 5D, 5E, 5F and 5G; 5B, 5C, 5D, 5E, 5F and 5H; 5B, 5C, 5D, 5E, 5F and 5I; 5B, 5C, 5D, 5E, 5F and 5J; 5B, 5C, 5D, 5E, 5G and 5H; 5B, 5C, 5D, 5E, 5G and 5I; 5B, 5C, 5D, 5E, 5G and 5J; 5B, 5C, 5D, 5E, 5H and 5I; 5B, 5C, 5D, 5E, 5H and 5I; 5B, 5C, 5D, 5E, 5I and 5J; 5B, 5C, 5D, 5F, 5G and 5H; 5B, 5C, 5D, 5F, 5G and 5I; 5B, 5C, 5D, 5F, 5G and 5J; 5B, 5C, 5D, 5F, 5H and 5I; 5B, 5C, 5D, 5F, 5H and 5J; 5B, 5C, 5D, 5F, 5I and 5J; 5B, 5C, 5D, 5G, 5H and 5I; 5B, 5C, 5D, 5G, 5H and 5J; 5B, 5C, 5D, 5G, 5I and 5J; 5B, 5C, 5D, 5H, 5I and 5J; 5B, 5C, 5E, 5F, 5G and 5H; 5B, 5C, 5E, 5F, 5G and 5I; 5B, 5C, 5E, 5F, 5G and 5J; 5B, 5C, 5E, 5F, 5H and 5I; 5B, 5C, 5E, 5F, 5H and 5J; 5B, 5C, 5E, 5F, 5I and 5J; 5B, 5C, 5E, 5G, 5H and 5I; 5B, 5C, 5E, 5G, 5H and 5J; 5B, 5C, 5E, 5G, 5I and 5J; 5B, 5C, 5E, 5H, 5I and 5J; 5B, 5C, 5F, 5G, 5H and 5I; 5B, 5C, 5F, 5G, 5H and 5J; 5B, 5C, 5F, 5G, 5I and 5J; 5B, 5C, 5F, 5H, 5I and 5J; 5B, 5C, 5G, 5H, 5I and 5J; 5B, 5D, 5E, 5F, 5G and 5H; 5B, 5D, 5E, 5F, 5G and 5I; 5B, 5D, 5E, 5F, 5G and 5J; 5B, 5D, 5E, 5F, 5H and 5I; 5B, 5D, 5E, 5F, 5H and 5J; 5B, 5D, 5E, 5F, 5I and 5J; 5B, 5D, 5E, 5G, 5H and 5I; 5B, 5D, 5E, 5G, 5H and 5J; 5B, 5D, 5E, 5G, 5I and 5J; 5B, 5D, 5E, 5H, 5I and 5J; 5B, 5D, 5F, 5G, 5H and 5I; 5B, 5D, 5F, 5G, 5H and 5J; 5B, 5D, 5F, 5G, 5I and 5J; 5B, 5D, 5F, 5H, 5I and 5J; 5B, 5E, 5F, 5G, 5H and 5I; 5B, 5E, 5F, 5G, 5H and 5J; 5B, 5E, 5F, 5G, 5I and 5J; 5B, 5E, 5F, 5H, 5I and 5J; 5B, 5E, 5G, 5H, 5I and 5J; 5B, 5F, 5G, 5H, 5I and 5J; 5C, 5D, 5E, 5F, 5H and 5I; 5C, 5D, 5E, 5F, 5H and 5J; 5C, 5D, 5E, 5F, 5I and 5J; 5C, 5D, 5E, 5G, 5H and 5I; 5C, 5D, 5E, 5G, 5H and 5J; 5C, 5D, 5E, 5G, 5I and 5J; 5C, 5D, 5E, 5H, 5I and 5J; 5C, 5D, 5F, 5G, 5H and 5I; 5C, 5D, 5F, 5G, 5H and 5J; 5C, 5D, 5F, 5G, 5I and 5J; 5C, 5D, 5F, 5H, 5I and 5J; 5C, 5D, 5G, 5H, 5I and 5J; 5D, 5E, 5F, 5G, 5H and 5I; 5D, 5E, 5F, 5G, 5H and 5J; 5D, 5E, 5F, 5G, 5I and 5J; 5D, 5E, 5F, 5H, 5I and 5J; 5D, 5E, 5G, 5H, 5I and 5J; or 5E, 5F, 5G, 5H, 5I and 5J. In some embodiments, the non-naturally occurring eukaryotic organism, comprises six or more exogenous nucleic acids, wherein each of the six or more exogenous nucleic acids encodes a different acetyl-CoA pathway enzyme.

In some embodiments, the acetyl-CoA pathway comprises: 5A, 5B, 5C, 5D, 5E, 5F and 5G; 5A, 5B, 5C, 5D, 5E, 5F and 5H; 5A, 5B, 5C, 5D, 5E, 5F and 5I; 5A, 5B, 5C, 5D, 5E, 5F and 5J; 5A, 5B, 5C, 5D, 5E, 5G and 5H; 5A, 5B, 5C, 5D, 5E, 5G and 5I; 5A, 5B, 5C, 5D, 5E, 5G and 5J; 5A, 5B, 5C, 5D, 5E, 5H and 5I; 5A, 5B, 5C, 5D, 5E, 5H and 5J; 5A, 5B, 5C, 5D, 5E, 5I and 5J; 5A, 5B, 5C, 5D, 5F, 5G and 5H; 5A, 5B, 5C, 5D, 5F, 5G and 5I; 5A, 5B, 5C, 5D, 5F, 5G and 5J; 5A, 5B, 5C, 5D, 5F, 5H and 5I; 5A, 5B, 5C, 5D, 5F, 5H and 5J; 5A, 5B, 5C, 5D, 5F, 5I and 5J; 5A, 5B, 5C, 5D, 5F, 5H and 5I; 5A, 5B, 5C, 5D, 5F, 5H and 5J; 5A, 5B, 5C, 5D, 5G, 5H and 5I; 5A, 5B, 5C, 5D, 5G, 5H and 5J; 5A, 5B, 5C, 5D, 5G, 5I and 5J; 5A, 5B, 5C, 5D, 5H, 5I and 5J; 5A, 5B, 5C, 5E, 5F, 5G and 5H; 5A, 5B, 5C, 5E, 5F, 5G and 5I; 5A, 5B, 5C, 5E, 5F, 5G and 5J; 5A, 5B, 5C, 5E, 5F, 5H and 5I; 5A, 5B, 5C, 5E, 5F, 5H and 5J; 5A, 5B, 5C, 5E, 5F, 5I and 5J; 5A, 5B, 5C, 5E, 5G, 5H and 5I; 5A, 5B, 5C, 5E, 5G, 5H and 5J; 5A, 5B, 5C, 5E, 5G, 5I and 5J; 5A, 5B, 5C, 5E, 5H, 5I and 5J; 5A, 5B, 5C, 5F, 5G, 5H and 5I; 5A, 5B, 5C, 5F, 5G, 5H and 5J; 5A, 5B, 5C, 5F, 5G, 5I and 5J; 5A, 5B, 5C, 5F, 5H, 5I and 5J; 5A, 5B, 5C, 5G, 5H, 5I and 5J; 5A, 5B, 5D, 5E, 5H, 5I and 5J; 5A, 5B, 5D, 5F, 5G, 5H and 5I; 5A, 5B, 5D, 5F, 5G, 5H and 5J; 5A, 5B, 5D, 5F, 5G, 5I and 5J; 5A, 5B, 5D, 5F, 5H, 5I and 5J; 5A, 5B, 5D, 5G, 5H, 5I and 5J; 5A, 5B, 5E, 5F, 5G, 5H and 5I; 5A, 5B, 5E, 5F, 5G, 5H and 5J; 5A, 5B, 5E, 5F, 5G, 5I and 5J; 5A, 5B, 5E, 5F, 5H, 5I and 5J; 5A, 5B, 5E, 5G, 5H, 5I and 5J; 5A, 5B, 5F, 5G, 5H, 5I and 5J; 5A, 5C, 5D, 5E, 5F, 5G and 5H; 5A, 5C, 5D, 5E, 5F, 5G and 5I; 5A, 5C, 5D, 5E, 5F, 5G and 5J; 5A, 5C, 5D, 5E, 5F, 5H and 5I; 5A, 5C, 5D, 5E, 5F, 5H and 5J; 5A, 5C, 5D, 5E, 5F, 5I and 5J; 5A, 5C, 5D, 5E, 5G, 5H and 5I; 5A, 5C, 5D, 5E, 5G, 5H and 5J; 5A, 5C, 5D, 5E, 5G, 5I and 5J; 5A, 5C, 5D, 5E, 5H, 5I and 5J; 5A, 5C, 5D, 5F, 5G, 5H and 5I; 5A, 5C, 5D, 5F, 5G, 5H and 5J; 5A, 5C, 5D, 5F, 5G, 5I and 5J; 5A, 5C, 5D, 5F, 5H, 5I and 5J; 5A, 5C, 5D, 5G, 5H, 5I and 5J; 5A, 5C, 5E, 5F, 5G, 5H and 5I; 5A, 5C, 5E, 5F, 5G, 5H and 5J; 5A, 5C, 5E, 5F, 5G, 5I and 5J; 5A, 5C, 5E, 5F, 5H, 5I and 5J; 5A, 5C, 5E, 5G, 5H, 5I and 5J; 5A, 5C, 5F, 5G, 5H, 5I and 5J; 5A, 5D, 5E, 5F, 5G, 5H and 5I; 5A, 5D, 5E, 5F, 5G, 5H and 5J; 5A, 5D, 5E, 5F, 5G, 5I and 5J; 5A, 5D, 5E, 5F, 5H, 5I and 5J; 5A, 5D, 5E, 5G, 5H, 5I and 5J; 5A, 5D, 5F, 5G, 5H, 5I and 5J; 5A, 5E, 5F, 5G, 5H, 5I and 5J; 5B, 5C, 5D, 5E, 5F, 5G and 5H; 5B, 5C, 5D, 5E, 5F, 5G and 5I; 5B, 5C, 5D, 5E, 5F, 5G and 5J; 5B, 5C, 5D, 5E, 5F, 5H and 5I; 5B, 5C, 5D, 5E, 5F, 5H and 5J; 5B, 5C, 5D, 5E, 5F, 5I and 5J; 5B, 5C, 5D, 5E, 5G, 5H and 5I; 5B, 5C, 5D, 5E, 5G, 5H and 5J; 5B, 5C, 5D, 5E, 5G, 5I and 5J; 5B, 5C, 5D, 5E, 5H, 5I and 5J; 5B, 5C, 5D, 5F, 5G, 5H and 5I; 5B, 5C, 5D, 5F, 5G, 5H and 5J; 5B, 5C, 5D, 5F, 5G, 5I and 5J; 5B, 5C, 5D, 5F, 5H, 5I and 5J; 5B, 5C, 5D, 5G, 5H, 5I and 5J; 5B, 5C, 5E, 5F, 5G, 5H and 5I; 5B, 5C, 5E, 5F, 5G, 5H and 5J; 5B, 5C, 5E, 5F, 5G, 5I and 5J; 5B, 5C, 5E, 5F, 5H, 5I and 5J; 5B, 5C, 5E, 5G, 5H, 5I and 5J; 5B, 5C, 5F, 5G, 5H, 5I and 5J; 5B, 5D, 5E, 5F, 5G, 5H and 5I; 5B, 5D, 5E, 5F, 5G, 5H and 5J; 5B, 5D, 5E, 5F, 5G, 5I and 5J; 5B, 5D, 5E, 5F, 5H, 5I and 5J; 5B, 5D, 5E, 5G, 5H, 5I and 5J; 5B, 5D, 5F, 5G, 5H, 5I and 5J; 5B, 5E, 5F, 5G, 5H, 5I and 5J; 5C, 5D, 5E, 5F, 5H, 5I and 5J; 5C, 5D, 5E, 5G, 5H, 5I and 5J; 5C, 5D, 5F, 5G, 5H, 5I and 5J; or 5D, 5E, 5F, 5G, 5H, 5I and 5J. In some embodiments, the non-naturally occurring eukaryotic organism, comprises seven or more exogenous nucleic acids, wherein each of the seven or more exogenous nucleic acids encodes a different acetyl-CoA pathway enzyme.

In certain embodiments, the acetyl-CoA pathway comprises: 5A, 5B, 5C, 5D, 5E, 5F, 5G and 5H; 5A, 5B, 5C, 5D, 5E, 5F, 5G and 5I; 5A, 5B, 5C, 5D, 5E, 5F, 5G and 5J; 5A, 5B, 5C, 5D, 5E, 5F, 5H and 5I; 5A, 5B, 5C, 5D, 5E, 5F, 5H and 5J; 5A, 5B, 5C, 5D, 5E, 5F, 5I and 5J; 5A, 5B, 5C, 5D, 5E, 5G, 5H and 5I; 5A, 5B, 5C, 5D, 5E, 5G, 5H and 5J; 5A, 5B, 5C, 5D, 5E, 5G, 5I and 5J; 5A, 5B, 5C, 5D, 5E, 5H, 5I and 5J; 5A, 5B, 5C, 5D, 5F, 5G, 5H and 5I; 5A, 5B, 5C, 5D, 5F, 5G, 5H and 5J; 5A, 5B, 5C, 5D, 5F, 5G. 5I and 5J; 5A, 5B, 5C, 5D, 5F, 5H, 5I and 5J; 5A, 5B, 5C, 5D, 5F, 5H, 5I and 5J; 5A, 5B, 5C, 5D, 5G, 5H, 5I and 5J; 5A, 5B, 5C, 5E, 5F, 5G, 5H and 5I; 5A, 5B, 5C, 5E, 5F, 5G, 5H and 5J; 5A, 5B, 5C, 5E, 5F, 5G, 5I and 5J; 5A, 5B, 5C, 5E, 5F, 5H, 5I and 5J; 5A, 5B, 5C, 5E, 5G, 5H, 5I and 5J; 5A, 5B, 5C, 5F, 5G, 5H, 5I and 5J; 5A, 5B, 5D, 5F, 5G, 5H, 5I and 5J; 5A, 5B, 5E, 5F, 5G, 5H, 5I and 5J; 5A, 5C, 5D, 5E, 5F, 5G, 5H and 5I; 5A, 5C, 5D, 5E, 5F, 5G, 5H and 5J; 5A, 5C, 5D, 5E, 5F, 5G, 5I and 5J; 5A, 5C, 5D, 5E, 5F, 5H, 5I and 5J; 5A, 5C, 5D, 5E, 5G, 5H, 5I and 5J; 5A, 5C, 5D, 5F, 5G, 5H, 5I and 5J; 5A, 5C, 5E, 5F, 5G, 5H, 5I and 5J; 5A, 5D, 5E, 5F, 5G, 5H, 5I and 5J; 5B, 5C, 5D, 5E, 5F, 5G, 5H and 5I; 5B, 5C, 5D, 5E, 5F, 5G, 5H and 5J; 5B, 5C, 5D, 5E, 5F, 5G, 5I and 5J; 5B, 5C, 5D, 5E, 5F, 5H, 5I and 5J; 5B, 5C, 5D, 5E, 5G, 5H, 5I and 5J; 5B, 5C, 5D, 5F, 5G, 5H, 5I and 5J; 5B, 5C, 5E, 5F, 5G, 5H, 5I and 5J; or 5B, 5D, 5E, 5F, 5G, 5H, 5I and 5J. In some embodiments, the non-naturally occurring eukaryotic organism, comprises eight or more exogenous nucleic acids, wherein each of the eight or more exogenous nucleic acids encodes a different acetyl-CoA pathway enzyme.

In some embodiments, the acetyl-CoA pathway comprises 5A, 5B, 5C, 5D, 5E, 5F, 5G, 5H and 5I; 5A, 5B, 5C, 5D, 5E, 5F, 5G, 5H and 5J; 5A, 5B, 5C, 5D, 5E, 5F, 5G, 5I and 5J; 5A, 5B, 5C, 5D, 5E, 5F, 5H, 5I and 5J; 5A, 5B, 5C, 5D, 5E, 5G, 5H, 5I and 5J; 5A, 5B, 5C, 5D, 5F, 5G, 5H, 5I and 5J; 5A, 5B, 5C, 5E, 5F, 5G, 5H, 5I and 5J; 5A, 5C, 5D, 5E, 5F, 5G, 5H, 5I and 5J; or 5B, 5C, 5D, 5E, 5F, 5G, 5H, 5I and 5J. In some embodiments, the non-naturally occurring eukaryotic organism, comprises nine or more exogenous nucleic acids, wherein each of the nine or more exogenous nucleic acids encodes a different acetyl-CoA pathway enzyme.

In other embodiments, the acetyl-CoA pathway comprises 5A, 5B, 5C, 5D, 5E, 5F, 5G, 5H, 5I and 5J. In some embodiments, the non-naturally occurring eukaryotic organism, comprises ten or more exogenous nucleic acids, wherein each of the ten or more exogenous nucleic acids encodes a different acetyl-CoA pathway enzyme.

In certain embodiments, the acetyl-CoA pathway comprises 6A, 6B, 6C, 6D or 6E, or any combination of 6A, 6B, 6C, 6D and 6E thereof, wherein 6A is mitochondrial acetylcarnitine transferase; 6B is a peroxisomal acetylcarnitine transferase; 6C is a cytosolic acetylcarnitine transferase; 6D is a mitochondrial acetylcarnitine translocase; and 6E. is peroxisomal acetylcarnitine translocase.

In some embodiments, the acetyl-CoA pathway is an acetyl-CoA pathway depicted in FIG. 6. In a specific embodiment, the acetyl-CoA pathway comprises 6A, 6D and 6C. In another specific embodiment, the acetyl-CoA pathway comprises 6B, 6E and 6C.

In one embodiment, the acetyl-CoA pathway comprises 6A. In another embodiment, the acetyl-CoA pathway comprises 6B. In some embodiments, the 6C. In other embodiments, 6D. In yet other embodiments, 6E. In some embodiments, the non-naturally occurring eukaryotic organism, comprises one or more exogenous nucleic acids, wherein each of the one or more exogenous nucleic acids encodes a different acetyl-CoA pathway enzyme.

In some embodiments, the acetyl-CoA pathway comprises: 6A and 6B; 6A and 6C; 6A and 6D; 6A and 6E; 6B and 6C; 6B and 6D; 6B and 6E; 6C and 6D; 6C and 6E; or 6D and 6E. In some embodiments, the non-naturally occurring eukaryotic organism comprises two or more exogenous nucleic acids, wherein each of the two or more exogenous nucleic acids encodes a different acetyl-CoA pathway enzyme.

In other embodiments, the acetyl-CoA pathway comprises: 6A, 6B and 6C; 6A, 6B and 6D; 6A, 6B and 6E; 6A, 6C and 6D; 6A, 6C and 6E; 6A, 6D and 6E; 6B, 6C and 6D; 6B, 6C and 6E; or 6C, 6D and 6E. In some embodiments, the non-naturally occurring eukaryotic organism, comprises three or more exogenous nucleic acids, wherein each of the three or more exogenous nucleic acids encodes a different acetyl-CoA pathway enzyme.

In another embodiment, the acetyl-CoA pathway comprises: 6A, 6B, 6C and 6D; 6A, 6B, 6C and 6E; or 6B, 6C, 6D and 6E. In some embodiments, the non-naturally occurring eukaryotic organism, comprises four or more exogenous nucleic acids, wherein each of the four or more exogenous nucleic acids encodes a different acetyl-CoA pathway enzyme.

In yet another embodiment, the acetyl-CoA pathway comprises 6A, 6B, 6C, 6D and 6E. In some embodiments, the non-naturally occurring eukaryotic organism, comprises five or more exogenous nucleic acids, wherein each of the five or more exogenous nucleic acids encodes a different acetyl-CoA pathway enzyme.

In some embodiments, the acetyl-CoA pathway comprises 10A, 10B, 10C, 10D, 10F, 10G, 10H. 10J, 10K, 10L, 10M, 10N, or any combination of 10A, 10B, 10C, 10D, 10F, 10G, 10H. 10J, 10K, 10L, 10M, 10N thereof, wherein 10A is a PEP carboxylase or PEP carboxykinase; 10B is an oxaloacetate decarboxylase; 10C is a malonate semialdehyde dehydrogenase (acetylating); 10D is a malonyl-CoA decarboxylase; 10F is an oxaloacetate dehydrogenase or oxaloacetate oxidoreductase; 10G is a malonyl-CoA reductase; 10H is a pyruvate carboxylase; 10J is a malonate semialdehyde dehydrogenase; 10K is a malonyl-CoA synthetase or transferase; 10L is a malic enzyme; 10M is a malate dehydrogenase or oxidoreductase; and 10N is a pyruvate kinase or PEP phosphatase. In one embodiment, 10A is a PEP carboxylase. In another embodiment, 10A is a PEP carboxykinase. In an embodiment, 1° F. is an oxaloacetate dehydrogenase. In other embodiments, 1° F. is an oxaloacetate oxidoreductase. In one embodiment, 10K is a malonyl-CoA synthetase. In another embodiment, 10K is a malonyl-CoA transferase. In one embodiment, 10M is a malate dehydrogenase. In another embodiment, 10M is a malate oxidoreductase. In other embodiments, 10N is a pyruvate kinase. In some embodiments, 10N is a PEP phosphatase.

In one embodiment, the acetyl-CoA pathway comprises 10A. In some embodiments, the acetyl-CoA pathway comprises 10B. In other embodiments, the acetyl-CoA pathway comprises 10C. In another embodiment, the acetyl-CoA pathway comprises 10D. In some embodiments, the acetyl-CoA pathway comprises 10F. In one embodiment, the acetyl-CoA pathway comprises 10G. In other embodiments, the acetyl-CoA pathway comprises 10H. In yet other embodiments, the acetyl-CoA pathway comprises 10J. In some embodiments, the acetyl-CoA pathway comprises 10K. In certain embodiments, the acetyl-CoA pathway comprises 10L. In other embodiments, the acetyl-CoA pathway comprises 10M. In another embodiment, the acetyl-CoA pathway comprises 10N.

In some embodiments, the acetyl-CoA pathway further comprises 7A, 7E or 7F, or any combination of 7A, 7E and 7F thereof, wherein 7A is an acetoacetyl-CoA thiolase (FIG. 10, step I), 7E is an acetyl-CoA carboxylase (FIG. 10, step D); and 7F is an acetoacetyl-CoA synthase (FIG. 10, step E).

In some embodiments, the acetyl-CoA pathway is an acetyl-CoA pathway depicted in FIG. 10. In a specific embodiment, the acetyl-CoA pathway comprises 10A, 10B and 10C. In some embodiments, the acetyl-CoA pathway comprises 10N, 10H, 10B and 10C. In other embodiments, the acetyl-CoA pathway comprises 10N, 10L, 10M, 10B and 10C. In another embodiment, the acetyl-CoA pathway comprises 10A, 10B, 10G and 10D. In some embodiments, the acetyl-CoA pathway comprises 10N, 10H, 10B, 10G and 10D. In one embodiment, the acetyl-CoA pathway comprises 10N, 10L, 10M, 10B, 10G and 10D. In other embodiments, the acetyl-CoA pathway comprises 10A, 10B, 10J, 10K and 10D. In yet other embodiments, the acetyl-CoA pathway comprises 10N, 10H, 10B, 10J, 10K and 10D. In some embodiments, the acetyl-CoA pathway comprises 10N, 10L, 10M, 10B, 10J, 10K and 10D. In certain embodiments, the acetyl-CoA pathway comprises 10A, 10F and 10D. In other embodiments, the acetyl-CoA pathway comprises 10N, 10H, 10F and 10D. In another embodiment, the acetyl-CoA pathway comprises 10N, 10L, 10M, 10F and 10D.

While generally described herein as a eukaryotic organism that contains an acetyl-CoA pathway, it is understood that also provided herein is a non-naturally occurring eukaryotic organism comprising at least one exogenous nucleic acid encoding an acetyl-CoA pathway enzyme expressed in a sufficient amount to produce an intermediate of an acetyl-CoA pathway. For example, as disclosed herein, an acetyl-CoA pathway is exemplified in FIGS. 2, 3, 5, 6, 7,8 and 10. Therefore, in addition to a eukaryotic organism containing an acetyl-CoA pathway that is capable of producing cytosolic acetyl-CoA in said organism, transporting acetyl-CoA from a mitochondrion or peroxisome of said organism to the cytosol of said organism and/or increasing acetyl-CoA in the cytosol of said organism, also provided herein is a non-naturally occurring eukaryotic organism comprising at least one exogenous nucleic acid encoding an acetyl-CoA pathway enzyme, where the eukaryotic organism produces an acetyl-CoA pathway intermediate, for example, citrate, citramalate, oxaloacetate, acetate, malate, acetaldehyde, acetylphosphate or acetylcarnitine.

It is understood that any of the pathways disclosed herein, as described in the Examples and exemplified in the figures, including the pathways of FIG. 2, 3, 4, 5, 6, 7, 8 9 or 10, can be utilized to generate a non-naturally occurring eukaryotic organism that produces any pathway intermediate or product, as desired. As disclosed herein, such a eukaryotic organism that produces an intermediate can be used in combination with another eukaryotic organism expressing downstream pathway enzymes to produce a desired product. However, it is understood that a non-naturally occurring eukaryotic organism that produces an acetyl-CoA pathway intermediate can be utilized to produce the intermediate as a desired product.

Any non-naturally occurring eukaryotic organism comprising an acetyl-CoA pathway and engineered to comprise an acetyl-CoA pathway enzyme, such as those provided herein, can be engineered to further comprise one or more 1,3-BDO pathway enzymes. In some embodiments, the non-naturally occurring eukaryotic organisms having a 1,3-BDO pathway include a set of 1,3-BDO pathway enzymes. A set of 1,3-BDO pathway enzymes represents a group of enzymes that can convert acetyl-CoA to 1,3-BDO, e.g., as shown in FIG. 4 or FIG. 7.

In some embodiments, provided herein is a non-naturally occurring eukaryotic organism, comprising (1) an acetyl-CoA pathway, wherein said organism comprises at least one exogenous nucleic acid encoding an acetyl-CoA pathway enzyme expressed in a sufficient amount to (i) transport acetyl-CoA from a mitochondrion and/or peroxisome of said organism to the cytosol of said organism, (ii) produce acetyl-CoA in the cytoplasm of said organism, and/or (iii) increase acetyl-CoA in the cytosol of said organism; and (2) a 1,3-BDO pathway, comprising at least one exogenous nucleic acid encoding a 1,3-BDO pathway enzyme expressed in a sufficient amount to produce 1,3-BDO. In one embodiment, the at least one acetyl-CoA pathway enzyme expressed in a sufficient amount to transport acetyl-CoA from a mitochondrion and/or peroxisome of said organism to the cytosol of the organism. In one embodiment, the at least one acetyl-CoA pathway enzyme is expressed in a sufficient amount to produce cytosolic acetyl-CoA in said organism. In another embodiment, the at least one acetyl-CoA pathway enzyme is expressed in a sufficient amount to increase acetyl-CoA in the cytosol of said organism. In some embodiments, the acetyl CoA pathway comprises any of the various combinations of acetyl-CoA pathway enzymes described above or elsewhere herein. In certain embodiments, 1,3-BDO byproduct pathways are deleted.

In certain embodiments, (1) the acetyl-CoA pathway comprises: 2A, 2B, 2C, 2D, 2E, 2F, 2G, 2K, 2L, 3H, 3I or 3J, or any combination of 2A, 2B, 2C, 2D, 2E, 2F, 2G, 3H, 3I and 3J, thereof; wherein 2A is a citrate synthase; 2B is a citrate transporter; 2C is a citrate/oxaloacetate transporter or a citrate/malate transporter; 2D is an ATP citrate lyase; 2E is a citrate lyase; 2F is an acetyl-CoA synthetase; 2G is an oxaloacetate transporter; 2K is an acetate kinase; 2L is a phosphotransacetylase; 3H is a cytosolic malate dehydrogenase; 3I is a malate transporter; and 3J is a mitochondrial malate dehydrogenase; and (2) the 1,3-BDO pathway comprises 4A, 4B, 4C, 4D, 4E, 4F, 4G, 4H, 4I, 4J, 4K, 4L, 4M, 4N or 4O, or any combination of 4A, 4B, 4C, 4D, 4E, 4F, 4G, 4H, 4I, 4J, 4K, 4L, 4M, 4N and 4O thereof; wherein 4A is an acetoacetyl-CoA thiolase; wherein 4B is an acetoacetyl-CoA reductase (CoA-dependent, alcohol forming); wherein 4C is a 3-oxobutyraldehyde reductase (aldehyde reducing); wherein 4D is a 4-hydroxy,2-butanone reductase; wherein 4E is an acetoacetyl-CoA reductase (CoA-dependent, aldehyde forming); wherein 4F is a 3-oxobutyraldehyde reductase (ketone reducing); wherein 4G is a 3-hydroxybutyraldehyde reductase; wherein 4H is an acetoacetyl-CoA reductase (ketone reducing); wherein 4I is a 3-hydroxybutyryl-CoA reductase (aldehyde forming); wherein 4J is a 3-hydroxybutyryl-CoA reductase (alcohol forming); wherein 4K is an acetoacetyl-CoA transferase, an acetoacetyl-CoA hydrolase, an acetoacetyl-CoA synthetase, or a phosphotransacetoacetylase and acetoacetate kinase; wherein 4L is an acetoacetate reductase; wherein 4M is a 3-hydroxybutyryl-CoA transferase, hydrolase, or synthetase; wherein 4N is a 3-hydroxybutyrate reductase; and wherein 4O is a 3-hydroxybutyrate dehydrogenase. In some embodiments, 2C is a citrate/oxaloacetate transporter. In other embodiments, 2C is a citrate/malate transporter. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In one embodiment, the 1,3-BDO pathway comprises 4A. In another embodiment, the 1,3-BDO pathway comprises 4B. In an embodiment, the 1,3-BDO pathway comprises 4C. In another embodiment, the 1,3-BDO pathway comprises 4D. In one embodiment, the 1,3-BDO pathway comprises 4E. In yet another embodiment, the 1,3-BDO pathway comprises 4F. In some embodiments, the 1,3-BDO pathway comprises 4G. In other embodiments, the 1,3-BDO pathway comprises 4H. In another embodiment, the 1,3-BDO pathway comprises 4I. In one embodiment, the 1,3-BDO pathway comprises 4J. In one embodiment, the 1,3-BDO pathway comprises 4K. In another embodiment, the 1,3-BDO pathway comprises 4L. In an embodiment, the 1,3-BDO pathway comprises 4M. In another embodiment, the 1,3-BDO pathway comprises 4N. In one embodiment, the 1,3-BDO pathway comprises 4O.

In some embodiments, the acetyl-CoA pathway is an acetyl-CoA pathway depicted in FIG. 2, and the 1,3-BDO pathway is a 1,3-BDO pathway depicted in FIG. 4. In other embodiments, the acetyl-CoA pathway is an acetyl-CoA pathway depicted in FIG. 3, and the 1,3-BDO pathway is a 1,3-BDO pathway depicted in FIG. 4. In yet other embodiments, the acetyl-CoA pathway is an acetyl-CoA pathway depicted in FIG. 7, and the 1,3-BDO pathway is a 1,3-BDO pathway depicted in FIG. 4 or FIG. 7. Exemplary sets of 1,3-BDO pathway enzymes to convert acetyl-CoA to 1,3-BDO, according to FIG. 4, include 4A, 4E, 4F and 4G; 4A, 4B and 4D; 4A, 4E, 4C and 4D; 4A, 4H and 4J; 4A, 4H, 4I and 4G; 4A, 4H, 4M, 4N and 4G; 4A, 4K, 4O, 4N and 4G; or 4A, 4K, 4L, 4F and 4G.

In one embodiment, the acetyl-CoA pathway comprises 2A, 2B and 2D. In another embodiment, the acetyl-CoA pathway comprises 2A, 2C and 2D. In yet another embodiment, the acetyl-CoA pathway comprises 2A, 2B, 2C and 2D. In an embodiment, the acetyl-CoA pathway comprises 2A, 2B, 2E and 2F. In another embodiment, the acetyl-CoA pathway comprises 2A, 2C, 2E and 2F. In other embodiments, the acetyl-CoA pathway comprises 2A, 2B, 2C, 2E and 2F. In some embodiments, the acetyl CoA pathway comprises 2A, 2B, 2E, 2K and 2L. In another embodiment, the acetyl CoA pathway comprises 2A, 2C, 2E, 2K and 2L. In other embodiments, the acetyl CoA pathway comprises 2A, 2B, 2C, 2E, 2K and 2L. In some embodiments, the acetyl-CoA pathway further comprises 2G, 3H, 3I, 3J, or any combination thereof. In certain embodiments, the acetyl-CoA pathway further comprises 2G. In some embodiments, the acetyl-CoA pathway further comprises 3H. In other embodiments, the acetyl-CoA pathway further comprises 3I. In yet other embodiments, the acetyl-CoA pathway further comprises 3J. In some embodiments, the acetyl-CoA pathway further comprises 2G and 3H. In an embodiment, the acetyl-CoA pathway further comprises 2G and 3I. In one embodiment, the acetyl-CoA pathway further comprises 2G and 3J. In some embodiments, the acetyl-CoA pathway further comprises 3H and 3I. In other embodiments, the acetyl-CoA pathway further comprises 3H and 3J. In certain embodiments, the acetyl-CoA pathway further comprises 3I and 3J. In another embodiment, the acetyl-CoA pathway further comprises 2G, 3H and 3I. In yet another embodiment, the acetyl-CoA pathway further comprises 2G, 3H and 3J. In some embodiments, the acetyl-CoA pathway further comprises 2G, 3I and 3J. In other embodiments, the acetyl-CoA pathway further comprises 3H, 3I and 3J.

Any of the acetyl-CoA pathway enzymes provided herein can be in combination with any of the 1,3-BDO pathway enzymes provided herein.

In one embodiment, the 1,3-BDO pathway comprises 4A, 4E, 4F and 4G. In another embodiment, the 1,3-BDO pathway comprises 4A, 4B and 4D. In other embodiments, the 1,3-BDO pathway comprises 4A, 4E, 4C and 4D. In some embodiments, the 1,3-BDO pathway comprises 4A, 4H and 4J. In other embodiments, the 1,3-BDO pathway comprises 4A, 4H, 4I and 4G. In certain embodiments, the 1,3-BDO pathway comprises 4A, 4H, 4M, 4N and 4G. In another embodiment, the 1,3-BDO pathway comprises 4A, 4K, 4O, 4N and 4G. In yet another embodiment, the 1,3-BDO pathway comprises 4A, 4K, 4L, 4F and 4G.

In certain embodiments, (1) the acetyl-CoA pathway comprises (i) 2A, 2B and 2D; (ii) 2A, 2C and 2D; (iii) 2A, 2B, 2C and 2D; (iv) 2A, 2B, 2E and 2F; (v) 2A, 2C, 2E and 2F; (vi) 2A, 2B, 2C, 2E and 2F; (vii) 2A, 2B, 2E, 2K and 2L; (viii) 2A, 2C, 2E, 2K and 2L or (ix) 2A, 2B, 2C, 2E, 2K and 2L, and wherein the acetyl-CoA pathway optionally further comprises 2G, 3H, 3I, 3J, or any combination thereof; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G.

In some embodiments, (1) the acetyl-CoA pathway comprises 2A, 2B and 2D; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B and 2D, and the 1,3-BDO pathway comprises 4A, 4E, 4F and 4G. In other embodiments, the acetyl-CoA pathway comprises 2A, 2B and 2D, and the 1,3-BDO pathway comprises 4A, 4B and 4D. In one embodiment, the acetyl-CoA pathway comprises 2A, 2B and 2D, and the 1,3-BDO pathway comprises 4A, 4E, 4C and 4D. In some embodiments, the acetyl-CoA pathway comprises 2A, 2B and 2D, and the 1,3-BDO pathway comprises 4A, 4H and 4J. In other embodiments, the acetyl-CoA pathway comprises 2A, 2B and 2D, and the 1,3-BDO pathway comprises 4A, 4H, 4I and 4G. In certain embodiments, the acetyl-CoA pathway comprises 2A, 2B and 2D, and the 1,3-BDO pathway comprises 4A, 4H, 4M, 4N and 4G. In another embodiment, the acetyl-CoA pathway comprises 2A, 2B and 2D, and the 1,3-BDO pathway comprises 4A, 4K, 4O, 4N and 4G. In yet another embodiment, the acetyl-CoA pathway comprises 2A, 2B and 2D, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2C and 2D; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2C and 2D, and the 1,3-BDO pathway comprises 4A, 4E, 4F and 4G. In other embodiments, the acetyl-CoA pathway comprises 2A, 2C and 2D, and the 1,3-BDO pathway comprises 4A, 4B and 4D. In one embodiment, the acetyl-CoA pathway comprises 2A, 2C and 2D, and the 1,3-BDO pathway comprises 4A, 4E, 4C and 4D. In some embodiments, the acetyl-CoA pathway comprises 2A, 2C and 2D, and the 1,3-BDO pathway comprises 4A, 4H and 4J. In other embodiments, the acetyl-CoA pathway comprises 2A, 2C and 2D, and the 1,3-BDO pathway comprises 4A, 4H, 4I and 4G. In certain embodiments, the acetyl-CoA pathway comprises 2A, 2C and 2D, and the 1,3-BDO pathway comprises 4A, 4H, 4M, 4N and 4G. In another embodiment, the acetyl-CoA pathway comprises 2A, 2C and 2D, and the 1,3-BDO pathway comprises 4A, 4K, 4O, 4N and 4G. In yet another embodiment, the acetyl-CoA pathway comprises 2A, 2C and 2D, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2B, 2C and 2D; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B, 2C and 2D, and the 1,3-BDO pathway comprises 4A, 4E, 4F and 4G. In other embodiments, the acetyl-CoA pathway comprises 2A, 2B, 2C and 2D, and the 1,3-BDO pathway comprises 4A, 4B and 4D. In one embodiment, the acetyl-CoA pathway comprises 2A, 2B, 2C and 2D, and the 1,3-BDO pathway comprises 4A, 4E, 4C and 4D. In some embodiments, the acetyl-CoA pathway comprises 2A, 2B, 2C and 2D, and the 1,3-BDO pathway comprises 4A, 4H and 4J. In other embodiments, the acetyl-CoA pathway comprises 2A, 2B, 2C and 2D, and the 1,3-BDO pathway comprises 4A, 4H, 4I and 4G. In certain embodiments, the acetyl-CoA pathway comprises 2A, 2B, 2C and 2D, and the 1,3-BDO pathway comprises 4A, 4H, 4M, 4N and 4G. In another embodiment, the acetyl-CoA pathway comprises 2A, 2B, 2C and 2D, and the 1,3-BDO pathway comprises 4A, 4K, 4O, 4N and 4G. In yet another embodiment, the acetyl-CoA pathway comprises 2A, 2B, 2C and 2D, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2B, 2E and 2F; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B, 2E and 2F, and the 1,3-BDO pathway comprises 4A, 4E, 4F and 4G. In other embodiments, the acetyl-CoA pathway comprises 2A, 2B, 2E and 2F, and the 1,3-BDO pathway comprises 4A, 4B and 4D. In one embodiment, the acetyl-CoA pathway comprises 2A, 2B, 2E and 2F, and the 1,3-BDO pathway comprises 4A, 4E, 4C and 4D. In some embodiments, the acetyl-CoA pathway comprises 2A, 2B, 2E and 2F, and the 1,3-BDO pathway comprises 4A, 4H and 4J. In other embodiments, the acetyl-CoA pathway comprises 2A, 2B, 2E and 2F, and the 1,3-BDO pathway comprises 4A, 4H, 4I and 4G. In certain embodiments, the acetyl-CoA pathway comprises 2A, 2B, 2E and 2F, and the 1,3-BDO pathway comprises 4A, 4H, 4M, 4N and 4G. In another embodiment, the acetyl-CoA pathway comprises 2A, 2B, 2E and 2F, and the 1,3-BDO pathway comprises 4A, 4K, 4O, 4N and 4G. In yet another embodiment, the acetyl-CoA pathway comprises 2A, 2B, 2E and 2F, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2C, 2E and 2F; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2C, 2E and 2F, and the 1,3-BDO pathway comprises 4A, 4E, 4F and 4G. In other embodiments, the acetyl-CoA pathway comprises 2A, 2C, 2E and 2F, and the 1,3-BDO pathway comprises 4A, 4B and 4D. In one embodiment, the acetyl-CoA pathway comprises 2A, 2C, 2E and 2F, and the 1,3-BDO pathway comprises 4A, 4E, 4C and 4D. In some embodiments, the acetyl-CoA pathway comprises 2A, 2C, 2E and 2F, and the 1,3-BDO pathway comprises 4A, 4H and 4J. In other embodiments, the acetyl-CoA pathway comprises 2A, 2C, 2E and 2F, and the 1,3-BDO pathway comprises 4A, 4H, 4I and 4G. In certain embodiments, the acetyl-CoA pathway comprises 2A, 2C, 2E and 2F, and the 1,3-BDO pathway comprises 4A, 4H, 4M, 4N and 4G. In another embodiment, the acetyl-CoA pathway comprises 2A, 2C, 2E and 2F, and the 1,3-BDO pathway comprises 4A, 4K, 4O, 4N and 4G. In yet another embodiment, the acetyl-CoA pathway comprises 2A, 2C, 2E and 2F, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2B, 2C, 2E and 2F; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B, 2C, 2E and 2F, and the 1,3-BDO pathway comprises 4A, 4E, 4F and 4G. In other embodiments, the acetyl-CoA pathway comprises 2A, 2B, 2C, 2E and 2F, and the 1,3-BDO pathway comprises 4A, 4B and 4D. In one embodiment, the acetyl-CoA pathway comprises 2A, 2B, 2C, 2E and 2F, and the 1,3-BDO pathway comprises 4A, 4E, 4C and 4D. In some embodiments, the acetyl-CoA pathway comprises 2A, 2B, 2C, 2E and 2F, and the 1,3-BDO pathway comprises 4A, 4H and 4J. In other embodiments, the acetyl-CoA pathway comprises 2A, 2B, 2C, 2E and 2F, and the 1,3-BDO pathway comprises 4A, 4H, 4I and 4G. In certain embodiments, the acetyl-CoA pathway comprises 2A, 2B, 2C, 2E and 2F, and the 1,3-BDO pathway comprises 4A, 4H, 4M, 4N and 4G. In another embodiment, the acetyl-CoA pathway comprises 2A, 2B, 2C, 2E and 2F, and the 1,3-BDO pathway comprises 4A, 4K, 4O, 4N and 4G. In yet another embodiment, the acetyl-CoA pathway comprises 2A, 2B, 2C, 2E and 2F, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In some embodiments, (1) the acetyl-CoA pathway comprises 2A, 2B, 2E, 2K and 2L; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B, 2E, 2K and 2L, and the 1,3-BDO pathway comprises 4A, 4E, 4F and 4G. In other embodiments, the acetyl-CoA pathway comprises 2A, 2B, 2E, 2K and 2L, and the 1,3-BDO pathway comprises 4A, 4B and 4D. In one embodiment, the acetyl-CoA pathway comprises 2A, 2B, 2E, 2K and 2L, and the 1,3-BDO pathway comprises 4A, 4E, 4C and 4D. In some embodiments, the acetyl-CoA pathway comprises 2A, 2B, 2E, 2K and 2L, and the 1,3-BDO pathway comprises 4A, 4H and 4J. In other embodiments, the acetyl-CoA pathway comprises 2A, 2B, 2E, 2K and 2L, and the 1,3-BDO pathway comprises 4A, 4H, 4I and 4G. In certain embodiments, the acetyl-CoA pathway comprises 2A, 2B, 2E, 2K and 2L, and the 1,3-BDO pathway comprises 4A, 4H, 4M, 4N and 4G. In another embodiment, the acetyl-CoA pathway comprises 2A, 2B, 2E, 2K and 2L, and the 1,3-BDO pathway comprises 4A, 4K, 4O, 4N and 4G. In yet another embodiment, the acetyl-CoA pathway comprises 2A, 2B, 2E, 2K and 2L and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In some embodiments, (1) the acetyl-CoA pathway comprises 2A, 2C, 2E, 2K and 2L; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B, 2E, 2K and 2L, and the 1,3-BDO pathway comprises 4A, 4E, 4F and 4G. In other embodiments, the acetyl-CoA pathway comprises 2A, 2B, 2E, 2K and 2L, and the 1,3-BDO pathway comprises 4A, 4B and 4D. In one embodiment, the acetyl-CoA pathway comprises 2A, 2C, 2E, 2K and 2L, and the 1,3-BDO pathway comprises 4A, 4E, 4C and 4D. In some embodiments, the acetyl-CoA pathway comprises 2A, 2C, 2E, 2K and 2L, and the 1,3-BDO pathway comprises 4A, 4H and 4J. In other embodiments, the acetyl-CoA pathway comprises 2A, 2C, 2E, 2K and 2L, and the 1,3-BDO pathway comprises 4A, 4H, 4I and 4G. In certain embodiments, the acetyl-CoA pathway comprises 2A, 2C, 2E, 2K and 2L, and the 1,3-BDO pathway comprises 4A, 4H, 4M, 4N and 4G. In another embodiment, the acetyl-CoA pathway comprises 2A, 2C, 2E, 2K and 2L, and the 1,3-BDO pathway comprises 4A, 4K, 4O, 4N and 4G. In yet another embodiment, the acetyl-CoA pathway comprises 2A, 2C, 2E, 2K and 2L and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the acetyl-CoA pathway further comprises 2G, 3H, 3I, 3J, or any combination thereof. In certain embodiments, the acetyl-CoA pathway further comprises 2G. In some embodiments, the acetyl-CoA pathway further comprises 3H. In other embodiments, the acetyl-CoA pathway further comprises 3I. In yet other embodiments, the acetyl-CoA pathway further comprises 3J. In some embodiments, the acetyl-CoA pathway further comprises 2G and 3H. In an embodiment, the acetyl-CoA pathway further comprises 2G and 3I. In one embodiment, the acetyl-CoA pathway further comprises 2G and 3J. In some embodiments, the acetyl-CoA pathway further comprises 3H and 3I. In other embodiments, the acetyl-CoA pathway further comprises 3H and 3J. In certain embodiments, the acetyl-CoA pathway further comprises 3I and 3J. In another embodiment, the acetyl-CoA pathway further comprises 2G, 3H and 3I. In yet another embodiment, the acetyl-CoA pathway further comprises 2G, 3H and 3J. In some embodiments, the acetyl-CoA pathway further comprises 2G, 3I and 3J. In other embodiments, the acetyl-CoA pathway further comprises 3H, 3I and 3J. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In some embodiments, (1) the acetyl-CoA pathway comprises 2A, 2B, 2C, 2E, 2K and 2L; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G. In some embodiments, the acetyl-CoA pathway comprises 2A, 2B, 2C, 2E, 2K and 2L, and the 1,3-BDO pathway comprises 4A, 4E, 4F and 4G. In other embodiments, the acetyl-CoA pathway comprises 2A, 2B, 2C, 2E, 2K and 2L, and the 1,3-BDO pathway comprises 4A, 4B and 4D. In one embodiment, the acetyl-CoA pathway comprises 2A, 2B, 2C, 2E, 2K and 2L, and the 1,3-BDO pathway comprises 4A, 4E, 4C and 4D. In some embodiments, the acetyl-CoA pathway comprises 2A, 2B, 2C, 2E, 2K and 2L, and the 1,3-BDO pathway comprises 4A, 4H and 4J. In other embodiments, the acetyl-CoA pathway comprises 2A, 2B, 2C, 2E, 2K and 2L, and the 1,3-BDO pathway comprises 4A, 4H, 4I and 4G. In certain embodiments, the acetyl-CoA pathway comprises 2A, 2B, 2C, 2E, 2K and 2L, and the 1,3-BDO pathway comprises 4A, 4H, 4M, 4N and 4G. In another embodiment, the acetyl-CoA pathway comprises 2A, 2B, 2C, 2E, 2K and 2L, and the 1,3-BDO pathway comprises 4A, 4K, 4O, 4N and 4G. In yet another embodiment, the acetyl-CoA pathway comprises 2A, 2B, 2C, 2E, 2K and 2L, and the 1,3-BDO pathway comprises 4A, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In certain embodiments, (1) the acetyl-CoA pathway comprises 5A, 5B, 5C, 5D 5E, 5F, 5G, 5H, 5I, 5J or any combination of 5A, 5B, 5C, 5D, 5E, 5F, 5G, 5H, 5I and 5J thereof, wherein 5A is a pyruvate oxidase (acetate forming); 5B is an acetyl-CoA synthetase, ligase or transferase; 5C is an acetate kinase; 5D is a phosphotransacetylase; 5E is a pyruvate decarboxylase; 5F is an acetaldehyde dehydrogenase; 5G is a pyruvate oxidase (acetyl-phosphate forming); 5H is a pyruvate dehydrogenase, pyruvate:ferredoxin oxidoreductase or pyruvate formate lyase; 5I acetaldehyde dehydrogenase (acylating); and 5J is a threonine aldolase; and (2) the 1,3-BDO pathway comprises 4A, 4B, 4C, 4D, 4E, 4F, 4G, 4H, 4I, 4J, 4K, 4L, 4M, 4N or 4O, or any combination of 4A, 4B, 4C, 4D, 4E, 4F, 4G, 4H, 4I, 4J, 4K, 4L, 4M, 4N and 4O thereof; wherein 4A is an acetoacetyl-CoA thiolase; wherein 4B is an acetoacetyl-CoA reductase (CoA-dependent, alcohol forming); wherein 4C is a 3-oxobutyraldehyde reductase (aldehyde reducing); wherein 4D is a 4-hydroxy,2-butanone reductase; wherein 4E is an acetoacetyl-CoA reductase (CoA-dependent, aldehyde forming); wherein 4F is a 3-oxobutyraldehyde reductase (ketone reducing); wherein 4G is a 3-hydroxybutyraldehyde reductase; wherein 4H is an acetoacetyl-CoA reductase (ketone reducing); wherein 4I is a 3-hydroxybutyryl-CoA reductase (aldehyde forming); wherein 4J is a 3-hydroxybutyryl-CoA reductase (alcohol forming); wherein 4K is an acetoacetyl-CoA transferase, an acetoacetyl-CoA hydrolase, an acetoacetyl-CoA synthetase, or a phosphotransacetoacetylase and acetoacetate kinase; wherein 4L is an acetoacetate reductase; wherein 4M is a 3-hydroxybutyryl-CoA transferase, hydrolase, or synthetase; wherein 4N is a 3-hydroxybutyrate reductase; and wherein 4O is a 3-hydroxybutyrate dehydrogenase. In certain embodiments, 5B is an acetyl-CoA synthetase. In another embodiment, 5B is an acetyl-CoA ligase. In other embodiments, 5B is an acetyl-CoA transferase. In some embodiments, 5H is a pyruvate dehydrogenase. In other embodiments, 5H is a pyruvate:ferredoxin oxidoreductase. In yet other embodiments, 5H is a pyruvate formate lyase. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In some embodiments, the acetyl-CoA pathway is an acetyl-CoA pathway depicted in FIG. 5, and the 1,3-BDO pathway is a 1,3-BDO pathway depicted in FIG. 4. Exemplary sets of acetyl-CoA pathway enzymes, according to FIG. 5, are 5A and 5B; 5A, 5C and 5D; 5G and 5D; 5E, 5F, 5C and 5D; 5J and 5I; 5J, 5F and 5B; and 5H. Exemplary sets of 1,3-BDO pathway enzymes to convert acetyl-CoA to 1,3-BDO, according to FIG. 4, include 4A, 4E, 4F and 4G; 4A, 4B and 4D; 4A, 4E, 4C and 4D; 4A, 4H and 4J; 4A, 4H, 4I and 4G; 4A, 4H, 4M, 4N and 4G; 4A, 4K, 4O, 4N and 4G; or 4A, 4K, 4L, 4F and 4G.

In some embodiments, (1) the acetyl-CoA pathway comprises (i) 5A and 5B; (ii) 5A, 5C and 5D; (iii) 5E, 5F, 5C and 5D; (iv) 5G and 5D; (v) 5J and 5I; (vi) 5J, 5F and 5B; or (vii) 5H; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G.

In some embodiments, the acetyl-CoA pathway comprises 5A and 5B; and the 1,3-BDO pathway comprises 4A, 4E, 4F and 4G. In other embodiments, the acetyl-CoA pathway comprises 5A and 5B; and the 1,3-BDO pathway comprises 4A, 4B and 4D. In some embodiments, the acetyl-CoA pathway comprises 5A and 5B; and the 1,3-BDO pathway comprises 4A, 4E, 4C and 4D. In some embodiments, the acetyl-CoA pathway comprises 5A and 5B; and the 1,3-BDO pathway comprises 4A, 4H and 4J. In some embodiments, the acetyl-CoA pathway comprises 5A and 5B; and the 1,3-BDO pathway comprises 4A, 4H, 4I and 4G. In some embodiments, the acetyl-CoA pathway comprises 5A and 5B; and the 1,3-BDO pathway comprises 4A, 4H, 4M, 4N and 4G. In some embodiments, the acetyl-CoA pathway comprises 5A and 5B; and the 1,3-BDO pathway comprises 4A, 4K, 4O, 4N and 4G. In some embodiments, the acetyl-CoA pathway comprises 5A and 5B; and the 1,3-BDO pathway comprises 4A, 4K, 4L, 4F and 4G.

In some embodiments, the acetyl-CoA pathway comprises 5A, 5C and 5D; and the 1,3-BDO pathway comprises 4A, 4E, 4F and 4G. In other embodiments, the acetyl-CoA pathway comprises 5A, 5C and 5D; and the 1,3-BDO pathway comprises 4A, 4B and 4D. In some embodiments, the acetyl-CoA pathway comprises 5A, 5C and 5D; and the 1,3-BDO pathway comprises 4A, 4E, 4C and 4D. In some embodiments, the acetyl-CoA pathway comprises 5A, 5C and 5D; and the 1,3-BDO pathway comprises 4A, 4H and 4J. In some embodiments, the acetyl-CoA pathway comprises 5A, 5C and 5D; and the 1,3-BDO pathway comprises 4A, 4H, 4I and 4G. In some embodiments, the acetyl-CoA pathway comprises 5A, 5C and 5D; and the 1,3-BDO pathway comprises 4A, 4H, 4M, 4N and 4G. In some embodiments, the acetyl-CoA pathway comprises 5A, 5C and 5D; and the 1,3-BDO pathway comprises 4A, 4K, 4O, 4N and 4G. In some embodiments, the acetyl-CoA pathway comprises 5A, 5C and 5D; and the 1,3-BDO pathway comprises 4A, 4K, 4L, 4F and 4G.

In some embodiments, the acetyl-CoA pathway comprises 5E, 5F, 5C and 5D; and the 1,3-BDO pathway comprises 4A, 4E, 4F and 4G. In other embodiments, the acetyl-CoA pathway comprises 5E, 5F, 5C and 5D; and the 1,3-BDO pathway comprises 4A, 4B and 4D. In some embodiments, the acetyl-CoA pathway comprises 5E, 5F, 5C and 5D; and the 1,3-BDO pathway comprises 4A, 4E, 4C and 4D. In some embodiments, the acetyl-CoA pathway comprises 5E, 5F, 5C and 5D; and the 1,3-BDO pathway comprises 4A, 4H and 4J. In some embodiments, the acetyl-CoA pathway comprises 5E, 5F, 5C and 5D; and the 1,3-BDO pathway comprises 4A, 4H, 4I and 4G. In some embodiments, the acetyl-CoA pathway comprises 5E, 5F, 5C and 5D; and the 1,3-BDO pathway comprises 4A, 4H, 4M, 4N and 4G. In some embodiments, the acetyl-CoA pathway comprises 5E, 5F, 5C and 5D; and the 1,3-BDO pathway comprises 4A, 4K, 4O, 4N and 4G. In some embodiments, the acetyl-CoA pathway comprises 5E, 5F, 5C and 5D; and the 1,3-BDO pathway comprises 4A, 4K, 4L, 4F and 4G.

In some embodiments, the acetyl-CoA pathway comprises 5G and 5D; and the 1,3-BDO pathway comprises 4A, 4E, 4F and 4G. In other embodiments, the acetyl-CoA pathway comprises 5G and 5D; and the 1,3-BDO pathway comprises 4A, 4B and 4D. In some embodiments, the acetyl-CoA pathway comprises 5G and 5D; and the 1,3-BDO pathway comprises 4A, 4E, 4C and 4D. In some embodiments, the acetyl-CoA pathway comprises 5G and 5D and the 1,3-BDO pathway comprises 4A, 4H and 4J. In some embodiments, the acetyl-CoA pathway comprises 5G and 5D; and the 1,3-BDO pathway comprises 4A, 4H, 4I and 4G. In some embodiments, the acetyl-CoA pathway comprises 5G and 5D; and the 1,3-BDO pathway comprises 4A, 4H, 4M, 4N and 4G. In some embodiments, the acetyl-CoA pathway comprises 5G and 5D; and the 1,3-BDO pathway comprises 4A, 4K, 4O, 4N and 4G. In some embodiments, the acetyl-CoA pathway comprises 5G and 5D; and the 1,3-BDO pathway comprises 4A, 4K, 4L, 4F and 4G.

In some embodiments, the acetyl-CoA pathway comprises 5J and 5I; and the 1,3-BDO pathway comprises 4A, 4E, 4F and 4G. In other embodiments, the acetyl-CoA pathway comprises 5J and 5I; and the 1,3-BDO pathway comprises 4A, 4B and 4D. In some embodiments, the acetyl-CoA pathway comprises 5J and 5I; and the 1,3-BDO pathway comprises 4A, 4E, 4C and 4D. In some embodiments, the acetyl-CoA pathway comprises 5J and 5I; and the 1,3-BDO pathway comprises 4A, 4H and 4J. In some embodiments, the acetyl-CoA pathway comprises 5J and 5I; and the 1,3-BDO pathway comprises 4A, 4H, 4I and 4G. In some embodiments, the acetyl-CoA pathway comprises 5J and 5I; and the 1,3-BDO pathway comprises 4A, 4H, 4M, 4N and 4G. In some embodiments, the acetyl-CoA pathway comprises 5J and 5I; and the 1,3-BDO pathway comprises 4A, 4K, 4O, 4N and 4G. In some embodiments, the acetyl-CoA pathway comprises 5J and 5I; and the 1,3-BDO pathway comprises 4A, 4K, 4L, 4F and 4G.

In some embodiments, the acetyl-CoA pathway comprises 5J, 5F and 5B; and the 1,3-BDO pathway comprises 4A, 4E, 4F and 4G. In other embodiments, the acetyl-CoA pathway comprises 5J, 5F and 5B; and the 1,3-BDO pathway comprises 4A, 4B and 4D. In some embodiments, the acetyl-CoA pathway comprises 5J, 5F and 5B; and the 1,3-BDO pathway comprises 4A, 4E, 4C and 4D. In some embodiments, the acetyl-CoA pathway comprises 5J, 5F and 5B; and the 1,3-BDO pathway comprises 4A, 4H and 4J. In some embodiments, the acetyl-CoA pathway comprises 5J, 5F and 5B; and the 1,3-BDO pathway comprises 4A, 4H, 4I and 4G. In some embodiments, the acetyl-CoA pathway comprises 5J, 5F and 5B; and the 1,3-BDO pathway comprises 4A, 4H, 4M, 4N and 4G. In some embodiments, the acetyl-CoA pathway comprises 5J, 5F and 5B; and the 1,3-BDO pathway comprises 4A, 4K, 4O, 4N and 4G. In some embodiments, the acetyl-CoA pathway comprises 5J, 5F and 5B; and the 1,3-BDO pathway comprises 4A, 4K, 4L, 4F and 4G.

In some embodiments, the acetyl-CoA pathway comprises 5H; and the 1,3-BDO pathway comprises 4A, 4E, 4F and 4G. In other embodiments, the acetyl-CoA pathway comprises 5H; and the 1,3-BDO pathway comprises 4A, 4B and 4D. In some embodiments, the acetyl-CoA pathway comprises 5H; and the 1,3-BDO pathway comprises 4A, 4E, 4C and 4D. In some embodiments, the acetyl-CoA pathway comprises 5H; and the 1,3-BDO pathway comprises 4A, 4H and 4J. In some embodiments, the acetyl-CoA pathway comprises 5H; and the 1,3-BDO pathway comprises 4A, 4H, 4I and 4G. In some embodiments, the acetyl-CoA pathway comprises 5H; and the 1,3-BDO pathway comprises 4A, 4H, 4M, 4N and 4G. In some embodiments, the acetyl-CoA pathway comprises 5H; and the 1,3-BDO pathway comprises 4A, 4K, 4O, 4N and 4G. In some embodiments, the acetyl-CoA pathway comprises 5H; and the 1,3-BDO pathway comprises 4A, 4K, 4L, 4F and 4G.

In certain embodiments, (1) the acetyl-CoA pathway comprises 6A, 6B, 6C, 6D or 6E, or any combination of 6A, 6B, 6C, 6D and 6E thereof, wherein 6A is mitochondrial acetylcarnitine transferase; 6B is a peroxisomal acetylcarnitine transferase; 6C is a cytosolic acetylcarnitine transferase; 6D is a mitochondrial acetylcarnitine translocase; and 6E. is peroxisomal acetylcarnitine translocase; and (2) the 1,3-BDO pathway comprises 4A, 4B, 4C, 4D, 4E, 4F, 4G, 4H, 4I, 4J, 4K, 4L, 4M, 4N or 4O, or any combination of 4A, 4B, 4C, 4D, 4E, 4F, 4G, 4H, 4I, 4J, 4K, 4L, 4M, 4N and 4O thereof; wherein 4A is an acetoacetyl-CoA thiolase; wherein 4B is an acetoacetyl-CoA reductase (CoA-dependent, alcohol forming); wherein 4C is a 3-oxobutyraldehyde reductase (aldehyde reducing); wherein 4D is a 4-hydroxy,2-butanone reductase; wherein 4E is an acetoacetyl-CoA reductase (CoA-dependent, aldehyde forming); wherein 4F is a 3-oxobutyraldehyde reductase (ketone reducing); wherein 4G is a 3-hydroxybutyraldehyde reductase; wherein 4H is an acetoacetyl-CoA reductase (ketone reducing); wherein 4I is a 3-hydroxybutyryl-CoA reductase (aldehyde forming); wherein 4J is a 3-hydroxybutyryl-CoA reductase (alcohol forming); wherein 4K is an acetoacetyl-CoA transferase, an acetoacetyl-CoA hydrolase, an acetoacetyl-CoA synthetase, or a phosphotransacetoacetylase and acetoacetate kinase; wherein 4L is an acetoacetate reductase; wherein 4M is a 3-hydroxybutyryl-CoA transferase, hydrolase, or synthetase; wherein 4N is a 3-hydroxybutyrate reductase; and wherein 4O is a 3-hydroxybutyrate dehydrogenase. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In some embodiments, the acetyl-CoA pathway is an acetyl-CoA pathway depicted in FIG. 6, and the 1,3-BDO pathway is a 1,3-BDO pathway depicted in FIG. 4. Exemplary sets of acetyl-CoA pathway enzymes, according to FIG. 6, are 6A, 6D and 6C; and 6B, 6E and 6C. Exemplary sets of 1,3-BDO pathway enzymes to convert acetyl-CoA to 1,3-BDO, according to FIG. 4, include 4A, 4E, 4F and 4G; 4A, 4B and 4D; 4A, 4E, 4C and 4D; 4A, 4H and 4J; 4A, 4H, 4I and 4G; 4A, 4H, 4M, 4N and 4G; 4A, 4K, 4O, 4N and 4G; or 4A, 4K, 4L, 4F and 4G.

In one embodiment, (1) the acetyl-CoA pathway comprises (i) 6A, 6D and 6C; or (ii) 6B, 6E and 6C; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G.

In some embodiments, the acetyl-CoA pathway comprises 6A, 6D and 6C; and the 1,3-BDO pathway comprises 4A, 4E, 4F and 4G. In other embodiments, the acetyl-CoA pathway comprises 6A, 6D and 6C; and the 1,3-BDO pathway comprises 4A, 4B and 4D. In some embodiments, the acetyl-CoA pathway comprises 6A, 6D and 6C; and the 1,3-BDO pathway comprises 4A, 4E, 4C and 4D. In some embodiments, the acetyl-CoA pathway comprises 6A, 6D and 6C; and the 1,3-BDO pathway comprises 4A, 4H and 4J. In some embodiments, the acetyl-CoA pathway comprises 6A, 6D and 6C; and the 1,3-BDO pathway comprises 4A, 4H, 4I and 4G. In some embodiments, the acetyl-CoA pathway comprises 6A, 6D and 6C; and the 1,3-BDO pathway comprises 4A, 4H, 4M, 4N and 4G. In some embodiments, the acetyl-CoA pathway comprises 6A, 6D and 6C; and the 1,3-BDO pathway comprises 4A, 4K, 4O, 4N and 4G. In some embodiments, the acetyl-CoA pathway comprises 6A, 6D and 6C; and the 1,3-BDO pathway comprises 4A, 4K, 4L, 4F and 4G.

In some embodiments, the acetyl-CoA pathway comprises 6B, 6E and 6C; and the 1,3-BDO pathway comprises 4A, 4E, 4F and 4G. In other embodiments, the acetyl-CoA pathway comprises 6B, 6E and 6C; and the 1,3-BDO pathway comprises 4A, 4B and 4D. In some embodiments, the acetyl-CoA pathway comprises 6B, 6E and 6C; and the 1,3-BDO pathway comprises 4A, 4E, 4C and 4D. In some embodiments, the acetyl-CoA pathway comprises 6B, 6E and 6C; and the 1,3-BDO pathway comprises 4A, 4H and 4J. In some embodiments, the acetyl-CoA pathway comprises 6B, 6E and 6C; and the 1,3-BDO pathway comprises 4A, 4H, 4I and 4G. In some embodiments, the acetyl-CoA pathway comprises 6B, 6E and 6C; and the 1,3-BDO pathway comprises 4A, 4H, 4M, 4N and 4G. In some embodiments, the acetyl-CoA pathway comprises 6B, 6E and 6C; and the 1,3-BDO pathway comprises 4A, 4K, 4O, 4N and 4G. In some embodiments, the acetyl-CoA pathway comprises 6B, 6E and 6C; and the 1,3-BDO pathway comprises 4A, 4K, 4L, 4F and 4G.

In certain embodiments, (1) the acetyl-CoA pathway comprises 10A, 10B, 10C, 10D, 10F, 10G, 10H. 10J, 10K, 10L, 10M, 10N, or any combination of 10A, 10B, 10C, 10D, 10F, 10G, 10H. 10J, 10K, 10L, 10M, 10N thereof; and (2) the 1,3-BDO pathway comprises 4A (see also FIG. 10, step I), 4B, 4C, 4D, 4E, 4F, 4G, 4H, 4I, 4J, 4K, 4L, 4M, 4N or 4O, or any combination of 4A, 4B, 4C, 4D, 4E, 4F, 4G, 4H, 4I, 4J, 4K, 4L, 4M, 4N and 4O thereof. In one embodiment, 10A is a PEP carboxylase. In another embodiment, 10A is a PEP carboxykinase. In an embodiment, 1° F. is an oxaloacetate dehydrogenase. In other embodiments, 1° F. is an oxaloacetate oxidoreductase. In one embodiment, 10K is a malonyl-CoA synthetase. In another embodiment, 10K is a malonyl-CoA transferase. In one embodiment, 10M is a malate dehydrogenase. In another embodiment, 10M is a malate oxidoreductase. In other embodiments, 10N is a pyruvate kinase. In some embodiments, 10N is a PEP phosphatase. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In some embodiments, the acetyl-CoA pathway is an acetyl-CoA pathway depicted in FIG. 10, and the 1,3-BDO pathway is a 1,3-BDO pathway depicted in FIG. 4. Exemplary sets of acetyl-CoA pathway enzymes, according to FIG. 10, are 10A, 10B and 10C; 10N, 10H, 10B and 10C; 10N, 10L, 10M, 10B and 10C; 10A, 10B, 10G and 10D; 10N, 10H, 10B, 10G and 10D; 10N, 10L, 10M, 10B, 10G and 10D; 10A, 10B, 10J, 10K and 10D; 10N, 10H, 10B, 10J, 10K and 10D; 10N, 10L, 10M, 10B, 10J, 10K and 10D; 10A, 10F and 10D; 10N, 10H, 10F and 10D; and 10N, 10L, 10M, 10F and 10D. Exemplary sets of 1,3-BDO pathway enzymes to convert acetyl-CoA to 1,3-BDO, according to FIG. 4, include 4A, 4E, 4F and 4G; 4A, 4B and 4D; 4A, 4E, 4C and 4D; 4A, 4H and 4J; 4A, 4H, 4I and 4G; 4A, 4H, 4M, 4N and 4G; 4A, 4K, 4O, 4N and 4G; or 4A, 4K, 4L, 4F and 4G.

In one embodiment, (1) the acetyl-CoA pathway comprises (i) 10A, 10B and 10C; (ii) 10N, 10H, 10B and 10C; (iii) 10N, 10L, 10M, 10B and 10C; (iv) 10A, 10B, 10G and 10D; (v) 10N, 10H, 10B, 10G and 10D; (vi) 10N, 10L, 10M, 10B, 10G and 10D; (vii) 10A, 10B, 10J, 10K and 10D; (viii) 10N, 10H, 10B, 10J, 10K and 10D; (ix) 10N, 10L, 10M, 10B, 10J, 10K and 10D; (x) 10A, 10F and 10D; (xi) 10N, 10H, 10F and 10D; or (xii) 10N, 10L, 10M, 10F and 10D; and (2) the 1,3-BDO pathway comprises (i) 4A, 4E, 4F and 4G; (ii) 4A, 4B and 4D; (iii) 4A, 4E, 4C and 4D; (iv) 4A, 4H and 4J; (v) 4A, 4H, 4I and 4G; (vi) 4A, 4H, 4M, 4N and 4G; (vii) 4A, 4K, 4O, 4N and 4G; or (viii) 4A, 4K, 4L, 4F and 4G.

In some embodiments, the acetyl-CoA pathway comprises 10A, 10B and 10C; and the 1,3-BDO pathway comprises 4A, 4E, 4F and 4G. In other embodiments, the acetyl-CoA pathway comprises 10A, 10B and 10C; and the 1,3-BDO pathway comprises 4A, 4B and 4D. In some embodiments, the acetyl-CoA pathway comprises 10A, 10B and 10C; and the 1,3-BDO pathway comprises 4A, 4E, 4C and 4D. In some embodiments, the acetyl-CoA pathway comprises 10A, 10B and 10C; and the 1,3-BDO pathway comprises 4A, 4H and 4J. In some embodiments, the acetyl-CoA pathway comprises 10A, 10B and 10C; and the 1,3-BDO pathway comprises 4A, 4H, 4I and 4G. In some embodiments, the acetyl-CoA pathway comprises 10A, 10B and 10C; and the 1,3-BDO pathway comprises 4A, 4H, 4M, 4N and 4G. In some embodiments, the acetyl-CoA pathway comprises 10A, 10B and 10C; and the 1,3-BDO pathway comprises 4A, 4K, 4O, 4N and 4G. In some embodiments, the acetyl-CoA pathway comprises 10A, 10B and 10C; and the 1,3-BDO pathway comprises 4A, 4K, 4L, 4F and 4G.

In some embodiments, the acetyl-CoA pathway comprises 10N, 10H, 10B and 10C; and the 1,3-BDO pathway comprises 4A, 4E, 4F and 4G. In other embodiments, the acetyl-CoA pathway comprises 10N, 10H, 10B and 10C; and the 1,3-BDO pathway comprises 4A, 4B and 4D. In some embodiments, the acetyl-CoA pathway comprises 10N, 10H, 10B and 10C; and the 1,3-BDO pathway comprises 4A, 4E, 4C and 4D. In some embodiments, the acetyl-CoA pathway comprises 10N, 10H, 10B and 10C; and the 1,3-BDO pathway comprises 4A, 4H and 4J. In some embodiments, the acetyl-CoA pathway comprises 10N, 10H, 10B and 10C; and the 1,3-BDO pathway comprises 4A, 4H, 4I and 4G. In some embodiments, the acetyl-CoA pathway comprises 10N, 10H, 10B and 10C; and the 1,3-BDO pathway comprises 4A, 4H, 4M, 4N and 4G. In some embodiments, the acetyl-CoA pathway comprises 10N, 10H, 10B and 10C; and the 1,3-BDO pathway comprises 4A, 4K, 4O, 4N and 4G. In some embodiments, the acetyl-CoA pathway comprises 10N, 10H, 10B and 10C; and the 1,3-BDO pathway comprises 4A, 4K, 4L, 4F and 4G.

In some embodiments, the acetyl-CoA pathway comprises 10N, 10L, 10M, 10B and 10C; and the 1,3-BDO pathway comprises 4A, 4E, 4F and 4G. In other embodiments, the acetyl-CoA pathway comprises 10N, 10L, 10M, 10B and 10C; and the 1,3-BDO pathway comprises 4A, 4B and 4D. In some embodiments, the acetyl-CoA pathway comprises 10N, 10L, 10M, 10B and 10C; and the 1,3-BDO pathway comprises 4A, 4E, 4C and 4D. In some embodiments, the acetyl-CoA pathway comprises 10N, 10L, 10M, 10B and 10C; and the 1,3-BDO pathway comprises 4A, 4H and 4J. In some embodiments, the acetyl-CoA pathway comprises 10N, 10L, 10M, 10B and 10C; and the 1,3-BDO pathway comprises 4A, 4H, 4I and 4G. In some embodiments, the acetyl-CoA pathway comprises 10N, 10L, 10M, 10B and 10C; and the 1,3-BDO pathway comprises 4A, 4H, 4M, 4N and 4G. In some embodiments, the acetyl-CoA pathway comprises 10N, 10L, 10M, 10B and 10C; and the 1,3-BDO pathway comprises 4A, 4K, 4O, 4N and 4G. In some embodiments, the acetyl-CoA pathway comprises 10N, 10L, 10M, 10B and 10C; and the 1,3-BDO pathway comprises 4A, 4K, 4L, 4F and 4G.

In some embodiments, the acetyl-CoA pathway comprises 10A, 10B, 10G and 10D; and the 1,3-BDO pathway comprises 4A, 4E, 4F and 4G. In other embodiments, the acetyl-CoA pathway comprises 10A, 10B, 10G and 10D; and the 1,3-BDO pathway comprises 4A, 4B and 4D. In some embodiments, the acetyl-CoA pathway comprises 10A, 10B, 10G and 10D; and the 1,3-BDO pathway comprises 4A, 4E, 4C and 4D. In some embodiments, the acetyl-CoA pathway comprises 10A, 10B, 10G and 10D; and the 1,3-BDO pathway comprises 4A, 4H and 4J. In some embodiments, the acetyl-CoA pathway comprises 10A, 10B, 10G and 10D; and the 1,3-BDO pathway comprises 4A, 4H, 4I and 4G. In some embodiments, the acetyl-CoA pathway comprises 10A, 10B, 10G and 10D; and the 1,3-BDO pathway comprises 4A, 4H, 4M, 4N and 4G. In some embodiments, the acetyl-CoA pathway comprises 10A, 10B, 10G and 10D; and the 1,3-BDO pathway comprises 4A, 4K, 4O, 4N and 4G. In some embodiments, the acetyl-CoA pathway comprises 10A, 10B, 10G and 10D; and the 1,3-BDO pathway comprises 4A, 4K, 4L, 4F and 4G.

In some embodiments, the acetyl-CoA pathway comprises 10N, 10H, 10B, 10G and 10D; and the 1,3-BDO pathway comprises 4A, 4E, 4F and 4G. In other embodiments, the acetyl-CoA pathway comprises 10N, 10H, 10B, 10G and 10D; and the 1,3-BDO pathway comprises 4A, 4B and 4D. In some embodiments, the acetyl-CoA pathway comprises 10N, 10H, 10B, 10G and 10D; and the 1,3-BDO pathway comprises 4A, 4E, 4C and 4D. In some embodiments, the acetyl-CoA pathway comprises 10N, 10H, 10B, 10G and 10D; and the 1,3-BDO pathway comprises 4A, 4H and 4J. In some embodiments, the acetyl-CoA pathway comprises 10N, 10H, 10B, 10G and 10D; and the 1,3-BDO pathway comprises 4A, 4H, 4I and 4G. In some embodiments, the acetyl-CoA pathway comprises 10N, 10H, 10B, 10G and 10D; and the 1,3-BDO pathway comprises 4A, 4H, 4M, 4N and 4G. In some embodiments, the acetyl-CoA pathway comprises 10N, 10H, 10B, 10G and 10D; and the 1,3-BDO pathway comprises 4A, 4K, 4O, 4N and 4G. In some embodiments, the acetyl-CoA pathway comprises 10N, 10H, 10B, 10G and 10D; and the 1,3-BDO pathway comprises 4A, 4K, 4L, 4F and 4G.

In some embodiments, the acetyl-CoA pathway comprises 10N, 10L, 10M, 10B, 10G and 10D; and the 1,3-BDO pathway comprises 4A, 4E, 4F and 4G. In other embodiments, the acetyl-CoA pathway comprises 10N, 10L, 10M, 10B, 10G and 10D; and the 1,3-BDO pathway comprises 4A, 4B and 4D. In some embodiments, the acetyl-CoA pathway comprises 10N, 10L, 10M, 10B, 10G and 10D; and the 1,3-BDO pathway comprises 4A, 4E, 4C and 4D. In some embodiments, the acetyl-CoA pathway comprises 10N, 10L, 10M, 10B, 10G and 10D; and the 1,3-BDO pathway comprises 4A, 4H and 4J. In some embodiments, the acetyl-CoA pathway comprises 10N, 10L, 10M, 10B, 10G and 10D; and the 1,3-BDO pathway comprises 4A, 4H, 4I and 4G. In some embodiments, the acetyl-CoA pathway comprises 10N, 10L, 10M, 10B, 10G and 10D; and the 1,3-BDO pathway comprises 4A, 4H, 4M, 4N and 4G. In some embodiments, the acetyl-CoA pathway comprises 10N, 10L, 10M, 10B, 10G and 10D; and the 1,3-BDO pathway comprises 4A, 4K, 4O, 4N and 4G. In some embodiments, the acetyl-CoA pathway comprises 10N, 10L, 10M, 10B, 10G and 10D; and the 1,3-BDO pathway comprises 4A, 4K, 4L, 4F and 4G.

In some embodiments, the acetyl-CoA pathway comprises 10A, 10B, 10J, 10K and 10D; and the 1,3-BDO pathway comprises 4A, 4E, 4F and 4G. In other embodiments, the acetyl-CoA pathway comprises 10A, 10B, 10J, 10K and 10D; and the 1,3-BDO pathway comprises 4A, 4B and 4D. In some embodiments, the acetyl-CoA pathway comprises 10A, 10B, 10J, 10K and 10D; and the 1,3-BDO pathway comprises 4A, 4E, 4C and 4D. In some embodiments, the acetyl-CoA pathway comprises 10A, 10B, 10J, 10K and 10D; and the 1,3-BDO pathway comprises 4A, 4H and 4J. In some embodiments, the acetyl-CoA pathway comprises 10A, 10B, 10J, 10K and 10D; and the 1,3-BDO pathway comprises 4A, 4H, 4I and 4G. In some embodiments, the acetyl-CoA pathway comprises 10A, 10B, 10J, 10K and 10D; and the 1,3-BDO pathway comprises 4A, 4H, 4M, 4N and 4G. In some embodiments, the acetyl-CoA pathway comprises 10A, 10B, 10J, 10K and 10D; and the 1,3-BDO pathway comprises 4A, 4K, 4O, 4N and 4G. In some embodiments, the acetyl-CoA pathway comprises 10A, 10B, 10J, 10K and 10D; and the 1,3-BDO pathway comprises 4A, 4K, 4L, 4F and 4G.

In some embodiments, the acetyl-CoA pathway comprises 10N, 10H, 10B, 10J, 10K and 10D; and the 1,3-BDO pathway comprises 4A, 4E, 4F and 4G. In other embodiments, the acetyl-CoA pathway comprises 10N, 10H, 10B, 10J, 10K and 10D; and the 1,3-BDO pathway comprises 4A, 4B and 4D. In some embodiments, the acetyl-CoA pathway comprises 10N, 10H, 10B, 10J, 10K and 10D; and the 1,3-BDO pathway comprises 4A, 4E, 4C and 4D. In some embodiments, the acetyl-CoA pathway comprises 10N, 10H, 10B, 10J, 10K and 10D; and the 1,3-BDO pathway comprises 4A, 4H and 4J. In some embodiments, the acetyl-CoA pathway comprises 10N, 10H, 10B, 10J, 10K and 10D; and the 1,3-BDO pathway comprises 4A, 4H, 4I and 4G. In some embodiments, the acetyl-CoA pathway comprises 10N, 10H, 10B, 10J, 10K and 10D; and the 1,3-BDO pathway comprises 4A, 4H, 4M, 4N and 4G. In some embodiments, the acetyl-CoA pathway comprises 10N, 10H, 10B, 10J, 10K and 10D; and the 1,3-BDO pathway comprises 4A, 4K, 4O, 4N and 4G. In some embodiments, the acetyl-CoA pathway comprises 10N, 10H, 10B, 10J, 10K and 10D; and the 1,3-BDO pathway comprises 4A, 4K, 4L, 4F and 4G.

In some embodiments, the acetyl-CoA pathway comprises 10N, 10L, 10M, 10B, 10J, 10K and 10D; and the 1,3-BDO pathway comprises 4A, 4E, 4F and 4G. In other embodiments, the acetyl-CoA pathway comprises 10N, 10L, 10M, 10B, 10J, 10K and 10D; and the 1,3-BDO pathway comprises 4A, 4B and 4D. In some embodiments, the acetyl-CoA pathway comprises 10N, 10L, 10M, 10B, 10J, 10K and 10D; and the 1,3-BDO pathway comprises 4A, 4E, 4C and 4D. In some embodiments, the acetyl-CoA pathway comprises 10N, 10L, 10M, 10B, 10J, 10K and 10D; and the 1,3-BDO pathway comprises 4A, 4H and 4J. In some embodiments, the acetyl-CoA pathway comprises 10N, 10L, 10M, 10B, 10J, 10K and 10D; and the 1,3-BDO pathway comprises 4A, 4H, 4I and 4G. In some embodiments, the acetyl-CoA pathway comprises 10N, 10L, 10M, 10B, 10J, 10K and 10D; and the 1,3-BDO pathway comprises 4A, 4H, 4M, 4N and 4G. In some embodiments, the acetyl-CoA pathway comprises 10N, 10L, 10M, 10B, 10J, 10K and 10D; and the 1,3-BDO pathway comprises 4A, 4K, 4O, 4N and 4G. In some embodiments, the acetyl-CoA pathway comprises 10N, 10L, 10M, 10B, 10J, 10K and 10D; and the 1,3-BDO pathway comprises 4A, 4K, 4L, 4F and 4G.

In some embodiments, the acetyl-CoA pathway comprises 10A, 10F and 10D; and the 1,3-BDO pathway comprises 4A, 4E, 4F and 4G. In other embodiments, the acetyl-CoA pathway comprises 10A, 10F and 10D; and the 1,3-BDO pathway comprises 4A, 4B and 4D. In some embodiments, the acetyl-CoA pathway comprises 10A, 10F and 10D; and the 1,3-BDO pathway comprises 4A, 4E, 4C and 4D. In some embodiments, the acetyl-CoA pathway comprises 10A, 10F and 10D; and the 1,3-BDO pathway comprises 4A, 4H and 4J. In some embodiments, the acetyl-CoA pathway comprises 10A, 10F and 10D; and the 1,3-BDO pathway comprises 4A, 4H, 4I and 4G. In some embodiments, the acetyl-CoA pathway comprises 10A, 1° F. and 10D; and the 1,3-BDO pathway comprises 4A, 4H, 4M, 4N and 4G. In some embodiments, the acetyl-CoA pathway comprises 10A, 10F and 10D; and the 1,3-BDO pathway comprises 4A, 4K, 4O, 4N and 4G. In some embodiments, the acetyl-CoA pathway comprises 10A, 10F and 10D; and the 1,3-BDO pathway comprises 4A, 4K, 4L, 4F and 4G.

In some embodiments, the acetyl-CoA pathway comprises 10N, 10H, 10F and 10D; and the 1,3-BDO pathway comprises 4A, 4E, 4F and 4G. In other embodiments, the acetyl-CoA pathway comprises 10N, 10H, 10F and 10D; and the 1,3-BDO pathway comprises 4A, 4B and 4D. In some embodiments, the acetyl-CoA pathway comprises 10N, 10H, 10F and 10D; and the 1,3-BDO pathway comprises 4A, 4E, 4C and 4D. In some embodiments, the acetyl-CoA pathway comprises 10N, 10H, 10F and 10D; and the 1,3-BDO pathway comprises 4A, 4H and 4J. In some embodiments, the acetyl-CoA pathway comprises 10N, 10H, 10F and 10D; and the 1,3-BDO pathway comprises 4A, 4H, 4I and 4G. In some embodiments, the acetyl-CoA pathway comprises 10N, 10H, 10F and 10D; and the 1,3-BDO pathway comprises 4A, 4H, 4M, 4N and 4G. In some embodiments, the acetyl-CoA pathway comprises 10N, 10H, 10F and 10D; and the 1,3-BDO pathway comprises 4A, 4K, 4O, 4N and 4G. In some embodiments, the acetyl-CoA pathway comprises 10N, 10H, 10F and 10D; and the 1,3-BDO pathway comprises 4A, 4K, 4L, 4F and 4G.

In some embodiments, the acetyl-CoA pathway comprises 10N, 10L. 10M, 10F and 10D; and the 1,3-BDO pathway comprises 4A, 4E, 4F and 4G. In other embodiments, the acetyl-CoA pathway comprises 10N, 10L. 10M, 10F and 10D; and the 1,3-BDO pathway comprises 4A, 4B and 4D. In some embodiments, the acetyl-CoA pathway comprises 10N, 10L. 10M, 10F and 10D; and the 1,3-BDO pathway comprises 4A, 4E, 4C and 4D. In some embodiments, the acetyl-CoA pathway comprises 10N, 10L. 10M, 10F and 10D; and the 1,3-BDO pathway comprises 4A, 4H and 4J. In some embodiments, the acetyl-CoA pathway comprises 10N, 10L. 10M, 10F and 10D; and the 1,3-BDO pathway comprises 4A, 4H, 4I and 4G. In some embodiments, the acetyl-CoA pathway comprises 10N, 10L. 10M, 10F and 10D; and the 1,3-BDO pathway comprises 4A, 4H, 4M, 4N and 4G. In some embodiments, the acetyl-CoA pathway comprises 10N, 10L. 10M, 10F and 10D; and the 1,3-BDO pathway comprises 4A, 4K, 4O, 4N and 4G. In some embodiments, the acetyl-CoA pathway comprises 10N, 10L. 10M, 10F and 10D; and the 1,3-BDO pathway comprises 4A, 4K, 4L, 4F and 4G.

In an additional embodiment, provided herein is a non-naturally occurring eukaryotic organism having a 1,3-BDO pathway, wherein the non-naturally occurring eukaryotic organism comprises at least one exogenous nucleic acid encoding an enzyme or protein that converts a substrate to a product selected from the group consisting of acetyl-CoA to acetoacetyl-CoA (e.g., 4A); acetoacetyl-CoA to 4-hydroxy-2-butanone (e.g., 4B); 3-oxobutyraldehyde to 4-hydroxy-2-butanone (e.g., 4C); 4-hydroxy-2-butanone to 1,3-BDO (e.g., 4D); acetoacetyl-CoA to 3-oxobutyraldehyde (e.g., 4E); 3-oxobutyraldehyde to 3-hydroxybutyrldehyde (e.g., 4F); 3-hydroxybutyrldehyde to 1,3-BDO (e.g., 4G); acetoacetyl-CoA to 3-hydroxybutyryl-CoA (e.g., 4H); 3-hydroxybutyryl-CoA to 3-hydroxybutyraldehyde (e.g., 4I), 3-hydroxybutyryl-CoA to 1,3-BDO (e.g., 4J); acetoacetyl-CoA to acetoacetate (e.g., 4K); acetoacetate to 3-oxobutyraldehyde (e.g., 4L); 3-hydroxybutyrl-CoA to 3-hydroxybutyrate (e.g., 4M); 3-hydroxybutyrate to 3-hydroxybutyraldehyde (e.g., 4N); and acetoacetate to 3-hydroxybutyrate (e.g., 4O). One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, provided herein are non-naturally occurring eukaryotic organisms comprising at least one exogenous nucleic acid encoding an enzyme or protein, where the enzyme or protein converts the substrates and products of a 1,3-BDO pathway, such as that shown in FIG. 4.

Also provided herein are non-naturally occurring eukaryotic organisms comprising at least one exogenous nucleic acid encoding an acetyl-CoA carboxylase (7E), an acetoacetyl-CoA synthase (7B) or a combination thereof. In certain embodiments of the 1,3-BDO pathways provided herein, including those exemplified in FIG. 4, acetyl-CoA is converted to malonyl-CoA by acetyl-CoA carboxylase, and acetoacetyl-CoA is synthesized from acetyl-CoA and malonyl-CoA by acetoacetyl-CoA synthetase (see FIG. 7 (steps E and F) and FIG. 9). Also provided herein are non-naturally occurring eukaryotic organisms comprising at least one exogenous nucleic acid encoding an enzyme or protein, wherein the enzyme or protein converts the substrates and products of a 1,3-BDO pathway, such as shown in FIG. 7.

In certain embodiments, (1) the acetyl-CoA pathway comprises: 2A, 2B, 2C, 2D, 2E, 2F, 2G, 2K, 2L, 3H, 3I or 3J, or any combination of 2A, 2B, 2C, 2D, 2E, 2F, 2G, 3H, 3I and 3J, thereof; and (2) the 1,3-BDO pathway comprises 7E, 7F, 4B, 4C, 4D, 4E, 4F, 4G, 4H, 4I, 4J, 4K, 4L, 4M, 4N or 4O, or any combination of 7E, 7F, 4B, 4C, 4D, 4E, 4F, 4G, 4H, 4I, 4J, 4K, 4L, 4M, 4N and 4O thereof; wherein 7E is acetyl-CoA carboxylase; wherein 7F is an acetoacetyl-CoA synthase. In one embodiment, the 1,3-BDO pathway comprises 7E. In one embodiment, the 1,3-BDO pathway comprises 7B.

Exemplary sets of 1,3-BDO pathway enzymes to convert acetyl-CoA to 1,3-BDO, according to FIGS. 4 and 7, include 7E, 7F, 4E, 4F and 4G; 7E, 7F, 4B and 4D; 7E, 7F, 4E, 4C and 4D; 7E, 7F, 4H and 4J; 7E, 7F, 4H, 4I and 4G; 7E, 7F, 4H, 4M, 4N and 4G; 7E, 7F, 4K, 4O, 4N and 4G; or 7E, 7F, 4K, 4L, 4F and 4G.

In one embodiment, the 1,3-BDO pathway comprises 7E, 7F, 4E, 4F and 4G. In another embodiment, the 1,3-BDO pathway comprises 7E, 7F, 4B and 4D. In other embodiments, the 1,3-BDO pathway comprises 7E, 7F, 4E, 4C and 4D. In some embodiments, the 1,3-BDO pathway comprises 7E, 7F, 4H and 4J. In other embodiments, the 1,3-BDO pathway comprises 7E, 7F, 4H, 4I and 4G. In certain embodiments, the 1,3-BDO pathway comprises 7E, 7F, 4H, 4M, 4N and 4G. In another embodiment, the 1,3-BDO pathway comprises 7E, 7F, 4K, 4O, 4N and 4G. In yet another embodiment, the 1,3-BDO pathway comprises 7E, 7F, 4K, 4L, 4F and 4G.

In certain embodiments, (1) the acetyl-CoA pathway comprises (i) 2A, 2B and 2D; (ii) 2A, 2C and 2D; (iii) 2A, 2B, 2C and 2D; (iv) 2A, 2B, 2E and 2F; (v) 2A, 2C, 2E and 2F; (vi) 2A, 2B, 2C, 2E and 2F; (vii) 2A, 2B, 2E, 2K and 2L; (viii) 2A, 2C, 2E, 2K and 2L or (ix) 2A, 2B, 2C, 2E, 2K and 2L, and wherein the acetyl-CoA pathway optionally further comprises 2G, 3H, 3I, 3J, or any combination thereof; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G.

In some embodiments, (1) the acetyl-CoA pathway comprises 2A, 2B and 2D; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B and 2D, and the 1,3-BDO pathway comprises 7E, 7F, 4E, 4F and 4G. In other embodiments, the acetyl-CoA pathway comprises 2A, 2B and 2D, and the 1,3-BDO pathway comprises 7E, 7F, 4B and 4D. In one embodiment, the acetyl-CoA pathway comprises 2A, 2B and 2D, and the 1,3-BDO pathway comprises 7E, 7F, 4E, 4C and 4D. In some embodiments, the acetyl-CoA pathway comprises 2A, 2B and 2D, and the 1,3-BDO pathway comprises 7E, 7F, 4H and 4J. In other embodiments, the acetyl-CoA pathway comprises 2A, 2B and 2D, and the 1,3-BDO pathway comprises 7E, 7F, 4H, 4I and 4G. In certain embodiments, the acetyl-CoA pathway comprises 2A, 2B and 2D, and the 1,3-BDO pathway comprises 7E, 7F, 4H, 4M, 4N and 4G. In another embodiment, the acetyl-CoA pathway comprises 2A, 2B and 2D, and the 1,3-BDO pathway comprises 7E, 7F, 4K, 4O, 4N and 4G. In yet another embodiment, the acetyl-CoA pathway comprises 2A, 2B and 2D, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2C and 2D; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2C and 2D, and the 1,3-BDO pathway comprises 7E, 7F, 4E, 4F and 4G. In other embodiments, the acetyl-CoA pathway comprises 2A, 2C and 2D, and the 1,3-BDO pathway comprises 7E, 7F, 4B and 4D. In one embodiment, the acetyl-CoA pathway comprises 2A, 2C and 2D, and the 1,3-BDO pathway comprises 7E, 7F, 4E, 4C and 4D. In some embodiments, the acetyl-CoA pathway comprises 2A, 2C and 2D, and the 1,3-BDO pathway comprises 7E, 7F, 4H and 4J. In other embodiments, the acetyl-CoA pathway comprises 2A, 2C and 2D, and the 1,3-BDO pathway comprises 7E, 7F, 4H, 4I and 4G. In certain embodiments, the acetyl-CoA pathway comprises 2A, 2C and 2D, and the 1,3-BDO pathway comprises 7E, 7F, 4H, 4M, 4N and 4G. In another embodiment, the acetyl-CoA pathway comprises 2A, 2C and 2D, and the 1,3-BDO pathway comprises 7E, 7F, 4K, 4O, 4N and 4G. In yet another embodiment, the acetyl-CoA pathway comprises 2A, 2C and 2D, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2B, 2C and 2D; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B, 2C and 2D, and the 1,3-BDO pathway comprises 7E, 7F, 4E, 4F and 4G. In other embodiments, the acetyl-CoA pathway comprises 2A, 2B, 2C and 2D, and the 1,3-BDO pathway comprises 7E, 7F, 4B and 4D. In one embodiment, the acetyl-CoA pathway comprises 2A, 2B, 2C and 2D, and the 1,3-BDO pathway comprises 7E, 7F, 4E, 4C and 4D. In some embodiments, the acetyl-CoA pathway comprises 2A, 2B, 2C and 2D, and the 1,3-BDO pathway comprises 7E, 7F, 4H and 4J. In other embodiments, the acetyl-CoA pathway comprises 2A, 2B, 2C and 2D, and the 1,3-BDO pathway comprises 7E, 7F, 4H, 4I and 4G. In certain embodiments, the acetyl-CoA pathway comprises 2A, 2B, 2C and 2D, and the 1,3-BDO pathway comprises 7E, 7F, 4H, 4M, 4N and 4G. In another embodiment, the acetyl-CoA pathway comprises 2A, 2B, 2C and 2D, and the 1,3-BDO pathway comprises 7E, 7F, 4K, 4O, 4N and 4G. In yet another embodiment, the acetyl-CoA pathway comprises 2A, 2B, 2C and 2D, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2B, 2E and 2F; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B, 2E and 2F, and the 1,3-BDO pathway comprises 7E, 7F, 4E, 4F and 4G. In other embodiments, the acetyl-CoA pathway comprises 2A, 2B, 2E and 2F, and the 1,3-BDO pathway comprises 7E, 7F, 4B and 4D. In one embodiment, the acetyl-CoA pathway comprises 2A, 2B, 2E and 2F, and the 1,3-BDO pathway comprises 7E, 7F, 4E, 4C and 4D. In some embodiments, the acetyl-CoA pathway comprises 2A, 2B, 2E and 2F, and the 1,3-BDO pathway comprises 7E, 7F, 4H and 4J. In other embodiments, the acetyl-CoA pathway comprises 2A, 2B, 2E and 2F, and the 1,3-BDO pathway comprises 7E, 7F, 4H, 4I and 4G. In certain embodiments, the acetyl-CoA pathway comprises 2A, 2B, 2E and 2F, and the 1,3-BDO pathway comprises 7E, 7F, 4H, 4M, 4N and 4G. In another embodiment, the acetyl-CoA pathway comprises 2A, 2B, 2E and 2F, and the 1,3-BDO pathway comprises 7E, 7F, 4K, 4O, 4N and 4G. In yet another embodiment, the acetyl-CoA pathway comprises 2A, 2B, 2E and 2F, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2C, 2E and 2F; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2C, 2E and 2F, and the 1,3-BDO pathway comprises 7E, 7F, 4E, 4F and 4G. In other embodiments, the acetyl-CoA pathway comprises 2A, 2C, 2E and 2F, and the 1,3-BDO pathway comprises 7E, 7F, 4B and 4D. In one embodiment, the acetyl-CoA pathway comprises 2A, 2C, 2E and 2F, and the 1,3-BDO pathway comprises 7E, 7F, 4E, 4C and 4D. In some embodiments, the acetyl-CoA pathway comprises 2A, 2C, 2E and 2F, and the 1,3-BDO pathway comprises 7E, 7F, 4H and 4J. In other embodiments, the acetyl-CoA pathway comprises 2A, 2C, 2E and 2F, and the 1,3-BDO pathway comprises 7E, 7F, 4H, 4I and 4G. In certain embodiments, the acetyl-CoA pathway comprises 2A, 2C, 2E and 2F, and the 1,3-BDO pathway comprises 7E, 7F, 4H, 4M, 4N and 4G. In another embodiment, the acetyl-CoA pathway comprises 2A, 2C, 2E and 2F, and the 1,3-BDO pathway comprises 7E, 7F, 4K, 4O, 4N and 4G. In yet another embodiment, the acetyl-CoA pathway comprises 2A, 2C, 2E and 2F, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In other embodiments, (1) the acetyl-CoA pathway comprises 2A, 2B, 2C, 2E and 2F; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B, 2C, 2E and 2F, and the 1,3-BDO pathway comprises 7E, 7F, 4E, 4F and 4G. In other embodiments, the acetyl-CoA pathway comprises 2A, 2B, 2C, 2E and 2F, and the 1,3-BDO pathway comprises 7E, 7F, 4B and 4D. In one embodiment, the acetyl-CoA pathway comprises 2A, 2B, 2C, 2E and 2F, and the 1,3-BDO pathway comprises 7E, 7F, 4E, 4C and 4D. In some embodiments, the acetyl-CoA pathway comprises 2A, 2B, 2C, 2E and 2F, and the 1,3-BDO pathway comprises 7E, 7F, 4H and 4J. In other embodiments, the acetyl-CoA pathway comprises 2A, 2B, 2C, 2E and 2F, and the 1,3-BDO pathway comprises 7E, 7F, 4H, 4I and 4G. In certain embodiments, the acetyl-CoA pathway comprises 2A, 2B, 2C, 2E and 2F, and the 1,3-BDO pathway comprises 7E, 7F, 4H, 4M, 4N and 4G. In another embodiment, the acetyl-CoA pathway comprises 2A, 2B, 2C, 2E and 2F, and the 1,3-BDO pathway comprises 7E, 7F, 4K, 4O, 4N and 4G. In yet another embodiment, the acetyl-CoA pathway comprises 2A, 2B, 2C, 2E and 2F, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In some embodiments, (1) the acetyl-CoA pathway comprises 2A, 2B, 2E, 2K and 2L; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B, 2E, 2K and 2L, and the 1,3-BDO pathway comprises 7E, 7F, 4E, 4F and 4G. In other embodiments, the acetyl-CoA pathway comprises 2A, 2B, 2E, 2K and 2L, and the 1,3-BDO pathway comprises 7E, 7F, 4B and 4D. In one embodiment, the acetyl-CoA pathway comprises 2A, 2B, 2E, 2K and 2L, and the 1,3-BDO pathway comprises 7E, 7F, 4E, 4C and 4D. In some embodiments, the acetyl-CoA pathway comprises 2A, 2B, 2E, 2K and 2L, and the 1,3-BDO pathway comprises 7E, 7F, 4H and 4J. In other embodiments, the acetyl-CoA pathway comprises 2A, 2B, 2E, 2K and 2L, and the 1,3-BDO pathway comprises 7E, 7F, 4H, 4I and 4G. In certain embodiments, the acetyl-CoA pathway comprises 2A, 2B, 2E, 2K and 2L, and the 1,3-BDO pathway comprises 7E, 7F, 4H, 4M, 4N and 4G. In another embodiment, the acetyl-CoA pathway comprises 2A, 2B, 2E, 2K and 2L, and the 1,3-BDO pathway comprises 7E, 7F, 4K, 4O, 4N and 4G. In yet another embodiment, the acetyl-CoA pathway comprises 2A, 2B, 2E, 2K and 2L and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In some embodiments, (1) the acetyl-CoA pathway comprises 2A, 2C, 2E, 2K and 2L; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B, 2E, 2K and 2L, and the 1,3-BDO pathway comprises 7E, 7F, 4E, 4F and 4G. In other embodiments, the acetyl-CoA pathway comprises 2A, 2B, 2E, 2K and 2L, and the 1,3-BDO pathway comprises 7E, 7F, 4B and 4D. In one embodiment, the acetyl-CoA pathway comprises 2A, 2C, 2E, 2K and 2L, and the 1,3-BDO pathway comprises 7E, 7F, 4E, 4C and 4D. In some embodiments, the acetyl-CoA pathway comprises 2A, 2C, 2E, 2K and 2L, and the 1,3-BDO pathway comprises 7E, 7F, 4H and 4J. In other embodiments, the acetyl-CoA pathway comprises 2A, 2C, 2E, 2K and 2L, and the 1,3-BDO pathway comprises 7E, 7F, 4H, 4I and 4G. In certain embodiments, the acetyl-CoA pathway comprises 2A, 2C, 2E, 2K and 2L, and the 1,3-BDO pathway comprises 7E, 7F, 4H, 4M, 4N and 4G. In another embodiment, the acetyl-CoA pathway comprises 2A, 2C, 2E, 2K and 2L, and the 1,3-BDO pathway comprises 7E, 7F, 4K, 4O, 4N and 4G. In yet another embodiment, the acetyl-CoA pathway comprises 2A, 2C, 2E, 2K and 2L and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In some embodiments, (1) the acetyl-CoA pathway comprises 2A, 2B, 2C, 2E, 2K and 2L; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G. In some embodiments, the acetyl-CoA pathway comprises 2A, 2B, 2C, 2E, 2K and 2L, and the 1,3-BDO pathway comprises 7E, 7F, 4E, 4F and 4G. In other embodiments, the acetyl-CoA pathway comprises 2A, 2B, 2C, 2E, 2K and 2L, and the 1,3-BDO pathway comprises 7E, 7F, 4B and 4D. In one embodiment, the acetyl-CoA pathway comprises 2A, 2B, 2C, 2E, 2K and 2L, and the 1,3-BDO pathway comprises 7E, 7F, 4E, 4C and 4D. In some embodiments, the acetyl-CoA pathway comprises 2A, 2B, 2C, 2E, 2K and 2L, and the 1,3-BDO pathway comprises 7E, 7F, 4H and 4J. In other embodiments, the acetyl-CoA pathway comprises 2A, 2B, 2C, 2E, 2K and 2L, and the 1,3-BDO pathway comprises 7E, 7F, 4H, 4I and 4G. In certain embodiments, the acetyl-CoA pathway comprises 2A, 2B, 2C, 2E, 2K and 2L, and the 1,3-BDO pathway comprises 7E, 7F, 4H, 4M, 4N and 4G. In another embodiment, the acetyl-CoA pathway comprises 2A, 2B, 2C, 2E, 2K and 2L, and the 1,3-BDO pathway comprises 7E, 7F, 4K, 4O, 4N and 4G. In yet another embodiment, the acetyl-CoA pathway comprises 2A, 2B, 2C, 2E, 2K and 2L, and the 1,3-BDO pathway comprises 7E, 7F, 4K, 4L, 4F and 4G. In certain embodiments, the acetyl-CoA pathway optionally further comprises 2G, 3H, 3I, 3J, or any combination thereof. In some embodiments, the non-naturally occurring eukaryotic organism comprises exogenous nucleic acids, wherein each of the exogenous nucleic acids encodes a different acetyl-CoA pathway or 1,3-BDO pathway enzyme.

In certain embodiments, (1) the acetyl-CoA pathway comprises 5A, 5B, 5C, 5D 5E, 5F, 5G, 5H, 5I, 5J or any combination of 5A, 5B, 5C, 5D, 5E, 5F, 5G, 5H, 5I and 5J thereof, wherein 5A is a pyruvate oxidase (acetate forming); 5B is an acetyl-CoA synthetase, ligase or transferase; 5C is an acetate kinase; 5D is a phosphotransacetylase; 5E is a pyruvate decarboxylase; 5F is an acetaldehyde dehydrogenase; 5G is a pyruvate oxidase (acetyl-phosphate forming); 5H is a pyruvate dehydrogenase, pyruvate:ferredoxin oxidoreductase or pyruvate formate lyase; 5I acetaldehyde dehydrogenase (acylating); and 5J is a threonine aldolase; and (2) the 1,3-BDO pathway comprises 7E, 7F, 4B, 4C, 4D, 4E, 4F, 4G, 4H, 4I, 4J, 4K, 4L, 4M, 4N or 4O, or any combination of 7E, 7F, 4B, 4C, 4D, 4E, 4F, 4G, 4H, 4I, 4J, 4K, 4L, 4M, 4N and 4O thereof wherein 7E, 7F is an acetoacetyl-CoA thiolase; wherein 4B is an acetoacetyl-CoA reductase (CoA-dependent, alcohol forming); wherein 4C is a 3-oxobutyraldehyde reductase (aldehyde reducing); wherein 4D is a 4-hydroxy,2-butanone reductase; wherein 4E is an acetoacetyl-CoA reductase (CoA-dependent, aldehyde forming); wherein 4F is a 3-oxobutyraldehyde reductase (ketone reducing); wherein 4G is a 3-hydroxybutyraldehyde reductase; wherein 4H is an acetoacetyl-CoA reductase (ketone reducing); wherein 4I is a 3-hydroxybutyryl-CoA reductase (aldehyde forming); wherein 4J is a 3-hydroxybutyryl-CoA reductase (alcohol forming); wherein 4K is an acetoacetyl-CoA transferase, an acetoacetyl-CoA hydrolase, an acetoacetyl-CoA synthetase, or a phosphotransacetoacetylase and acetoacetate kinase; wherein 4L is an acetoacetate reductase; wherein 4M is a 3-hydroxybutyryl-CoA transferase, hydrolase, or synthetase; wherein 4N is a 3-hydroxybutyrate reductase; and wherein 4O is a 3-hydroxybutyrate dehydrogenase. In certain embodiments, 5B is an acetyl-CoA synthetase. In another embodiment, 5B is an acetyl-CoA ligase. In other embodiments, 5B is an acetyl-CoA transferase. In some embodiments, 5H is a pyruvate dehydrogenase. In other embodiments, 5H is a pyruvate:ferredoxin oxidoreductase. In yet other embodiments, 5H is a pyruvate formate lyase. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In some embodiments, the acetyl-CoA pathway is an acetyl-CoA pathway depicted in FIG. 5, and the 1,3-BDO pathway is a 1,3-BDO pathway depicted in FIGS. 4 and/or 7. Exemplary sets of acetyl-CoA pathway enzymes, according to FIG. 5, are 5A and 5B; 5A, 5C and 5D; 5G and 5D; 5E, 5F, 5C and 5D; 5J and 5I; 5J, 5F and 5B; and 5H. Exemplary sets of 1,3-BDO pathway enzymes to convert acetyl-CoA to 1,3-BDO, according to FIGS. 4 and 7, include 7E, 7F, 4E, 4F and 4G; 7E, 7F, 4B and 4D; 7E, 7F, 4E, 4C and 4D; 7E, 7F, 4H and 4J; 7E, 7F, 4H, 4I and 4G; 7E, 7F, 4H, 4M, 4N and 4G; 7E, 7F, 4K, 4O, 4N and 4G; or 7E, 7F, 4K, 4L, 4F and 4G.

In some embodiments, (1) the acetyl-CoA pathway comprises (i) 5A and 5B; (ii) 5A, 5C and 5D; (iii) 5E, 5F, 5C and 5D; (iv) 5G and 5D; (v) 5J and 5I; (vi) 5J, 5F and 5B; or (vii) 5H; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G.

In some embodiments, the acetyl-CoA pathway comprises 5A and 5B; and the 1,3-BDO pathway comprises 7E, 7F, 4E, 4F and 4G. In other embodiments, the acetyl-CoA pathway comprises 5A and 5B; and the 1,3-BDO pathway comprises 7E, 7F, 4B and 4D. In some embodiments, the acetyl-CoA pathway comprises 5A and 5B; and the 1,3-BDO pathway comprises 7E, 7F, 4E, 4C and 4D. In some embodiments, the acetyl-CoA pathway comprises 5A and 5B; and the 1,3-BDO pathway comprises 7E, 7F, 4H and 4J. In some embodiments, the acetyl-CoA pathway comprises 5A and 5B; and the 1,3-BDO pathway comprises 7E, 7F, 4H, 4I and 4G. In some embodiments, the acetyl-CoA pathway comprises 5A and 5B; and the 1,3-BDO pathway comprises 7E, 7F, 4H, 4M, 4N and 4G. In some embodiments, the acetyl-CoA pathway comprises 5A and 5B; and the 1,3-BDO pathway comprises 7E, 7F, 4K, 4O, 4N and 4G. In some embodiments, the acetyl-CoA pathway comprises 5A and 5B; and the 1,3-BDO pathway comprises 7E, 7F, 4K, 4L, 4F and 4G.

In some embodiments, the acetyl-CoA pathway comprises 5A, 5C and 5D; and the 1,3-BDO pathway comprises 7E, 7F, 4E, 4F and 4G. In other embodiments, the acetyl-CoA pathway comprises 5A, 5C and 5D; and the 1,3-BDO pathway comprises 7E, 7F, 4B and 4D. In some embodiments, the acetyl-CoA pathway comprises 5A, 5C and 5D; and the 1,3-BDO pathway comprises 7E, 7F, 4E, 4C and 4D. In some embodiments, the acetyl-CoA pathway comprises 5A, 5C and 5D; and the 1,3-BDO pathway comprises 7E, 7F, 4H and 4J. In some embodiments, the acetyl-CoA pathway comprises 5A, 5C and 5D; and the 1,3-BDO pathway comprises 7E, 7F, 4H, 4I and 4G. In some embodiments, the acetyl-CoA pathway comprises 5A, 5C and 5D; and the 1,3-BDO pathway comprises 7E, 7F, 4H, 4M, 4N and 4G. In some embodiments, the acetyl-CoA pathway comprises 5A, 5C and 5D; and the 1,3-BDO pathway comprises 7E, 7F, 4K, 4O, 4N and 4G. In some embodiments, the acetyl-CoA pathway comprises 5A, 5C and 5D; and the 1,3-BDO pathway comprises 7E, 7F, 4K, 4L, 4F and 4G.

In some embodiments, the acetyl-CoA pathway comprises 5E, 5F, 5C and 5D; and the 1,3-BDO pathway comprises 7E, 7F, 4E, 4F and 4G. In other embodiments, the acetyl-CoA pathway comprises 5E, 5F, 5C and 5D; and the 1,3-BDO pathway comprises 7E, 7F, 4B and 4D. In some embodiments, the acetyl-CoA pathway comprises 5E, 5F, 5C and 5D; and the 1,3-BDO pathway comprises 7E, 7F, 4E, 4C and 4D. In some embodiments, the acetyl-CoA pathway comprises 5E, 5F, 5C and 5D; and the 1,3-BDO pathway comprises 7E, 7F, 4H and 4J. In some embodiments, the acetyl-CoA pathway comprises 5E, 5F, 5C and 5D; and the 1,3-BDO pathway comprises 7E, 7F, 4H, 4I and 4G. In some embodiments, the acetyl-CoA pathway comprises 5E, 5F, 5C and 5D; and the 1,3-BDO pathway comprises 7E, 7F, 4H, 4M, 4N and 4G. In some embodiments, the acetyl-CoA pathway comprises 5E, 5F, 5C and 5D; and the 1,3-BDO pathway comprises 7E, 7F, 4K, 4O, 4N and 4G. In some embodiments, the acetyl-CoA pathway comprises 5E, 5F, 5C and 5D; and the 1,3-BDO pathway comprises 7E, 7F, 4K, 4L, 4F and 4G.

In some embodiments, the acetyl-CoA pathway comprises 5G and 5D; and the 1,3-BDO pathway comprises 7E, 7F, 4E, 4F and 4G. In other embodiments, the acetyl-CoA pathway comprises 5G and 5D; and the 1,3-BDO pathway comprises 7E, 7F, 4B and 4D. In some embodiments, the acetyl-CoA pathway comprises 5G and 5D; and the 1,3-BDO pathway comprises 7E, 7F, 4E, 4C and 4D. In some embodiments, the acetyl-CoA pathway comprises 5G and 5D and the 1,3-BDO pathway comprises 7E, 7F, 4H and 4J. In some embodiments, the acetyl-CoA pathway comprises 5G and 5D; and the 1,3-BDO pathway comprises 7E, 7F, 4H, 4I and 4G. In some embodiments, the acetyl-CoA pathway comprises 5G and 5D; and the 1,3-BDO pathway comprises 7E, 7F, 4H, 4M, 4N and 4G. In some embodiments, the acetyl-CoA pathway comprises 5G and 5D; and the 1,3-BDO pathway comprises 7E, 7F, 4K, 4O, 4N and 4G. In some embodiments, the acetyl-CoA pathway comprises 5G and 5D; and the 1,3-BDO pathway comprises 7E, 7F, 4K, 4L, 4F and 4G.

In some embodiments, the acetyl-CoA pathway comprises 5J and 5I; and the 1,3-BDO pathway comprises 7E, 7F, 4E, 4F and 4G. In other embodiments, the acetyl-CoA pathway comprises 5J and 5I; and the 1,3-BDO pathway comprises 7E, 7F, 4B and 4D. In some embodiments, the acetyl-CoA pathway comprises 5J and 5I; and the 1,3-BDO pathway comprises 7E, 7F, 4E, 4C and 4D. In some embodiments, the acetyl-CoA pathway comprises 5J and 5I; and the 1,3-BDO pathway comprises 7E, 7F, 4H and 4J. In some embodiments, the acetyl-CoA pathway comprises 5J and 5I; and the 1,3-BDO pathway comprises 7E, 7F, 4H, 4I and 4G. In some embodiments, the acetyl-CoA pathway comprises 5J and 5I; and the 1,3-BDO pathway comprises 7E, 7F, 4H, 4M, 4N and 4G. In some embodiments, the acetyl-CoA pathway comprises 5J and 5I; and the 1,3-BDO pathway comprises 7E, 7F, 4K, 4O, 4N and 4G. In some embodiments, the acetyl-CoA pathway comprises 5J and 5I; and the 1,3-BDO pathway comprises 7E, 7F, 4K, 4L, 4F and 4G.

In some embodiments, the acetyl-CoA pathway comprises 5J, 5F and 5B; and the 1,3-BDO pathway comprises 7E, 7F, 4E, 4F and 4G. In other embodiments, the acetyl-CoA pathway comprises 5J, 5F and 5B; and the 1,3-BDO pathway comprises 7E, 7F, 4B and 4D. In some embodiments, the acetyl-CoA pathway comprises 5J, 5F and 5B; and the 1,3-BDO pathway comprises 7E, 7F, 4E, 4C and 4D. In some embodiments, the acetyl-CoA pathway comprises 5J, 5F and 5B; and the 1,3-BDO pathway comprises 7E, 7F, 4H and 4J. In some embodiments, the acetyl-CoA pathway comprises 5J, 5F and 5B; and the 1,3-BDO pathway comprises 7E, 7F, 4H, 4I and 4G. In some embodiments, the acetyl-CoA pathway comprises 5J, 5F and 5B; and the 1,3-BDO pathway comprises 7E, 7F, 4H, 4M, 4N and 4G. In some embodiments, the acetyl-CoA pathway comprises 5J, 5F and 5B; and the 1,3-BDO pathway comprises 7E, 7F, 4K, 4O, 4N and 4G. In some embodiments, the acetyl-CoA pathway comprises 5J, 5F and 5B; and the 1,3-BDO pathway comprises 7E, 7F, 4K, 4L, 4F and 4G.

In some embodiments, the acetyl-CoA pathway comprises 5H; and the 1,3-BDO pathway comprises 7E, 7F, 4E, 4F and 4G. In other embodiments, the acetyl-CoA pathway comprises 5H; and the 1,3-BDO pathway comprises 7E, 7F, 4B and 4D. In some embodiments, the acetyl-CoA pathway comprises 5H; and the 1,3-BDO pathway comprises 7E, 7F, 4E, 4C and 4D. In some embodiments, the acetyl-CoA pathway comprises 5H; and the 1,3-BDO pathway comprises 7E, 7F, 4H and 4J. In some embodiments, the acetyl-CoA pathway comprises 5H; and the 1,3-BDO pathway comprises 7E, 7F, 4H, 4I and 4G. In some embodiments, the acetyl-CoA pathway comprises 5H; and the 1,3-BDO pathway comprises 7E, 7F, 4H, 4M, 4N and 4G. In some embodiments, the acetyl-CoA pathway comprises 5H; and the 1,3-BDO pathway comprises 7E, 7F, 4K, 4O, 4N and 4G. In some embodiments, the acetyl-CoA pathway comprises 5H; and the 1,3-BDO pathway comprises 7E, 7F, 4K, 4L, 4F and 4G.

In certain embodiments, (1) the acetyl-CoA pathway comprises 6A, 6B, 6C, 6D or 6E, or any combination of 6A, 6B, 6C, 6D and 6E thereof, wherein 6A is mitochondrial acetylcarnitine transferase; 6B is a peroxisomal acetylcarnitine transferase; 6C is a cytosolic acetylcarnitine transferase; 6D is a mitochondrial acetylcarnitine translocase; and 6E. is peroxisomal acetylcarnitine translocase; and (2) the 1,3-BDO pathway comprises 7E, 7F, 4B, 4C, 4D, 4E, 4F, 4G, 4H, 4I, 4J, 4K, 4L, 4M, 4N or 4O, or any combination of 7E, 7F, 4B, 4C, 4D, 4E, 4F, 4G, 4H, 4I, 4J, 4K, 4L, 4M, 4N and 4O thereof; wherein 7E, 7F is an acetoacetyl-CoA thiolase; wherein 4B is an acetoacetyl-CoA reductase (CoA-dependent, alcohol forming); wherein 4C is a 3-oxobutyraldehyde reductase (aldehyde reducing); wherein 4D is a 4-hydroxy,2-butanone reductase; wherein 4E is an acetoacetyl-CoA reductase (CoA-dependent, aldehyde forming); wherein 4F is a 3-oxobutyraldehyde reductase (ketone reducing); wherein 4G is a 3-hydroxybutyraldehyde reductase; wherein 4H is an acetoacetyl-CoA reductase (ketone reducing); wherein 4I is a 3-hydroxybutyryl-CoA reductase (aldehyde forming); wherein 4J is a 3-hydroxybutyryl-CoA reductase (alcohol forming); wherein 4K is an acetoacetyl-CoA transferase, an acetoacetyl-CoA hydrolase, an acetoacetyl-CoA synthetase, or a phosphotransacetoacetylase and acetoacetate kinase; wherein 4L is an acetoacetate reductase; wherein 4M is a 3-hydroxybutyryl-CoA transferase, hydrolase, or synthetase; wherein 4N is a 3-hydroxybutyrate reductase; and wherein 4O is a 3-hydroxybutyrate dehydrogenase. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In some embodiments, the acetyl-CoA pathway is an acetyl-CoA pathway depicted in FIG. 6, and the 1,3-BDO pathway is a 1,3-BDO pathway depicted in FIGS. 4 and/or 7. Exemplary sets of acetyl-CoA pathway enzymes, according to FIG. 6, are 6A, 6D and 6C; and 6B, 6E and 6C. Exemplary sets of 1,3-BDO pathway enzymes to convert acetyl-CoA to 1,3-BDO, according to FIGS. 4 and 7, include 7E, 7F, 4E, 4F and 4G; 7E, 7F, 4B and 4D; 7E, 7F, 4E, 4C and 4D; 7E, 7F, 4H and 4J; 7E, 7F, 4H, 4I and 4G; 7E, 7F, 4H, 4M, 4N and 4G; 7E, 7F, 4K, 4O, 4N and 4G; or 7E, 7F, 4K, 4L, 4F and 4G.

In one embodiment, (1) the acetyl-CoA pathway comprises (i) 6A, 6D and 6C; or (ii) 6B, 6E and 6C; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G.

In some embodiments, the acetyl-CoA pathway comprises 6A, 6D and 6C; and the 1,3-BDO pathway comprises 7E, 7F, 4E, 4F and 4G. In other embodiments, the acetyl-CoA pathway comprises 6A, 6D and 6C; and the 1,3-BDO pathway comprises 7E, 7F, 4B and 4D. In some embodiments, the acetyl-CoA pathway comprises 6A, 6D and 6C; and the 1,3-BDO pathway comprises 7E, 7F, 4E, 4C and 4D. In some embodiments, the acetyl-CoA pathway comprises 6A, 6D and 6C; and the 1,3-BDO pathway comprises 7E, 7F, 4H and 4J. In some embodiments, the acetyl-CoA pathway comprises 6A, 6D and 6C; and the 1,3-BDO pathway comprises 7E, 7F, 4H, 4I and 4G. In some embodiments, the acetyl-CoA pathway comprises 6A, 6D and 6C; and the 1,3-BDO pathway comprises 7E, 7F, 4H, 4M, 4N and 4G. In some embodiments, the acetyl-CoA pathway comprises 6A, 6D and 6C; and the 1,3-BDO pathway comprises 7E, 7F, 4K, 4O, 4N and 4G. In some embodiments, the acetyl-CoA pathway comprises 6A, 6D and 6C; and the 1,3-BDO pathway comprises 7E, 7F, 4K, 4L, 4F and 4G.

In some embodiments, the acetyl-CoA pathway comprises 6B, 6E and 6C; and the 1,3-BDO pathway comprises 7E, 7F, 4E, 4F and 4G. In other embodiments, the acetyl-CoA pathway comprises 6B, 6E and 6C; and the 1,3-BDO pathway comprises 7E, 7F, 4B and 4D. In some embodiments, the acetyl-CoA pathway comprises 6B, 6E and 6C; and the 1,3-BDO pathway comprises 7E, 7F, 4E, 4C and 4D. In some embodiments, the acetyl-CoA pathway comprises 6B, 6E and 6C; and the 1,3-BDO pathway comprises 7E, 7F, 4H and 4J. In some embodiments, the acetyl-CoA pathway comprises 6B, 6E and 6C; and the 1,3-BDO pathway comprises 7E, 7F, 4H, 4I and 4G. In some embodiments, the acetyl-CoA pathway comprises 6B, 6E and 6C; and the 1,3-BDO pathway comprises 7E, 7F, 4H, 4M, 4N and 4G. In some embodiments, the acetyl-CoA pathway comprises 6B, 6E and 6C; and the 1,3-BDO pathway comprises 7E, 7F, 4K, 4O, 4N and 4G. In some embodiments, the acetyl-CoA pathway comprises 6B, 6E and 6C; and the 1,3-BDO pathway comprises 7E, 7F, 4K, 4L, 4F and 4G.

In certain embodiments, (1) the acetyl-CoA pathway comprises 10A, 10B, 10C, 10D, 10F, 10G, 10H. 10J, 10K, 10L, 10M, 10N, or any combination of 10A, 10B, 10C, 10D, 10F, 10G, 10H. 10J, 10K, 10L, 10M, 10N thereof; and (2) the 1,3-BDO pathway comprises 7E (see also FIG. 10, step D), 7F (see also FIG. 10, step E), 4B, 4C, 4D, 4E, 4F, 4G, 4H, 4I, 4J, 4K, 4L, 4M, 4N or 4O, or any combination of 7E, 7F, 4B, 4C, 4D, 4E, 4F, 4G, 4H, 4I, 4J, 4K, 4L, 4M, 4N and 4O thereof. In certain embodiments, 4K is an acetoacetyl-CoA transferase. In one embodiment, 10A is a PEP carboxylase. In another embodiment, 10A is a PEP carboxykinase. In an embodiment, 1° F. is an oxaloacetate dehydrogenase. In other embodiments, 1° F. is an oxaloacetate oxidoreductase. In one embodiment, 10K is a malonyl-CoA synthetase. In another embodiment, 10K is a malonyl-CoA transferase. In one embodiment, 10M is a malate dehydrogenase. In another embodiment, 10M is a malate oxidoreductase. In other embodiments, 10N is a pyruvate kinase. In some embodiments, 10N is a PEP phosphatase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In some embodiments, the acetyl-CoA pathway is an acetyl-CoA pathway depicted in FIG. 10, and the 1,3-BDO pathway is a 1,3-BDO pathway depicted in FIGS. 4 and/or 7. Exemplary sets of acetyl-CoA pathway enzymes, according to FIG. 10, are 10A, 10B and 10C; 10N, 10H, 10B and 10C; 10N, 10L, 10M, 10B and 10C; 10A, 10B, 10G and 10D; 10N, 10H, 10B, 10G and 10D; 10N, 10L, 10M, 10B, 10G and 10D; 10A, 10B, 10J, 10K and 10D; 10N, 10H, 10B, 10J, 10K and 10D; 10N, 10L, 10M, 10B, 10J, 10K and 10D; 10A, 10F and 10D; 10N, 10H, 10F and 10D; and 10N, 10L, 10M, 10F and 10D. Exemplary sets of 1,3-BDO pathway enzymes to convert acetyl-CoA to 1,3-BDO, according to FIGS. 4 and 7, include 7E, 7F, 4E, 4F and 4G; 7E, 7F, 4B and 4D; 7E, 7F, 4E, 4C and 4D; 7E, 7F, 4H and 4J; 7E, 7F, 4H, 4I and 4G; 7E, 7F, 4H, 4M, 4N and 4G; 7E, 7F, 4K, 4O, 4N and 4G; or 7E, 7F, 4K, 4L, 4F and 4G.

In one embodiment, (1) the acetyl-CoA pathway comprises (i) 10A, 10B and 10C; (ii) 10N, 10H, 10B and 10C; (iii) 10N, 10L, 10M, 10B and 10C; (iv) 10A, 10B, 10G and 10D; (v) 10N, 10H, 10B, 10G and 10D; (vi) 10N, 10L, 10M, 10B, 10G and 10D; (vii) 10A, 10B, 10J, 10K and 10D; (viii) 10N, 10H, 10B, 10J, 10K and 10D; (ix) 10N, 10L, 10M, 10B, 10J, 10K and 10D; (x) 10A, 10F and 10D; (xi) 10N, 10H, 10F and 10D; or (xii) 10N, 10L, 10M, 10F and 10D; and (2) the 1,3-BDO pathway comprises (i) 7E, 7F, 4E, 4F and 4G; (ii) 7E, 7F, 4B and 4D; (iii) 7E, 7F, 4E, 4C and 4D; (iv) 7E, 7F, 4H and 4J; (v) 7E, 7F, 4H, 4I and 4G; (vi) 7E, 7F, 4H, 4M, 4N and 4G; (vii) 7E, 7F, 4K, 4O, 4N and 4G; or (viii) 7E, 7F, 4K, 4L, 4F and 4G.

In some embodiments, the acetyl-CoA pathway comprises 10A, 10B and 10C; and the 1,3-BDO pathway comprises 7E, 7F, 4E, 4F and 4G. In other embodiments, the acetyl-CoA pathway comprises 10A, 10B and 10C; and the 1,3-BDO pathway comprises 7E, 7F, 4B and 4D. In some embodiments, the acetyl-CoA pathway comprises 10A, 10B and 10C; and the 1,3-BDO pathway comprises 7E, 7F, 4E, 4C and 4D. In some embodiments, the acetyl-CoA pathway comprises 10A, 10B and 10C; and the 1,3-BDO pathway comprises 7E, 7F, 4H and 4J. In some embodiments, the acetyl-CoA pathway comprises 10A, 10B and 10C; and the 1,3-BDO pathway comprises 7E, 7F, 4H, 4I and 4G. In some embodiments, the acetyl-CoA pathway comprises 10A, 10B and 10C; and the 1,3-BDO pathway comprises 7E, 7F, 4H, 4M, 4N and 4G. In some embodiments, the acetyl-CoA pathway comprises 10A, 10B and 10C; and the 1,3-BDO pathway comprises 7E, 7F, 4K, 4O, 4N and 4G. In some embodiments, the acetyl-CoA pathway comprises 10A, 10B and 10C; and the 1,3-BDO pathway comprises 7E, 7F, 4K, 4L, 4F and 4G.

In some embodiments, the acetyl-CoA pathway comprises 10N, 10H, 10B and 10C; and the 1,3-BDO pathway comprises 7E, 7F, 4E, 4F and 4G. In other embodiments, the acetyl-CoA pathway comprises 10N, 10H, 10B and 10C; and the 1,3-BDO pathway comprises 7E, 7F, 4B and 4D. In some embodiments, the acetyl-CoA pathway comprises 10N, 10H, 10B and 10C; and the 1,3-BDO pathway comprises 7E, 7F, 4E, 4C and 4D. In some embodiments, the acetyl-CoA pathway comprises 10N, 10H, 10B and 10C; and the 1,3-BDO pathway comprises 7E, 7F, 4H and 4J. In some embodiments, the acetyl-CoA pathway comprises 10N, 10H, 10B and 10C; and the 1,3-BDO pathway comprises 7E, 7F, 4H, 4I and 4G. In some embodiments, the acetyl-CoA pathway comprises 10N, 10H, 10B and 10C; and the 1,3-BDO pathway comprises 7E, 7F, 4H, 4M, 4N and 4G. In some embodiments, the acetyl-CoA pathway comprises 10N, 10H, 10B and 10C; and the 1,3-BDO pathway comprises 7E, 7F, 4K, 4O, 4N and 4G. In some embodiments, the acetyl-CoA pathway comprises 10N, 10H, 10B and 10C; and the 1,3-BDO pathway comprises 7E, 7F, 4K, 4L, 4F and 4G.

In some embodiments, the acetyl-CoA pathway comprises 10N, 10L, 10M, 10B and 10C; and the 1,3-BDO pathway comprises 7E, 7F, 4E, 4F and 4G. In other embodiments, the acetyl-CoA pathway comprises 10N, 10L, 10M, 10B and 10C; and the 1,3-BDO pathway comprises 7E, 7F, 4B and 4D. In some embodiments, the acetyl-CoA pathway comprises 10N, 10L, 10M, 10B and 10C; and the 1,3-BDO pathway comprises 7E, 7F, 4E, 4C and 4D. In some embodiments, the acetyl-CoA pathway comprises 10N, 10L, 10M, 10B and 10C; and the 1,3-BDO pathway comprises 7E, 7F, 4H and 4J. In some embodiments, the acetyl-CoA pathway comprises 10N, 10L, 10M, 10B and 10C; and the 1,3-BDO pathway comprises 7E, 7F, 4H, 4I and 4G. In some embodiments, the acetyl-CoA pathway comprises 10N, 10L, 10M, 10B and 10C; and the 1,3-BDO pathway comprises 7E, 7F, 4H, 4M, 4N and 4G. In some embodiments, the acetyl-CoA pathway comprises 10N, 10L, 10M, 10B and 10C; and the 1,3-BDO pathway comprises 7E, 7F, 4K, 4O, 4N and 4G. In some embodiments, the acetyl-CoA pathway comprises 10N, 10L, 10M, 10B and 10C; and the 1,3-BDO pathway comprises 7E, 7F, 4K, 4L, 4F and 4G.

In some embodiments, the acetyl-CoA pathway comprises 10A, 10B, 10G and 10D; and the 1,3-BDO pathway comprises 7E, 7F, 4E, 4F and 4G. In other embodiments, the acetyl-CoA pathway comprises 10A, 10B, 10G and 10D; and the 1,3-BDO pathway comprises 7E, 7F, 4B and 4D. In some embodiments, the acetyl-CoA pathway comprises 10A, 10B, 10G and 10D; and the 1,3-BDO pathway comprises 7E, 7F, 4E, 4C and 4D. In some embodiments, the acetyl-CoA pathway comprises 10A, 10B, 10G and 10D; and the 1,3-BDO pathway comprises 7E, 7F, 4H and 4J. In some embodiments, the acetyl-CoA pathway comprises 10A, 10B, 10G and 10D; and the 1,3-BDO pathway comprises 7E, 7F, 4H, 4I and 4G. In some embodiments, the acetyl-CoA pathway comprises 10A, 10B, 10G and 10D; and the 1,3-BDO pathway comprises 7E, 7F, 4H, 4M, 4N and 4G. In some embodiments, the acetyl-CoA pathway comprises 10A, 10B, 10G and 10D; and the 1,3-BDO pathway comprises 7E, 7F, 4K, 4O, 4N and 4G. In some embodiments, the acetyl-CoA pathway comprises 10A, 10B, 10G and 10D; and the 1,3-BDO pathway comprises 7E, 7F, 4K, 4L, 4F and 4G.

In some embodiments, the acetyl-CoA pathway comprises 10N, 10H, 10B, 10G and 10D; and the 1,3-BDO pathway comprises 7E, 7F, 4E, 4F and 4G. In other embodiments, the acetyl-CoA pathway comprises 10N, 10H, 10B, 10G and 10D; and the 1,3-BDO pathway comprises 7E, 7F, 4B and 4D. In some embodiments, the acetyl-CoA pathway comprises 10N, 10H, 10B, 10G and 10D; and the 1,3-BDO pathway comprises 7E, 7F, 4E, 4C and 4D. In some embodiments, the acetyl-CoA pathway comprises 10N, 10H, 10B, 10G and 10D; and the 1,3-BDO pathway comprises 7E, 7F, 4H and 4J. In some embodiments, the acetyl-CoA pathway comprises 10N, 10H, 10B, 10G and 10D; and the 1,3-BDO pathway comprises 7E, 7F, 4H, 4I and 4G. In some embodiments, the acetyl-CoA pathway comprises 10N, 10H, 10B, 10G and 10D; and the 1,3-BDO pathway comprises 7E, 7F, 4H, 4M, 4N and 4G. In some embodiments, the acetyl-CoA pathway comprises 10N, 10H, 10B, 10G and 10D; and the 1,3-BDO pathway comprises 7E, 7F, 4K, 4O, 4N and 4G. In some embodiments, the acetyl-CoA pathway comprises 10N, 10H, 10B, 10G and 10D; and the 1,3-BDO pathway comprises 7E, 7F, 4K, 4L, 4F and 4G.

In some embodiments, the acetyl-CoA pathway comprises 10N, 10L, 10M, 10B, 10G and 10D; and the 1,3-BDO pathway comprises 7E, 7F, 4E, 4F and 4G. In other embodiments, the acetyl-CoA pathway comprises 10N, 10L, 10M, 10B, 10G and 10D; and the 1,3-BDO pathway comprises 7E, 7F, 4B and 4D. In some embodiments, the acetyl-CoA pathway comprises 10N, 10L, 10M, 10B, 10G and 10D; and the 1,3-BDO pathway comprises 7E, 7F, 4E, 4C and 4D. In some embodiments, the acetyl-CoA pathway comprises 10N, 10L, 10M, 10B, 10G and 10D; and the 1,3-BDO pathway comprises 7E, 7F, 4H and 4J. In some embodiments, the acetyl-CoA pathway comprises 10N, 10L, 10M, 10B, 10G and 10D; and the 1,3-BDO pathway comprises 7E, 7F, 4H, 4I and 4G. In some embodiments, the acetyl-CoA pathway comprises 10N, 10L, 10M, 10B, 10G and 10D; and the 1,3-BDO pathway comprises 7E, 7F, 4H, 4M, 4N and 4G. In some embodiments, the acetyl-CoA pathway comprises 10N, 10L, 10M, 10B, 10G and 10D; and the 1,3-BDO pathway comprises 7E, 7F, 4K, 4O, 4N and 4G. In some embodiments, the acetyl-CoA pathway comprises 10N, 10L, 10M, 10B, 10G and 10D; and the 1,3-BDO pathway comprises 7E, 7F, 4K, 4L, 4F and 4G.

In some embodiments, the acetyl-CoA pathway comprises 10A, 10B, 10J, 10K and 10D; and the 1,3-BDO pathway comprises 7E, 7F, 4E, 4F and 4G. In other embodiments, the acetyl-CoA pathway comprises 10A, 10B, 10J, 10K and 10D; and the 1,3-BDO pathway comprises 7E, 7F, 4B and 4D. In some embodiments, the acetyl-CoA pathway comprises 10A, 10B, 10J, 10K and 10D; and the 1,3-BDO pathway comprises 7E, 7F, 4E, 4C and 4D. In some embodiments, the acetyl-CoA pathway comprises 10A, 10B, 10J, 10K and 10D; and the 1,3-BDO pathway comprises 7E, 7F, 4H and 4J. In some embodiments, the acetyl-CoA pathway comprises 10A, 10B, 10J, 10K and 10D; and the 1,3-BDO pathway comprises 7E, 7F, 4H, 4I and 4G. In some embodiments, the acetyl-CoA pathway comprises 10A, 10B, 10J, 10K and 10D; and the 1,3-BDO pathway comprises 7E, 7F, 4H, 4M, 4N and 4G. In some embodiments, the acetyl-CoA pathway comprises 10A, 10B, 10J, 10K and 10D; and the 1,3-BDO pathway comprises 7E, 7F, 4K, 4O, 4N and 4G. In some embodiments, the acetyl-CoA pathway comprises 10A, 10B, 10J, 10K and 10D; and the 1,3-BDO pathway comprises 7E, 7F, 4K, 4L, 4F and 4G.

In some embodiments, the acetyl-CoA pathway comprises 10N, 10H, 10B, 10J, 10K and 10D; and the 1,3-BDO pathway comprises 7E, 7F, 4E, 4F and 4G. In other embodiments, the acetyl-CoA pathway comprises 10N, 10H, 10B, 10J, 10K and 10D; and the 1,3-BDO pathway comprises 7E, 7F, 4B and 4D. In some embodiments, the acetyl-CoA pathway comprises 10N, 10H, 10B, 10J, 10K and 10D; and the 1,3-BDO pathway comprises 7E, 7F, 4E, 4C and 4D. In some embodiments, the acetyl-CoA pathway comprises 10N, 10H, 10B, 10J, 10K and 10D; and the 1,3-BDO pathway comprises 7E, 7F, 4H and 4J. In some embodiments, the acetyl-CoA pathway comprises 10N, 10H, 10B, 10J, 10K and 10D; and the 1,3-BDO pathway comprises 7E, 7F, 4H, 4I and 4G. In some embodiments, the acetyl-CoA pathway comprises 10N, 10H, 10B, 10J, 10K and 10D; and the 1,3-BDO pathway comprises 7E, 7F, 4H, 4M, 4N and 4G. In some embodiments, the acetyl-CoA pathway comprises 10N, 10H, 10B, 10J, 10K and 10D; and the 1,3-BDO pathway comprises 7E, 7F, 4K, 4O, 4N and 4G. In some embodiments, the acetyl-CoA pathway comprises 10N, 10H, 10B, 10J, 10K and 10D; and the 1,3-BDO pathway comprises 7E, 7F, 4K, 4L, 4F and 4G.

In some embodiments, the acetyl-CoA pathway comprises 10N, 10L, 10M, 10B, 10J, 10K and 10D; and the 1,3-BDO pathway comprises 7E, 7F, 4E, 4F and 4G. In other embodiments, the acetyl-CoA pathway comprises 10N, 10L, 10M, 10B, 10J, 10K and 10D; and the 1,3-BDO pathway comprises 7E, 7F, 4B and 4D. In some embodiments, the acetyl-CoA pathway comprises 10N, 10L, 10M, 10B, 10J, 10K and 10D; and the 1,3-BDO pathway comprises 7E, 7F, 4E, 4C and 4D. In some embodiments, the acetyl-CoA pathway comprises 10N, 10L, 10M, 10B, 10J, 10K and 10D; and the 1,3-BDO pathway comprises 7E, 7F, 4H and 4J. In some embodiments, the acetyl-CoA pathway comprises 10N, 10L, 10M, 10B, 10J, 10K and 10D; and the 1,3-BDO pathway comprises 7E, 7F, 4H, 4I and 4G. In some embodiments, the acetyl-CoA pathway comprises 10N, 10L, 10M, 10B, 10J, 10K and 10D; and the 1,3-BDO pathway comprises 7E, 7F, 4H, 4M, 4N and 4G. In some embodiments, the acetyl-CoA pathway comprises 10N, 10L, 10M, 10B, 10J, 10K and 10D; and the 1,3-BDO pathway comprises 7E, 7F, 4K, 4O, 4N and 4G. In some embodiments, the acetyl-CoA pathway comprises 10N, 10L, 10M, 10B, 10J, 10K and 10D; and the 1,3-BDO pathway comprises 7E, 7F, 4K, 4L, 4F and 4G.

In some embodiments, the acetyl-CoA pathway comprises 10A, 10F and 10D; and the 1,3-BDO pathway comprises 7E, 7F, 4E, 4F and 4G. In other embodiments, the acetyl-CoA pathway comprises 10A, 10F and 10D; and the 1,3-BDO pathway comprises 7E, 7F, 4B and 4D. In some embodiments, the acetyl-CoA pathway comprises 10A, 10F and 10D; and the 1,3-BDO pathway comprises 7E, 7F, 4E, 4C and 4D. In some embodiments, the acetyl-CoA pathway comprises 10A, 10F and 10D; and the 1,3-BDO pathway comprises 7E, 7F, 4H and 4J. In some embodiments, the acetyl-CoA pathway comprises 10A, 10F and 10D; and the 1,3-BDO pathway comprises 7E, 7F, 4H, 4I and 4G. In some embodiments, the acetyl-CoA pathway comprises 10A, 10F and 10D; and the 1,3-BDO pathway comprises 7E, 7F, 4H, 4M, 4N and 4G. In some embodiments, the acetyl-CoA pathway comprises 10A, 10F and 10D; and the 1,3-BDO pathway comprises 7E, 7F, 4K, 4O, 4N and 4G. In some embodiments, the acetyl-CoA pathway comprises 10A, 10F and 10D; and the 1,3-BDO pathway comprises 7E, 7F, 4K, 4L, 4F and 4G.

In some embodiments, the acetyl-CoA pathway comprises 10N, 10H, 10F and 10D; and the 1,3-BDO pathway comprises 7E, 7F, 4E, 4F and 4G. In other embodiments, the acetyl-CoA pathway comprises 10N, 10H, 10F and 10D; and the 1,3-BDO pathway comprises 7E, 7F, 4B and 4D. In some embodiments, the acetyl-CoA pathway comprises 10N, 10H, 10F and 10D; and the 1,3-BDO pathway comprises 7E, 7F, 4E, 4C and 4D. In some embodiments, the acetyl-CoA pathway comprises 10N, 10H, 10F and 10D; and the 1,3-BDO pathway comprises 7E, 7F, 4H and 4J. In some embodiments, the acetyl-CoA pathway comprises 10N, 10H, 10F and 10D; and the 1,3-BDO pathway comprises 7E, 7F, 4H, 4I and 4G. In some embodiments, the acetyl-CoA pathway comprises 10N, 10H, 10F and 10D; and the 1,3-BDO pathway comprises 7E, 7F, 4H, 4M, 4N and 4G. In some embodiments, the acetyl-CoA pathway comprises 10N, 10H, 1° F. and 10D; and the 1,3-BDO pathway comprises 7E, 7F, 4K, 4O, 4N and 4G. In some embodiments, the acetyl-CoA pathway comprises 10N, 10H, 10F and 10D; and the 1,3-BDO pathway comprises 7E, 7F, 4K, 4L, 4F and 4G.

In some embodiments, the acetyl-CoA pathway comprises 10N, 10L. 10M, 10F and 10D; and the 1,3-BDO pathway comprises 7E, 7F, 4E, 4F and 4G. In other embodiments, the acetyl-CoA pathway comprises 10N, 10L. 10M, 10F and 10D; and the 1,3-BDO pathway comprises 7E, 7F, 4B and 4D. In some embodiments, the acetyl-CoA pathway comprises 10N, 10L. 10M, 10F and 10D; and the 1,3-BDO pathway comprises 7E, 7F, 4E, 4C and 4D. In some embodiments, the acetyl-CoA pathway comprises 10N, 10L. 10M, 10F and 10D; and the 1,3-BDO pathway comprises 7E, 7F, 4H and 4J. In some embodiments, the acetyl-CoA pathway comprises 10N, 10L. 10M, 10F and 10D; and the 1,3-BDO pathway comprises 7E, 7F, 4H, 4I and 4G. In some embodiments, the acetyl-CoA pathway comprises 10N, 10L. 10M, 10F and 10D; and the 1,3-BDO pathway comprises 7E, 7F, 4H, 4M, 4N and 4G. In some embodiments, the acetyl-CoA pathway comprises 10N, 10L. 10M, 10F and 10D; and the 1,3-BDO pathway comprises 7E, 7F, 4K, 4O, 4N and 4G. In some embodiments, the acetyl-CoA pathway comprises 10N, 10L. 10M, 10F and 10D; and the 1,3-BDO pathway comprises 7E, 7F, 4K, 4L, 4F and 4G.

In an additional embodiment, provided herein is a non-naturally occurring eukaryotic organism having a 1,3-BDO pathway, wherein the non-naturally occurring eukaryotic organism comprises at least one exogenous nucleic acid encoding an enzyme or protein that converts a substrate to a product selected from the group consisting of acetyl-CoA to acetoacetyl-CoA (e.g., 7E, 7F); acetoacetyl-CoA to 4-hydroxy-2-butanone (e.g., 4B); 3-oxobutyraldehyde to 4-hydroxy-2-butanone (e.g., 4C); 4-hydroxy-2-butanone to 1,3-BDO (e.g., 4D); acetoacetyl-CoA to 3-oxobutyraldehyde (e.g., 4E); 3-oxobutyraldehyde to 3-hydroxybutyrldehyde (e.g., 4F); 3-hydroxybutyrldehyde to 1,3-BDO (e.g., 4G); acetoacetyl-CoA to 3-hydroxybutyryl-CoA (e.g., 4H); 3-hydroxybutyryl-CoA to 3-hydroxybutyraldehyde (e.g., 4I), 3-hydroxybutyryl-CoA to 1,3-BDO (e.g., 4J); acetoacetyl-CoA to acetoacetate (e.g., 4K); acetoacetate to 3-oxobutyraldehyde (e.g., 4L); 3-hydroxybutyrl-CoA to 3-hydroxybutyrate (e.g., 4M); 3-hydroxybutyrate to 3-hydroxybutyraldehyde (e.g., 4N); and acetoacetate to 3-hydroxybutyrate (e.g., 4O). One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, provided herein are non-naturally occurring eukaryotic organisms comprising at least one exogenous nucleic acid encoding an enzyme or protein, where the enzyme or protein converts the substrates and products of a 1,3-BDO pathway, such as that shown in FIG. 4 or 7.

Any combination and any number of the aforementioned enzymes and/or nucleic acids encoding the enzymes thereof, can be introduced into a host eukaryotic organism to complete a 1,3-BDO pathway, as exemplified in FIG. 4 or FIG. 7. For example, the non-naturally occurring eukaryotic organism can include one, two, three, four, five, up to all of the nucleic acids in a 1,3-BDO pathway, each nucleic acid encoding a 1,3-BDO pathway enzyme. Such nucleic acids can include heterologous nucleic acids, additional copies of existing genes, and gene regulatory elements, as explained further below. The pathways of the non-naturally occurring eukaryotic organisms provided herein are also suitably engineered to be cultured in a substantially anaerobic culture medium.

In certain embodiments of the methods provided herein for increasing cytosolic acetyl-CoA involves deleting or attenuating competing pathways that utilize acetyl-CoA. Deletion or attenuation of competing byproduct pathways that utilize acetyl-CoA can be carried out by any method known to those skilled in the art. For example, attenuation of such a competing pathway can be achieved by replacing an endogenous nucleic acid encoding an enzyme of the pathway for a mutated form of the nucleic acid that encodes for a variant of the enzyme with decreased enzymatic activity as compared to wild-type. Deletion of such a pathway can be achieved, for example, by deletion of one or more endogenous nucleic acids encoding for one or more enzymes of the pathway or by replacing the endogenous one or more nucleic acids with null allele variants. Exemplary methods for genetic manipulation of endogenous nucleic acids in host eukaryotic organisms, including Saccharomyces cerevisiae, are described below and in Example X.

For example, one such enzyme in a competing pathway that utilizes acetyl-CoA is the mitochondrial pyruvate dehydrogenase complex. Under anaerobic conditions and in conditions where glucose concentrations are high in the medium, the capacity of this mitochondrial enzyme is very limited and there is no significant flux through it. However, in some embodiments, any of the non-naturally occurring eukaryotic organisms described herein can be engineered to express an attenuated mitochondrial pyruvate dehydrogenase or a null phenotype to increase 1,3-BDO production. Exemplary pyruvate dehydrogenase genes include PDB1, PDA1, LAT1 and LPD1. Exemplary competing acetyl-CoA consuming pathways whose attenuation or deletion can improve 1,3-BDO production include, but are not limited to, the mitochondrial TCA cycle and metabolic pathways, such as fatty acid biosynthesis and amino acid biosynthesis.

In certain embodiments, any of the eukaryotic organism provided herein is optionally further engineered to attenuate or delete one or more byproduct pathways, such as one or more of those exemplary byproduct pathways marked with an “X” in FIG. 7 or the conversion of 3-oxobutyraldehyde to acetoacetate by 3-oxobutyraldehyde dehydrogenase. For example, in one embodiment, the byproduct pathway comprises G3P phosphatase that converts G3P to glycerol. In another embodiment, the byproduct pathway comprises G3P dehydrogenase that converts dihydroxyacetone to G3P, and G3P phosphatase that converts G3P to glycerol. In other embodiments, the byproduct pathway comprises pyruvate decarboxylase that converts pyruvate to acetaldehyde. In another embodiment, the byproduct pathway comprises an ethanol dehydrogenase that converts acetaldehyde to ethanol. In other embodiments, the byproduct pathway comprises an acetaldehyde dehydrogenase (acylating) that converts acetyl-CoA to acetaldehyde and an ethanol dehydrogenase that converts acetaldehyde to ethanol. In other embodiments, the byproduct pathway comprises a pyruvate decarboxylase that converts pyruvate to acetaldehyde; and an ethanol dehydrogenase that converts acetaldehyde to ethanol. In other embodiments, the byproduct pathway comprises an acetaldehyde dehydrogenase (acylating) that converts acetyl-CoA to acetaldehyde and an ethanol dehydrogenase that converts acetaldehyde to ethanol. In certain embodiments, the byproduct pathway comprises an acetoacetyl-CoA hydrolase or transferase that converts acetoacetyl-CoA to acetoacetate. In another embodiment, the byproduct pathway comprises a 3-hydroxybutyrl-CoA-hydrolase that converts 3-hydroxybutyryl-CoA (3-HBCoA) to 3-hydroxybutyrate. In another embodiment, the byproduct pathway comprises a 3-hydroxybutyraldehyde dehydrogenase that converts 3-hydroxybutyraldehyde to 3-hydroxybutyrate. In another embodiment, the byproduct pathway comprises a 1,3-butanediol dehydrogenase that converts 1,3-butanediol to 3-oxobutanol. In another embodiment, the byproduct pathway comprises a 3-oxobutyraldehyde dehydrogenase that converts 3-oxobutyraldehyde to acetoacetate. In another embodiment, the byproduct pathway comprises a mitochondrial pyruvate dehydrogenase. In another embodiment, the byproduct pathway comprises an acetoacetyl-CoA thiolase.

In an additional embodiment, provided herein is a non-naturally occurring eukaryotic organism having a 1,3-BDO pathway, wherein the non-naturally occurring eukaryotic organism comprises at least one exogenous nucleic acid encoding an enzyme or protein that converts a substrate to a product selected from the group consisting of 4B, 4C, 4D, 4E, 4F, 4G, 4H, 4I, 4J, 4L, 4N and 4O. In some embodiments, the organism comprises a 1,3-BDO pathway comprising 4A, 4H, 4I and 4G. In other embodiments, the organism comprises a 1,3-BDO pathway comprising 7E, 7F, 4H, 4I and 4G. In some embodiments, the eukaryotic organism is further engineered to delete one or more of byproduct pathways as described herein.

One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, provided herein are non-naturally occurring eukaryotic organisms comprising at least one exogenous nucleic acid encoding an enzyme or protein, where the enzyme or protein converts the substrates and products of a 1,3-BDO pathway, such as those shown in FIG. 4 and FIG. 7.

Any combination and any number of the aforementioned enzymes can be introduced into a host eukaryotic organism to complete a 1,3-BDO pathway, as exemplified in FIG. 4 or 7. For example, the non-naturally occurring eukaryotic organism can include one, two, three, four, up to all of the nucleic acids in a 1,3-BDO pathway, each nucleic acid encoding a 1,3-BDO pathway enzyme. Such nucleic acids can include heterologous nucleic acids, additional copies of existing genes, and gene regulatory elements, as explained further below. The pathways of the non-naturally occurring eukaryotic organisms provided herein are also suitably engineered to be cultured in a substantially anaerobic culture medium.

While, in certain embodiments, a eukaryotic organism is said to further comprise a 1,3-BDO pathway, it is understood that also provided herein is a non-naturally occurring eukaryotic organism comprising at least one exogenous nucleic acid encoding a 1,3-BDO pathway enzyme expressed in a sufficient amount to produce an intermediate of a 1,3-BDO pathway. For example, as disclosed herein, a 1,3-BDO pathway is exemplified in FIG. 4 or 7. Therefore, in addition to a eukaryotic organism containing a 1,3-BDO pathway that produces 1,3-BDO, provided herein is a non-naturally occurring eukaryotic organism comprising at least one exogenous nucleic acid encoding a 1,3-BDO pathway enzyme, where the eukaryotic organism produces a 1,3-BDO pathway intermediate, for example, acetoacetyl-CoA, acetoacetate, 3-oxobutyraldehyde, 3-hydroxybuturaldehyde, 4-hydroxy-2-butanone, 3-hydroxybutyrl-CoA, or 3-hydroxybutyrate.

It is understood that any of the pathways disclosed herein, as described in the Examples and exemplified in the figures, including the pathways of FIG. 4 or 7, can be utilized to generate a non-naturally occurring eukaryotic organism that produces any pathway intermediate or product, as desired. As disclosed herein, such a eukaryotic organism that produces an intermediate can be used in combination with another eukaryotic organism expressing downstream pathway enzymes to produce a desired product. However, it is understood that a non-naturally occurring eukaryotic organism that produces a 1,3-BDO pathway intermediate can be utilized to produce the intermediate as a desired product.

The conversion of acetyl-CoA to 1,3-BDO can be accomplished by a number of pathways involving about three to five enzymatic steps as shown in FIG. 4. In the first step of all pathways (Step A), acetyl-CoA is converted to acetoacetyl-CoA by enzyme 4A. Alternatively, acetyl-CoA is converted to malonyl-CoA by acetyl-CoA carboxylase (FIG. 7, step E), and acetoacetyl-CoA is synthesized from acetyl-CoA and malonyl-CoA by acetoacetyl-CoA synthase (FIG. 7, step F).

In one route, 4A converts acetyl-CoA to acetoacetyl-CoA; 4E converts acetoacetyl-CoA to 3-oxobutyraldehyde; 4F converts 3-oxobutyraldehyde to 3-hydroxybutyrldehyde, and 4G converts 3-hydroxybutyrldehyde to 1,3-BDO. In another route, 4A converts acetyl-CoA to acetoacetyl-CoA; 4B converts acetoacetyl-CoA to 4-hydroxy-2-butanone; and 4D converts 4-hydroxy-2-butanone to 1,3-BDO. In one route, 4A converts acetyl-CoA to acetoacetyl-CoA; 4E converts acetoacetyl-CoA to 3-oxobutyraldehyde; 4C converts 3-oxobutyraldehyde to 4-hydroxy-2-butanone; and 4D converts 4-hydroxy-2-butanone to 1,3-BDO. In another route, 4A converts acetyl-CoA to acetoacetyl-CoA; 4H converts acetoacetyl-CoA to 3-hydroxybutyryl-CoA; and 4J converts 3-hydroxybutyryl-CoA to 1,3-BDO. In yet another route, 4A converts acetyl-CoA to acetoacetyl-CoA; 4H converts acetoacetyl-CoA to 3-hydroxybutyryl-CoA; 4I converts 3-hydroxybutyryl-CoA to 3-hydroxybutyraldehyde; and 4G converts 3-hydroxybutyrldehyde to 1,3-BDO. In another route, 4A converts acetyl-CoA to acetoacetyl-CoA; 4H converts acetoacetyl-CoA to 3-hydroxybutyryl-CoA; 4M converts 3-hydroxybutyrl-CoA to 3-hydroxybutyrate; 4N converts 3-hydroxybutyrate to 3-hydroxybutyraldehyde; and 4G converts 3-hydroxybutyrldehyde to 1,3-BDO. In one route, 4A converts acetyl-CoA to acetoacetyl-CoA; 4K converts acetoacetyl-CoA to acetoacetate; 4O converts acetoacetate to 3-hydroxybutyrate; 4N converts 3-hydroxybutyrate to 3-hydroxybutyraldehyde; and 4G converts 3-hydroxybutyrldehyde to 1,3-BDO. In another route, 4A converts acetyl-CoA to acetoacetyl-CoA; 4K converts acetoacetyl-CoA to acetoacetate; 4L converts acetoacetate to 3-oxobutyraldehyde; 4F converts 3-oxobutyraldehyde to 3-hydroxybutyrldehyde; and 4G converts 3-hydroxybutyrldehyde to 1,3-BDO.

Based on the routes described above for the production of 1,3-BDO from acetyl-CoA, in some embodiments, the non-naturally occurring eukaryotic organism has a set of 1,3-BDO pathway enzymes that includes 4A, 4E, 4F and 4G; 4A, 4B and 4D; 4A, 4E, 4C and 4D; 4A, 4H and 4J; 4A, 4H, 4I and 4G; 4A, 4H, 4M, 4N and 4G; 4A, 4K, 4O, 4N and 4G; or 4A, 4K, 4L, 4F and 4G. Any number of nucleic acids encoding these enzymes can be introduced into the host organism including one, two, three, four or up to all five of the nucleic acids that encode these enzymes. Where one, two, three or four exogenous nucleic acids are introduced, for example, such nucleic acids can be any permutation of the five nucleic acids. The same holds true for any other number of exogenous nucleic acids that is less than the number of enzymes being encoded.

In another route, 7E converts acetyl-CoA to malonyl-CoA and 7F converts malonyl-CoA and acetyl-CoA to acetoacetyl-CoA; 4E converts acetoacetyl-CoA to 3-oxobutyraldehyde; 4F converts 3-oxobutyraldehyde to 3-hydroxybutyrldehyde, and 4G converts 3-hydroxybutyrldehyde to 1,3-BDO. In another route, 7E converts acetyl-CoA to malonyl-CoA and 7F converts malonyl-CoA and acetyl-CoA to acetoacetyl-CoA; 4B converts acetoacetyl-CoA to 4-hydroxy-2-butanone; and 4D converts 4-hydroxy-2-butanone to 1,3-BDO. In one route, 7E converts acetyl-CoA to malonyl-CoA and 7F converts malonyl-CoA and acetyl-CoA to acetoacetyl-CoA; 4E converts acetoacetyl-CoA to 3-oxobutyraldehyde; 4C converts 3-oxobutyraldehyde to 4-hydroxy-2-butanone; and 4D converts 4-hydroxy-2-butanone to 1,3-BDO. In another route, 7E converts acetyl-CoA to malonyl-CoA and 7F converts malonyl-CoA and acetyl-CoA to acetoacetyl-CoA; 4H converts acetoacetyl-CoA to 3-hydroxybutyryl-CoA; and 4J converts 3-hydroxybutyryl-CoA to 1,3-BDO. In yet another route, 7E converts acetyl-CoA to malonyl-CoA and 7F converts malonyl-CoA and acetyl-CoA to acetoacetyl-CoA; 4H converts acetoacetyl-CoA to 3-hydroxybutyryl-CoA; 4I converts 3-hydroxybutyryl-CoA to 3-hydroxybutyraldehyde; and 4G converts 3-hydroxybutyrldehyde to 1,3-BDO. In another route, 7E converts acetyl-CoA to malonyl-CoA and 7F converts malonyl-CoA and acetyl-CoA to acetoacetyl-CoA; 4H converts acetoacetyl-CoA to 3-hydroxybutyryl-CoA; 4M converts 3-hydroxybutyrl-CoA to 3-hydroxybutyrate; 4N converts 3-hydroxybutyrate to 3-hydroxybutyraldehyde; and 4G converts 3-hydroxybutyrldehyde to 1,3-BDO. In one route, 7E converts acetyl-CoA to malonyl-CoA and 7F converts malonyl-CoA and acetyl-CoA to acetoacetyl-CoA; 4K converts acetoacetyl-CoA to acetoacetate; 4O converts acetoacetate to 3-hydroxybutyrate; 4N converts 3-hydroxybutyrate to 3-hydroxybutyraldehyde; and 4G converts 3-hydroxybutyrldehyde to 1,3-BDO. In another route, 7E converts acetyl-CoA to malonyl-CoA and 7F converts malonyl-CoA and acetyl-CoA to acetoacetyl-CoA; 4K converts acetoacetyl-CoA to acetoacetate; 4L converts acetoacetate to 3-oxobutyraldehyde; 4F converts 3-oxobutyraldehyde to 3-hydroxybutyrldehyde; and 4G converts 3-hydroxybutyrldehyde to 1,3-BDO.

Based on the routes described above for the production of 1,3-BDO from acetyl-CoA, in some embodiments, the non-naturally occurring eukaryotic organism has a set of 1,3-BDO pathway enzymes that includes 7E, 7F, 4E, 4F and 4G; 7E, 7F, 4B and 4D; 7E, 7F, 4E, 4C and 4D; 7E, 7F, 4H and 4J; 7E, 7F, 4H, 4I and 4G; 7E, 7F, 4H, 4M, 4N and 4G; 7E, 7F, 4K, 4O, 4N and 4G; or 7E, 7F, 4K, 4L, 4F and 4G. Any number of nucleic acids encoding these enzymes can be introduced into the host organism including one, two, three, four or up to all five of the nucleic acids that encode these enzymes. Where one, two, three or four exogenous nucleic acids are introduced, for example, such nucleic acids can be any permutation of the five nucleic acids. The same holds true for any other number of exogenous nucleic acids that is less than the number of enzymes being encoded.

The organism can optionally be further engineered to delete one or more of the exemplary byproduct pathways (“X”) as described elsewhere herein. Based on these routes for the production of 1,3-BDO from acetyl-CoA, in some embodiments, the non-naturally occurring eukaryotic organism has a set of 1,3-BDO pathway enzymes that includes 4A, 4H, 4I and 4G; or 7E, 7F, 4H, 4I and 4G. Any number of nucleic acids encoding these enzymes can be introduced into the host organism including one, two, three, four or up to all five of the nucleic acids that encode these enzymes. Where one, two, or three exogenous nucleic acids are introduced, for example, such nucleic acids can be any permutation of the four or five nucleic acids. The same holds true for any other number of exogenous nucleic acids that is less than the number of enzymes being encoded.

### 4.3 Combined Cytosolic/Mitochondrial 1,3-BDO Pathways

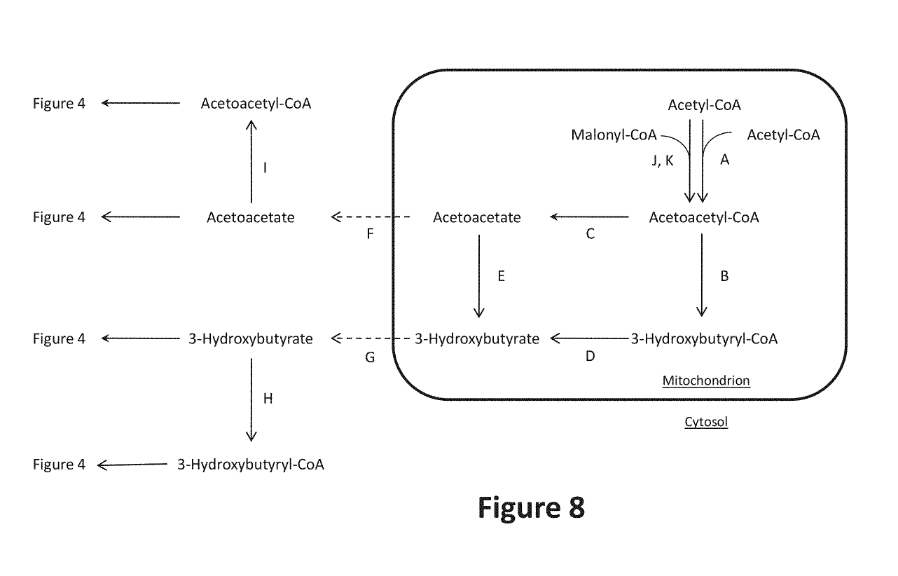

A eukaryotic organism, as provided herein, can also be engineered to efficiently direct carbon and reducing equivalents into a combined mitochondrial/cytosolic 1,3-BDO pathway. Such a pathway would require synthesis of a monocarboxylic 1,3-BDO pathway intermediate such as acetoacetate or 3-hydroxybutyrate in the mitochondria, export of the pathway intermediate to the cytosol, and subsequent conversion of that intermediate to 1,3-BDO in the cytosol. Exemplary combined mitochondrial/cytosolic 1,3-BDO pathways are depicted in FIG. 8.

There are several advantages to producing 1,3-BDO using a combined mitochondrial/cytosolic 1,3-BDO production pathway. One advantage is the naturally abundant mitochondrial pool of acetyl-CoA, the key 1,3-BDO pathway precursor. Having a 1,3-BDO pathway span multiple compartments can also be advantageous if pathway enzymes are not adequately selective for their substrates. For example, 3-hydroxybutyryl-CoA reductase and 3-hydroxybutyryaldehyde enzymes may also reduce acetyl-CoA to ethanol. Sequestration of the acetyl-CoA pool in the mitochondria could therefore reduce formation of byproducts derived from acetyl-CoA. A combined mitochondrial/cytosolic 1,3-BDO pathway could benefit from attenuation of mitochondrial acetyl-CoA consuming enzymes or pathways such as the TCA cycle.

Acetoacetate and 3-hydroxybutyrate are readily transported out of the mitochondria by pyruvate and/or monocarboxylate transporters. The existence of a proton symporter for the uptake of pyruvate and also for acetoacetate was demonstrated in isolated mitochondria (Briquet, Biochem Biophys Acta 459:290-99 (1977)). However, the gene encoding this transporter has not been identified to date. S. cerevisiae encodes five putative monocarboxylate transporters (MCH1-5), several of which may be localized to the mitochondrial membrane (Makuc et al, Yeast 18:1131-43 (2001)). NDT1 is another putative pyruvate transporter, although the role of this protein is disputed in the literature (Todisco et al, J Biol Chem 20:1524-31 (2006)). Exemplary monocarboxylate transporters are shown in the table below:

TABLE 1

| Protein | GenBank ID | GI number | Organism |
|---|---|---|---|
| MCH1 | NP_010229.1 | 6320149 | Saccharomyces |
| | | | cerevisiae |
| MCH2 | NP_012701.2 | 330443640 | Saccharomyces |
| | | | cerevisiae |
| MCH3 | NP_014274.1 | 6324204 | Saccharomyces |
| | | | cerevisiae |
| MCH5 | NP_014951.2 | 330443742 | Saccharomyces |
| | | | cerevisiae |
| NDT1 | NP_012260.1 | 6322185 | Saccharomyces |
| | | | cerevisiae |
| ANI_1_1592184 | XP_001401484.2 | 317038471 | Aspergillus niger |
| CaJ7_0216 | XP_888808.1 | 77022728 | Candida albicans |
| YALI0E16478g | XP_504023.1 | 50553226 | Yarrowia lipolytica |
| KLLA0D14036g | XP_453688.1 | 50307419 | Kluyveromyces |
| | | | lactis |

In certain embodiments, the combined mitochondrial/cytosolic 1,3-BDO pathway comprises 8A, 8B, 8C, 8D, 8E, 8F, 8G, 8H, 8I, 8J, 8K, 7E, 7F, 4A, 4B, 4C, 4D, 4E, 4F, 4G, 4H, 4I, 4J, 4K, 4L, 4M, 4N, and 4O, or any combination of 8A, 8B, 8C, 8D, 8E, 8F, 8G, 8H, 8I, 8J, 8K, 7E, 7F, 4A, 4B, 4C, 4D, 4E, 4F, 4G, 4H, 4I, 4J, 4K, 4L, 4M, 4N, and 4O thereof, wherein 8A is a mitochondrial acetoacetyl-CoA thiolase; 8B is a mitochondrial acetoacetyl-CoA reductase; 8C is a mitochondrial acetoacetyl-CoA hydrolase, transferase or synthetase; 8D is a mitochondrial 3-hydroxybutyryl-CoA hydrolase, transferase or synthetase; 8E is a mitochondrial. 3-hydroxybutyrate dehydrogenase; 8F is an acetoacetate transporter; 8G is a 3-hydroxybutyrate transporter; 8H is a 3-hydroxybutyryl-CoA transferase or synthetase, 8I is a cytosolic acetoacetyl-CoA transferase or synthetase, 8J is a mitochondrial acetyl-CoA carboxylase; 8K is a mitochondrial acetoacetyl-CoA synthase; 7E is acetyl-CoA carboxylase, 7F is acetoacetyl-CoA synthase, 4A is an acetoacetyl-CoA thiolase; 4B is an acetoacetyl-CoA reductase (CoA-dependent, alcohol forming); 4C is a 3-oxobutyraldehyde reductase (aldehyde reducing); 4D is a 4-hydroxy,2-butanone reductase; 4E is an acetoacetyl-CoA reductase (CoA-dependent, aldehyde forming); 4F is a 3-oxobutyraldehyde reductase (ketone reducing); 4G is a 3-hydroxybutyraldehyde reductase; 4H is an acetoacetyl-CoA reductase (ketone reducing); 4I is a 3-hydroxybutyryl-CoA reductase (aldehyde forming); 4J is a 3-hydroxybutyryl-CoA reductase (alcohol forming); 4K is an acetoacetyl-CoA transferase, an acetoacetyl-CoA hydrolase, an acetoacetyl-CoA synthetase, or a phosphotransacetoacetylase and acetoacetate kinase; 4L is an acetoacetate reductase; 4M is a 3-hydroxybutyryl-CoA transferase, hydrolase, or synthetase; 4N is a 3-hydroxybutyrate reductase; and wherein 4O is a 3-hydroxybutyrate dehydrogenase. In certain embodiments, 8C is a mitochondrial acetoacetyl-CoA hydrolase. In other embodiments, 8C is a mitochondrial acetoacetyl-CoA transferase. In certain embodiments, 8C is a mitochondrial acetoacetyl-CoA synthetase. In certain embodiments 8D is a mitochondrial 3-hydroxybutyryl-CoA hydrolase. In other embodiments 8D is a mitochondrial 3-hydroxybutyryl-CoA transferase. In certain embodiments 8D is a mitochondrial 3-hydroxybutyryl-CoA synthetase. In certain embodiments, 8H is a 3-hydroxybutyryl-CoA transferase. In other embodiments, 8H is a 3-hydroxybutyryl-CoA synthetase. In certain embodiments, 8I is a cytosolic acetoacetyl-CoA transferase. In other embodiments, 8I is a cytosolic acetoacetyl-CoA synthetase. In certain embodiments, 4K is an acetoacetyl-CoA transferase. In other embodiments, 4K is an acetoacetyl-CoA hydrolase. In some embodiments, 4K is an acetoacetyl-CoA synthetase. In other embodiments, 4K is a phosphotransacetoacetylase and acetoacetate kinase. In certain embodiments, 4M is a 3-hydroxybutyryl-CoA transferase. In some embodiments, 4M is a 3-hydroxybutyryl-CoA, hydrolase. In yet other embodiments, 4M is a 3-hydroxybutyryl-CoA synthetase.

In another aspect, provided herein is a non-naturally occurring eukaryotic organism comprising: (1) an acetoacetate pathway, wherein said organism comprises at least one exogenous nucleic acid encoding an acetoacetate pathway enzyme expressed in a sufficient amount to increase acetoacetate in the cytosol of said organism, wherein said acetoacetate pathway comprises 8A, 8C, and 8F, wherein 8A is a mitochondrial acetoacetyl-CoA thiolase; 8C is a mitochondrial acetoacetyl-CoA hydrolase, transferase or synthetase; and 8F is an acetoacetate transporter; and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises a pathway selected from: (i) 4O, 4N, and 4G; and (ii) 4L, 4F, and 4G; wherein 4F is a 3-oxobutyraldehyde reductase (ketone reducing); 4G is a 3-hydroxybutyraldehyde reductase; 4L is an acetoacetate reductase; 4N is a 3-hydroxybutyrate reductase; and 4O is a 3-hydroxybutyrate dehydrogenase. In some embodiments, the 1,3-BDO pathway comprises 4O, 4N and 4G. In other embodiments, the 1,3-BDO pathway comprises 4L, 4F, and 4G.

In another aspect, provided herein is a non-naturally occurring eukaryotic organism comprising: (1) an acetoacetate pathway, wherein said organism comprises at least one exogenous nucleic acid encoding an acetoacetate pathway enzyme expressed in a sufficient amount to increase acetoacetate in the cytosol of said organism, wherein said acetoacetate pathway comprises 8J, 8K, 8C, and 8F, wherein 8J is a mitochondrial acetyl-CoA carboxylase; 8K is a mitochondrial acetoacetyl-CoA synthase; 8C is a mitochondrial acetoacetyl-CoA hydrolase, transferase or synthetase; and 8F is an acetoacetate transporter; and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises a pathway selected from: (i) 4O, 4N, and 4G; and (ii) 4L, 4F, and 4G; wherein 4F is a 3-oxobutyraldehyde reductase (ketone reducing); 4G is a 3-hydroxybutyraldehyde reductase; 4L is an acetoacetate reductase; 4N is a 3-hydroxybutyrate reductase; and 4O is a 3-hydroxybutyrate dehydrogenase. In some embodiments, the 1,3-BDO pathway comprises 4O, 4N and 4G. In other embodiments, the 1,3-BDO pathway comprises 4L, 4F, and 4G.

In another aspect, provided herein is a non-naturally occurring eukaryotic organism comprising: (1) an acetoacetyl-CoA pathway, wherein said organism comprises at least one exogenous nucleic acid encoding an acetoacetyl-CoA pathway enzyme expressed in a sufficient amount to increase acetoacetyl-CoA in the cytosol of said organism, wherein said acetoacetyl-CoA pathway comprises 8A, 8C, 8F and 8I, wherein 8A is a mitochondrial acetoacetyl-CoA thiolase; 8C is a mitochondrial acetoacetyl-CoA hydrolase, transferase or synthetase; 8F is an acetoacetate transporter; and 8I is a cytosolic acetoacetyl-CoA transferase or synthetase; and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises a pathway selected from: (i) 4E, 4F and 4G; (ii) 4B and 4D; (iii) 4E, 4C and 4D; (iv) 4H and 4J; (v) 4H, 4I and 4G; and (vi) 4H, 4M, 4N and 4G; wherein 4B is an acetoacetyl-CoA reductase (CoA-dependent, alcohol forming); 4C is a 3-oxobutyraldehyde reductase (aldehyde reducing); 4D is a 4-hydroxy,2-butanone reductase; 4E is an acetoacetyl-CoA reductase (CoA-dependent, aldehyde forming); 4F is a 3-oxobutyraldehyde reductase (ketone reducing); 4G is a 3-hydroxybutyraldehyde reductase; 4H is an acetoacetyl-CoA reductase (ketone reducing); 4I is a 3-hydroxybutyryl-CoA reductase (aldehyde forming); 4J is a 3-hydroxybutyryl-CoA reductase (alcohol forming); 4L is an acetoacetate reductase; 4M is a 3-hydroxybutyryl-CoA transferase, hydrolase, or synthetase; and 4N is a 3-hydroxybutyrate reductase. In some embodiments, the 1,3-BDO pathway comprises 4E, 4F and 4G. In some embodiments, the 1,3-BDO pathway comprises 4B and 4D. In other embodiments, 1,3-BDO pathway comprises 4E, 4C and 4D. In another embodiment, 1,3-BDO pathway comprises 4H and 4J. In another embodiment, the 1,3-BDO pathway comprises 4H, 4I and 4G. In other embodiments, the 1,3-BDO pathway comprises 4H, 4M, 4N and 4G.

In another aspect, provided herein is a non-naturally occurring eukaryotic organism comprising: (1) an acetoacetyl-CoA pathway, wherein said organism comprises at least one exogenous nucleic acid encoding an acetoacetyl-CoA pathway enzyme expressed in a sufficient amount to increase acetoacetyl-CoA in the cytosol of said organism, wherein said acetoacetyl-CoA pathway comprises 8J, 8K, 8C, 8F and 8I, wherein 8J is a mitochondrial acetyl-CoA carboxylase; 8K is a mitochondrial acetoacetyl-CoA synthase; 8C is a mitochondrial acetoacetyl-CoA hydrolase, transferase or synthetase; 8F is an acetoacetate transporter; and 8I is a cytosolic acetoacetyl-CoA transferase or synthetase; and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises a pathway selected from: (i) 4E, 4F and 4G; (ii) 4B and 4D; (iii) 4E, 4C and 4D; (iv) 4H and 4J; (v) 4H, 4I and 4G; and (vi) 4H, 4M, 4N and 4G; wherein 4B is an acetoacetyl-CoA reductase (CoA-dependent, alcohol forming); 4C is a 3-oxobutyraldehyde reductase (aldehyde reducing); 4D is a 4-hydroxy,2-butanone reductase; 4E is an acetoacetyl-CoA reductase (CoA-dependent, aldehyde forming); 4F is a 3-oxobutyraldehyde reductase (ketone reducing); 4G is a 3-hydroxybutyraldehyde reductase; 4H is an acetoacetyl-CoA reductase (ketone reducing); 4I is a 3-hydroxybutyryl-CoA reductase (aldehyde forming); 4J is a 3-hydroxybutyryl-CoA reductase (alcohol forming); 4L is an acetoacetate reductase; 4M is a 3-hydroxybutyryl-CoA transferase, hydrolase, or synthetase; and 4N is a 3-hydroxybutyrate reductase. In some embodiments, the 1,3-BDO pathway comprises 4E, 4F and 4G. In some embodiments, the 1,3-BDO pathway comprises 4B and 4D. In other embodiments, 1,3-BDO pathway comprises 4E, 4C and 4D. In another embodiment, 1,3-BDO pathway comprises 4H and 4J. In another embodiment, the 1,3-BDO pathway comprises 4H, 4I and 4G. In other embodiments, the 1,3-BDO pathway comprises 4H, 4M, 4N and 4G.

In another aspect, provided herein is a non-naturally occurring eukaryotic organism comprising: (1) a 3-hydroxybutyrate pathway, wherein said organism comprises at least one exogenous nucleic acid encoding a 3-hydroxybutyrate pathway enzyme expressed in a sufficient amount to increase 3-hydroxybutyrate in the cytosol of said organism, wherein said 3-hydroxybutyrate pathway comprises a pathway selected from: (i) 8A, 8B, 8D and 8G; and (ii) 8A, 8C, 8E and 8G; wherein 8A is a mitochondrial acetoacetyl-CoA thiolase; 8B is a mitochondrial acetoacetyl-CoA reductase; 8C is a mitochondrial acetoacetyl-CoA hydrolase, transferase or synthetase; 8D is a mitochondrial 3-hydroxybutyryl-CoA hydrolase, transferase or synthetase; 8E is a mitochondrial 3-hydroxybutyrate dehydrogenase; and 8G is a 3-hydroxybutyrate transporter; and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises 4N and 4G, wherein 4G is a 3-hydroxybutyraldehyde reductase; and 4N is a 3-hydroxybutyrate reductase. In one embodiment, the 3-hydroxybutyrate pathway comprises 8A, 8B, 8D and 8G. In another embodiment, the 3-hydroxybutyrate pathway comprises 8A, 8C, 8E and 8G.

In another aspect, provided herein is a non-naturally occurring eukaryotic organism comprising: (1) a 3-hydroxybutyrate pathway, wherein said organism comprises at least one exogenous nucleic acid encoding a 3-hydroxybutyrate pathway enzyme expressed in a sufficient amount to increase 3-hydroxybutyrate in the cytosol of said organism, wherein said 3-hydroxybutyrate pathway comprises a pathway selected from: (i) 8J, 8K, 8B, 8D and 8G; and (ii) 8J, 8K, 8C, 8E and 8G; wherein 8J is a mitochondrial acetyl-CoA carboxylase; 8K is a mitochondrial acetoacetyl-CoA synthase; 8B is a mitochondrial acetoacetyl-CoA reductase; 8C is a mitochondrial acetoacetyl-CoA hydrolase, transferase or synthetase; 8D is a mitochondrial 3-hydroxybutyryl-CoA hydrolase, transferase or synthetase; 8E is a mitochondrial 3-hydroxybutyrate dehydrogenase; and 8G is a 3-hydroxybutyrate transporter; and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises 4N and 4G, wherein 4G is a 3-hydroxybutyraldehyde reductase; and 4N is a 3-hydroxybutyrate reductase. In one embodiment, the 3-hydroxybutyrate pathway comprises 8J, 8K, 8B, 8D and 8G. In another embodiment, the 3-hydroxybutyrate pathway comprises 8J, 8K, 8C, 8E and 8G.

In another aspect, provided herein is a non-naturally occurring eukaryotic organism comprising: (1) a 3-hydroxybutyryl-CoA pathway, wherein said organism comprises at least one exogenous nucleic acid encoding a 3-hydroxybutyryl-CoA pathway enzyme expressed in a sufficient amount to increase 3-hydroxybutyryl-CoA in the cytosol of said organism, wherein said 3-hydroxybutyryl-CoA pathway comprises a pathway selected from: (i) 8A, 8B, 8D, 8G and 8H; and (ii) 8A, 8C, 8E, 8G and 8H; wherein 8A is a mitochondrial acetoacetyl-CoA thiolase; 8B is a mitochondrial acetoacetyl-CoA reductase; 8C is a mitochondrial acetoacetyl-CoA hydrolase, transferase or synthetase; 8D is a mitochondrial 3-hydroxybutyryl-CoA hydrolase, transferase or synthetase; 8E is a mitochondrial 3-hydroxybutyrate dehydrogenase; 8G is a 3-hydroxybutyrate transporter; and 8H is a 3-hydroxybutyryl-CoA transferase or synthetase, and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises a pathway selected from: (i) 4I and 4G; and (ii) 4J; wherein 4I is a 3-hydroxybutyryl-CoA reductase (aldehyde forming); wherein 4G is a 3-hydroxybutyraldehyde reductase; and 4J is a 3-hydroxybutyryl-CoA reductase (alcohol forming). In certain embodiments, the 3-hydroxybutyryl-CoA pathway comprises 8A, 8B, 8D, 8G, and 8H, and the 1,3-BDO pathway comprises 4I and 4G. In other embodiments, the 3-hydroxybutyryl-CoA pathway comprises 8A, 8B, 8D, 8G, and 8H, and the 1,3-BDO pathway comprises 4J. In another embodiment, the 3-hydroxybutyryl-CoA pathway comprises 8A, 8C, 8E, 8G, and 8H, and the 1,3-BDO pathway comprises 4I and 4G. In yet another embodiment, the 3-hydroxybutyryl-CoA pathway comprises 8A, 8C, 8E, 8G, and 8H, and the 1,3-BDO pathway comprises 4J.

In another aspect, provided herein is a non-naturally occurring eukaryotic organism comprising: (1) a 3-hydroxybutyryl-CoA pathway, wherein said organism comprises at least one exogenous nucleic acid encoding a 3-hydroxybutyryl-CoA pathway enzyme expressed in a sufficient amount to increase 3-hydroxybutyryl-CoA in the cytosol of said organism, wherein said 3-hydroxybutyryl-CoA pathway comprises a pathway selected from: (i) 8J. 8K, 8B, 8D, 8G and 8H; and (ii) 8J, 8K, 8C, 8E, 8G and 8H; wherein 8J is a mitochondrial acetyl-CoA carboxylase; 8K is a mitochondrial acetoacetyl-CoA synthase; 8B is a mitochondrial acetoacetyl-CoA reductase; 8C is a mitochondrial acetoacetyl-CoA hydrolase, transferase or synthetase; 8D is a mitochondrial 3-hydroxybutyryl-CoA hydrolase, transferase or synthetase; 8E is a mitochondrial 3-hydroxybutyrate dehydrogenase; 8G is a 3-hydroxybutyrate transporter; and 8H is a 3-hydroxybutyryl-CoA transferase or synthetase, and (2) a 1,3-BDO pathway, wherein said organism comprises at least one exogenous nucleic acid encoding a 1,3-BDO pathway enzyme expressed in a sufficient amount to produce 1,3-BDO in the cytosol of said organism, and wherein the 1,3-BDO pathway comprises a pathway selected from: (i) 4I and 4G; and (ii) 4J; wherein 4I is a 3-hydroxybutyryl-CoA reductase (aldehyde forming); wherein 4G is a 3-hydroxybutyraldehyde reductase; and 4J is a 3-hydroxybutyryl-CoA reductase (alcohol forming). In certain embodiments, the 3-hydroxybutyryl-CoA pathway comprises 8A, 8B, 8D, 8G, and 8H, and the 1,3-BDO pathway comprises 4I and 4G. In other embodiments, the 3-hydroxybutyryl-CoA pathway comprises 8A, 8B, 8D, 8G, and 8H, and the 1,3-BDO pathway comprises 4J. In another embodiment, the 3-hydroxybutyryl-CoA pathway comprises 8J, 8K, 8C, 8E, 8G, and 8H, and the 1,3-BDO pathway comprises 4I and 4G. In yet another embodiment, the 3-hydroxybutyryl-CoA pathway comprises 8J, 8K, 8C, 8E, 8G, and 8H, and the 1,3-BDO pathway comprises 4J.

One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, provided herein are non-naturally occurring eukaryotic organisms comprising at least one exogenous nucleic acid encoding an enzyme or protein, where the enzyme or protein converts the substrates and products of a combined mitochondrial/cytosolic 1,3-BDO pathway, such as those shown in FIG. 8.

Any combination and any number of the aforementioned enzymes can be introduced into a host eukaryotic organism to complete a combined mitochondrial/cytosolic 1,3-BDO pathway, as exemplified in FIG. 8. For example, the non-naturally occurring eukaryotic organism can include one, two, three, four, five, six, seven, up to all of the nucleic acids in a combined mitochondrial/cytosolic 1,3-BDO pathway, each nucleic acid encoding a combined mitochondrial/cytosolic 1,3-BDO pathway enzyme. Such nucleic acids can include heterologous nucleic acids, additional copies of existing genes, and gene regulatory elements, as explained further below. The pathways of the non-naturally occurring eukaryotic organisms provided herein are also suitably engineered to be cultured in a substantially anaerobic culture medium.

### 4.4 Balancing Co-Factor Usage

1,3-BDO production pathways, such as those depicted in FIG. 4, require reduced cofactors such as NAD(P)H. Therefore, increased production of 1,3-BDO can be achieved, in part, by engineering any of the non-naturally occurring eukaryotic organisms described herein to comprise pathways that supply NAD(P)H cofactors used in 1,3-BDO production pathways. In several organisms, including eukaryotic organisms, such as several Saccharomyces, Kluyveromyces, Candida, Aspergillus, and Yarrowia species, NADH is more abundant than NADPH in the cytosol as NADH is produced in large quantities by glycolysis. Levels of NADH can be increased in these eukaryotic organisms by converting pyruvate to acetyl-CoA through any of the following enzymes or enzyme sets: 1) an NAD-dependent pyruvate dehydrogenase; 2) a pyruvate formate lyase and an NAD-dependent formate dehydrogenase; 3) a pyruvate:ferredoxin oxidoreductase and an NADH:ferredoxin oxidoreductase; 4) a pyruvate decarboxylase and an NAD-dependent acylating acetylaldehyde dehydrogenase; 5) a pyruvate decarboxylase, a NAD-dependent acylating acetaldehyde dehydrogenase, an acetate kinase, and a phosphotransacetylase; and 6) a pyruvate decarboxylase, an NAD-dependent acylating acetaldehyde dehydrogenase, and an acetyl-CoA synthetase.

As shown in FIG. 4, the conversion of acetyl-CoA to 1,3-BDO can occur, in part, through three reduction steps. Each of these three reduction steps utilize either NADPH or NADH as the reducing agents, which, in turn, is converted into molecules of NADP or NAD, respectively. Given the abundance of NADH in the cytosol of some organisms, it can be beneficial in some embodiments for all reduction steps of the 1,3-BDO pathway to accept NADH as the reducing agent. High yields of 1,3-BDO can therefore be accomplished by: 1) identifying and implementing endogenous or exogenous 1,3-BDO pathway enzymes with a stronger preference for NADH than other reducing equivalents such as NADPH; 2) attenuating one or more endogenous 1,3-BDO pathway enzymes that contribute NADPH-dependent reduction activity; 3) altering the cofactor specificity of endogenous or exogenous 1,3-BDO pathway enzymes so that they have a stronger preference for NADH than their natural versions, and/or 4) altering the cofactor specificity of endogenous or exogenous 1,3-BDO pathway enzymes so that they have a weaker preference for NADPH than their natural versions.

In another aspect, provided herein is a method for selecting an exogenous 1,3-BDO pathway enzyme to be introduced into a non-naturally occurring eukaryotic organism, wherein the exogenous 1,3-BDO pathway enzyme is expressed in a sufficient amount in the organism to produce 1,3-BDO, said method comprising (i) measuring the activity of at least one 1,3-BDO pathway enzyme that uses NADH as a cofactor; (ii) measuring the activity of at least 1,3-BDO pathway enzyme that uses NADPH as a cofactor; and (iii) introducing into the organism at least one 1,3-BDO pathway enzyme that has a greater preference for NADH than NADPH as a cofactor as determined in steps (i) and (ii).

In another aspect, provided herein is a non-naturally eukaryotic organism comprising a 1,3-BDO pathway, wherein said organism further comprises: (1) a 1,3-BDO pathway, wherein said organism comprises at least one endogenous and/or exogenous nucleic acid encoding a 1,3-BDO pathway enzyme expressed in a sufficient amount to produce 1,3-BDO; and (2) an acetyl-CoA pathway, wherein said organism comprises at least one endogenous and/or exogenous nucleic acid encoding an acetyl-CoA pathway enzyme expressed in a sufficient amount to increase NADH in the organism; wherein the acetyl-CoA pathway comprises (i.) an NAD-dependent pyruvate dehydrogenase; (ii.) a pyruvate formate lyase and an NAD-dependent formate dehydrogenase; (iii.) a pyruvate:ferredoxin oxidoreductase and an NADH:ferredoxin oxidoreductase; (iv.) a pyruvate decarboxylase and an NAD-dependent acylating acetylaldehyde dehydrogenase; (v.) a pyruvate decarboxylase, a NAD-dependent acylating acetaldehyde dehydrogenase, an acetate kinase, and a phosphotransacetylase; or (vi.) a pyruvate decarboxylase, an NAD-dependent acylating acetaldehyde dehydrogenase, and an acetyl-CoA synthetase. In some embodiments, the acetyl-CoA pathway comprises an NAD-dependent pyruvate dehydrogenase. In other embodiments, the acetyl-CoA pathway comprises an a pyruvate formate lyase and an NAD-dependent formate dehydrogenase. In other embodiments, the acetyl-CoA pathway comprises a pyruvate:ferredoxin oxidoreductase and an NADH:ferredoxin oxidoreductase. In other embodiments, the acetyl-CoA pathway comprises a pyruvate decarboxylase and an NAD-dependent acylating acetylaldehyde dehydrogenase. In other embodiments, the acetyl-CoA pathway comprises a pyruvate decarboxylase, a NAD-dependent acylating acetaldehyde dehydrogenase, an acetate kinase, and a phosphotransacetylase. In yet other embodiments, the acetyl-CoA pathway comprises a pyruvate decarboxylase, an NAD-dependent acylating acetaldehyde dehydrogenase, and an acetyl-CoA synthetase.

In another aspect, provided herein is a non-naturally eukaryotic organism comprising a 1,3-BDO pathway, wherein said organism further comprises one or more endogenous and/or exogenous nucleic acids encoding a 1,3-BDO pathway enzyme selected from the group consisting of 4B, 4C, 4D, 4E, 4F, 4G, 4H, 4I, 4J, 4L, 4N, and 4O; wherein at least one nucleic acid has been altered such that the 1,3-BDO pathway enzyme encoded by the nucleic acid has a greater affinity for NADH than the 1,3-BDO pathway enzyme encoded by an unaltered or wild-type nucleic acid. In some embodiments, the eukaryotic organism comprises a nucleic acid encoding 4B. In some embodiments, the eukaryotic organism comprises a nucleic acid encoding 4C. In some embodiments, the eukaryotic organism comprises a nucleic acid encoding 4D. In some embodiments, the eukaryotic organism comprises a nucleic acid encoding 4E. In some embodiments, the eukaryotic organism comprises a nucleic acid encoding 4F. In some embodiments, the eukaryotic organism comprises a nucleic acid encoding 4G. In some embodiments, the eukaryotic organism comprises a nucleic acid encoding 4H. In some embodiments, the eukaryotic organism comprises a nucleic acid encoding 4I. In some embodiments, the eukaryotic organism comprises a nucleic acid encoding 4J. In some embodiments, the eukaryotic organism comprises a nucleic acid encoding 4L. In some embodiments, the eukaryotic organism comprises a nucleic acid encoding 4N. In some embodiments, the eukaryotic organism comprises a nucleic acid encoding 4O. In some embodiments, the eukaryotic organism comprises nucleic acids encoding 4B and 4D. In some embodiments, the eukaryotic organism comprises nucleic acids encoding 4E, 4C and 4D. In some embodiments, the eukaryotic organism comprises nucleic acids encoding 4E, 4F and 4G. In some embodiments, the eukaryotic organism comprises nucleic acids encoding 4L, 4F and 4G. In some embodiments, the eukaryotic organism comprises nucleic acids encoding 4H, 4N and 4G. In some embodiments, the eukaryotic organism comprises nucleic acids encoding 4H and 4J. In some embodiments, the eukaryotic organism comprises nucleic acids encoding 4H, 4I and 4G. In some embodiments, the eukaryotic organism comprises nucleic acids encoding 4L, 4F and 4G. In some embodiments, the eukaryotic organism comprises nucleic acids encoding 4O, 4N and 4G. In some embodiments, the eukaryotic organism comprises nucleic acids encoding 4A, 4N and 4G.

In another aspect, provided herein is a non-naturally eukaryotic organism comprising a 1,3-BDO pathway, wherein said organism further comprises one or more endogenous and/or exogenous nucleic acids encoding an attenuated 1,3-BDO pathway enzyme selected from the group consisting of 4B, 4C, 4D, 4E, 4F, 4G, 4H, 4I, 4J, 4L, 4N and 4O; wherein the attenuated 1,3-BDO pathway enzyme is NAPDH-dependent and has lower enzymatic activity as compared to the 1,3-BDO pathway enzyme encoded by an unaltered or wild-type nucleic acid. In some embodiments, the eukaryotic organism comprises a nucleic acid encoding 4B. In some embodiments, the eukaryotic organism comprises a nucleic acid encoding 4C. In some embodiments, the eukaryotic organism comprises a nucleic acid encoding 4D. In some embodiments, the eukaryotic organism comprises a nucleic acid encoding 4E. In some embodiments, the eukaryotic organism comprises a nucleic acid encoding 4F. In some embodiments, the eukaryotic organism comprises a nucleic acid encoding 4G. In some embodiments, the eukaryotic organism comprises a nucleic acid encoding 4H. In some embodiments, the eukaryotic organism comprises a nucleic acid encoding 4I. In some embodiments, the eukaryotic organism comprises a nucleic acid encoding 4J. In some embodiments, the eukaryotic organism comprises a nucleic acid encoding 4N. In some embodiments, the eukaryotic organism comprises a nucleic acid encoding 4O. In some embodiments, the eukaryotic organism comprises nucleic acids encoding 4B and 4D. In some embodiments, the eukaryotic organism comprises nucleic acids encoding 4E, 4C and 4D. In some embodiments, the eukaryotic organism comprises nucleic acids encoding 4E, 4F and 4G. In some embodiments, the eukaryotic organism comprises nucleic acids encoding 4L, 4F and 4G. In some embodiments, the eukaryotic organism comprises nucleic acids encoding 4H, 4N and 4G. In some embodiments, the eukaryotic organism comprises nucleic acids encoding 4H and 4J. In some embodiments, the eukaryotic organism comprises nucleic acids encoding 4H, 4I and 4G. In some embodiments, the eukaryotic organism comprises nucleic acids encoding 4L, 4F and 4G. In some embodiments, the eukaryotic organism comprises nucleic acids encoding 4O, 4N and 4G. In some embodiments, the eukaryotic organism comprises nucleic acids encoding 4A, 4N and 4G.

In another aspect, provided herein is a non-naturally eukaryotic organism comprising a 1,3-BDO pathway, wherein said organism further comprises one or more endogenous and/or exogenous nucleic acids encoding a 1,3-BDO pathway enzyme selected from the group consisting of 4B, 4C, 4D, 4E, 4F, 4G, 4H, 4I, 4J, 4L, 4N, and 4O; wherein at least one nucleic acid has been altered such that the 1,3-BDO pathway enzyme encoded by the nucleic acid has a lesser affinity for NADPH than the 1,3-BDO pathway enzyme encoded by an unaltered or wild-type nucleic acid. In some embodiments, the eukaryotic organism comprises a nucleic acid encoding 4B. In some embodiments, the eukaryotic organism comprises a nucleic acid encoding 4C. In some embodiments, the eukaryotic organism comprises a nucleic acid encoding 4D. In some embodiments, the eukaryotic organism comprises a nucleic acid encoding 4E. In some embodiments, the eukaryotic organism comprises a nucleic acid encoding 4F. In some embodiments, the eukaryotic organism comprises a nucleic acid encoding 4G. In some embodiments, the eukaryotic organism comprises a nucleic acid encoding 4H. In some embodiments, the eukaryotic organism comprises a nucleic acid encoding 4I. In some embodiments, the eukaryotic organism comprises a nucleic acid encoding 4J. In some embodiments, the eukaryotic organism comprises a nucleic acid encoding 4N. In some embodiments, the eukaryotic organism comprises a nucleic acid encoding 4O. In some embodiments, the eukaryotic organism comprises nucleic acids encoding 4B and 4D. In some embodiments, the eukaryotic organism comprises nucleic acids encoding 4E, 4C and 4D. In some embodiments, the eukaryotic organism comprises nucleic acids encoding 4E, 4F and 4G. In some embodiments, the eukaryotic organism comprises nucleic acids encoding 4L, 4F and 4G. In some embodiments, the eukaryotic organism comprises nucleic acids encoding 4H, 4N and 4G. In some embodiments, the eukaryotic organism comprises nucleic acids encoding 4H and 4J. In some embodiments, the eukaryotic organism comprises nucleic acids encoding 4H, 4I and 4G. In some embodiments, the eukaryotic organism comprises nucleic acids encoding 4L, 4F and 4G. In some embodiments, the eukaryotic organism comprises nucleic acids encoding 4O, 4N and 4G. In some embodiments, the eukaryotic organism comprises nucleic acids encoding 4A, 4N and 4G.

In another aspect, provided herein is a non-naturally occurring eukaryotic organism comprising a 1,3-BDO pathway wherein said organism further comprises one or more endogenous and/or exogenous nucleic acids encoding a 1,3-BDO pathway enzyme selected from the group consisting of 4B, 4C, 4D, 4E, 4F, 4G, 4H, 4I, 4J, 4L, 4N and 4O; wherein the eukaryotic organism comprises one or more gene disruptions that attenuate the activity of an endogenous NADPH-dependent 1,3-BDO pathway enzyme.

Alternatively, in some embodiments, the eukaryotic organism comprises a 1,3-BDO pathway, wherein one or more of the 1,3-BDO pathway enzymes utilizes NADPH as the cofactor. Therefore, it can be beneficial to increase the production of NADPH in these eukaryotic organisms to achieve greater yields of 1,3-BDO. Several approaches for increasing cytosolic production of NADPH can be implemented including channeling an increased amount of flux through the oxidative branch of the pentose phosphate pathway relative to wild-type, channeling an increased amount of flux through the Entner Doudoroff pathway relative to wild-type, introducing a soluble or membrane-bound transhydrogenase to convert NADH to NADPH, or employing NADP-dependent versions of the following enzymes: phosphorylating or non-phosphorylating glyceraldehyde-3-phosphate dehydrogenase, pyruvate dehydrogenase, formate dehydrogenase, or acylating acetylaldehyde dehydrogenase. Methods for increasing cytosolic production of NADPH can be augmented by eliminating or attenuating native NAD-dependent enzymes including glyceraldehyde-3-phosphate dehydrogenase, pyruvate dehydrogenase, formate dehydrogenase, or acylating acetylaldehyde dehydrogenase. Methods for engineering increased NADPH availability are described in Example IX.

In another aspect provided herein, is a non-naturally eukaryotic organism comprising: (1) a 1,3-BDO pathway, wherein said organism comprises at least one endogenous and/or exogenous nucleic acid encoding an NADPH-dependent 1,3-BDO pathway enzyme expressed in a sufficient amount to produce 1,3-BDO; and (2) a pentose phosphate pathway, wherein said organism comprises at least one endogenous and/or exogenous nucleic acid encoding a pentose phosphate pathway enzyme selected from the group consisting of glucose-6-phosphate dehydrogenase, 6-phosphogluconolactonase, and 6 phosphogluconate dehydrogenase (decarboxylating). In certain embodiments, the organism further comprises a genetic alteration that increases metabolic flux into the pentose phosphate pathway.

In another aspect provided herein, is a non-naturally eukaryotic organism comprising: (1) a 1,3-BDO pathway, wherein said organism comprises at least one endogenous and/or exogenous nucleic acid encoding an NADPH-dependent 1,3-BDO pathway enzyme expressed in a sufficient amount to produce 1,3-BDO; and (2) an Entner Doudoroff pathway, wherein said organism comprises at least one endogenous and/or exogenous nucleic acid encoding an Entner Doudoroff pathway enzyme selected from the group consisting of glucose-6-phosphate dehydrogenase, 6-phosphogluconolactonase, phosphogluconate dehydratase, and 2-keto-3-deoxygluconate 6-phosphate aldolase. In certain embodiments, the organism further comprises a genetic alteration that increases metabolic flux into the Entner Doudoroff pathway.

In another aspect, provided herein is a non-naturally eukaryotic organism comprising a 1,3-BDO pathway, wherein said organism further comprises: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) an endogenous and/or exogenous nucleic acid encoding a soluble or membrane-bound transhydrogenase, wherein the transhydrogenase is expressed at a sufficient level to convert NADH to NADPH.

In another aspect, provided herein is a non-naturally eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) an endogenous and/or exogenous nucleic acid encoding an NADP-dependent phosphorylating or non-phosphorylating glyceraldehyde-3-phosphate dehydrogenase.

In another aspect, provided herein is a non-naturally eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) an acetyl-CoA pathway, wherein said organism comprises at least one endogenous and/or exogenous nucleic acid encoding an acetyl-CoA pathway enzyme expressed in a sufficient amount to increase NADPH in the organism; wherein the acetyl-CoA pathway comprises (i) an NADP-dependent pyruvate dehydrogenase; (ii) a pyruvate formate lyase and an NADP-dependent formate dehydrogenase; (iii) a pyruvate:ferredoxin oxidoreductase and an NADPH:ferredoxin oxidoreductase; (iv) a pyruvate decarboxylase and an NADP-dependent acylating acetylaldehyde dehydrogenase; (v) a pyruvate decarboxylase, a NADP-dependent acylating acetaldehyde dehydrogenase, an acetate kinase, and a phosphotransacetylase; or (vi) a pyruvate decarboxylase, an NADP-dependent acylating acetaldehyde dehydrogenase, and an acetyl-CoA synthetase. In one embodiment, the acetyl-COA pathway comprises an NADP-dependent pyruvate dehydrogenase. In another embodiment, the acetyl-COA pathway comprises a pyruvate formate lyase and an NADP-dependent formate dehydrogenase. In other embodiments, the acetyl-COA pathway comprises a pyruvate:ferredoxin oxidoreductase and an NADPH:ferredoxin oxidoreductase. In another embodiment, the acetyl-COA pathway comprises a pyruvate decarboxylase and an NADP-dependent acylating acetylaldehyde dehydrogenase. In another embodiment, the acetyl-COA pathway comprises a pyruvate decarboxylase, a NADP-dependent acylating acetaldehyde dehydrogenase, an acetate kinase, and a phosphotransacetylase. In another embodiment, the acetyl-COA pathway comprises a pyruvate decarboxylase, an NADP-dependent acylating acetaldehyde dehydrogenase, and an acetyl-CoA synthetase. In another embodiment, the organism further comprises one or more gene disruptions that attenuate the activity of an endogenous NAD-dependant pyruvate dehydrogenase, NAD-dependent formate dehydrogenase, NADH:ferredoxin oxidoreductase, NAD-dependent acylating acetylaldehyde dehydrogenase, or NAD-dependent acylating acetaldehyde dehydrogenase. In some embodiments, the organism further comprising one or more gene disruptions that attenuate the activity of an endogenous NAD-dependant pyruvate dehydrogenase, NAD-dependent formate dehydrogenase, NADH:ferredoxin oxidoreductase, NAD-dependent acylating acetylaldehyde dehydrogenase, or NAD-dependent acylating acetaldehyde dehydrogenase.

In another aspect, provided herein is a non-naturally eukaryotic organism comprising: (1) a 1,3-BDO pathway, wherein said organism comprises at least one endogenous and/or exogenous nucleic acid encoding a NADPH-dependent 1,3-BDO pathway enzyme expressed in a sufficient amount to produce 1,3-BDO; and (2) one or more endogenous and/or exogenous nucleic acids encoding a NAD(P)H cofactor enzyme selected from the group consisting of phosphorylating or non-phosphorylating glyceraldehyde-3-phosphate dehydrogenase; pyruvate dehydrogenase; formate dehydrogenase; and acylating acetylaldehyde dehydrogenase; wherein the one or more nucleic acids encoding a NAD(P)H cofactor enzyme has been altered such that the NAD(P)H cofactor enzyme encoded by the nucleic acid has a greater affinity for NADPH than the NAD(P)H cofactor enzyme encoded by an unaltered or wild-type nucleic acid. In one embodiment, the NAD(P)H cofactor enzyme is a phosphorylating or non-phosphorylating glyceraldehyde-3-phosphate dehydrogenase. In another embodiment, the NAD(P)H cofactor enzyme is a pyruvate dehydrogenase. In another embodiment, the NAD(P)H cofactor enzyme is a formate dehydrogenase. In yet another embodiment, the NAD(P)H cofactor enzyme is an acylating acetylaldehyde dehydrogenase.

In another aspect, provided herein is a non-naturally eukaryotic organism comprising a 1,3-BDO pathway, wherein said organism further comprises: (1) a 1,3-BDO pathway, wherein said organism comprises at least one endogenous and/or exogenous nucleic acid encoding a NADPH dependent 1,3-BDO pathway enzyme expressed in a sufficient amount to produce 1,3-BDO; and (2) one or more endogenous and/or exogenous nucleic acids encoding a NAD(P)H cofactor enzyme selected from the group consisting of a phosphorylating or non-phosphorylating glyceraldehyde-3-phosphate dehydrogenase; a pyruvate dehydrogenase; a formate dehydrogenase; and an acylating acetylaldehyde dehydrogenase; wherein the one or more nucleic acids encoding NAD(P)H cofactor enzyme nucleic acid has been altered such that the NAD(P)H cofactor enzyme that it encodes for has a lesser affinity for NADH than the NAD(P)H cofactor enzyme encoded by an unaltered or wild-type nucleic acid. In one embodiment, the NAD(P)H cofactor enzyme is a phosphorylating or non-phosphorylating glyceraldehyde-3-phosphate dehydrogenase. In another embodiment, the NAD(P)H cofactor enzyme is a pyruvate dehydrogenase. In another embodiment, the NAD(P)H cofactor enzyme is a formate dehydrogenase. In yet another embodiment, the NAD(P)H cofactor enzyme is an acylating acetylaldehyde dehydrogenase.

In one embodiment of the eukaryotic organisms provided above, the 1,3-BDO pathway comprises 4A, 4E, 4F and 4G. In another embodiment, the 1,3-BDO pathway comprises 4A, 4B and 4D. In other embodiments, the 1,3-BDO pathway comprises 4A, 4E, 4C and 4D. In some embodiments, the 1,3-BDO pathway comprises 4A, 4H and 4J. In other embodiments, the 1,3-BDO pathway comprises 4A, 4H, 4I and 4G. In certain embodiments, the 1,3-BDO pathway comprises 4A, 4H, 4M, 4N and 4G. In another embodiment, the 1,3-BDO pathway comprises 4A, 4K, 4O, 4N and 4G. In yet another embodiment, the 1,3-BDO pathway comprises 4A, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

In another embodiment of the eukaryotic organisms provided above, the 1,3-BDO pathway comprises 7E, 7F, 4E, 4F and 4G. In another embodiment, the 1,3-BDO pathway comprises 7E, 7F, 4B and 4D. In other embodiments, the 1,3-BDO pathway comprises 7E, 7F, 4E, 4C and 4D. In some embodiments, the 1,3-BDO pathway comprises 7E, 7F, 4H and 4J. In other embodiments, the 1,3-BDO pathway comprises 7E, 7F, 4H, 4I and 4G. In certain embodiments, the 1,3-BDO pathway comprises 7E, 7F, 4H, 4M, 4N and 4G. In another embodiment, the 1,3-BDO pathway comprises 7E, 7F, 4K, 4O, 4N and 4G. In yet another embodiment, the 1,3-BDO pathway comprises 7E, 7F, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

### 4.5 Increase of Redox Ratio

Synthesis of 1,3-BDO, in the cytosol of eukaryotic organisms requires the availability of sufficient carbon and reducing equivalents. Therefore, without being bound to any particular theory of operation, increasing the redox ratio of NAD(P)H to NAD(P) can help drive the 1,3-BDO pathway in the forward direction. Methods for increasing the redox ratio of NAD(P)H to NAD(P) include limiting respiration, attenuating or eliminating competing pathways that produce reduced byproducts, attenuating or eliminating the use of NADH by NADH dehydrogenases, and attenuating or eliminating redox shuttles between compartments.

One exemplary method to provide an increased number of reducing equivalents, such as NAD(P)H, for enabling the formation of 1,3-BDO is to constrain the use of such reducing equivalents during respiration. Respiration can be limited by: reducing the availability of oxygen, attenuating NADH dehydrogenases and/or cytochrome oxidase activity, attenuating G3P dehydrogenase, and/or providing excess glucose to Crabtree positive organisms.

Restricting oxygen availability by culturing the non-naturally occurring eukaryotic organisms in a fermenter is one approach for limiting respiration and thereby increasing the ratio of NAD(P)H to NAD(P). The ratio of NAD(P)H/NAD(P) increases as culture conditions get more anaerobic, with completely anaerobic conditions providing the highest ratios of the reduced cofactors to the oxidized ones. For example, it has been reported that the ratio of NADH/NAD=0.02 in aerobic conditions and 0.75 in anaerobic conditions in E. coli (de Graes et al, J Bacteriol 181:2351-57 (1999)).

Respiration can also be limited by reducing expression or activity of NADH dehydrogenases and/or cytochrome oxidases in the cell under aerobic conditions. In this case, respiration will be limited by the capacity of the electron transport chain. Such an approach has been used to enable anaerobic metabolism of E. coli under completely aerobic conditions (Portnoy et al, AEM 74:7561-9 (2008)). S. cerevisiae can oxidize cytosolic NADH directly using external NADH dehydrogenases, encoded by NDE1 and NDE2. One such NADH dehydrogenase in Yarrowia lipolytica is encoded by NDH2 (Kerscher et al, J Cell Sci 112:2347-54 (1999)). These and other NADH dehydrogenase enzymes are listed in the table below.

TABLE 2

| Protein | GenBank ID | GI number | Organism |
|---|---|---|---|
| NDE1 | NP_013865.1 | 6323794 | Saccharomyces |
| | | | cerevisiae s288c |
| NDE2 | NP_010198.1 | 6320118 | Saccharomyces |
| | | | cerevisiae s288c |
| NDH2 | AJ006852.1 | 3718004 | Yarrowia lipolytica |
| ANI_1_610074 | XP_001392541.2 | 317030427 | Aspergillus niger |
| ANI_1_2462094 | XP_001394893.2 | 317033119 | Aspergillus niger |
| KLLA0E21891g | XP_454942.1 | 50309857 | Kluyveromyces |
| | | | lactis |
| KLLA0C06336g | XP_452480.1 | 50305045 | Kluyveromyces |
| | | | lactis |
| NDE1 | XP_720034.1 | 68471982 | Candida albicans |
| NDE2 | XP_717986.1 | 68475826 | Candida albicans |

Cytochrome oxidases of Saccharomyces cerevisiae include the COX gene products. COX1-3 are the three core subunits encoded by the mitochondrial genome, whereas COX4-13 are encoded by nuclear genes. Attenuation or deletion of any of the cytochrome genes results in a decrease or block in respiratory growth (Hermann and Funes, Gene 354:43-52 (2005)). Cytochrome oxidase genes in other organisms can be inferred by sequence homology.

TABLE 3

| Protein | GenBank ID | GI number | Organism |
|---|---|---|---|
| COX1 | CAA09824.1 | 4160366 | Saccharomyces cerevisiae s288c |
| COX2 | CAA09845.1 | 4160387 | Saccharomyces cerevisiae s288c |
| COX3 | CAA09846.1 | 4160389 | Saccharomyces cerevisiae s288c |
| COX4 | NP_011328.1 | 6321251 | Saccharomyces cerevisiae s288c |
| COX5A | NP_014346.1 | 6324276 | Saccharomyces cerevisiae s288c |
| COX5B | NP_012155.1 | 6322080 | Saccharomyces cerevisiae s288c |
| COX6 | NP_011918.1 | 6321842 | Saccharomyces cerevisiae s288c |
| COX7 | NP_013983.1 | 6323912 | Saccharomyces cerevisiae s288c |
| COX8 | NP_013499.1 | 6323427 | Saccharomyces cerevisiae s288c |
| COX9 | NP_010216.1 | 6320136 | Saccharomyces cerevisiae s288c |
| COX12 | NP_013139.1 | 6323067 | Saccharomyces cerevisiae s288c |
| COX13 | NP_011324.1 | 6321247 | Saccharomyces cerevisiae s288c |

In one aspect provided herein, is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in a endogenous and/or exogenous nucleic acid encoding a NADH dehydrogenase; (ii) expresses an attenuated NADH dehydrogenase; and/or (iii) has lower or no NADH dehydrogenase enzymatic activity as compared to a wild-type version of the eukaryotic organism. In one embodiment, the organism (i) comprises a disruption in a endogenous and/or exogenous nucleic acid encoding a NADH dehydrogenase; and (ii) expresses an attenuated NADH dehydrogenase. In another embodiment, the organism (i) comprises a disruption in a endogenous and/or exogenous nucleic acid encoding a NADH dehydrogenase; and (iii) has lower or no NADH dehydrogenase enzymatic activity as compared to a wild-type version of the eukaryotic organism. In another embodiment, the organism (ii) expresses an attenuated NADH dehydrogenase; and (iii) has lower or no NADH dehydrogenase enzymatic activity as compared to a wild-type version of the eukaryotic organism. In yet another embodiment, the organism (i) comprises a disruption in a endogenous and/or exogenous nucleic acid encoding a NADH dehydrogenase; (ii) expresses an attenuated NADH dehydrogenase; and (iii) has lower or no NADH dehydrogenase enzymatic activity as compared to a wild-type version of the eukaryotic organism.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a cytochrome oxidase; (ii) expresses an attenuated cytochrome oxidase; and/or (iii) has lower or no cytochrome oxidase enzymatic activity as compared to a wild-type version of the eukaryotic organism. In one embodiment, the organism (i) comprises a disruption in an endogenous and/or exogenous nucleic acid encoding a cytochrome oxidase; and (ii) expresses an attenuated cytochrome oxidase. In another embodiment, the organism (i) comprises a disruption in an endogenous and/or exogenous nucleic acid encoding a cytochrome oxidase; and (iii) has lower or no cytochrome oxidase enzymatic activity as compared to a wild-type version of the eukaryotic organism. In another embodiment, the organism (ii) expresses an attenuated cytochrome oxidase; and (iii) has lower or no cytochrome oxidase enzymatic activity as compared to a wild-type version of the eukaryotic organism. In yet another embodiment, the organism (i) comprises a disruption in an endogenous and/or exogenous nucleic acid encoding a cytochrome oxidase; (ii) expresses an attenuated cytochrome oxidase; and (iii) has lower or no cytochrome oxidase enzymatic activity as compared to a wild-type version of the eukaryotic organism.

In certain embodiments, cytosolic NADH can also be oxidized by the respiratory chain via the G3P dehydrogenase shuttle, consisting of cytosolic NADH-linked G3P dehydrogenase and a membrane-bound G3P:ubiquinone oxidoreductase. The deletion or attenuation of G3P dehydrogenase enzymes will also prevent the oxidation of NADH for respiration. S. cerevisiae has three G3P dehydrogenase enzymes encoded by GPD1 and GDP2 in the cytosol and GUT2 in the mitochondrion. GPD2 is known to encode the enzyme responsible for the majority of the glycerol formation and is responsible for maintaining the redox balance under anaerobic conditions. GPD1 is primarily responsible for adaptation of S. cerevisiae to osmotic stress (Bakker et al., FEMS Microbiol Rev 24:15-37 (2001)). Attenuation of GPD1, GPD2 and/or GUT2 will reduce glycerol formation. GPD1 and GUT2 encode G3P dehydrogenases in Yarrowia lipolytica (Beopoulos et al, AEM 74:7779-89 (2008)). GPD1 and GPD2 encode for G3P dehydrogenases in S. pombe. Similarly, G3P dehydrogenase is encoded by CTRG_02011 in Candida tropicalis and a gene represented by GI:20522022 in Candida albicans.

TABLE 4

| Protein | GenBank ID | GI number | Organism |
|---|---|---|---|
| GPD1 | CAA98582.1 | 1430995 | Saccharomyces cerevisiae |
| GPD2 | NP_014582.1 | 6324513 | Saccharomyces cerevisiae |
| GUT2 | NP_012111.1 | 6322036 | Saccharomyces cerevisiae |
| GPD1 | CAA22119.1 | 6066826 | Yarrowia lipolytica |
| GUT2 | CAG83113.1 | 49646728 | Yarrowia lipolytica |
| GPD1 | CAA22119.1 | 3873542 | Schizosaccharomyces pombe |
| GPD2 | CAA91239.1 | 1039342 | Schizosaccharomyces pombe |
| ANI_1_786014 | XP_001389035.2 | 317025419 | Aspergillus niger |
| ANI_1_1768134 | XP_001397265.1 | 145251503 | Aspergillus niger |
| KLLA0C04004g | XP_452375.1 | 50304839 | Kluyveromyces lactis |
| CTRG_02011 | XP_002547704.1 | 255725550 | Candida tropicalis |
| GPD1 | XP_714362.1 | 68483412 | Candida albicans |
| GPD2 | XP_713824.1 | 68484586 | Candida albicans |

In another aspect, provided herein is a non-naturally occurring eukaryotic organism comprising a 1,3-BDO pathway, wherein said organism comprises at least one exogenous nucleic acid encoding a 1,3-BDO pathway enzyme expressed in a sufficient amount to produce 1,3-BDO, wherein the non-naturally occurring eukaryotic organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a G3P dehydrogenase; (ii) expresses an attenuated G3P dehydrogenase; (iii) has lower or no G3P dehydrogenase enzymatic activity as compared to a wild-type version of the eukaryotic organism; and/or (iv) produces lower levels of glycerol as compared to a wild-type version of the eukaryotic organism. In one embodiment, the organism (i) comprises a disruption in an endogenous and/or exogenous nucleic acid encoding a G3P dehydrogenase; and (ii) expresses an attenuated G3P dehydrogenase. In another embodiment, the organism (i) comprises a disruption in an endogenous and/or exogenous nucleic acid encoding a G3P dehydrogenase; and (iii) has lower or no G3P dehydrogenase enzymatic activity as compared to a wild-type version of the eukaryotic organism. In another embodiment, the organism (i) comprises a disruption in an endogenous and/or exogenous nucleic acid encoding a G3P dehydrogenase and (iv) produces lower levels of glycerol as compared to a wild-type version of the eukaryotic organism. In another embodiment, the organism (ii) expresses an attenuated G3P dehydrogenase and (iii) has lower or no G3P dehydrogenase enzymatic activity as compared to a wild-type version of the eukaryotic organism. In another embodiment, the organism (ii) expresses an attenuated G3P dehydrogenase; and (iv) produces lower levels of glycerol as compared to a wild-type version of the eukaryotic organism. In another embodiment, the organism (iii) has lower or no G3P dehydrogenase enzymatic activity as compared to a wild-type version of the eukaryotic organism; and (iv) produces lower levels of glycerol as compared to a wild-type version of the eukaryotic organism. In another embodiment, the organism (i) comprises a disruption in an endogenous and/or exogenous nucleic acid encoding a G3P dehydrogenase; (ii) expresses an attenuated G3P dehydrogenase; and (iii) has lower or no G3P dehydrogenase enzymatic activity as compared to a wild-type version of the eukaryotic organism. In another embodiment, the organism (i) comprises a disruption in an endogenous and/or exogenous nucleic acid encoding a G3P dehydrogenase; (ii) expresses an attenuated G3P dehydrogenase; and (iv) produces lower levels of glycerol as compared to a wild-type version of the eukaryotic organism. In yet another embodiment, the organism (i) comprises a disruption in an endogenous and/or exogenous nucleic acid encoding a G3P dehydrogenase; (ii) expresses an attenuated G3P dehydrogenase; (iii) has lower or no G3P dehydrogenase enzymatic activity as compared to a wild-type version of the eukaryotic organism; and (iv) produces lower levels of glycerol as compared to a wild-type version of the eukaryotic organism.

Additionally, in Crabtree positive organisms, fermentative metabolism can be achieved in the presence of excess of glucose. For example, S. cerevisiae makes ethanol even under aerobic conditions. The formation of ethanol and glycerol can be reduced/eliminated and replaced by the production of 1,3-BDO in a Crabtree positive organism by feeding excess glucose to the Crabtree positive organism. In another aspect provided herein is a method for producing 1,3-BDO, comprising culturing a non-naturally occurring eukaryotic organism under conditions and for a sufficient period of time to produce 1,3-BDO, wherein the eukaryotic organism is a Crabtree positive organism that comprises at least one exogenous nucleic acid encoding a 1,3-BDO pathway enzyme and wherein eukaryotic organism is in a culture medium comprising excess glucose.

Preventing formation of reduced fermentation byproducts can also increase the availability of both carbon and reducing equivalents for 1,3-BDO. Two key reduced byproducts under anaerobic and microaerobic conditions are ethanol and glycerol. Ethanol can be formed from pyruvate in two enzymatic steps catalyzed by pyruvate decarboxylase and ethanol dehydrogenase. Glycerol can be formed from the glycolytic intermediate dihydroxyacetone phosphate by the enzymes G3P dehydrogenase and G3P phosphatase. Attenuation of one or more of these enzyme activities in the eukaryotic organisms provided herein can increase the yield of 1,3-BDO. Methods for strain engineering for reducing or eliminating ethanol and glycerol formation are described in further detail elsewhere herein.

The conversion of acetyl-CoA into ethanol can be detrimental to the production of 1,3-BDO because the conversion process can draw away both carbon and reducing equivalents from the 1,3-BDO pathway. Ethanol can be formed from pyruvate in two enzymatic steps catalyzed by pyruvate decarboxylase and ethanol dehydrogenase. Saccharomyces cerevisiae has three pyruvate decarboxylases (PDC1, PDC5 and PDC6) and two of them (PDC1, PDC5) are strongly expressed. Deleting two of these PDCs can reduce ethanol production significantly. Deletion of all three eliminates ethanol formation completely but also can cause a growth defect because of inability of the cells to form acetyl-CoA for biomass formation. This, however, can be overcome by evolving cells in the presence of reducing amounts of C2 carbon source (ethanol or acetate) (van Maris et al, AEM 69:2094-9 (2003)). It has also been reported that deletion of the positive regulator PDC2 of pyruvate decarboxylases PDC1 and PDC5, reduced ethanol formation to ˜10% of that made by wild-type (Hohmann et al, Mol Gen Genet 241:657-66 (1993)). Protein sequences and identifiers of PDC enzymes are listed in Example II.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a pyruvate decarboxylase; (ii) expresses an attenuated pyruvate decarboxylase; (iii) has lower or no pyruvate decarboxylase enzymatic activity as compared to a wild-type version of the eukaryotic organism; and/or (iv) produces lower levels of ethanol from pyruvate as compared to a wild-type version of the eukaryotic organism. In one embodiment, the organism (i) comprises a disruption in an endogenous and/or exogenous nucleic acid encoding a pyruvate decarboxylase; and (ii) expresses an attenuated pyruvate decarboxylase. In another embodiment, the organism (i) comprises a disruption in an endogenous and/or exogenous nucleic acid encoding a pyruvate decarboxylase; and (iii) has lower or no pyruvate decarboxylase enzymatic activity as compared to a wild-type version of the eukaryotic organism. In another embodiment, the organism (ii) expresses an attenuated pyruvate decarboxylase; and (iv) produces lower levels of ethanol from pyruvate as compared to a wild-type version of the eukaryotic organism. In another embodiment, the organism (ii) expresses an attenuated pyruvate decarboxylase; and (iii) has lower or no pyruvate decarboxylase enzymatic activity as compared to a wild-type version of the eukaryotic organism. In another embodiment, the organism (ii) expresses an attenuated pyruvate decarboxylase; and (iv) produces lower levels of ethanol from pyruvate as compared to a wild-type version of the eukaryotic organism. In another embodiment, the organism (iii) has lower or no pyruvate decarboxylase enzymatic activity as compared to a wild-type version of the eukaryotic organism; and (iv) produces lower levels of ethanol from pyruvate as compared to a wild-type version of the eukaryotic organism. In another embodiment, the organism (i) comprises a disruption in an endogenous and/or exogenous nucleic acid encoding a pyruvate decarboxylase; (ii) expresses an attenuated pyruvate decarboxylase; and (iii) has lower or no pyruvate decarboxylase enzymatic activity as compared to a wild-type version of the eukaryotic organism. In another embodiment, the organism (i) comprises a disruption in an endogenous and/or exogenous nucleic acid encoding a pyruvate decarboxylase; (iii) has lower or no pyruvate decarboxylase enzymatic activity as compared to a wild-type version of the eukaryotic organism; and (iv) produces lower levels of ethanol from pyruvate as compared to a wild-type version of the eukaryotic organism. In another embodiment, the organism (ii) expresses an attenuated pyruvate decarboxylase; (iii) has lower or no pyruvate decarboxylase enzymatic activity as compared to a wild-type version of the eukaryotic organism; and (iv) produces lower levels of ethanol from pyruvate as compared to a wild-type version of the eukaryotic organism. In yet another embodiment, the organism (i) comprises a disruption in an endogenous and/or exogenous nucleic acid encoding a pyruvate decarboxylase; (ii) expresses an attenuated pyruvate decarboxylase; (iii) has lower or no pyruvate decarboxylase enzymatic activity as compared to a wild-type version of the eukaryotic organism; and (iv) produces lower levels of ethanol from pyruvate as compared to a wild-type version of the eukaryotic organism.

Alternatively, ethanol dehydrogenases that convert acetaldehyde into ethanol can be deleted or attenuated to provide carbon and reducing equivalents for the 1,3-BDO pathway. To date, seven alcohol dehydrogenases, ADHI-ADHVII, have been reported in S. cerevisiae (de Smidt et al, FEMS Yeast Res 8:967-78 (2008)). ADH1 (GI:1419926) is the key enzyme responsible for reducing acetaldehyde to ethanol in the cytosol under anaerobic conditions. It has been reported that a yeast strain deficient in ADH1 cannot grow anaerobically because an active respiratory chain is the only alternative path to regenerate NADH and lead to a net gain of ATP (Drewke et al, J Bacteriol 172:3909-17 (1990)). This enzyme is an ideal candidate for downregulation to limit ethanol production. ADH2 is severely repressed in the presence of glucose. In K. lactis, two NAD-dependent cytosolic alcohol dehydrogenases have been identified and characterized. These genes also show activity for other aliphatic alcohols. The genes ADH1 (GI:113358) and ADHII (GI:51704293) are preferentially expressed in glucose-grown cells (Bozzi et al, Biochim Biophys Acta 1339:133-142 (1997)). Cytosolic alcohol dehydrogenases are encoded by ADH1 (GI:608690) in C. albicans, ADH1 (GI:3810864) in S. pombe, ADH1 (GI:5802617) in Y. lipolytica, ADH1 (GI:2114038) and ADHII (GI:2143328) in Pichia stipitis or Scheffersomyces stipitis (Passoth et al, Yeast 14:1311-23 (1998)). Candidate alcohol dehydrogenases are shown the table below.

TABLE 5

| Protein | GenBank ID | GI number | Organism |
|---|---|---|---|
| SADH | BAA24528.1 | 2815409 | Candida parapsilosis |
| ADH1 | NP_014555.1 | 6324486 | Saccharomyces |
| | | | cerevisiae s288c |
| ADH2 | NP_014032.1 | 6323961 | Saccharomyces |
| | | | cerevisiae s288c |
| ADH3 | NP_013800.1 | 6323729 | Saccharomyces |
| | | | cerevisiae s288c |
| ADH4 | NP_011258.2 | 269970305 | Saccharomyces |
| | | | cerevisiae s288c |
| ADH5 (SFA1) | NP_010113.1 | 6320033 | Saccharomyces |
| | | | cerevisiae s288c |
| ADH6 | NP_014051.1 | 6323980 | Saccharomyces |
| | | | cerevisiae s288c |
| ADH7 | NP_010030.1 | 6319949 | Saccharomyces |
| | | | cerevisiae s288c |
| adhP | CAA44614.1 | 2810 | Kluyveromyces lactis |
| ADH1 | P20369.1 | 113358 | Kluyveromyces lactis |
| ADH2 | CAA45739.1 | 2833 | Kluyveromyces lactis |
| ADH3 | P49384.2 | 51704294 | Kluyveromyces lactis |

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an ethanol dehydrogenase; (ii) expresses an attenuated ethanol dehydrogenase; (iii) has lower or no ethanol dehydrogenase enzymatic activity as compared to a wild-type version of the eukaryotic organism; and/or (iv) produces lower levels of ethanol as compared to a wild-type version of the eukaryotic organism. In one embodiment, the organism (i) comprises a disruption in an endogenous and/or exogenous nucleic acid encoding an ethanol dehydrogenase; and (ii) expresses an attenuated ethanol dehydrogenase. In another embodiment, the organism (i) comprises a disruption in an endogenous and/or exogenous nucleic acid encoding an ethanol dehydrogenase; and (iii) has lower or no ethanol dehydrogenase enzymatic activity as compared to a wild-type version of the eukaryotic organism. In another embodiment, the organism (i) comprises a disruption in an endogenous and/or exogenous nucleic acid encoding an ethanol dehydrogenase; and (iv) produces lower levels of ethanol as compared to a wild-type version of the eukaryotic organism. In another embodiment, the organism (ii) expresses an attenuated ethanol dehydrogenase; and (iii) has lower or no ethanol dehydrogenase enzymatic activity as compared to a wild-type version of the eukaryotic organism. In another embodiment, the organism (ii) expresses an attenuated ethanol dehydrogenase; and (iv) produces lower levels of ethanol as compared to a wild-type version of the eukaryotic organism. In another embodiment, the organism (iii) has lower or no ethanol dehydrogenase enzymatic activity as compared to a wild-type version of the eukaryotic organism; and (iv) produces lower levels of ethanol as compared to a wild-type version of the eukaryotic organism. In another embodiment, the organism (i) comprises a disruption in an endogenous and/or exogenous nucleic acid encoding an ethanol dehydrogenase; (ii) expresses an attenuated ethanol dehydrogenase; and (iii) has lower or no ethanol dehydrogenase enzymatic activity as compared to a wild-type version of the eukaryotic organism. In another embodiment, the organism (i) comprises a disruption in an endogenous and/or exogenous nucleic acid encoding an ethanol dehydrogenase; (ii) expresses an attenuated ethanol dehydrogenase; and (iv) produces lower levels of ethanol as compared to a wild-type version of the eukaryotic organism. In another embodiment, the organism (i) comprises a disruption in an endogenous and/or exogenous nucleic acid encoding an ethanol dehydrogenase; (iii) has lower or no ethanol dehydrogenase enzymatic activity as compared to a wild-type version of the eukaryotic organism; and (iv) produces lower levels of ethanol as compared to a wild-type version of the eukaryotic organism. In another embodiment, the organism (i) comprises a disruption in an endogenous and/or exogenous nucleic acid encoding an ethanol dehydrogenase; (ii) expresses an attenuated ethanol dehydrogenase; (iii) has lower or no ethanol dehydrogenase enzymatic activity as compared to a wild-type version of the eukaryotic organism; and (iv) produces lower levels of ethanol as compared to a wild-type version of the eukaryotic organism.

Yeast such as S. cerevisiae can produce glycerol to allow for regeneration of NAD(P) under anaerobic conditions. Glycerol is formed from the glycolytic intermediate dihydroxyacetone phosphate by the enzymes G3P dehydrogenase and G3P phosphatase. Without being bound by a particular theory of operation, it is believed that attenuation or deletion of one or more of these enzymes can eliminate or reduce the formation of glycerol, and thereby conserve reducing equivalents for production of 1,3-BDO. Exemplary G3P dehydrogenase enzymes were described above. G3P phosphatase catalyzes the hydrolysis of G3P to glycerol. Enzymes with this activity include the glycerol-1-phosphatase (EC 3.1.3.21) enzymes of Saccharomyces cerevisiae (GPP1 and GPP2), Candida albicans and Dunaleilla parva (Popp et al, Biotechnol Bioeng 100:497-505 (2008); Fan et al, FEMS Microbiol Lett 245:107-16 (2005)). The D. parva gene has not been identified to date. These and additional G3P phosphatase enzymes are shown in the table below.

TABLE 6

| Protein | GenBank ID | GI Number | Organism |
|---|---|---|---|
| GPP1 | DAA08494.1 | 285812595 | Saccharomyces |
| | | | cerevisiae |
| GPP2 | NP_010984.1 | 6320905 | Saccharomyces |
| | | | cerevisiae |
| GPP1 | XP_717809.1 | 68476319 | Candida albicans |
| KLLA0C08217g | XP_452565.1 | 50305213 | Kluyveromyces |
| | | | lactis |
| KLLA0C11143g | XP_452697.1 | 50305475 | Kluyveromyces |
| | | | lactis |
| ANI_1_380074 | XP_001392369.1 | 145239445 | Aspergillus niger |
| ANI_1_444054 | XP_001390913.2 | 317029125 | Aspergillus niger |

In another aspect, provided herein is a non-naturally occurring eukaryotic organism comprising a 1,3-BDO pathway, comprising at least one exogenous nucleic acid encoding a 1,3-BDO pathway enzyme expressed in a sufficient amount to produce 1,3-BDO, wherein the non-naturally occurring eukaryotic organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a G3P dehydrogenase; (ii) expresses an attenuated G3P dehydrogenase; (iii) has lower or no G3P dehydrogenase enzymatic activity as compared to a wild-type version of the eukaryotic organism; and/or (iv) produces lower levels of glycerol as compared to a wild-type version of the eukaryotic organism. In one embodiment, the organism (i) comprises a disruption in an endogenous and/or exogenous nucleic acid encoding a G3P phosphatase; and (ii) expresses an attenuated G3P phosphatase. In another embodiment, the organism (i) comprises a disruption in an endogenous and/or exogenous nucleic acid encoding a G3P phosphatase; and (iii) has lower or no G3P phosphatase enzymatic activity as compared to a wild-type version of the eukaryotic organism. In another embodiment, the organism (i) comprises a disruption in an endogenous and/or exogenous nucleic acid encoding a G3P phosphatase and (iv) produces lower levels of glycerol as compared to a wild-type version of the eukaryotic organism. In another embodiment, the organism (ii) expresses an attenuated G3P phosphatase and (iii) has lower or no G3P phosphatase enzymatic activity as compared to a wild-type version of the eukaryotic organism. In another embodiment, the organism (ii) expresses an attenuated G3P phosphatase; and (iv) produces lower levels of glycerol as compared to a wild-type version of the eukaryotic organism. In another embodiment, the organism (iii) has lower or no G3P phosphatase enzymatic activity as compared to a wild-type version of the eukaryotic organism; and (iv) produces lower levels of glycerol as compared to a wild-type version of the eukaryotic organism. In another embodiment, the organism (i) comprises a disruption in an endogenous and/or exogenous nucleic acid encoding a G3P phosphatase; (ii) expresses an attenuated G3P phosphatase; and (iii) has lower or no G3P phosphatase enzymatic activity as compared to a wild-type version of the eukaryotic organism. In another embodiment, the organism (i) comprises a disruption in an endogenous and/or exogenous nucleic acid encoding a G3P phosphatase; (ii) expresses an attenuated G3P phosphatase; and (iv) produces lower levels of glycerol as compared to a wild-type version of the eukaryotic organism. In yet another embodiment, the organism (i) comprises a disruption in an endogenous and/or exogenous nucleic acid encoding a G3P phosphatase; (ii) expresses an attenuated G3P phosphatase; (iii) has lower or no G3P phosphatase enzymatic activity as compared to a wild-type version of the eukaryotic organism; and (iv) produces lower levels of glycerol as compared to a wild-type version of the eukaryotic organism.

Another way to eliminate glycerol production is by oxygen-limited cultivation (Bakker et al, supra). Glycerol formation only sets in when the specific oxygen uptake rates of the cells decrease below the rate that is required to reoxidize the NADH formed in biosynthesis.

In addition to the redox sinks listed above, malate dehydrogenase can potentially draw away reducing equivalents when it functions in the reductive direction. Several redox shuttles believed to be functional in S. cerevisiae utilize this enzyme to transfer reducing equivalents between the cytosol and the mitochondria. This transfer of redox can be prevented by eliminating malate dehydrogenase and/or malic enzyme activity. The redox shuttles that can be blocked by the elimination of mdh include (i) malate-asparate shuttle, (ii) malate-oxaloacetate shuttle, and (iii) malate-pyruvate shuttle. Genes encoding malate dehydrogenase and malic enzymes are listed in the table below:

TABLE 7

| Protein | GenBank ID | GI Number | Organism |
|---|---|---|---|
| MDH1 | NP_012838.1 | 6322765 | Saccharomyces |
| | | | cerevisiae |
| MDH2 | NP_014515.2 | 116006499 | Saccharomyces |
| | | | cerevisiae |
| MDH3 | NP_010205.1 | 6320125 | Saccharomyces |
| | | | cerevisiae |
| MAE1 | NP_012896.1 | 6322823 | Saccharomyces |
| | | | cerevisiae |
| MDH1 | XP_722674.1 | 68466384 | Candida albicans |
| MDH2 | XP_718638.1 | 68474530 | Candida albicans |
| MAE1 | XP_716669.1 | 68478574 | Candida albicans |
| KLLA0F25960g | XP_456236.1 | 50312405 | Kluyveromyces |
| | | | lactis |
| KLLA0E18635g | XP_454793.1 | 50309563 | Kluyveromyces |
| | | | lactis |
| KLLA0E07525g | XP_454288.1 | 50308571 | Kluyveromyces |
| | | | lactis |
| YALI0D16753p | XP_502909.1 | 50550873 | Yarrowia lipolytica |
| YALI0E18634p | XP_504112.1 | 50553402 | Yarrowia lipolytica |
| ANI_1_268064 | XP_001391302.1 | 145237310 | Aspergillus niger |
| ANI_1_12134 | XP_001396546.1 | 145250065 | Aspergillus niger |
| ANI_1_22104 | XP_001395105.2 | 317033225 | Aspergillus niger |

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a malate dehydrogenase; (ii) expresses an attenuated malate dehydrogenase; (iii) has lower or no malate dehydrogenase enzymatic activity as compared to a wild-type version of the eukaryotic organism; and/or (iv) has an attenuation or blocking of a malate-asparate shuttle, a malate oxaloacetate shuttle, and/or a malate-pyruvate shuttle. In one embodiment, the organism (i) comprises a disruption in an endogenous and/or exogenous nucleic acid encoding a malate dehydrogenase; and (ii) expresses an attenuated malate dehydrogenase. In another embodiment, the organism (i) comprises a disruption in an endogenous and/or exogenous nucleic acid encoding a malate dehydrogenase; and (iii) has lower or no malate dehydrogenase enzymatic activity as compared to a wild-type version of the eukaryotic organism. In another embodiment, the organism (i) comprises a disruption in an endogenous and/or exogenous nucleic acid encoding a malate dehydrogenase; and (iv) has an attenuation or blocking of a malate-asparate shuttle, a malate oxaloacetate shuttle, and/or a malate-pyruvate shuttle. In another embodiment, the organism (ii) expresses an attenuated malate dehydrogenase; and (iii) has lower or no malate dehydrogenase enzymatic activity as compared to a wild-type version of the eukaryotic organism. In another embodiment, the organism (ii) expresses an attenuated malate dehydrogenase; and (iv) has an attenuation or blocking of a malate-asparate shuttle, a malate oxaloacetate shuttle, and/or a malate-pyruvate shuttle. In another embodiment, the organism (iii) has lower or no malate dehydrogenase enzymatic activity as compared to a wild-type version of the eukaryotic organism; and (iv) has an attenuation or blocking of a malate-asparate shuttle, a malate oxaloacetate shuttle, and/or a malate-pyruvate shuttle. In another embodiment, the organism (i) comprises a disruption in an endogenous and/or exogenous nucleic acid encoding a malate dehydrogenase; (ii) expresses an attenuated malate dehydrogenase; and (iii) has lower or no malate dehydrogenase enzymatic activity as compared to a wild-type version of the eukaryotic organism. In another embodiment, the organism (i) comprises a disruption in an endogenous and/or exogenous nucleic acid encoding a malate dehydrogenase; (ii) expresses an attenuated malate dehydrogenase; and (iv) has an attenuation or blocking of a malate-asparate shuttle, a malate oxaloacetate shuttle, and/or a malate-pyruvate shuttle. In another embodiment, the organism (i) comprises a disruption in an endogenous and/or exogenous nucleic acid encoding a malate dehydrogenase; (iii) has lower or no malate dehydrogenase enzymatic activity as compared to a wild-type version of the eukaryotic organism; and (iv) has an attenuation or blocking of a malate-asparate shuttle, a malate oxaloacetate shuttle, and/or a malate-pyruvate shuttle. In yet another embodiment, the organism (i) comprises a disruption in an endogenous and/or exogenous nucleic acid encoding a malate dehydrogenase; (ii) expresses an attenuated malate dehydrogenase; (iii) has lower or no malate dehydrogenase enzymatic activity as compared to a wild-type version of the eukaryotic organism; and (iv) has an attenuation or blocking of a malate-asparate shuttle, a malate oxaloacetate shuttle, and/or a malate-pyruvate shuttle.

Overall, deletion of the aforementioned sinks for redox either individually or in combination with the other redox sinks will eliminate the use of reducing power for respiration or byproduct formation. It has been reported that the deletion of the external NADH dehydrogenases (NDE1 and NDE2) and the mitochondrial G3P dehydrogenase (GUT2) almost completely eliminates cytosolic NAD+ regeneration in S. cerevisiae (Overkamp et al, J Bacterial 182:2823-30 (2000)).

In one embodiment of the eukaryotic organisms provided above, the 1,3-BDO pathway comprises 4A, 4E, 4F and 4G. In another embodiment, the 1,3-BDO pathway comprises 4A, 4B and 4D. In other embodiments, the 1,3-BDO pathway comprises 4A, 4E, 4C and 4D. In some embodiments, the 1,3-BDO pathway comprises 4A, 4H and 4J. In other embodiments, the 1,3-BDO pathway comprises 4A, 4H, 4I and 4G. In certain embodiments, the 1,3-BDO pathway comprises 4A, 4H, 4M, 4N and 4G. In another embodiment, the 1,3-BDO pathway comprises 4A, 4K, 4O, 4N and 4G. In yet another embodiment, the 1,3-BDO pathway comprises 4A, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

In one embodiment of the eukaryotic organisms provided above, the 1,3-BDO pathway comprises 7E, 7F, 4E, 4F and 4G. In another embodiment, the 1,3-BDO pathway comprises 7E, 7F, 4B and 4D. In other embodiments, the 1,3-BDO pathway comprises 7E, 7F, 4E, 4C and 4D. In some embodiments, the 1,3-BDO pathway comprises 7E, 7F, 4H and 4J. In other embodiments, the 1,3-BDO pathway comprises 7E, 7F, 4H, 4I and 4G. In certain embodiments, the 1,3-BDO pathway comprises 7E, 7F, 4H, 4M, 4N and 4G. In another embodiment, the 1,3-BDO pathway comprises 7E, 7F, 4K, 4O, 4N and 4G. In yet another embodiment, the 1,3-BDO pathway comprises 7E, 7F, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

### 4.6 Attenuation of Competing Byproduct Production Pathways

In certain embodiments, carbon flux towards 1,3-BDO formation is improved by deleting or attenuating competing pathways. Typical fermentation products of yeast include ethanol and glycerol. The deletion or attenuation of these byproducts can be accomplished by approaches delineated above.

Additionally, in the 1,3-BDO pathway, some byproducts can be formed because of the non-specific enzymes acting on the pathway intermediates. For example, CoA hydrolases and CoA transferases can act on acetoacetyl-CoA and 3-hydroxybutyryl-CoA to form acetoacetate and 3-hydroxybutyrate respectively. Accordingly, in certain embodiments, deletion or attenuation of pathways acting on 1,3-BDO pathway intermediates within any of the non-naturally occurring eukaryotic organisms provided herein can help to increase production of 1,3-BDO in these organisms.

The conversion of 3-hydroxybutyryl-CoA to 3-hydroxybutyrate can be catalyzed by an enzyme with 3-hydroxybutyratyl-CoA transferase or hydrolase activity. Similarly, the conversion of acetoacetyl-CoA to acetoacetate can be catalyzed by an enzyme with acetoacetyl-CoA transferase or hydrolase activity. These side reactions that divert 1,3-BDO pathway intermediates from 1,3-BDO production can be prevented by deletion or attenuation of enzymes with these activities. Exemplary CoA hydrolases and CoA transferases are shown in the table below.

TABLE 8

| Protein | GenBank ID | GI number | Organism |
|---|---|---|---|
| Tes1 | NP_012553.1 | 6322480 | Saccharomyces |
| | | | cerevisiae s288c |
| ACH1 | NP_009538.1 | 6319456 | Saccharomyces |
| | | | cerevisiae s288c |
| YALI0F14729p | XP_505426.1 | 50556036 | Yarrowia lipolytica |
| YALI0E30965p | XP_504613.1 | 50554409 | Yarrowia lipolytica |
| KLLA0E16523g | XP_454694.1 | 50309373 | Kluyveromyces |
| | | | lactis |
| KLLA0E10561g | XP_454427.1 | 50308845 | Kluyveromyces |
| | | | lactis |
| ACH1 | P83773.2 | 229462795 | Candida albicans |
| CaO19.10681 | XP_714720.1 | 68482646 | Candida albicans |
| ANI_1_318184 | XP_001401512.1 | 145256774 | Aspergillus niger |
| ANI_1_1594124 | XP_001401252.2 | 317035188 | Aspergillus niger |
| tesB | NP_414986.1 | 16128437 | Escherichia coli |

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an acetoacetyl-CoA hydrolase or transferase; (ii) expresses an attenuated acetoacetyl-CoA hydrolase or transferase; and/or (iii) has lower or no acetoacetyl-CoA hydrolase or transferase enzymatic activity as compared to a wild-type version of the eukaryotic organism. In one embodiment, the organism (i) comprises a disruption in an endogenous and/or exogenous nucleic acid encoding an acetoacetyl-CoA hydrolase or transferase; and (ii) expresses an attenuated acetoacetyl-CoA hydrolase or transferase. In another embodiment, the organism i) comprises a disruption in an endogenous and/or exogenous nucleic acid encoding an acetoacetyl-CoA hydrolase or transferase; and (iii) has lower or no acetoacetyl-CoA hydrolase or transferase enzymatic activity as compared to a wild-type version of the eukaryotic organism. In another embodiment, the organism (ii) expresses an attenuated acetoacetyl-CoA hydrolase or transferase; and (iii) has lower or no acetoacetyl-CoA hydrolase or transferase enzymatic activity as compared to a wild-type version of the eukaryotic organism. In yet another embodiment, the organism i) comprises a disruption in an endogenous and/or exogenous nucleic acid encoding an acetoacetyl-CoA hydrolase or transferase; (ii) expresses an attenuated acetoacetyl-CoA hydrolase or transferase; and (iii) has lower or no acetoacetyl-CoA hydrolase or transferase enzymatic activity as compared to a wild-type version of the eukaryotic organism.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 3-hydroxybutyryl-CoA hydrolase or transferase; (ii) expresses an attenuated 3-hydroxybutyryl-CoA hydrolase or transferase; and/or (iii) has lower or no 3-hydroxybutyryl-CoA hydrolase or transferase enzymatic activity as compared to a wild-type version of the eukaryotic organism. In one embodiment, the organism (i) comprises a disruption in an endogenous and/or exogenous nucleic acid encoding a 3-hydroxybutyryl-CoA hydrolase or transferase; and (ii) expresses an attenuated 3-hydroxybutyryl-CoA hydrolase or transferase. In another embodiment, the organism (i) comprises a disruption in an endogenous and/or exogenous nucleic acid encoding an 3-hydroxybutyryl-CoA hydrolase or transferase; and (iii) has lower or no 3-hydroxybutyryl-CoA hydrolase or transferase enzymatic activity as compared to a wild-type version of the eukaryotic organism. In another embodiment, the organism (ii) expresses an attenuated 3-hydroxybutyryl-CoA hydrolase or transferase; and (iii) has lower or no 3-hydroxybutyryl-CoA hydrolase or transferase enzymatic activity as compared to a wild-type version of the eukaryotic organism. In yet another embodiment, the organism (i) comprises a disruption in an endogenous and/or exogenous nucleic acid encoding a 3-hydroxybutyryl-CoA hydrolase or transferase; (ii) expresses an attenuated 3-hydroxybutyryl-CoA hydrolase or transferase; and (iii) has lower or no 3-hydroxybutyryl-CoA hydrolase or transferase enzymatic activity as compared to a wild-type version of the eukaryotic organism.

Non-specific native aldehyde dehydrogenases are another example of enzymes that acts on 1,3-BDO pathway intermediates. Such enzymes can, for example, convert acetyl-CoA into acetaldehyde or 3-hydroxybutyraldehyde to 3-hydroxybutyrate or 3-oxobutyraldehyde to acetoacetate. Acylating acetaldehyde dehydrogenase enzymes are described in Example II. Several Saccharomyces cerevisiae enzymes catalyze the oxidation of aldehydes to acids including ALD1 (ALD6), ALD2 and ALD3 (Navarro-Avino et al, Yeast 15:829-42 (1999); Quash et al, Biochem Pharmacol 64:1279-92 (2002)). The mitochondrial proteins ALD4 and ALD5 catalyze similar transformations (Wang et al, J Bacteriol 180:822-30 (1998); Boubekeur et al, Eur J Biochem 268:5057-65 (2001)). Aldehyde dehydrogenase enzymes in E. coli that catalyze the conversion of acetaldehyde to acetate include YdcW, BetB, FeaB and AldA (Gruez et al, J Mol Biol 343:29-41 (2004); Yilmaz et al, Biotechnol Prog 18:1176-82 (2002); Rodriguez-Zavala et al, Protein Sci 15:1387-96 (2006)). Acid-forming aldehyde dehydrogenase enzymes are listed in the table below.

TABLE 9

| Protein | GenBank ID | GI number | Organism |
|---|---|---|---|
| ALD2 | NP_013893.1 | 6323822 | Saccharomyces |
| | | | cerevisiae s288c |
| ALD3 | NP_013892.1 | 6323821 | Saccharomyces |
| | | | cerevisiae s288c |
| ALD4 | NP_015019.1 | 6324950 | Saccharomyces |
| | | | cerevisiae s288c |
| ALD5 | NP_010996.2 | 330443526 | Saccharomyces |
| | | | cerevisiae s288c |
| ALD6 | NP_015264.1 | 6325196 | Saccharomyces |
| | | | cerevisiae s288c |
| HFD1 | NP_013828.1 | 6323757 | Saccharomyces |
| | | | cerevisiae s288c |
| CaO19.8361 | XP_710976.1 | 68490403 | Candida albicans |
| CaO19.742 | XP_710989.1 | 68490378 | Candida albicans |
| YALI0C03025 | CAG81682.1 | 49647250 | Yarrowia lipolytica |
| ANI_1_1334164 | XP_001398871.1 | 145255133 | Aspergillus niger |
| ANI_1_2234074 | XP_001392964.2 | 317031176 | Aspergillus niger |
| ANI_1_226174 | XP_001402476.1 | 145256256 | Aspergillus niger |
| ALDH | P41751.1 | 1169291 | Aspergillus niger |
| KLLA0D09999 | CAH00602.7 | 49642640 | Kluyveromyces |
| | | | lactis |
| ydcW | NP_415961.1 | 16129403 | Escherichia coli |
| betB | NP_414846.1 | 16128297 | Escherichia coli |
| feaB | AAC74467.2 | 87081896 | Escherichia coli |
| aldA | NP_415933.1 | 16129376 | Escherichia coli |

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an acetaldehyde dehydrogenase (acylating); (ii) expresses an attenuated acetaldehyde dehydrogenase (acylating); and/or (iii) has lower or no acetaldehyde dehydrogenase (acylating) enzymatic activity as compared to a wild-type version of the eukaryotic organism. In one embodiment, the organism (i) comprises a disruption in an endogenous and/or exogenous nucleic acid encoding an acetaldehyde dehydrogenase (acylating); and (ii) expresses an attenuated acetaldehyde dehydrogenase (acylating). In another embodiment the organism (i) comprises a disruption in an endogenous and/or exogenous nucleic acid encoding an acetaldehyde dehydrogenase (acylating); and (iii) has lower or no acetaldehyde dehydrogenase (acylating) enzymatic activity as compared to a wild-type version of the eukaryotic organism. In another embodiment the organism (ii) expresses an attenuated acetaldehyde dehydrogenase (acylating); and (iii) has lower or no acetaldehyde dehydrogenase (acylating) enzymatic activity as compared to a wild-type version of the eukaryotic organism. In yet another embodiment the organism (i) comprises a disruption in an endogenous and/or exogenous nucleic acid encoding an acetaldehyde dehydrogenase (acylating); (ii) expresses an attenuated acetaldehyde dehydrogenase (acylating); and (iii) has lower or no acetaldehyde dehydrogenase (acylating) enzymatic activity as compared to a wild-type version of the eukaryotic organism.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 3-hydroxybutyraldehyde dehydrogenase; (ii) expresses an attenuated 3-hydroxybutyraldehyde dehydrogenase; and/or (iii) has lower or no 3-hydroxybutyraldehyde dehydrogenase enzymatic activity as compared to a wild-type version of the eukaryotic organism. In one embodiment, the organism (i) comprises a disruption in an endogenous and/or exogenous nucleic acid encoding an 3-hydroxybutyraldehyde dehydrogenase; and (ii) expresses an attenuated 3-hydroxybutyraldehyde dehydrogenase. In another embodiment the organism (i) comprises a disruption in an endogenous and/or exogenous nucleic acid encoding a 3-hydroxybutyraldehyde dehydrogenase; and (iii) has lower or no 3-hydroxybutyraldehyde dehydrogenase enzymatic activity as compared to a wild-type version of the eukaryotic organism. In another embodiment the organism (ii) expresses an attenuated 3-hydroxybutyraldehyde dehydrogenase; and (iii) has lower or no 3-hydroxybutyraldehyde dehydrogenase enzymatic activity as compared to a wild-type version of the eukaryotic organism. In yet another embodiment the organism (i) comprises a disruption in an endogenous and/or exogenous nucleic acid encoding a 3-hydroxybutyraldehyde dehydrogenase; (ii) expresses an attenuated 3-hydroxybutyraldehyde dehydrogenase; and (iii) has lower or no 3-hydroxybutyraldehyde dehydrogenase enzymatic activity as compared to a wild-type version of the eukaryotic organism.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding a 3-oxobutyraldehyde dehydrogenase; (ii) expresses an attenuated 3-oxobutyraldehyde dehydrogenase; and/or (iii) has lower or no 3-oxobutyraldehyde dehydrogenase enzymatic activity as compared to a wild-type version of the eukaryotic organism. In one embodiment, the organism (i) comprises a disruption in an endogenous and/or exogenous nucleic acid encoding an 3-oxobutyraldehyde dehydrogenase; and (ii) expresses an attenuated 3-oxobutyraldehyde dehydrogenase. In another embodiment the organism (i) comprises a disruption in an endogenous and/or exogenous nucleic acid encoding a 3-oxobutyraldehyde dehydrogenase; and (iii) has lower or no 3-oxobutyraldehyde dehydrogenase enzymatic activity as compared to a wild-type version of the eukaryotic organism. In another embodiment the organism (ii) expresses an attenuated 3-oxobutyraldehyde dehydrogenase; and (iii) has lower or no 3-oxobutyraldehyde dehydrogenase enzymatic activity as compared to a wild-type version of the eukaryotic organism. In yet another embodiment the organism (i) comprises a disruption in an endogenous and/or exogenous nucleic acid encoding an 3-oxobutyraldehyde dehydrogenase; (ii) expresses an attenuated 3-oxobutyraldehyde dehydrogenase; and (iii) has lower or no 3-oxobutyraldehyde dehydrogenase enzymatic activity as compared to a wild-type version of the eukaryotic organism.

Other enzymes that act on 1,3-BDO pathway intermediates include ethanol dehydrogenases that convert acetaldehyde into ethanol, as discussed above and 1,3-butanediol into 3-oxobutanol. A number of organisms encode genes that catalyze the interconversion of 3-oxobutanol and 1,3-butanediol, including those belonging to the genus Bacillus, Brevibacterium, Candida, and Klebsiella, as described by Matsuyama et al. J Mol Cat B Enz, 11:513-521 (2001). One of these enzymes, SADH from Candida parapsilosis, was cloned and characterized in E. coli. A mutated Rhodococcus phenylacetaldehyde reductase (Sar268) and a Leifonia alcohol dehydrogenase have also been shown to catalyze this transformation (Itoh et al., Appl. Microbiol Biotechnol. 75:1249-1256 (2007)). These enzymes and those previously described for conversion of acetaldehyde to ethanol are suitable candidates for deletion and/or attenuation. Gene candidates are listed above.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an ethanol dehydrogenase; (ii) expresses an attenuated ethanol dehydrogenase; and/or (iii) has lower or no ethanol dehydrogenase enzymatic activity as compared to a wild-type version of the eukaryotic organism. In one embodiment, the organism (i) comprises a disruption in an endogenous and/or exogenous nucleic acid encoding an ethanol dehydrogenase; and (ii) expresses an attenuated ethanol dehydrogenase. In another embodiment, the organism (i) comprises a disruption in an endogenous and/or exogenous nucleic acid encoding an ethanol dehydrogenase; and (iii) has lower or no ethanol dehydrogenase enzymatic activity as compared to a wild-type version of the eukaryotic organism. In another embodiment, the organism (ii) expresses an attenuated ethanol dehydrogenase; and (iiii) has lower or no ethanol dehydrogenase enzymatic activity as compared to a wild-type version of the eukaryotic organism. In yet another embodiment, the organism (i) comprises a disruption in an endogenous and/or exogenous nucleic acid encoding an ethanol dehydrogenase; (ii) expresses an attenuated ethanol dehydrogenase; and (iii) has lower or no ethanol dehydrogenase enzymatic activity as compared to a wild-type version of the eukaryotic organism. In some embodiments, one or more other alcohol deydrogenases are used in place of the ethanol dehydrogenase.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an 1,3-butanediol dehydrogenase; (ii) expresses an attenuated 1,3-butanediol dehydrogenase; and/or (iii) has lower or no 1,3-butanediol dehydrogenase enzymatic activity as compared to a wild-type version of the eukaryotic organism. In one embodiment, the organism (i) comprises a disruption in an endogenous and/or exogenous nucleic acid encoding an 1,3-butanediol dehydrogenase; and (ii) expresses an attenuated 1,3-butanediol dehydrogenase. In another embodiment, the organism (i) comprises a disruption in an endogenous and/or exogenous nucleic acid encoding an 1,3-butanediol dehydrogenase; and (iii) has lower or no 1,3-butanediol dehydrogenase enzymatic activity as compared to a wild-type version of the eukaryotic organism. In another embodiment, the organism (ii) expresses an attenuated 1,3-butanediol dehydrogenase; and (iiii) has lower or no 1,3-butanediol dehydrogenase enzymatic activity as compared to a wild-type version of the eukaryotic organism. In yet another embodiment, the organism (i) comprises a disruption in an endogenous and/or exogenous nucleic acid encoding an 1,3-butanediol dehydrogenase; (ii) expresses an attenuated 1,3-butanediol dehydrogenase; and (iii) has lower or no 1,3-butanediol dehydrogenase enzymatic activity as compared to a wild-type version of the eukaryotic organism.

In an organism expressing a 1,3-BDO pathway comprising an acetyl-CoA carboxylase and acetoacetyl-CoA synthase (7E/7F), in some embodiments, it may be advantageous to delete or attenuate endogenous acetoacetyl-CoA thiolase activity. Acetoacetyl-CoA thiolase enzymes are typically reversible, whereas acetoacetyl-CoA synthase catalyzes an irreversible reaction. Deletion of acetoacetyl-CoA thiolase would therefore reduce backflux of acetoacetyl-CoA to acetyl-CoA and thereby improve flux toward the 1,3-BDO product.

In another aspect, provided herein is a non-naturally eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and wherein the organism: (i) comprises a disruption in an endogenous and/or exogenous nucleic acid encoding an acetoacetyl-CoA thiolase; (ii) expresses an attenuated acetoacetyl-CoA thiolase; and/or (iii) has lower or no acetoacetyl-CoA thiolase enzymatic activity as compared to a wild-type version of the eukaryotic organism. In one embodiment, the organism (i) comprises a disruption in an endogenous and/or exogenous nucleic acid encoding an acetoacetyl-CoA thiolase; and (ii) expresses an attenuated 1 acetoacetyl-CoA thiolase. In another embodiment, the organism (i) comprises a disruption in an endogenous and/or exogenous nucleic acid encoding an acetoacetyl-CoA thiolase; and (iii) has lower or no acetoacetyl-CoA thiolase enzymatic activity as compared to a wild-type version of the eukaryotic organism. In another embodiment, the organism (ii) expresses an attenuated acetoacetyl-CoA thiolase; and (iiii) has lower or no acetoacetyl-CoA thiolase enzymatic activity as compared to a wild-type version of the eukaryotic organism. In yet another embodiment, the organism (i) comprises a disruption in an endogenous and/or exogenous nucleic acid encoding an acetoacetyl-CoA thiolase; (ii) expresses an attenuated acetoacetyl-CoA thiolase; and (iii) has lower or no acetoacetyl-CoA thiolase enzymatic activity as compared to a wild-type version of the eukaryotic organism.

In one embodiment of the eukaryotic organisms provided above, the 1,3-BDO pathway comprises 4A, 4E, 4F and 4G. In another embodiment, the 1,3-BDO pathway comprises 4A, 4B and 4D. In other embodiments, the 1,3-BDO pathway comprises 4A, 4E, 4C and 4D. In some embodiments, the 1,3-BDO pathway comprises 4A, 4H and 4J. In other embodiments, the 1,3-BDO pathway comprises 4A, 4H, 4I and 4G. In certain embodiments, the 1,3-BDO pathway comprises 4A, 4H, 4M, 4N and 4G. In another embodiment, the 1,3-BDO pathway comprises 4A, 4K, 4O, 4N and 4G. In yet another embodiment, the 1,3-BDO pathway comprises 4A, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

In another embodiment of the eukaryotic organisms provided above, the 1,3-BDO pathway comprises 7E, 7F, 4E, 4F and 4G. In another embodiment, the 1,3-BDO pathway comprises 7E, 7F, 4B and 4D. In other embodiments, the 1,3-BDO pathway comprises 7E, 7F, 4E, 4C and 4D. In some embodiments, the 1,3-BDO pathway comprises 7E, 7F, 4H and 4J. In other embodiments, the 1,3-BDO pathway comprises 7E, 7F, 4H, 4I and 4G. In certain embodiments, the 1,3-BDO pathway comprises 7E, 7F, 4H, 4M, 4N and 4G. In another embodiment, the 1,3-BDO pathway comprises 7E, 7F, 4K, 4O, 4N and 4G. In yet another embodiment, the 1,3-BDO pathway comprises 7E, 7F, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

### 4.7 1,3-BDO Exportation

In certain embodiments, 1,3-butanediol exits a production organism provided herein in order to be recovered and/or dehydrated to butadiene. Examples of genes encoding enzymes that can facilitate the transport of 1,3-butanediol include glycerol facilitator protein homologs are provided in Example XI.

In one aspect, provided herein is a non-naturally occurring eukaryotic organism comprising a 1,3-BDO pathway, wherein said organism comprises at least one endogenous and/or exogenous nucleic acid encoding a 1,3-BDO pathway enzyme expressed in a sufficient amount to produce 1,3-BDO; and wherein said organism further comprises an endogenous and/or exogenous nucleic acid encoding a 1,3-BDO transporter, wherein the nucleic acid encoding the 1,3-BDO transporter is expressed in a sufficient amount for the exportation of 1,3-BDO from the eukaryotic organism.

In one embodiment of the eukaryotic organisms provided above, the 1,3-BDO pathway comprises 4A, 4E, 4F and 4G. In another embodiment, the 1,3-BDO pathway comprises 4A, 4B and 4D. In other embodiments, the 1,3-BDO pathway comprises 4A, 4E, 4C and 4D. In some embodiments, the 1,3-BDO pathway comprises 4A, 4H and 4J. In other embodiments, the 1,3-BDO pathway comprises 4A, 4H, 4I and 4G. In certain embodiments, the 1,3-BDO pathway comprises 4A, 4H, 4M, 4N and 4G. In another embodiment, the 1,3-BDO pathway comprises 4A, 4K, 4O, 4N and 4G. In yet another embodiment, the 1,3-BDO pathway comprises 4A, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

In another embodiment of the eukaryotic organisms provided above, the 1,3-BDO pathway comprises 7E, 7F, 4E, 4F and 4G. In another embodiment, the 1,3-BDO pathway comprises 7E, 7F, 4B and 4D. In other embodiments, the 1,3-BDO pathway comprises 7E, 7F, 4E, 4C and 4D. In some embodiments, the 1,3-BDO pathway comprises 7E, 7F, 4H and 4J. In other embodiments, the 1,3-BDO pathway comprises 7E, 7F, 4H, 4I and 4G. In certain embodiments, the 1,3-BDO pathway comprises 7E, 7F, 4H, 4M, 4N and 4G. In another embodiment, the 1,3-BDO pathway comprises 7E, 7F, 4K, 4O, 4N and 4G. In yet another embodiment, the 1,3-BDO pathway comprises 7E, 7F, 4K, 4L, 4F and 4G. In another embodiment, the eukaryotic organism further comprises an acetyl-CoA pathway selected from the group consisting of: (i) 2A, 2B and 2D; (ii) 2A, 2C and 2D; (iii) 2A, 2B, 2E and 2F; (iv) 2A, 2C, 2E and 2F; (v) 2A, 2B, 2E, 2K, and 2L; (vi.) 2A, 2C, 2E, 2K and 2L; (vii) 5A and 5B; (viii) 5A, 5C and 5D; (ix) 5E, 5F, 5C and 5D; (x) 5G and 5D; (xi) 6A, 6D and 6C; (xii) 6B, 6E and 6C; (xiii) 10A, 10B and 10C; (xiv) 10N, 10H, 10B and 10C; (xv) 10N, 10L, 10M, 10B and 10C; (xvi) 10A, 10B, 10G and 10D; (xvii) 10N, 10H, 10B, 10G and 10D; (xviii) 10N, 10L, 10M, 10B, 10G and 10D; (xix) 10A, 10B, 10J, 10K and 10D; (xx) 10N, 10H, 10B, 10J, 10K and 10D; (xxi) 10N, 10L, 10M, 10B, 10J, 10K and 10D; (xxii) 10A, 10F and 10D; (xxiii) 10N, 10H, 10F and 10D; and (xxiv) 10N, 10L, 10M, 10F and 10D.

### 4.8 Mitochondrial Production of 1,3-BDO

In some embodiments, a eukaryotic organism provided herein is engineered to efficiently direct carbon and reducing equivalents into a mitochondrial 1,3-BDO production pathway. One advantage of producing 1,3-BDO in the mitochondria is the naturally abundant mitochondrial pool of acetyl-CoA, the key 1,3-BDO pathway precursor. Efficient conversion of acetyl-CoA to 1,3-BDO in the mitochondria requires expressing 1,3-BDO pathway enzymes in the mitochondria. It also requires an excess of reducing equivalents to drive the pathway forward. Exemplary methods for increasing the amount of reduced NAD(P)H in the mitochondria are similar to those employed in the cytosol and are described in further detail below. To further increase the availability of the acetyl-CoA precursor, pathways that consume acetyl-CoA in the mitochondria and cytosol can be attenuated as needed. If the 1,3-BDO product is not exported out of the mitochondria by native enzymes or by diffusion, expression of a heterologous 1,3-BDO transporter, such as the glycerol facilitator, can also improve 1,3-BDO production.

In some embodiments, targeting genes to the mitochondria is be accomplished by adding a mitochondrial targeting sequence to 1,3-BDO pathway enzymes. Mitochondrial targeting sequences are well known in the art. For example, fusion of the mitochondrial targeting signal peptide from the yeast COX4 gene to valencene production pathway enzymes resulted in a mitochondrial valencene production pathway that yielded increased titers relative to the same pathway expressed in the cytosol (Farhi et al, Met Eng 13:474-81 (2011)). In one embodiment, the eukaryotic organism comprises a 1,3-BDO pathway, wherein said organism consists of 1,3-BDO pathway enzymes that are localized in the mitochondria of the eukaryotic organism.

In other embodiments, levels of metabolic cofactors in the mitochondria are manipulated to increase flux through the 1,3-BDO pathway, which can further improve mitochondrial production of 1,3-BDO. For example, increasing the availability of reduced NAD(P)H can help to drive the 1,3-BDO pathway forward. This can be accomplished, for example, by increasing the supply of NAD(P)H in the mitochondria and/or attenuating NAD(P)H sinks.

In eukaryotic cells, a significant portion of the cellular NAD pool is contained in the mitochondria (Di Lisa et al, FEBS Lett 492:4-8 (2001)). Increasing the supply of mitochondrial NAD(P)H can be accomplished in different ways. Pyrimidine nucleotides are synthesized in the cytosol and must be transported to the mitochondria in the form of NAD⁺ by carrier proteins. The NAD carrier proteins of Saccharomyces cerevisiae are encoded by NDT1 (GI: 6322185) and NDT2 (GI: 6320831) (Todisco et al, J Biol Chem 281:1524-31 (2006)). Reduced cofactors such as NAD(P)H are not transported across the inner mitochondrial membrane (von Jagow et al, Eur J Biochem 12:583-92 (1970); Lee et al, J Membr Biol 161:173-181 (1998)). NADH in the mitochondria is normally generated by the TCA cycle and the pyruvate dehydrogenase complex. NADPH is generated by the TCA cycle, and can also be generated from NADH if the organism expresses an endogenous or exogenous mitochondrial NADH transhydrogenase. NADH transhydrogenase enzyme candidates are described below.

TABLE 10

| Protein | GenBank ID | GI number | Organism |
|---|---|---|---|
| NDT1 | NP_012260.1 | 6322185 | Saccharomyces |
| | | | cerevisiae |
| ANI_1_1592184 | XP_001401484.2 | 317038471 | Aspergillus niger |
| CaJ7_0216 | XP_888808.1 | 77022728 | Candida albicans |
| YALI0E16478g | XP_504023.1 | 50553226 | Yarrowia lipolytica |
| KLLA0D14036g | XP_453688.1 | 50307419 | Kluyveromyces |
| | | | lactis |

Increasing the redox potential (NAD(P)H/NAD(P) ratio) of the mitochondria can be utilized to drive the 1,3-BDO pathway in the forward direction. Attenuation of mitochondrial redox sinks will increase the redox potential and hence the reducing equivalents available for 1,3-BDO. Exemplary NAD(P)H consuming enzymes or pathways for attenuation include the TCA cycle, NADH dehydrogenases or oxidases, alcohol dehydrogenases and aldehyde dehydrogenases.

The non-naturally occurring eukaryotic organisms provided herein can, in certain embodiments, be produced by introducing expressible nucleic acids encoding one or more of the enzymes or proteins participating in one or more 1,3-BDO or acetyl-CoA pathways. In some embodiments, the non-naturally occurring eukaryotic organisms provided herein can be produced by introducing expressible nucleic acids encoding one or more of the enzymes or proteins participating in one or more acetyl-CoA pathways and one or more 1,3-BDO pathways. Depending on the host eukaryotic organism chosen, nucleic acids for some or all of a particular acetyl-CoA pathway and/or 1,3-BDO can be expressed. In some embodiments, nucleic acids for some or all of a particular acetyl-CoA pathway are expressed. In other embodiments, the eukaryotic organism further comprises nucleic acids expressing some or all of a particular 1,3-BDO pathway. For example, if a chosen host is deficient in one or more enzymes or proteins for a desired pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve cytosolic acetyl-CoA production, or acetyl-CoA production in combination with 1,3-BDO production. Thus, in certain embodiments, a non-naturally occurring eukaryotic organism provided herein can be produced by introducing exogenous enzyme or protein activities to obtain a desired acetyl-CoA pathway and/or 1,3-BDO pathway. Alternatively, a desired acetyl-CoA pathway can be obtained by introducing one or more exogenous enzyme or protein activities that, together with one or more endogenous enzymes or proteins, allows for the transport of acetyl-CoA from a mitochondrion of the organism to the cytosol of the organism, production of cytosolic acetyl-CoA. In other embodiments, the organism further comprises a 1,3-BDO pathway that can be obtained by introducing one or more exogenous enzyme or protein activities that, together with one or more endogenous enzymes or proteins, allows for the production of 1,3-BDO in the organism.

Further genetic modifications described herein to facilitate and/or optimize 1,3-BDO production, for example, manipulation of particular endogenous nucleic acids of interest in the host cell to attenuate or delete competing byproduct pathways and enzymes, can be performed by any method known to those skilled in the art and as provided, for instance, in Example X.

Host eukaryotic organisms can be selected from, and the non-naturally occurring eukaryotic organisms generated in, for example, yeast, fungus or any of a variety of other eukaryotic applicable to fermentation processes. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. It is understood that any suitable eukaryotic host organism can be used to introduce metabolic and/or genetic modifications to produce a desired product. In certain embodiments, the eukaryotic organism is a yeast, such as Saccharomyces cerevisiae. In some embodiments, the eukaryotic organism is a fungus.

Organisms and methods described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

As disclosed herein, intermediates en route to 1,3-BDO can be carboxylic acids or CoA esters thereof, such as 4-hydroxy butyrate, 3-hydroxybutyrate, their CoA esters, as well as crotonyl-CoA. Any carboxylic acid intermediate can occur in various ionized forms, including fully protonated, partially protonated, and fully deprotonated forms. Accordingly, the suffix “-ate,” or the acid form, can be used interchangeably to describe both the free acid form as well as any deprotonated form, in particular since the ionized form is known to depend on the pH in which the compound is found. It is understood that carboxylate intermediates includes ester forms of carboxylate products or pathway intermediates, such as O-carboxylate and S-carboxylate esters. O- and S-carboxylates can include lower alkyl, that is C1 to C6, branched or straight chain carboxylates. Some such O- or S-carboxylates include, without limitation, methyl, ethyl, n-propyl, n-butyl, i-propyl, sec-butyl, and tert-butyl, pentyl, hexyl O- or S-carboxylates, any of which can further possess an unsaturation, providing for example, propenyl, butenyl, pentyl, and hexenyl O- or S-carboxylates. O-carboxylates can be the product of a biosynthetic pathway. Exemplary O-carboxylates accessed via biosynthetic pathways can include, without limitation, methyl 4-hydroxybutyrate, methyl-3-hydroxybutyrate, ethyl 4-hydroxybutyrate, ethyl 3-hydroxybutyrate, n-propyl 4-hydroxybutyrate, and n-propyl 3-hydroxybutyrate. Other biosynthetically accessible O-carboxylates can include medium to long chain groups, that is C7-C22, O-carboxylate esters derived from fatty alcohols, such heptyl, octyl, nonyl, decyl, undecyl, lauryl, tridecyl, myristyl, pentadecyl, cetyl, palmitolyl, heptadecyl, stearyl, nonadecyl, arachidyl, heneicosyl, and behenyl alcohols, any one of which can be optionally branched and/or contain unsaturations. O-carboxylate esters can also be accessed via a biochemical or chemical process, such as esterification of a free carboxylic acid product or transesterification of an O- or S-carboxylate. S-carboxylates are exemplified by CoA S-esters, cysteinyl S-esters, alkylthioesters, and various aryl and heteroaryl thioesters.

Depending on the 1,3-BDO biosynthetic pathway constituents of a selected host eukaryotic organism comprising an 1,3-BDO pathway, the non-naturally occurring organisms provided herein comprising a 1,3-BDO pathway can include at least one exogenously expressed 1,3-BDO pathway-encoding nucleic acid and up to all encoding nucleic acids for one or more 1,3-BDO biosynthetic pathways. For example, 1,3-BDO biosynthesis can be established in a host deficient in a pathway enzyme or protein through exogenous expression of the corresponding encoding nucleic acid. In a host deficient in all enzymes or proteins of a 1,3-BDO pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of 1,3-BDO can be included.

In addition, depending on the acetyl-CoA pathway constituents of a selected host eukaryotic organism, the non-naturally occurring eukaryotic organisms provided herein can include at least one exogenously expressed acetyl-CoA pathway-encoding nucleic acid and up to all encoding nucleic acids for one or more acetyl-CoA pathways. For example, mitochondrial and/or peroxisomal acetyl-CoA exportation into the cytosol of a host and/or increase in cytosolic acetyl-CoA in the host can be established in a host deficient in a pathway enzyme or protein through exogenous expression of the corresponding encoding nucleic acid. In a host deficient in all enzymes or proteins of an acetyl-CoA pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of cytosolic acetyl-CoA can be included, such as a citrate synthase, a citrate transporter, a citrate/oxaloacetate transporter, a citrate/malate transporter, an ATP citrate lyase, a citrate lyase, an acetyl-CoA synthetase, an acetate kinase and phosphotransacetylase, an oxaloacetate transporter, a cytosolic malate dehydrogenase, a malate transporter a mitochondrial malate dehydrogenase; a pyruvate oxidase (acetate forming); an acetyl-CoA ligase or transferase; an acetate kinase; a phosphotransacetylase; a pyruvate decarboxylase; an acetaldehyde dehydrogenase; a pyruvate oxidase (acetyl-phosphate forming); a pyruvate dehydrogenase, a pyruvate:ferredoxin oxidoreductase or pyruvate formate lyase; a acetaldehyde dehydrogenase (acylating); a threonine aldolase; a mitochondrial acetylcarnitine transferase; a peroxisomal acetylcarnitine transferase; a cytosolic acetylcarnitine transferase; a mitochondrial acetylcarnitine translocase; a peroxisomal acetylcarnitine translocase; a PEP carboxylase; a PEP carboxykinase; an oxaloacetate decarboxylase; a malonate semialdehyde dehydrogenase (acetylating); an acetyl-CoA carboxylase; a malonyl-CoA decarboxylase; an oxaloacetate dehydrogenase; an oxaloacetate oxidoreductase; a malonyl-CoA reductase; a pyruvate carboxylase; a malonate semialdehyde dehydrogenase; a malonyl-CoA synthetase; a malonyl-CoA transferase; a malic enzyme; a malate dehydrogenase; a malate oxidoreductase; a pyruvate kinase; or a PEP phosphatase.

Given the teachings and guidance provided herein, those skilled in the art will understand that the number of encoding nucleic acids to introduce in an expressible form will, at least, parallel the acetyl-CoA pathway deficiencies of the selected host eukaryotic organism. Therefore, a non-naturally occurring eukaryotic organism provided herein can have one, two, three, four, five, six, seven, eight, nine, ten, up to all nucleic acids encoding the enzymes or proteins constituting an acetyl-CoA pathway disclosed herein. In some embodiments, the non-naturally occurring eukaryotic organisms also can include other genetic modifications that facilitate or optimize production of cytosolic acetyl-CoA in the organism or that confer other useful functions onto the host eukaryotic organism. In addition, those skilled in the art will further understand that, in embodiments involving eukaryotic organisms comprising an acetyl-CoA pathway and 1,3-BDO pathway, the number of encoding nucleic acids to introduce in an expressible form will, at least, parallel the 1,3-BDO pathway deficiencies of the selected host eukaryotic organism. Therefore, a non-naturally occurring eukaryotic organism provided herein can have one, two, three, four, five, up to all nucleic acids encoding the enzymes or proteins constituting a 1,3-BDO biosynthetic pathway disclosed herein. In some embodiments, the non-naturally occurring eukaryotic organisms also can include other genetic modifications that facilitate or optimize 1,3-BDO biosynthesis or that confer other useful functions onto the host eukaryotic organism. One such other functionality can include, for example, augmentation of the synthesis of one or more of the 1,3-BDO pathway precursors such as acetyl-CoA.

Generally, a host eukaryotic organism is selected such that it produces the precursor of an acetyl-CoA pathway, either as a naturally produced molecule or as an engineered product that either provides de novo production of a desired precursor or increased production of a precursor naturally produced by the host eukaryotic organism. For example, mitochondrial acetyl-CoA is produced naturally in a host organism such as Saccharomyces cerevisiae. A host organism can be engineered to increase production of a precursor, as disclosed herein. In addition, a eukaryotic organism that has been engineered to produce a desired precursor can be used as a host organism and further engineered to express enzymes or proteins of an acetyl-CoA pathway, and optionally a 1,3-BDO pathway.

In some embodiments, a non-naturally occurring eukaryotic organism provided herein is generated from a host that contains the enzymatic capability to synthesize cytosolic acetyl-CoA. In this specific embodiment it can be useful to increase the synthesis or accumulation of an acetyl-CoA pathway product to, for example, drive acetyl-CoA pathway reactions toward cytosolic acetyl-CoA production. Increased synthesis or accumulation can be accomplished by, for example, overexpression of nucleic acids encoding one or more of the above-described acetyl-CoA pathway enzymes or proteins. Overexpression of the enzyme or enzymes and/or protein or proteins of the acetyl-CoA pathway can occur, for example, through exogenous expression of the endogenous gene or genes, or through exogenous expression of the heterologous gene or genes. Therefore, naturally occurring organisms can be readily generated to be non-naturally occurring eukaryotic organisms as provided herein, for example, producing cytosolic acetyl-CoA, through overexpression of one, two, three, four, five, six, seven, eight, nine or ten, that is, up to all nucleic acids encoding acetyl-CoA pathway enzymes or proteins. In addition, a non-naturally occurring organism can be generated by mutagenesis of an endogenous gene that results in an increase in activity of an enzyme in the acetyl-CoA pathway.

In certain embodiments, wherein the eukaryotic organism comprises an acetyl-CoA pathway and 1,3-BDO pathway, the organism is generated from a host that contains the enzymatic capability to synthesize both acetyl-CoA and 1,3-BDO. In this specific embodiment it can be useful to increase the synthesis or accumulation of a cytosolic acetyl-CoA and/or 1,3-BDO pathway product to, for example, drive 1,3-BDO pathway reactions toward 1,3-BDO production. Increased synthesis or accumulation can be accomplished by, for example, overexpression of nucleic acids encoding one or more of the above-described acetyl-CoA and/or 1,3-BDO pathway enzymes or proteins. Overexpression of the enzyme or enzymes and/or protein or proteins of the acetyl-CoA and/or 1,3-BDO pathways can occur, for example, through exogenous expression of the endogenous gene or genes, or through exogenous expression of the heterologous gene or genes. Therefore, naturally occurring organisms can be readily generated to be non-naturally occurring eukaryotic organisms provided herein, for example, producing 1,3-BDO, through overexpression of one, two, three, four, five, that is, up to all nucleic acids encoding 1,3-BDO biosynthetic pathway enzymes or proteins. In addition, a non-naturally occurring organism can be generated by mutagenesis of an endogenous gene that results in an increase in activity of an enzyme in the acetyl CoA and/or 1,3-BDO biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring eukaryotic organism.

It is understood that, in certain embodiments, any of the one or more exogenous nucleic acids can be introduced into a eukaryotic organism to produce a non-naturally occurring eukaryotic organism provided herein. The nucleic acid(s) can be introduced so as to confer, for example, an acetyl-CoA pathway onto the organism, for example, by expressing a polypeptide(s) having the given activity that is encoded by the nucleic acid(s). The nucleic acids can also be introduced so as to further a 1,3-BDO biosynthetic pathway onto the organism. Alternatively, encoding nucleic acids can be introduced to produce an intermediate organism having the biosynthetic capability to catalyze some of the required reactions to confer acetyl-CoA production or transport, or further 1,3-BDO biosynthetic capability. For example, a non-naturally occurring organism having an acetyl-CoA pathway, either alone or in combination with a 1,3-BDO biosynthetic pathway, can comprise at least two exogenous nucleic acids encoding desired enzymes or proteins. For example, the non-naturally occurring eukaryotic organism can comprise at least two exogenous nucleic acids encoding a pyruvate oxidase (acetate forming) and an acetyl-CoA synthetase (FIG. 5, steps A and B). Thus, it is understood that any combination of two or more enzymes or proteins of a biosynthetic pathway can be included in a non-naturally occurring organism provided herein. Similarly, it is understood that any combination of three or more enzymes or proteins of a biosynthetic pathway can be included in a non-naturally occurring organism of provided herein, and so forth, as desired, so long as the combination of enzymes and/or proteins of the desired biosynthetic pathway results in production of the corresponding desired product. For example, the non-naturally occurring eukaryotic organism can comprise at least three exogenous nucleic acids encoding a pyruvate oxidase (acetate forming), an acetate kinase, and a phosphotransacetylase (FIG. 5, steps A, C and D); or an acetoacetyl-CoA thiolase, an acetoacetyl-CoA reductase (ketone reducing), and a 3-hydroxybutyryl-CoA reductase (alcohol forming) (FIG. 4, steps A, H and J). Similarly, any combination of four or more enzymes or proteins of a biosynthetic pathway as disclosed herein can be included in a non-naturally occurring eukaryotic organism provided herein, as desired, so long as the combination of enzymes and/or proteins of the desired biosynthetic pathway results in production of the corresponding desired product. For example, the non-naturally occurring eukaryotic organism can comprise at least four exogenous nucleic acids encoding citrate synthase, a citrate transporter, a citrate lyase and an acetyl-CoA synthetase (FIG. 2, steps A, B, E and F); or an acetoacetyl-CoA thiolase, an acetoacetyl-CoA reductase (ketone reducing), a 3-hydroxybutyryl-CoA reductase (aldehyde forming), and 3-hydroxybutyraldehyde reductase (FIG. 4, steps A, H, I and G). Other individual pathways depicted in the figures are also contemplated embodiments of the compositions and methods provided herein. Similarly, it is understood that a non-naturally occurring eukaryotic organism can, for example, comprise at least six exogenous nucleic acids, with three exogenous nucleic acids encoding three acetyl-CoA pathway enzymes and three exogenous nucleic acids encoding three 1,3-BDO pathway enzymes. Other numbers and/or combinations of nucleic acids and pathway enzymes are likewise contemplated herein.

In some embodiments, the eukaryotic organism comprises exogenous nucleic acids encoding each of the enzymes of an acetyl Co-A pathway provided herein. In other embodiments, the eukaryotic organism comprises exogenous nucleic acids encoding each of the enzymes of a 1,3-BDO pathway provided herein. In yet other embodiments, the eukaryotic organism comprises exogenous nucleic acids encoding each of the enzymes of an acetyl Co-A pathway provided herein, and the eukaryotic organism further comprises exogenous nucleic acids encoding each of the enzymes of a 1,3-BDO pathway provided herein.

In addition to the biosynthesis of cytosolic acetyl-CoA, either alone or in combination with 1,3-BDO, as described herein, the non-naturally occurring eukaryotic organisms and methods provided herein also can be utilized in various combinations with each other and with other eukaryotic organisms and methods well known in the art to achieve product biosynthesis by other routes. For example, one alternative to produce cytosolic acetyl-CoA other than use of than cytosolic acetyl-CoA producers is through addition of another eukaryotic organism capable of converting an acetyl-CoA pathway intermediate to acetyl-CoA. One such procedure includes, for example, the culturing or fermenting of a eukaryotic organism that produces an acetyl-CoA pathway intermediate. The acetyl-CoA pathway intermediate can then be used as a substrate for a second eukaryotic organism that converts the acetyl-CoA pathway intermediate to cytosolic acetyl-CoA. The acetyl-CoA pathway intermediate can be added directly to another culture of the second organism or the original culture of the acetyl-CoA pathway intermediate producers can be depleted of these eukaryotic organisms by, for example, cell separation, and then subsequent addition of the second organism to the fermentation broth can be utilized to produce the final product without intermediate purification steps.

In other embodiments, wherein the non-naturally occurring eukaryotic organism further comprises a 1,3-BDO pathway, one potential alternative to produce 1,3-BDO other than use of the 1,3-BDO producers is through addition of another eukaryotic organism capable of converting 1,3-BDO pathway intermediate to 1,3-BDO. One such procedure includes, for example, the fermentation of a eukaryotic organism that produces 1,3-BDO pathway intermediate. The 1,3-BDO pathway intermediate can then be used as a substrate for a second eukaryotic organism that converts the 1,3-BDO pathway intermediate to 1,3-BDO. The 1,3-BDO pathway intermediate can be added directly to another culture of the second organism or the original culture of the 1,3-BDO pathway intermediate producers can be depleted of these eukaryotic organisms by, for example, cell separation, and then subsequent addition of the second organism to the fermentation broth can be utilized to produce the final product without intermediate purification steps.

In other embodiments, the non-naturally occurring eukaryotic organisms and methods provided herein can be assembled in a wide variety of subpathways to achieve biosynthesis of, for example, cytosolic acetyl-CoA. In these embodiments, biosynthetic pathways for a desired product can be segregated into different eukaryotic organisms, and the different eukaryotic organisms can be co-cultured to produce the final product. In such a biosynthetic scheme, the product of one eukaryotic organism is the substrate for a second eukaryotic organism until the final product is synthesized. For example, the biosynthesis of cytosolic acetyl-CoA can be accomplished by constructing a eukaryotic organism that contains biosynthetic pathways for conversion of one pathway intermediate to another pathway intermediate or the product. Alternatively, cytosolic acetyl-CoA also can be biosynthetically produced from eukaryotic organisms through co-culture or co-fermentation using two organisms in the same vessel, where the first eukaryotic organism produces a cytosolic acetyl-CoA intermediate and the second eukaryotic organism converts the intermediate to acetyl-CoA.

In certain embodiments, wherein the non-naturally occurring eukaryotic organisms further comprise a 1,3-BDO pathway, the organisms and methods provided herein can be assembled in a wide variety of subpathways to achieve biosynthesis of acetyl-CoA and/or 1,3-BDO. In these embodiments, biosynthetic pathways for a desired product provided herein can be segregated into different eukaryotic organisms, and the different eukaryotic organisms can be co-cultured to produce the final product. In such a biosynthetic scheme, the product of one eukaryotic organism is the substrate for a second eukaryotic organism until the final product is synthesized. For example, the biosynthesis of 1,3-BDO can be accomplished by constructing a eukaryotic organism that contains biosynthetic pathways for conversion of one pathway intermediate to another pathway intermediate or the product. Alternatively, 1,3-BDO also can be biosynthetically produced from eukaryotic organisms through co-culture or co-fermentation using two organisms in the same vessel, where the first eukaryotic organism produces 1,3-BDO intermediate and the second eukaryotic organism converts the intermediate to 1,3-BDO. Certain embodiments include any combination of acetyl-CoA and 1,3-BDO pathway components.

Given the teachings and guidance provided herein, those skilled in the art will understand that a wide variety of combinations and permutations exist for the non-naturally occurring eukaryotic organisms and methods provided herein, together with other eukaryotic organisms, with the co-culture of other non-naturally occurring eukaryotic organisms having subpathways and with combinations of other chemical and/or biochemical procedures well known in the art to produce cytosolic acetyl-CoA, either alone or in combination with a 1,3-BDO.

Sources of encoding nucleic acids for an acetyl-CoA pathway enzyme or protein can include, for example, any species where the encoded gene product is capable of catalyzing the referenced reaction. Similarly, sources of encoding nucleic acids for a 1,3-BDO pathway enzyme or protein or a related protein or enzyme that affects 1,3-BDO production as described herein (e.g., 1,3-BDO byproduct pathway enzymes) can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli Acidaminococcus fermentans, Acinetobacter baylyi, Acinetobacter calcoaceticus, Aquifex aeolicus, Arabidopsis thaliana, Archaeoglobus fulgidus, Aspergillus niger, Aspergillus terreus, Bacillus subtilis, Bos Taurus, Candida albicans, Candida tropicalis, Chlamydomonas reinhardtii, Chlorobium tepidum, Citrobacter koseri, Citrus junos, Clostridium acetobutylicum, Clostridium kluyveri, Clostridium saccharoperbutylacetonicum, Cyanobium PCC7001, Desulfatibacillum alkenivorans, Dictyostelium discoideum, Fusobacterium nucleatum, Haloarcula marismortui, Homo sapiens, Hydrogenobacter thermophilus, Klebsiella pneumoniae, Kluyveromyces lactis, Lactobacillus brevis, Leuconostoc mesenteroides, Metallosphaera sedula, Methanothermobacter thermautotrophicus, Mus musculus, Mycobacterium avium, Mycobacterium bovis, Mycobacterium marinum, Mycobacterium smegmatis, Nicotiana tabacum, Nocardia iowensis, Oryctolagus cuniculus, Penicillium chrysogenum, Pichia pastoris, Porphyromonas gingivalis, Porphyromonas gingivalis, Pseudomonas aeruginos, Pseudomonas putida, Pyrobaculum aerophilum, Ralstonia eutropha, Rattus norvegicus, Rhodobacter sphaeroides, Saccharomyces cerevisiae, Salmonella enteric, Salmonella typhimurium, Schizosaccharomyces pombe, Sulfolobus acidocaldarius, Sulfolobus solfataricus, Sulfolobus tokodaii, Thermoanaerobacter tengcongensis, Thermus thermophilus, Trypanosoma brucei, Tsukamurella paurometabola, Yarrowia lipolytica, Zoogloea ramigera and Zymomonas mobilis, as well as other exemplary species disclosed herein or available as source organisms for corresponding genes. However, with the complete genome sequence available for now more than 550 species (with more than half of these available on public databases such as the NCBI), including 395 eukaryotic organism genomes and a variety of yeast, fungi, plant, and mammalian genomes, the identification of genes encoding the requisite cytosolic acetyl-CoA and/or 1,3-BDO biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of cytosolic acetyl-CoA and/or 1,3-BDO described herein with reference to a particular organism can be readily applied to other eukaryotic organisms alike. Given the teachings and guidance provided herein, those skilled in the art will know that a metabolic alteration exemplified in one organism can be applied equally to other organisms.

In some instances, such as when an alternative cytosolic acetyl-CoA and/or 1,3-BDO biosynthetic pathway exists in an unrelated species, the cytosolic acetyl-CoA and/or 1,3-BDO biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms can differ. However, given the teachings and guidance provided herein, those skilled in the art also will understand that the teachings and methods provided herein can be applied to all eukaryotic organisms using the cognate metabolic alterations to those exemplified herein to construct a eukaryotic organism in a species of interest that will synthesize cytosolic acetyl-CoA, either alone or in combination with 1,3-BDO.

Methods for constructing and testing the expression levels of a non-naturally occurring cytosolic acetyl-CoA producing host can be performed, for example, by recombinant and detection methods well known in the art. Methods for constructing and testing the expression levels of a non-naturally occurring 1,3-BDO-producing host can also be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of cytosolic acetyl-CoA can be introduced stably or transiently into a host cell using techniques well known in the art including, but not limited to, conjugation, electroporation, chemical transformation, transduction, transfection, and ultrasound transformation. In embodiments, wherein the eukaryotic organism further comprises a 1,3-BDO pathway, exogenous nucleic acid sequences involved in a pathway for production of 1,3-BDO can also be introduced stably or transiently into a host cell using these same techniques. For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more cytosolic acetyl-CoA biosynthetic pathway encoding nucleic acids as exemplified herein operably linked to expression control sequences functional in the host organism. An expression vector or vectors can also be constructed to include one or more 1,3-BDO biosynthetic pathway encoding nucleic acids as exemplified herein operably linked to expression control sequences functional in the host organism. Expression vectors applicable for use in the eukaryotic host organisms provided herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments, provided herein is a method for producing cytosolic acetyl-CoA in a non-naturally occurring eukaryotic organism comprising an acetyl-CoA pathway comprising culturing any of the non-naturally occurring eukaryotic organisms comprising an acetyl-CoA pathway described herein under sufficient conditions for a sufficient period of time to produce cytosolic acetyl-CoA. In other embodiments, provided herein is a method for producing 1,3-BDO in a non-naturally occurring eukaryotic organism comprising an acetyl-CoA pathway and a 1,3-BDO pathway, comprising culturing any of the non-naturally occurring eukaryotic organisms comprising an 1,3-BDO pathway described herein under sufficient conditions for a sufficient period of time to produce cytosolic acetyl-CoA and 1,3-BDO.

Suitable purification and/or assays to test for the production of cytosolic acetyl-CoA and/or 1,3-BDO can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art. An increase in the availability of cytosolic acetyl-CoA can be demonstrated by an increased production of a metabolite that is formed form cytosolic acetyl-CoA (e.g., 1-3-butanediol). Alternatively, functional cytosolic acetyl-COA pathways can be screened using an organism (e.g., S. cerevisiae) engineered so that it cannot synthesize sufficient cytosolic acetyl-CoA to support growth on minimal media. See WO/2009/013159. Growth on minimal media is restored by introducing a functional non-native mechanism into the organism for cytosolic acetyl-CoA production.

The cytosolic acetyl-CoA and/or 1,3-BDO can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring eukaryotic organisms described herein can be cultured to produce and/or secrete the biosynthetic products provided herein. For example, the cytosolic acetyl-CoA producers can be cultured for the biosynthetic production of cytosolic acetyl-CoA and or 1,3-BDO.

For the production of cytosolic acetyl-CoA and/or 1,3-BDO, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

In addition to renewable feedstocks such as those exemplified above, the eukaryotic organisms provided herein also can be modified for growth on syngas as its source of carbon. In this specific embodiment, one or more proteins or enzymes are expressed in the eukaryotic organisms to provide a metabolic pathway for utilization of syngas or other gaseous carbon source.

Organisms provided herein can utilize, and the growth medium can include, for example, any carbohydrate source which can supply a source of carbon to the non-naturally occurring eukaryotic organism. Such sources include, for example, sugars such as glucose, xylose, arabinose, galactose, mannose, fructose, sucrose and starch. Other sources of carbohydrate include, for example, renewable feedstocks and biomass. Exemplary types of biomasses that can be used as feedstocks in the methods provided herein include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the eukaryotic organisms provided herein for the production of cytosolic acetyl-CoA and/or 1,3-BDO.

In addition to renewable feedstocks such as those exemplified above, the eukaryotic organisms provided herein also can be modified for growth on syngas as its source of carbon. In this specific embodiment, one or more proteins or enzymes are expressed in the cytosolic acetyl-CoA producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

Accordingly, given the teachings and guidance provided herein, those skilled in the art will understand that a non-naturally occurring eukaryotic organism can be produced that secretes the biosynthesized compounds provided herein when grown on a carbon source such as a carbohydrate. Such compounds include, for example, cytosolic acetyl-CoA and any of the intermediate metabolites in the acetyl-CoA pathway. Such compounds canals include, for example, 1,3-BDO and any of the intermediate metabolites in the 1,3-BDO pathway. All that is required is to engineer in one or more of the required enzyme or protein activities to achieve biosynthesis of the desired compound or intermediate including, for example, inclusion of some or all of the cytosolic acetyl-CoA and/or 1,3-BDO biosynthetic pathways. Accordingly, in some embodiments, provided herein is a non-naturally occurring eukaryotic organism that produces and/or secretes cytosolic acetyl-CoA when grown on a carbohydrate or other carbon source and produces and/or secretes any of the intermediate metabolites shown in the acetyl-CoA pathway when grown on a carbohydrate or other carbon source. The cytosolic acetyl-CoA producing eukaryotic organisms provided herein can initiate synthesis from an intermediate, for example, citrate and acetate. In other embodiments, provided herein is a non-naturally occurring eukaryotic organism that produces and/or secretes 1,3-BDO when grown on a carbohydrate or other carbon source and produces and/or secretes any of the intermediate metabolites shown in the 1,3-BDO pathway when grown on a carbohydrate or other carbon source. The 1,3-BDO producing organism can initiate synthesis of 1,3-BDO from acetyl-CoA, and, as such, a combination of pathways is possible.

The non-naturally occurring eukaryotic organisms provided herein are constructed using methods well known in the art as exemplified herein to exogenously express at least one nucleic acid encoding an acetyl-CoA pathway enzyme or protein in sufficient amounts to produce cytosolic acetyl-CoA. It is understood that the eukaryotic organisms provided herein are cultured under conditions sufficient to produce cytosolic acetyl-CoA. Following the teachings and guidance provided herein, the non-naturally occurring eukaryotic organisms provided herein can achieve biosynthesis of cytosolic acetyl-CoA resulting in intracellular concentrations between about 0.1-200 mM or more. Generally, the intracellular concentration of cytosolic acetyl-CoA is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring organisms provided herein.

In certain embodiments, wherein the non-naturally occurring eukaryotic organism comprises an acetyl-CoA pathway and a 1,3-BDO pathway, the organisms can be constructed using methods well known in the art as exemplified herein to exogenously express at least one nucleic acid encoding an acetyl-CoA pathway and/or 1,3-BDO pathway enzyme or protein in sufficient amounts to produce acetyl-CoA and/or 1,3-BDO. It is understood that the organisms provided herein can be cultured under conditions sufficient to produce cytosolic acetyl-CoA and/or 1,3-BDO. Following the teachings and guidance provided herein, the non-naturally occurring organisms provided herein can achieve biosynthesis of 1,3-BDO resulting in intracellular concentrations between about 0.1-2000 mM or more. Generally, the intracellular concentration of 1,3-BDO is between about 3-1800 mM, particularly between about 5-1700 mM and more particularly between about 8-1600 mM, including about 100 mM, 200 mM, 500 mM, 800 mM, or more. Intracellular concentrations between and above each of these exemplary ranges also can be achieved from the non-naturally occurring organisms provided herein.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring eukaryotic organisms as well as other anaerobic conditions well known in the art. Under such anaerobic or substantially anaerobic conditions, the cytosolic acetyl-CoA producers can synthesize cytosolic acetyl-CoA at intracellular concentrations of 0.005-1000 mM or more as well as all other concentrations exemplified herein. It is understood that, even though the above description refers to intracellular concentrations, cytosolic acetyl-CoA producing eukaryotic organisms can produce cytosolic acetyl-CoA intracellularly and/or secrete the product into the culture medium. In embodiments, wherein the non-naturally occurring eukaryotic organism further comprises a 1,3-BDO pathway, under such anaerobic conditions, the 1,3-BDO producers can synthesize 1,3-BDO at intracellular concentrations of 5-10 mM or more as well as all other concentrations exemplified herein. It is understood that, even though the above description refers to intracellular concentrations, 1,3-BDO producing eukaryotic organisms can produce 1,3-BDO intracellularly and/or secrete the product into the culture medium.

In addition to the culturing and fermentation conditions disclosed herein, growth condition for achieving biosynthesis of cytosolic acetyl-CoA and/or 1,3-BDO can include the addition of an osmoprotectant to the culturing conditions. In certain embodiments, the non-naturally occurring eukaryotic organisms provided herein can be sustained, cultured or fermented as described herein in the presence of an osmoprotectant. Briefly, an osmoprotectant refers to a compound that acts as an osmolyte and helps a eukaryotic organism as described herein survive osmotic stress. Osmoprotectants include, but are not limited to, betaines, amino acids, and the sugar trehalose. Non-limiting examples of such are glycine betaine, praline betaine, dimethylthetin, dimethyl slfonioproprionate, 3-dimethylsulfonio-2-methylproprionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a eukaryotic organism described herein from osmotic stress will depend on the eukaryotic organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

In some embodiments, the carbon feedstock and other cellular uptake sources such as phosphate, ammonia, sulfate, chloride and other halogens can be chosen to alter the isotopic distribution of the atoms present in cytosolic acetyl-CoA or any acetyl-CoA pathway intermediate. The various carbon feedstock and other uptake sources enumerated above will be referred to herein, collectively, as “uptake sources.” Uptake sources can provide isotopic enrichment for any atom present in the product cytosolic acetyl-CoA or acetyl-CoA pathway intermediate including any cytosolic acetyl-CoA impurities generated in diverging away from the pathway at any point. Uptake sources can also provide isotopic enrichment for any atom present in the product 1,3-BDO or 1,3-BDO pathway intermediate including any 1,3-BDO impurities generated by diverging away from the pathway at any point. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, a target isotopic ratio of an uptake source can be obtained via synthetic chemical enrichment of the uptake source. Such isotopically enriched uptake sources can be purchased commercially or prepared in the laboratory. In some embodiments, a target isotopic ratio of an uptake source can be obtained by choice of origin of the uptake source in nature. In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental carbon source, such as CO2, which can possess a larger amount of carbon-14 than its petroleum-derived counterpart.

The unstable carbon isotope carbon-14 or radiocarbon makes up for roughly 1 in 1012 carbon atoms in the earth's atmosphere and has a half-life of about 5700 years. The stock of carbon is replenished in the upper atmosphere by a nuclear reaction involving cosmic rays and ordinary nitrogen (14N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil. Olsson, The use of Oxalic acid as a Standard. in, Radiocarbon Variations and Absolute Chronology, Nobel Symposium, 12th Proc., John Wiley & Sons, New York (1970). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950)¹⁴C/¹²C ratio of 1.176±0.010×10⁻¹² (Karlen et al., Arkiv Geoftsik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mille. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content and/or prepared downstream products that utilize of the invention having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, provided herein is a cytosolic acetyl-CoA or a cytosolic acetyl-CoA intermediate that has a carbon-12, carbon-13, and carbon-14 ratio that reflects an atmospheric carbon uptake source. For example, in some aspects the cytosolic acetyl-CoA or cytosolic acetyl-CoA intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some embodiments, the uptake source is CO2. In some embodiments, the cytosolic acetyl-CoA or cytosolic acetyl-CoA intermediate has a carbon-12, carbon-13, and carbon-14 ratio that reflects petroleum-based carbon uptake source. In some embodiments, the cytosolic acetyl-CoA or cytosolic acetyl-CoA intermediate that has a carbon-12, carbon-13, and carbon-14 ratio that is obtained by a combination of an atmospheric carbon uptake source with a petroleum-based uptake source. In this aspect, the cytosolic acetyl-CoA or cytosolic acetyl-CoA intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, provided herein is a cytosolic acetyl-CoA or cytosolic acetyl-CoA intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

In other embodiments, wherein the eukaryotic organism further comprises a 1,3-BDO pathway, provided herein is a 1,3-BDO or 1,3-BDO intermediate that has a carbon-12, carbon-13, and carbon-14 ratio that reflects an atmospheric carbon uptake source. For example, in some aspects the 1,3-BDO or 1,3-BDO intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some embodiments, the uptake source is CO2. In some embodiments, the 1,3-BDO or 1,3-BDO intermediate has a carbon-12, carbon-13, and carbon-14 ratio that reflects petroleum-based carbon uptake source. In some embodiments, the 1,3-BDO or 1,3-BDO intermediate has a carbon-12, carbon-13, and carbon-14 ratio that is obtained by a combination of an atmospheric carbon uptake source with a petroleum-based uptake source. In this aspect, the 1,3-BDO or 1,3-BDO intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, provided herein is a 1,3-BDO or 1,3-BDO intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present invention relates to the biologically produced 1,3-BDO or 1,3-BDO intermediate as disclosed herein, and to the products derived therefrom, wherein the 1,3-BDO or a 1,3-BDO intermediate has a carbon-12, carbon-13, and carbon-14 isotope ratio of about the same value as the CO₂ that occurs in the environment. For example, in some aspects the invention provides: bioderived 1,3-BDO or a bioderived 1,3-BDO intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived 1,3-BDO or a bioderived 1,3-BDO intermediate as disclosed herein, wherein the bioderived product is chemically modified to generate a final product. Methods of chemically modifying a bioderived product of 1,3-BDO, or an intermediate thereof, to generate a desired product are well known to those skilled in the art, as described herein. The invention further provides organic solvents, polyurethane resins, polyester resins, hypoglycaemic agents, butadiene and/or butadiene-based products having a carbon-12 versus carbon-13 versus carbon-14 isotope ratio of about the same value as the CO₂ that occurs in the environment, wherein the organic solvents, polyurethane resins, polyester resins, hypoglycaemic agents, butadiene and/or butadiene-based products are generated directly from or in combination with bioderived 1,3-BDO or a bioderived 1,3-BDO intermediate as disclosed herein.

1,3-BDO is a chemical commonly used in many commercial and industrial applications, and is also used as a raw material in the production of a wide range of products. Non-limiting examples of such applications and products include organic solvents, polyurethane resins, polyester resins, hypoglycaemic agents, butadiene and/or butadiene-based products organic solvents, polyurethane resins, polyester resins, hypoglycaemic agents, butadiene and/or butadiene-based products. Accordingly, in some embodiments, the invention provides biobased used as a raw material in the production of a wide range of products comprising one or more bioderived 1,3-BDO or bioderived 1,3-BDO intermediate produced by a non-naturally occurring microorganism of the invention or produced using a method disclosed herein.

As used herein, the term “bioderived” means derived from or synthesized by a biological organism and can be considered a renewable resource since it can be generated by a biological organism. Such a biological organism, in particular the microbial organisms of the invention disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the invention. A biobased or bioderived product is in contrast to a petroleum derived product, wherein such a product is derived from or synthesized from petroleum or a petrochemical feedstock.

In some embodiments, the invention provides organic solvents, polyurethane resins, polyester resins, hypoglycaemic agents, butadiene and/or butadiene-based products comprising bioderived 1,3-BDO or bioderived 1,3-BDO intermediate, wherein the bioderived 1,3-BDO or bioderived 1,3-BDO intermediate includes all or part of the 1,3-BDO or 1,3-BDO intermediate used in the production of organic solvents, polyurethane resins, polyester resins, hypoglycaemic agents, butadiene and/or butadiene-based products. Thus, in some aspects, the invention provides biobased organic solvents, polyurethane resins, polyester resins, hypoglycaemic agents, butadiene and/or butadiene-based products comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived 1,3-BDO or bioderived 1,3-BDO intermediate as disclosed herein. Additionally, in some aspects, the invention provides biobased organic solvents, polyurethane resins, polyester resins, hypoglycaemic agents, butadiene and/or butadiene-based products wherein the 1,3-BDO or 1,3-BDO intermediate used in its production is a combination of bioderived and petroleum derived 1,3-BDO or 1,3-BDO intermediate. For example, biobased organic solvents, polyurethane resins, polyester resins, hypoglycaemic agents, butadiene and/or butadiene-based products can be produced using 50% bioderived 1,3-BDO and 50% petroleum derived 1,3-BDO or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing organic solvents, polyurethane resins, polyester resins, hypoglycaemic agents, butadiene and/or butadiene-based products using the bioderived 1,3-BDO or bioderived 1,3-BDO intermediate of the invention are well known in the art.

The culture conditions can include, for example, liquid culture procedures as well as fermentation and other large scale culture procedures. As described herein, particularly useful yields of cytosolic acetyl-CoA and/or biosynthetic products, such as 1,3-BDO and others, can be obtained under anaerobic or substantially anaerobic culture conditions.

As described herein, one exemplary growth condition for achieving biosynthesis of cytosolic acetyl-CoA and/or 1,3-BDO includes anaerobic culture or fermentation conditions. In certain embodiments, the non-naturally occurring eukaryotic organisms provided herein can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for producing cytosolic acetyl-CoA. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of cytosolic acetyl-CoA. Generally, and as with non-continuous culture procedures, the continuous and/or near-continuous production of cytosolic acetyl-CoA will include culturing a non-naturally occurring cytosolic acetyl-CoA producing organism provided herein further comprising a biosynthetic pathway for the production of a compound that can be synthesized using cytosolic acetyl-CoA in sufficient nutrients and medium to sustain and/or nearly sustain growth in an exponential phase. The culture conditions described herein can likewise be used, scaled up and grown continuously for manufacturing of 1,3-BDO. Fermentation procedures are particularly useful for the biosynthetic production of commercial quantities of 1,3-BDO. Generally, and as with non-continuous culture procedures, the continuous and/or near-continuous production of 1,3-BDO will include culturing a non-naturally occurring 1,3-BDO producing organism in sufficient nutrients and medium to sustain and/or nearly sustain growth in an exponential phase.

Continuous culture under such conditions can include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms provided herein can be cultured for hours, if suitable for a particular application. It is to be understood that the continuous and/or near-continuous culture conditions also can include all time intervals in between these exemplary periods. It is further understood that the time of culturing the eukaryotic organisms provided herein is for a sufficient period of time to produce a sufficient amount of product for a desired purpose.

Fermentation procedures are well known in the art. Briefly, fermentation for the biosynthetic production of cytosolic acetyl-CoA can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the cytosolic acetyl-CoA producers provided herein for continuous production of substantial quantities of cytosolic acetyl-CoA, the cytosolic acetyl-CoA producers also can be, for example, simultaneously subjected to chemical synthesis procedures to convert the product to other compounds or the product can be separated from the fermentation culture and sequentially subjected to chemical or enzymatic conversion to convert the product to other compounds, if desired. Likewise, 1,3-BDO producers also can be, for example, simultaneously subjected to chemical synthesis procedures to convert the product to other compounds or the product can be separated from the fermentation culture and sequentially subjected to chemical conversion to convert the product to other compounds, if desired. For example, 1,3-BDO can be dehydrated to provide 1,3-BDO. In some embodiments, a non-naturally occurring eukaryotic organism comprising an acetyl-CoA pathway further comprises a biosynthetic pathway for the production of a compound that uses cytosolic acetyl-CoA as a precursor, the biosynthetic pathway comprising at least one exogenous nucleic acid encoding an enzyme expressed in a sufficient amount to produce the compound. Compounds of interest that can be produced be produced using cytosolic acetyl-CoA as a precursor include 1,3-BDO and others.

In some embodiments, syngas can be used as a carbon feedstock. Important process considerations for a syngas fermentation are high biomass concentration and good gas-liquid mass transfer (Bredwell et al., Biotechnol Prog., 15:834-844 (1999). The solubility of CO in water is somewhat less than that of oxygen. Continuously gas-sparged fermentations can be performed in controlled fermenters with constant off-gas analysis by mass spectrometry and periodic liquid sampling and analysis by GC and HPLC. The liquid phase can function in batch mode. Fermentation products such as alcohols, organic acids, and residual glucose along with residual methanol are quantified by HPLC (Shimadzu, Columbia Md.), for example, using an Aminex® series of HPLC columns (for example, HPX-87 series) (BioRad, Hercules Calif.), using a refractive index detector for glucose and alcohols, and a UV detector for organic acids. The growth rate is determined by measuring optical density using a spectrophotometer (600 nm). All piping in these systems is glass or metal to maintain anaerobic conditions. The gas sparging is performed with glass frits to decrease bubble size and improve mass transfer. Various sparging rates are tested, ranging from about 0.1 to 1 vvm (vapor volumes per minute). To obtain accurate measurements of gas uptake rates, periodic challenges are performed in which the gas flow is temporarily stopped, and the gas phase composition is monitored as a function of time.

In order to achieve the overall target productivity, methods of cell retention or recycle are employed. One method to increase the microbial concentration is to recycle cells via a tangential flow membrane from a sidestream. Repeated batch culture can also be used, as previously described for production of acetate by Moorella (Sakai et al., J Biosci. Bioeng, 99:252-258 (2005)). Various other methods can also be used (Bredwell et al., Biotechnol Prog., 15:834-844 (1999); Datar et al., Biotechnol Bioeng, 86:587-594 (2004)). Additional optimization can be tested such as overpressure at 1.5 atm to improve mass transfer (Najafpour et al., Enzyme and Microbial Technology, 38[1-2], 223-228 (2006)).

Once satisfactory performance is achieved using pure H2/CO as the feed, synthetic gas mixtures are generated containing inhibitors likely to be present in commercial syngas. For example, a typical impurity profile is 4.5% CH4, 0.1% C2H2, 0.35% C2H6, 1.4% C2H4, and 150 ppm nitric oxide (Datar et al., Biotechnol Bioeng, 86:587-594 (2004)). Tars, represented by compounds such as benzene, toluene, ethylbenzene, p-xylene, o-xylene, and naphthalene, are added at ppm levels to test for any effect on production. For example, it has been shown that 4O ppm NO is inhibitory to C. carboxidivorans (Ahmed et al., Biotechnol Bioeng, 97:1080-1086 (2007)). Cultures are tested in shake-flask cultures before moving to a fermentor. Also, different levels of these potential inhibitory compounds are tested to quantify the effect they have on cell growth. This knowledge is used to develop specifications for syngas purity, which is utilized for scale up studies and production. If any particular component is found to be difficult to decrease or remove from syngas used for scale up, an adaptive evolution procedure is utilized to adapt cells to tolerate one or more impurities.

Advances in the field of protein engineering make it feasible to alter any of the enzymes disclosed herein to act efficiently on substrates not known to be natural to them. Below are several examples of broad-specificity enzymes from diverse classes of interest and methods that have been used for evolving such enzymes to act on non-natural substrates.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of cytosolic acetyl-CoA.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption Methods that result in genetically stable eukaryotic organisms which overproduce the target product. Specifically, the framework examines the complete metabolic and/or biochemical network of a eukaryotic organism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring eukaryotic organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host organisms. Such metabolic modeling and simulation methods include, for example, the computational systems exemplified above as SimPheny® and OptKnock. For illustration, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

As disclosed herein, a nucleic acid encoding a desired activity of an acetyl-CoA pathway and/or 1,3-BDO pathway can be introduced into a host organism. In some cases, it can be desirable to modify an activity of an acetyl-CoA pathway enzyme or protein and/or 1,3-BDO pathway enzyme or protein to increase production of cytosolic acetyl-CoA or 1,3-BDO, respectively. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution methods have made possible the modification of an enzyme to function on an array of unnatural substrates. The substrate specificity of the lipase in P. aeruginosa was broadened by randomization of amino acid residues near the active site. This allowed for the acceptance of alpha-substituted carboxylic acid esters by this enzyme Reetz et al., Angew. Chem. Int. Ed Engl. 44:4192-4196 (2005)). In another successful attempt, DNA shuffling was employed to create an Escherichia coli aminotransferase that accepted β-branched substrates, which were poorly accepted by the wild-type enzyme (Yano et al., Proc. Natl. Acad. Sci. U.S.A 95:5511-5515 (1998)). Specifically, at the end of four rounds of shuffling, the activity of aspartate aminotransferase for valine and 2-oxovaline increased by up to five orders of magnitude, while decreasing the activity towards the natural substrate, aspartate, by up to 30-fold. Recently, an algorithm was used to design a retro-aldolase that could be used to catalyze the carbon-carbon bond cleavage in a non-natural and non-biological substrate, 4-hydroxy-4-(6-methoxy-2-naphthyl)-2-butanone. These algorithms used different combinations of four different catalytic motifs to design new enzymes and 20 of the selected designs for experimental characterization had four-fold improved rates over the uncatalyzed reaction (Jiang et al., Science 319:1387-1391 (2008)). Thus, not only are these engineering approaches capable of expanding the array of substrates on which an enzyme can act, but allow the design and construction of very efficient enzymes. For example, a method of DNA shuffling (random chimeragenesis on transient templates or RACHITT) was reported to lead to an engineered monooxygenase that had an improved rate of desulfurization on complex substrates as well as 20-fold faster conversion of a non-natural substrate (Coco et al. Nat. Biotechnol. 19:354-359 (2001)). Similarly, the specific activity of a sluggish mutant triosephosphate isomerase enzyme was improved up to 19-fold from 1.3 fold (Hermes et al., Proc. Natl. Acad. Sci. U.S.A 87:696-700 (1990)). This enhancement in specific activity was accomplished by using random mutagenesis over the whole length of the protein and the improvement could be traced back to mutations in six amino acid residues.

The effectiveness of protein engineering approaches to alter the substrate specificity of an enzyme for a desired substrate has also been demonstrated. Isopropylmalate dehydrogenase from Thermus thermophilus was modified by changing residues close to the active site so that it could now act on malate and D-lactate as substrates (Fujita et al., Biosci. Biotechnol Biochem. 65:2695-2700 (2001)). In this study as well as in others, it was pointed out that one or a few residues could be modified to alter the substrate specificity. A case in point is the dihydroflavonol 4-reductase for which a single amino acid was changed in the presumed substrate-binding region that could preferentially reduce dihydrokaempferol Johnson et al., Plant 25:325-333 (2001)). The substrate specificity of a very specific isocitrate dehydrogenase from Escherichia coli was changed from isocitrate to isopropylmalate by changing one residue in the active site (Doyle et al., Biochemistry 40:4234-4241 (2001)). In a similar vein, the cofactor specificity of a NAD+-dependent 1,5-hydroxyprostaglandin dehydrogenase was altered to NADP+ by changing a few residues near the N-terminal end Cho et al., Arch. Biochem. Biophys. 419:139-146 (2003)). Sequence analysis and molecular modeling analysis were used to identify the key residues for modification, which were further studied by site-directed mutagenesis.

A fucosidase was evolved from a galactosidase in E. coli by DNA shuffling and screening (Zhang et al., Proc Natl Acad Sci US.A. 94:4504-4509 (1997)). Similarly, aspartate aminotransferase from E. coli was converted into a tyrosine aminotransferase using homology modeling and site-directed mutagenesis (Onuffer et al., Protein Sci. 4:1750-1757 (1995)). Site-directed mutagenesis of two residues in the active site of benzoylformate decarboxylase from P. putida reportedly altered the affinity (Km) towards natural and non-natural substrates Siegert et al., Protein Eng Des Sel 18:345-357 (2005)). Cytochrome c peroxidase (CCP) from Saccharomyces cerevisiae was subjected to directed molecular evolution to generate mutants with increased activity against the classical peroxidase substrate guaiacol, thus changing the substrate specificity of CCP from the protein cytochrome c to a small organic molecule. After three rounds of DNA shuffling and screening, mutants were isolated which possessed a 300-fold increased activity against guaiacol and up to 1000-fold increased specificity for this substrate relative to that for the natural substrate (Iffland et al., Biochemistry 39:10790-10798 (2000)).

In some cases, enzymes with different substrate preferences than both the parent enzymes have been obtained. For example, biphenyl-dioxygenase-mediated degradation of polychlorinated biphenyls was improved by shuffling genes from two bacteria, Pseudomonas pseudoalcaligens and Burkholderia cepacia (Kumamaru et al., Nat. Biotechnol. 16, 663-666 (1998)). The resulting chimeric biphenyl oxygenases showed different substrate preferences than both the parental enzymes and enhanced the degradation activity towards related biphenyl compounds and single aromatic ring hydrocarbons such as toluene and benzene which were originally poor substrates for the enzyme.

It is not only possible to change the enzyme specificity but also to enhance the activities on those substrates on which the enzymes naturally have low activities. One study demonstrated that amino acid racemase from P. putida that had broad substrate specificity (on lysine, arginine, alanine, serine, methionine, cysteine, leucine and histidine among others) but low activity towards tryptophan could be improved significantly by random mutagenesis Kino et al., Appl. Microbiol. Biotechnol. 73:1299-1305 (2007)). Similarly, the active site of the bovine BCKAD was engineered to favor alternate substrate acetyl-CoA (Meng et al., Biochemistry 33:12879-12885 (1994)). An interesting aspect of these approaches is that even when random methods have been applied to generate these mutated enzymes with efficacious activities, the exact mutations or structural changes that confer the improvement in activity can be identified. For example, in the aforementioned study, the mutations that facilitated improved activity on tryptophan could be traced back to two different positions.

Directed evolution has also been used to express proteins that are difficult to express. For example, by subjecting the horseradish peroxidase to random mutagenesis and gene recombination, mutants could be extracted that had more than 14-fold activity than the wild type (Lin et al., Biotechnol. Prog. 15:467-471 (1999)).

A final example of directed evolution shows the extensive modifications to which an enzyme can be subjected to achieve a range of desired functions. The enzyme, lactate dehydrogenase from Bacillus stearothermophilus was subjected to site-directed mutagenesis, and three amino acid substitutions were made at sites that were indicated to determine the specificity towards different hydroxyacids (Clarke et al., Biochem. Biophys. Res. Commun. 148:15-23 (1987)). After these mutations, the specificity for oxaloacetate over pyruvate was increased to 500 in contrast to the wild type enzyme that had a catalytic specificity for pyruvate over oxaloacetate of 1000. This enzyme was further engineered using site-directed mutagenesis to have activity towards branched-chain substituted pyruvates (Wilks et al., Biochemistry 29:8587-8591 (1990)). Specifically, the enzyme had a 55-fold improvement in Kcat for alpha-ketoisocaproate. Three structural modifications were made in the same enzyme to change its substrate specificity from lactate to malate. The enzyme was highly active and specific towards malate (Wilks et al., Science 242:1541-1544 (1988)). The same enzyme from B. stearothermophilus was subsequently engineered to have high catalytic activity towards alpha-keto acids with positively charged side chains, such as those containing ammonium groups (Hogan et al., Biochemistry 34:4225-4230 (1995)). Mutants with acidic amino acids introduced at position 102 of the enzyme favored binding of such side chain ammonium groups. The results obtained proved that the mutants showed up to 25-fold improvements in kcat/Km values for omega-amino-alpha-keto acid substrates. This enzyme was also structurally modified to function as a phenyllactate dehydrogenase instead of a lactate dehydrogenase (Wilks et al., Biochemistry 31:7802-7806 (1992)). Restriction sites were introduced into the gene for the enzyme which allowed a region of the gene to be excised. This region coded for a mobile surface loop of polypeptide (residues 98-110) which normally seals the active site vacuole from bulk solvent and is a major determinant of substrate specificity. The variable length and sequence loops were inserted into the cut gene and used to synthesize hydroxyacid dehydrogenases with altered substrate specificities. With one longer loop construction, activity with pyruvate was reduced one-million-fold but activity with phenylpyruvate was largely unaltered. A switch in specificity (kcat/Km) of 390,000-fold was achieved. The 1700:1 selectivity of this enzyme for phenylpyruvate over pyruvate is that required in a phenyllactate dehydrogenase.

As indicated above, directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened.

Numerous directed evolution technologies have been developed (for reviews, see Hibbert et al., Biomol. Eng 22:11-19 (2005); Huisman and Lalonde, Biocatalysis in the Pharmaceutical and Biotechnology Industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes.

Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of an acetyl-CoA pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include heteroduplex recombination, in which linearized plasmid DNA is used to form heteroduplexes that are repaired by mismatch repair (Volkov et al., Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional ts mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

It is understood that modifications which do not substantially affect the activity of the various embodiments provided herein are also provided within the definition provided herein. Accordingly, the following examples are intended to illustrate but not limit.

### Example I

### Pathways for Producing Cytosolic Acetyl-CoA from Mitochondrial Acetyl-CoA

The production of cytosolic acetyl-CoA from mitochondrial acetyl-CoA can be accomplished by a number of pathways, for example, in three to five enzymatic steps. In one exemplary pathway, mitochondrial acetyl-CoA and oxaloacetate are combined into citrate by a citrate synthase and the citrate is exported out of the mitochondrion by a citrate or citrate/oxaloacetate transporter. Enzymatic conversion of the citrate in the cytosol results in cytosolic acetyl-CoA and oxaloacetate. The cytosolic oxaloacetate can then optionally be transported back into the mitochondrion by an oxaloacetate transporter and/or a citrate/oxaloacetate transporter. In another exemplary pathway, the cytosolic oxaloacetate is first enzymatically converted into malate in the cytosol and then optionally transferred into the mitochondrion by a malate transporter and/or a malate/citrate transporter. Mitochondrial malate can then be converted into oxaloacetate with a mitochondrial malate dehydrogenase.

In yet another exemplary pathway, mitochondrial acetyl-CoA can be converted to cytosolic acetyl-CoA via a citramalate intermediate. For example, mitochondrial acetyl-CoA and pyruvate are converted to citramalate by citramalate synthase. Citramalate can then be transported into the cytosol by a citramalate or dicarboxylic acid transporter. Cytosolic acetyl-CoA and pyruvate are then regenerated from citramalate, directly or indirectly, and the pyruvate can re-enter the mitochondria.

Along these lines, several exemplary acetyl-CoA pathways for the production of cytosolic acetyl-CoA from mitochondrial acetyl-CoA are shown in FIGS. 2, 3 and 8. In one embodiment, mitochondrial oxaloacetate is combined with mitochondrial acetyl-CoA to form citrate by a citrate synthase (FIGS. 2, 3 and 8, A). The citrate is transported outside of the mitochondrion by a citrate transporter (FIGS. 2, 3 and 8, B), a citrate/oxaloacetate transporter (FIG. 2C) or a citrate/malate transporter (FIG. 3C). Cytosolic citrate is converted into cytosolic acetyl-CoA and oxaloacetate by an ATP citrate lyase (FIGS. 2, 3, D). In another pathway, cytosolic citrate is converted into acetate and oxaloacetate by a citrate lyase (FIGS. 2 and 3, E). Acetate can then be converted into cytosolic acetyl-CoA by an acetyl-CoA synthetase or transferase (FIGS. 2 and 3, F). Alternatively, acetate can be converted by an acetate kinase (FIGS. 2 and 3, K) to acetyl phosphate, and the acetyl phosphate can be converted to cytosolic acetyl-CoA by a phosphotransacetylase (FIGS. 2 and 3, L). Exemplary enzyme candidates for acetyl-CoA pathway enzymes are described below.

The conversion of oxaloacetate and mitochondrial acetyl-CoA is catalyzed by a citrate synthase (FIGS. 2, 3 and 8, A). In certain embodiments, the citrate synthase is expressed in a mitochondrion of a non-naturally occurring eukaryotic organism provided herein.

TABLE 11

| Protein | GenBank ID | GI number | Organism |
|---|---|---|---|
| CIT1 | NP_014398.1 | 6324328 | Saccharomyces |
| | | | cerevisiae S288c |
| CIT2 | NP_009931.1 | 6319850 | Saccharomyces |
| | | | cerevisiae S288c |
| CIT3 | NP_015325.1 | 6325257 | Saccharomyces |
| | | | cerevisiae S288c |
| YALI0E02684p | XP_503469.1 | 50551989 | Yarrowia lipolytica |
| YALI0E00638p | XP_503380.1 | 50551811 | Yarrowia lipolytica |
| ANI_1_876084 | XP_001393983.1 | 145242820 | Aspergillus niger |
| | | | CBS 513.88 |
| ANI_1_1474074 | XP_001393195.2 | 317030721 | Aspergillus niger |
| | | | CBS 513.88 |
| ANI_1_2950014 | XP_001389414.2 | 317026339 | Aspergillus niger |
| | | | CBS 513.88 |
| ANI_1_1226134 | XP_001396731.1 | 145250435 | Aspergillus niger |
| | | | CBS 513.88 |
| gltA | NP_415248.1 | 16128695 | Escherichia coli |
| | | | K-12 MG1655 |

Transport of citrate from the mitochondrion to the cytosol can be carried out by several transport proteins. Such proteins either export citrate directly (i.e., citrate transporter, FIGS. 2, 3 and 8, B) to the cytosol or export citrate to the cytosol while simultaneously transporting a molecule such as malate (i.e., citrate/malate transporter, FIG. 3C) or oxaloacetate (i.e., citrate/oxaloacetate transporter FIG. 2C) from the cytosol into the mitochondrion as shown in FIGS. 2, 3 and 8. Exemplary transport enzymes that carry out these transformations are provided in the table below.

TABLE 12

| Protein | GenBank ID | GI number | Organism |
|---|---|---|---|
| CTP1 | NP_009850.1 | 6319768 | Saccharomyces |
| | | | cerevisiae S288c |
| YALI0F26323p | XP_505902.1 | 50556988 | Yarrowia lipolytica |
| ATEG_09970 | EAU29419.1 | 114187719 | Aspergillus terreus |
| | | | NIH2624 |
| KLLA0E18723g | XP_454797.1 | 50309571 | Kluyveromyces |
| | | | lactis |
| | | | NRRL Y-1140 |
| CTRG_02320 | XP_002548023.1 | 255726194 | Candida tropicalis |
| | | | MYA-3404 |
| ANI_1_1474094 | XP_001395080.1 | 145245625 | Aspergillus niger |
| | | | CBS 513.88 |
| YHM2 | NP_013968.1 | 6323897 | Saccharomyces |
| | | | cerevisiae S288c |
| DTC | CAC84549.1 | 19913113 | Arabidopsis |
| | | | thaliana |
| DTC1 | CAC84545.1 | 19913105 | Nicotiana tabacum |
| DTC2 | CAC84546.1 | 19913107 | Nicotiana tabacum |
| DTC3 | CAC84547.1 | 19913109 | Nicotiana tabacum |
| DTC4 | CAC84548.1 | 19913111 | Nicotiana tabacum |
| DTC | AAR06239.1 | 37964368 | Citrus junos |

ATP citrate lyase (ACL, EC 2.3.3.8, FIGS. 2 and 3, D), also called ATP citrate synthase, catalyzes the ATP-dependent cleavage of citrate to oxaloacetate and acetyl-CoA. In certain embodiments, ATP citrate lyase is expressed in the cytosol of a eukaryotic organism. ACL is an enzyme of the RTCA cycle that has been studied in green sulfur bacteria Chlorobium limicola and Chlorobium tepidum. The alpha(4)beta(4) heteromeric enzyme from Chlorobium limicola was cloned and characterized in E. coli (Kanao et al., Eur. J. Biochem. 269:3409-3416 (2002). The C. limicola enzyme, encoded by ac1AB, is irreversible and activity of the enzyme is regulated by the ratio of ADP/ATP. The Chlorobium tepidum a recombinant ACL from Chlorobium tepidum was also expressed in E. coli and the holoenzyme was reconstituted in vitro, in a study elucidating the role of the alpha and beta subunits in the catalytic mechanism (Kim and Tabita, J. Bacteriol. 188:6544-6552 (2006). ACL enzymes have also been identified in Balnearium lithotrophicum, Sulfurihydrogenibium subterraneum and other members of the bacterial phylum Aquificae (Hugler et al., Environ. Microbiol. 9:81-92 (2007)). This activity has been reported in some fungi as well. Exemplary organisms include Sordaria macrospora (Nowrousian et al., Curr. Genet. 37:189-93 (2000)), Aspergillus nidulans and Yarrowia hpolytica (Hynes and Murray, Eukaryotic Cell, July: 1039-1048, (2010), and Aspergillus niger (Meijer et al. J. Ind. Microbiol. Biotechnol. 36:1275-1280 (2009). Other candidates can be found based on sequence homology. Information related to these enzymes is tabulated below.

TABLE 13

| Protein | GenBank ID | GI Number | Organism |
|---|---|---|---|
| aclA | BAB21376.1 | 12407237 | Chlorobium limicola |
| aclB | BAB21375.1 | 12407235 | Chlorobium limicola |
| aclA | AAM72321.1 | 21647054 | Chlorobium tepidum |
| aclB | AAM72322.1 | 21647055 | Chlorobium tepidum |
| aclB | ABI50084.1 | 114055039 | Sulfurihydrogenibium |
| | | | subterraneum |
| aclA | AAX76834.1 | 62199504 | Sulfurimonas |
| | | | denitrificans |
| aclB | AAX76835.1 | 62199506 | Sulfurimonas |
| | | | denitrificans |
| acl1 | XP_504787.1 | 50554757 | Yarrowia lipolytica |
| acl2 | XP_503231.1 | 50551515 | Yarrowia lipolytica |
| SPBC1703.07 | NP_596202.1 | 19112994 | Schizosaccharomyces |
| | | | pombe |
| SPAC22A12.16 | NP_593246.1 | 19114158 | Schizosaccharomyces |
| | | | pombe |
| acl1 | CAB76165.1 | 7160185 | Sordaria macrospora |
| acl2 | CAB76164.1 | 7160184 | Sordaria macrospora |
| aclA | CBF86850.1 | 259487849 | Aspergillus nidulans |
| aclB | CBF86848 | 259487848 | Aspergillus nidulans |

In some organisms the conversion of citrate to oxaloacetate and acetyl-CoA proceeds through a citryl-CoA intermediate and is catalyzed by two separate enzymes, citryl-CoA synthetase (EC 6.2.1.18) and citryl-CoA lyase (EC 4.1.3.34) (Aoshima, M., Appl. Microbiol. Biotechnol. 75:249-255 (2007). Citryl-CoA synthetase catalyzes the activation of citrate to citryl-CoA. The Hydrogenobacter thermophilus enzyme is composed of large and small subunits encoded by ccsA and ccsB, respectively (Aoshima et al., Mol. Micrbiol. 52:751-761 (2004)). The citryl-CoA synthetase of Aquifex aeolicus is composed of alpha and beta subunits encoded by sucC1 and sucD1 (Hugler et al., Environ. Microbiol. 9:81-92 (2007)). Citryl-CoA lyase splits citryl-CoA into oxaloacetate and acetyl-CoA. This enzyme is a homotrimer encoded by ccl in Hydrogenobacter thermophilus (Aoshima et al., Mol. Microbiol. 52:763-770 (2004)) and aq_150 in Aquifex aeolicus (Hugler et al., supra (2007)). The genes for this mechanism of converting citrate to oxaloacetate and citryl-CoA have also been reported recently in Chlorobium tepidum (Eisen et al., PNAS 99(14): 9509-14 (2002)).

TABLE 14

| Protein | GenBank ID | GI Number | Organism |
|---|---|---|---|
| ccsA | BAD17844.1 | 46849514 | Hydrogenobacter |
| | | | thermophilus |
| ccsB | BAD17846.1 | 46849517 | Hydrogenobacter |
| | | | thermophilus |
| sucC1 | AAC07285 | 2983723 | Aquifex aeolicus |
| sucD1 | AAC07686 | 2984152 | Aquifex aeolicus |
| ccl | BAD17841.1 | 46849510 | Hydrogenobacter |
| | | | thermophilus |
| aq_150 | AAC06486 | 2982866 | Aquifex aeolicus |
| CT0380 | NP_661284 | 21673219 | Chlorobium tepidum |
| CT0269 | NP_661173.1 | 21673108 | Chlorobium tepidum |
| CT1834 | AAM73055.1 | 21647851 | Chlorobium tepidum |

Citrate lyase (EC 4.1.3.6, FIGS. 2 and 3, E) catalyzes a series of reactions resulting in the cleavage of citrate to acetate and oxaloacetate. In certain embodiments, citrate lyase is expressed in the cytosol of a eukaryotic organism. The enzyme is active under anaerobic conditions and is composed of three subunits: an acyl-carrier protein (ACP, gamma), an ACP transferase (alpha), and an acyl lyase (beta). Enzyme activation uses covalent binding and acetylation of an unusual prosthetic group, 2′-(5″-phosphoribosyl)-3-′-dephospho-CoA, which is similar in structure to acetyl-CoA. Acylation is catalyzed by CitC, a citrate lyase synthetase. Two additional proteins, CitG and CitX, are used to convert the apo enzyme into the active holo enzyme (Schneider et al., Biochemistry 39:9438-9450 (2000)). Wild type E. coli does not have citrate lyase activity; however, mutants deficient in molybdenum cofactor synthesis have an active citrate lyase (Clark, FEMS Microbiol. Lett. 55:245-249 (1990)). The E. coli enzyme is encoded by citEFD and the citrate lyase synthetase is encoded by citC (Nilekani and SivaRaman, Biochemistry 22:4657-4663 (1983)). The Leuconostoc mesenteroides citrate lyase has been cloned, characterized and expressed in E. coli (Bekal et al., J. Bacteriol. 180:647-654 (1998)). Citrate lyase enzymes have also been identified in enterobacteria that utilize citrate as a carbon and energy source, including Salmonella typhimurium and Klebsiella pneumoniae (Bott, Arch. Microbiol. 167: 78-88 (1997); Bott and Dimroth, Mol. Microbiol. 14:347-356 (1994)). The aforementioned proteins are tabulated below.

TABLE 15

| Protein | GenBank ID | GI Number | Organism |
|---|---|---|---|
| citF | AAC73716.1 | 1786832 | Escherichia coli |
| cite | AAC73717.2 | 87081764 | Escherichia coli |
| citD | AAC73718.1 | 1786834 | Escherichia coli |
| citC | AAC73719.2 | 87081765 | Escherichia coli |
| citG | AAC73714.1 | 1786830 | Escherichia coli |
| citX | AAC73715.1 | 1786831 | Escherichia coli |
| citF | CAA71633.1 | 2842397 | Leuconostoc mesenteroides |
| citE | CAA71632.1 | 2842396 | Leuconostoc mesenteroides |
| citD | CAA71635.1 | 2842395 | Leuconostoc mesenteroides |
| citC | CAA71636.1 | 3413797 | Leuconostoc mesenteroides |
| citG | CAA71634.1 | 2842398 | Leuconostoc mesenteroides |
| citX | CAA71634.1 | 2842398 | Leuconostoc mesenteroides |
| citF | NP_459613.1 | 16763998 | Salmonella typhimurium |
| citE | AAL19573.1 | 16419133 | Salmonella typhimurium |
| citD | NP_459064.1 | 16763449 | Salmonella typhimurium |
| citC | NP_459616.1 | 16764001 | Salmonella typhimurium |
| citG | NP_459611.1 | 16763996 | Salmonella typhimurium |
| citX | NP_459612.1 | 16763997 | Salmonella typhimurium |
| citF | CAA56217.1 | 565619 | Klebsiella pneumoniae |
| citE | CAA56216.1 | 565618 | Klebsiella pneumoniae |
| citD | CAA56215.1 | 565617 | Klebsiella pneumoniae |
| citC | BAH66541.1 | 238774045 | Klebsiella pneumoniae |
| citG | CAA56218.1 | 565620 | Klebsiella pneumoniae |
| citX | AAL60463.1 | 18140907 | Klebsiella pneumoniae |

The acylation of acetate to acetyl-CoA is catalyzed by enzymes with acetyl-CoA synthetase activity (FIGS. 2 and 3, F). In certain embodiments, acetyl-CoA synthetase is expressed in the cytosol of a eukaryotic organism. Two enzymes that catalyze this reaction are AMP-forming acetyl-CoA synthetase (EC 6.2.1.1) and ADP-forming acetyl-CoA synthetase (EC 6.2.1.13). AMP-forming acetyl-CoA synthetase (ACS) is the predominant enzyme for activation of acetate to acetyl-CoA. Exemplary ACS enzymes are found in E. coli (Brown et al., J. Gen. Microbiol. 102:327-336 (1977)), Ralstonia eutropha (Priefert and Steinbuchel, J. Bacteriol. 174:6590-6599 (1992)), Methanothermobacter thermautotrophicus (Ingram-Smith and Smith, Archaea 2:95-107 (2007)), Salmonella enterica (Gulick et al., Biochemistry 42:2866-2873 (2003)) and Saccharomyces cerevisiae (Jogl and Tong, Biochemistry 43:1425-1431 (2004)).

TABLE 16

| Protein | GenBank ID | GI Number | Organism |
|---|---|---|---|
| acs | AAC77039.1 | 1790505 | Escherichia coli |
| acoE | AAA21945.1 | 141890 | Ralstonia eutropha |
| acs1 | ABC87079.1 | 86169671 | Methanothermobacter |
| | | | thermautotrophicus |
| acs1 | AAL23099.1 | 16422835 | Salmonella enterica |
| ACS1 | Q01574.2 | 257050994 | Saccharomyces cerevisiae |

ADP-forming acetyl-CoA synthetase (ACD, EC 6.2.1.13) is another candidate enzyme that couples the conversion of acyl-CoA esters to their corresponding acids with the concurrent synthesis of ATP. Several enzymes with broad substrate specificities have been described in the literature. ACD I from Archaeoglobus fulgidus, encoded by AF1211, was shown to operate on a variety of linear and branched-chain substrates including acetyl-CoA, propionyl-CoA, butyryl-CoA, acetate, propionate, butyrate, isobutyryate, isovalerate, succinate, fumarate, phenylacetate, indoleacetate (Musfeldt et al., J. Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al., supra (2004)). The enzymes from A. fulgidus, H. marismortui and P. aerophilum have all been cloned, functionally expressed, and characterized in E. coli (Musfeldt et al., supra; Brasen et al., supra (2004)). Additional candidates include the succinyl-CoA synthetase encoded by sucCD in E. coli (Buck et al., Biochemistry 24:6245-6252 (1985)) and the acyl-CoA ligase from Pseudomonas putida (Fernandez-Valverde et al., Appl. Environ. Microbiol. 59:1149-1154 (1993)). Information related to these proteins and genes is shown below.

TABLE 17

| Protein | GenBank ID | GI number | Organism |
|---|---|---|---|
| AF1211 | NP_070039.1 | 11498810 | Archaeoglobus fulgidus |
| | | | DSM 4304 |
| AF1983 | NP_070807.1 | 11499565 | Archaeoglobus fulgidus |
| | | | DSM 4304 |
| scs | YP_135572.1 | 55377722 | Haloarcula marismortui |
| | | | ATCC 43049 |
| PAE3250 | NP_560604.1 | 18313937 | Pyrobaculum aerophilum |
| | | | str. IM2 |
| sucC | NP_415256.1 | 16128703 | Escherichia coli |
| sucD | AAC73823.1 | 1786949 | Escherichia coli |
| paaF | AAC24333.2 | 22711873 | Pseudomonas putida |

An alternative method for adding the CoA moiety to acetate is to apply a pair of enzymes such as a phosphate-transferring acyltransferase and an acetate kinase (FIGS. 2 and 3, F, FIGS. 8E and 8F). This activity enables the net formation of acetyl-CoA with the simultaneous consumption of ATP. In certain embodiments, phosphotransacetylase is expressed in the cytosol of a eukaryotic organism. An exemplary phosphate-transferring acyltransferase is phosphotransacetylase, encoded by pta. Thepta gene from E. coli encodes an enzyme that can convert acetyl-CoA into acetyl-phosphate, and vice versa (Suzuki, T. Biochim. Biophys. Acta 191:559-569 (1969)). This enzyme can also utilize propionyl-CoA instead of acetyl-CoA forming propionate in the process (Hesslinger et al. Mol. Microbiol 27:477-492 (1998)). Homologs exist in several other organisms including Salmonella enterica and Chlamydomonas reinhardtii.

TABLE 18

| Protein | GenBank ID | GI number | Organism |
|---|---|---|---|
| Pta | NP_416800.1 | 16130232 | Escherichia coli |
| Pta | NP_461280.1 | 16765665 | Salmonella enterica subsp. |
| | | | enterica serovar Typhimurium |
| | | | str. LT2 |
| PAT2 | XP_001694504.1 | 159472743 | Chlamydomonas reinhardtii |
| PAT1 | XP_001691787.1 | 159467202 | Chlamydomonas reinhardtii |

An exemplary acetate kinase is the E. coli acetate kinase, encoded by ackA (Skarstedt and Silverstein J. Biol. Chem. 251:6775-6783 (1976)). Homologs exist in several other organisms including Salmonella enterica and Chlamydomonas reinhardtii. Information related to these proteins and genes is shown below:

TABLE 19

| Protein | GenBank ID | GI number | Organism |
|---|---|---|---|
| AckA | NP_416799.1 | 16130231 | Escherichia coli |
| AckA | NP_461279.1 | 16765664 | Salmonella enterica subsp. |
| | | | enterica serovar Typhimurium |
| | | | str. LT2 |
| ACK1 | XP_001694505.1 | 159472745 | Chlamydomonas reinhardtii |
| ACK2 | XP_001691682.1 | 159466992 | Chlamydomonas reinhardtii |

In some embodiments, cytosolic oxaloacetate is transported back into a mitochondrion by an oxaloacetate transporter. Oxaloacetate transported back into a mitochondrion can then be used in the acetyl-CoA pathways described herein.

Transport of oxaloacetate from the cytosol to the mitochondrion can be carried out by several transport proteins. Such proteins either import oxaloacetate directly (i.e., oxaloacetate transporter, FIGS. 2G and 8H) to the mitochondrion or import oxaloacetate to the cytosol while simultaneously transporting a molecule such as citrate (i.e., citrate/oxaloacetate transporter, FIGS. 2C and 8H) from the mitochondrion into the cytosol as shown in FIGS. 2 and 3. Exemplary transport enzymes that carry out these transformations are provided in the table below.

TABLE 20

| Protein | GenBank ID | GI number | Organism |
|---|---|---|---|
| OAC1 | NP_012802.1 | 6322729 | Saccharomyces |
| | | | cerevisiae S288c |
| KLLA0B12826g | XP_452102.1 | 50304305 | Kluyveromyces |
| | | | lactis |
| | | | NRRL Y-1140 |
| YALI0E04048g | XP_503525.1 | 50552101 | Yarrowia lipolytica |
| CTRG_02239 | XP_002547942.1 | 255726032 | Candida tropicalis |
| | | | MYA-3404 |
| DIC1 | NP_013452.1 | 6323381 | Saccharomyces |
| | | | cerevisiae S288c |
| YALI0B03344g | XP_500457.1 | 50545838 | Yarrowia lipolytica |
| CTRG_02122 | XP_002547815.1 | 255725772 | Candida tropicalis |
| | | | MYA-3404 |
| PAS_chr4_0877 | XP_002494326.1 | 254574434 | Pichia pastoris |
| | | | GS115 |
| DTC | CAC84549.1 | 19913113 | Arabidopsis |
| | | | thaliana |
| DTC1 | CAC84545.1 | 19913105 | Nicotiana tabacum |
| DTC2 | CAC84546.1 | 19913107 | Nicotiana tabacum |
| DTC3 | CAC84547.1 | 19913109 | Nicotiana tabacum |
| DTC4 | CAC84548.1 | 19913111 | Nicotiana tabacum |
| DTC | AAR06239.1 | 37964368 | Citrus junos |

In some embodiments, cytosolic oxaloacetate is first converted to malate by a cytosolic malate dehydrogenase (FIGS. 3H and 8J). Cytosolic malate is transported into a mitochondrion by a malate transporter or a citrate/malate transporter (FIGS. 3 and 8, I). Mitochondrial malate is then converted to oxaloacetate by a mitochondrial malate dehydrogenase (FIGS. 3J and 8K). Mitochondrial oxaloacetate can then be used in the acetyl-CoA pathways described herein. Exemplary examples of each of these enzymes are provided below.

Oxaloacetate is converted into malate by malate dehydrogenase (EC 1.1.1.37, FIGS. 3H and 8J). When malate is the dicarboxylate transported from the cytosol to mitochondrion, expression of both a cytosolic and mitochondrial version of malate dehydrogenase, e.g., as shown in FIG. 3, can be used. S. cerevisiae possesses three copies of malate dehydrogenase, MDH1 (McAlister-Henn and Thompson, J. Bacteriol. 169:5157-5166 (1987), MDH2 (Minard and McAlister-Henn, Mol. Cell. Biol. 11:370-380 (1991); Gibson and McAlister-Henn, J. Biol. Chem. 278:25628-25636 (2003)), and MDH3 (Steffan and McAlister-Henn, J. Biol. Chem. 267:24708-24715 (1992)), which localize to the mitochondrion, cytosol, and peroxisome, respectively. Close homologs to the cytosolic malate dehydrogenase, MDH2, from S. cerevisiae are found in several organisms including Kluyveromyces lactis and Candida tropicalis. E. coli is also known to have an active malate dehydrogenase encoded by mdh.

TABLE 21

| Protein | GenBank ID | GI Number | Organism |
|---|---|---|---|
| MDH1 | NP_012838 | 6322765 | Saccharomyces |
| | | | cerevisiae |
| MDH2 | NP_014515 | 116006499 | Saccharomyces |
| | | | cerevisiae |
| MDH3 | NP_010205 | 6320125 | Saccharomyces |
| | | | cerevisiae |
| Mdh | NP_417703.1 | 16131126 | Escherichia coli |
| KLLA0E07525p | XP_454288.1 | 50308571 | Kluyveromyces |
| | | | lactis |
| | | | NRRL Y-1140 |
| YALI0D16753g | XP_502909.1 | 50550873 | Yarrowia lipolytica |
| CTRG_01021 | XP_002546239.1 | 255722609 | Candida tropicalis |
| | | | MYA-3404 |

Transport of malate from the cytosol to the mitochondrion can be carried out by several transport proteins. Such proteins either import malate directly (i.e., malate transporter) to the mitochondrion or import malate to the cytosol while simultaneously transporting a molecule such as citrate (i.e., citrate/malate transporter) from the mitochondrion into the cytosol as shown in FIGS. 2, 3 and 8. Exemplary transport enzymes that carry out these transformations are provided in the table below.

TABLE 22

| Protein | GenBank ID | GI number | Organism |
|---|---|---|---|
| OAC1 | NP_012802.1 | 6322729 | Saccharomyces |
| | | | cerevisiae S288c |
| KLLA0B12826g | XP_452102.1 | 50304305 | Kluyveromyces |
| | | | lactis |
| | | | NRRL Y-1140 |
| YALI0E04048g | XP_503525.1 | 50552101 | Yarrowia lipolytica |
| CTRG_02239 | XP_002547942.1 | 255726032 | Candida tropicalis |
| | | | MYA-3404 |
| DIC1 | NP_013452.1 | 6323381 | Saccharomyces |
| | | | cerevisiae S288c |
| YALI0B03344g | XP_500457.1 | 50545838 | Yarrowia lipolytica |
| CTRG_02122 | XP_002547815.1 | 255725772 | Candida tropicalis |
| | | | MYA-3404 |
| PAS_chr4_0877 | XP_002494326.1 | 254574434 | Pichia pastoris |
| | | | GS115 |
| DTC | CAC84549.1 | 19913113 | Arabidopsis |
| | | | thaliana |
| DTC1 | CAC84545.1 | 19913105 | Nicotiana tabacum |
| DTC2 | CAC84546.1 | 19913107 | Nicotiana tabacum |
| DTC3 | CAC84547.1 | 19913109 | Nicotiana tabacum |
| DTC4 | CAC84548.1 | 19913111 | Nicotiana tabacum |
| DTC | AAR06239.1 | 37964368 | Citrus junos |

Malate can be converted into oxaloacetate by malate dehydrogenase (EC 1.1.1.37, FIG. 3, J). When malate is the dicarboxylate transported from the cytosol to mitochondrion, in certain embodiments, both a cytosolic and mitochondrial version of malate dehydrogenase is expressed, as shown in FIG. 3. S. cerevisiae possesses three copies of malate dehydrogenase, MDH1 (McAlister-Henn and Thompson, J. Bacteriol. 169:5157-5166 (1987), MDH2 (Minard and McAlister-Henn, Mol. Cell. Biol. 11:370-380 (1991); Gibson and McAlister-Henn, J. Biol. Chem. 278:25628-25636 (2003)), and MDH3 (Steffan and McAlister-Henn, J. Biol. Chem. 267:24708-24715 (1992)), which localize to the mitochondrion, cytosol, and peroxisome, respectively. Close homologs to the mitochondrial malate dehydrogenase, MDH1, from S. cerevisiae are found in several organisms including Kluyveromyces lactis, Yarrowia lipolytica, Candida tropicalis. E. coli is also known to have an active malate dehydrogenase encoded by mdh.

TABLE 23

| Protein | GenBank ID | GI Number | Organism |
|---|---|---|---|
| MDH1 | NP_012838 | 6322765 | Saccharomyces |
| | | | cerevisiae |
| MDH2 | NP_014515 | 116006499 | Saccharomyces |
| | | | cerevisiae |
| MDH3 | NP_010205 | 6320125 | Saccharomyces |
| | | | cerevisiae |
| Mdh | NP_417703.1 | 16131126 | Escherichia coli |
| KLLA0F25960g | XP_456236.1 | 50312405 | Kluyveromyces |
| | | | lactis |
| | | | NRRL Y-1140 |
| YALI0D16753g | XP_502909.1 | 50550873 | Yarrowia lipolytica |
| CTRG_00226 | XP_002545445.1 | 255721021 | Candida tropicalis |
| | | | MYA-3404 |

### Example II

### Pathways for Producing Cytosolic Acetyl-CoA from Cytosolic Pyruvate

The following example describes exemplary pathways for the conversion of cytosolic pyruvate and threonine to cytosolic acetyl-CoA, as shown in FIG. 5.

Direct conversion of pyruvate to acetyl-CoA can be catalyzed by pyruvate dehydrogenase, pyruvate formate lyase, pyruvate:NAD(P) oxidoreductase or pyruvate:ferredoxin oxidoreductase (FIG. 5H).

Indirect conversion of pyruvate to acetyl-CoA can proceed through several alternate routes. Pyruvate can be converted to acetaldehyde by a pyruvate decarboxylase. Acetaldehyde can then converted to acetyl-CoA by an acylating (CoA-dependent) acetaldehyde dehydrogenase. Alternately, acetaldehyde generated by pyruvate decarboxylase can be converted to acetyl-CoA by the “PDH bypass” pathway. In this pathway, acetaldehyde is oxidized by acetaldehyde dehydrogenase to acetate, which is then converted to acetyl-CoA by a CoA ligase, synthetase or transferase. In another embodiment, the acetate intermediate is converted by an acetate kinase to acetyl-phosphate that is then converted to acetyl-CoA by a phosphotransacetylase. In yet another embodiment, pyruvate is directly converted to acetyl-phosphate by a pyruvate oxidase (acetyl-phosphate forming). Conversion of pyruvate to an acetate intermediate can also catalyzed by acetate-forming pyruvate oxidase.

FIG. 5 depicts several pathways for the indirect conversion of cytosolic pyruvate to cytosolic acetyl-CoA (5A/5B, 5A/5C/5D, 5E/5F/5C/5D, 5G/1D). In the first route, pyruvate is converted to acetate by a pyruvate oxidase (acetate forming) (step A). Acetate can then subsequently converted to acetyl-CoA either directly, by an acetyl-CoA synthetase, ligase or transferase (step B), or indirectly via an acetyl-phosphate intermediate (steps C, D). In an alternate route, pyruvate is decarboxylated to acetaldehyde by a pyruvate decarboxylase (step E). An acetaldehyde dehydrogenase oxidizes acetaldehyde to form acetate (step F). Acetate can then be converted to acetyl-CoA by an acetate kinase and phosphotransacetylase (steps C and D). In yet another route, pyruvate can be oxidized to acetylphosphate by pyruvate oxidase (acetyl-phosphate forming) (step G). A phosphotransacetylase can then convert acetylphopshate to acetyl-CoA (step D).

Cytosolic acetyl-CoA can also be synthesized from threonine by expressing a native or heterologous threonine aldolase (FIG. 5J) (van Maris et al, AEM 69:2094-9 (2003)). Threonine aldolase can convert threonine into acetaldehyde and glycine. The acetaldehyde product can then be converted to acetyl-CoA by various pathways described above.

Gene candidates for the acetyl-CoA forming enzymes shown in FIG. 5 are described below.

Pyruvate oxidase (acetate-forming) (FIG. 5A) or pyruvate:quinone oxidoreductase (PQO) can catalyze the oxidative decarboxylation of pyruvate into acetate, using ubiquione (EC 1.2.5.1) or quinone (EC 1.2.2.1) as an electron acceptor. The E. coli enzyme, PoxB, is localized on the inner membrane (Abdel-Hamid et al., Microbiol 147:1483-98 (2001)). The enzyme has thiamin pyrophosphate and flavin adenine dinucleotide (FAD) cofactors (Koland and Gennis, Biochemistry 21:4438-4442 (1982)); O'Brien et al., Biochemistry 16:3105-3109 (1977); O'Brien and Gennis, J. Biol. Chem. 255:3302-3307 (1980)). PoxB has similarity to pyruvate decarboxylase of S. cerevisiae and Zymomonas mobilis. The pqo transcript of Corynebacterium glutamicum encodes a quinone-dependent and acetate-forming pyruvate oxidoreductase (Schreiner et al., J Bacteriol 188:1341-50 (2006)). Similar enzymes can be inferred by sequence homology.

TABLE 24

| Protein | GenBank ID | GI Number | Organism |
|---|---|---|---|
| poxB | NP_415392.1 | 16128839 | Escherichia coli |
| pqo | YP_226851.1 | 62391449 | Corynebacterium glutamicum |
| poxB | YP_309835.1 | 74311416 | Shigella sonnei |
| poxB | ZP_03065403.1 | 194433121 | Shigella dysenteriae |

The acylation of acetate to acetyl-CoA (FIG. 5B) can be catalyzed by enzymes with acetyl-CoA synthetase, ligase or transferase activity. Two enzymes that can catalyze this reaction are AMP-forming acetyl-CoA synthetase or ligase (EC 6.2.1.1) and ADP-forming acetyl-CoA synthetase (EC 6.2.1.13). AMP-forming acetyl-CoA synthetase (ACS) is the predominant enzyme for activation of acetate to acetyl-CoA. Exemplary ACS enzymes are found in E. coli (Brown et al., J. Gen. Microbiol. 102:327-336 (1977)), Ralstonia eutropha (Priefert and Steinbuchel, J. Bacteriol. 174:6590-6599 (1992)), Methanothermobacter thermautotrophicus (Ingram-Smith and Smith, Archaea 2:95-107 (2007)), Salmonella enterica (Gulick et al., Biochemistry 42:2866-2873 (2003)) and Saccharomyces cerevisiae (Jogl and Tong, Biochemistry 43:1425-1431 (2004)). ADP-forming acetyl-CoA synthetases are reversible enzymes with a generally broad substrate range (Musfeldt and Schonheit, J. Bacteriol. 184:636-644 (2002)). Two isozymes of ADP-forming acetyl-CoA synthetases are encoded in the Archaeoglobus fulgidus genome by are encoded by AF1211 and AF1983 (Musfeldt and Schonheit, supra (2002)). The enzyme from Haloarcula marismortui (annotated as a succinyl-CoA synthetase) also accepts acetate as a substrate and reversibility of the enzyme was demonstrated (Brasen and Schonheit, Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetate, isobutyryl-CoA (preferred substrate) and phenylacetyl-CoA (Brasen and Schonheit, supra (2004)). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, supra (2004); Musfeldt and Schonheit, supra (2002)). Additional candidates include the succinyl-CoA synthetase encoded by sucCD in E. coli (Buck et al., Biochemistry 24:6245-6252 (1985)) and the acyl-CoA ligase from Pseudomonas putida (Fernandez-Valverde et al., Appl. Environ. Microbiol. 59:1149-1154 (1993)). The aforementioned proteins are shown below.

TABLE 25

| Protein | GenBank ID | GI Number | Organism |
|---|---|---|---|
| acs | AAC77039.1 | 1790505 | Escherichia coli |
| acoE | AAA21945.1 | 141890 | Ralstonia eutropha |
| acs1 | ABC87079.1 | 86169671 | Methanothermobacter |
| | | | thermautotrophicus |
| acs1 | AAL23099.1 | 16422835 | Salmonella enterica |
| ACS1 | Q01574.2 | 257050994 | Saccharomyces cerevisiae |
| AF1211 | NP_070039.1 | 11498810 | Archaeoglobus fulgidus |
| AF1983 | NP_070807.1 | 11499565 | Archaeoglobus fulgidus |
| scs | YP_135572.1 | 55377722 | Haloarcula marismortui |
| PAE3250 | NP_560604.1 | 18313937 | Pyrobaculum aerophilum str. |
| | | | IM2 |
| sucC | NP_415256.1 | 16128703 | Escherichia coli |
| sucD | AAC73823.1 | 1786949 | Escherichia coli |
| paaF | AAC24333.2 | 22711873 | Pseudomonas putida |

The acylation of acetate to acetyl-CoA can also be catalyzed by CoA transferase enzymes (FIG. 5B). Numerous enzymes employ acetate as the CoA acceptor, resulting in the formation of acetyl-CoA. An exemplary CoA transferase is acetoacetyl-CoA transferase, encoded by the E. coli atoA (alpha subunit) and atoD (beta subunit) genes (Korolev et al., Acta Crystallo. D. Biol. Crystallo. 58:2116-2121 (2002); Vanderwinkel et al., 33:902-908 (1968)). This enzyme has a broad substrate range (Sramek et al., Arch Biochem Biophys 171:14-26 (1975)) and has been shown to transfer the CoA moiety to acetate from a variety of branched and linear acyl-CoA substrates, including isobutyrate (Matthies et al., Appl Environ. Microbiol 58:1435-1439 (1992)), valerate (Vanderwinkel et al., Biochem. Biophys. Res. Commun. 33:902-908 (1968)) and butanoate (Vanderwinkel et al., Biochem. Biophys. Res. Commun. 33:902-908 (1968)). Similar enzymes exist in Corynebacterium glutamicum ATCC 13032 (Duncan et al., 68:5186-5190 (2002)), Clostridium acetobutylicum (Cary et al., Appl Environ Microbiol 56:1576-1583 (1990); Wiesenborn et al., Appl Environ Microbiol 55:323-329 (1989)), and Clostridium saccharoperbutylacetonicum (Kosaka et al., Biosci. Biotechnol Biochem. 71:58-68 (2007)).

TABLE 26

| Gene | GI # | Accession No. | Organism |
|---|---|---|---|
| atoA | 2492994 | P76459.1 | Escherichia coli |
| atoD | 2492990 | P76458.1 | Escherichia coli |
| actA | 62391407 | YP_226809.1 | Corynebacterium glutamicum |
| cg0592 | 62389399 | YP_224801.1 | Corynebacterium glutamicum |
| ctfA | 15004866 | NP_149326.1 | Clostridium acetobutylicum |
| ctfB | 15004867 | NP_149327.1 | Clostridium acetobutylicum |
| ctfA | 31075384 | AAP42564.1 | Clostridium |
| | | | saccharoperbutylacetonicum |
| ctfB | 31075385 | AAP42565.1 | Clostridium |
| | | | saccharoperbutylacetonicum |

Acetate kinase (EC 2.7.2.1) can catalyzes the reversible ATP-dependent phosphorylation of acetate to acetylphosphate (FIG. 5C). Exemplary acetate kinase enzymes have been characterized in many organisms including E. coli, Clostridium acetobutylicum and Methanosarcina thermophila (Ingram-Smith et al., J. Bacteriol. 187:2386-2394 (2005); Fox and Roseman, J. Biol. Chem. 261:13487-13497 (1986); Winzer et al., Microbioloy 143 (Pt 10):3279-3286 (1997)). Acetate kinase activity has also been demonstrated in the gene product of E. coli purT (Marolewski et al., Biochemistry 33:2531-2537 (1994). Some butyrate kinase enzymes (EC 2.7.2.7), for example buk1 and buk2 from Clostridium acetobutylicum, also accept acetate as a substrate (Hartmanis, M. G., J. Biol. Chem. 262:617-621 (1987)). Homologs exist in several other organisms including Salmonella enterica and Chlamydomonas reinhardtii.

TABLE 27

| Protein | GenBank ID | GI Number | Organism |
|---|---|---|---|
| ackA | NP_416799.1 | 16130231 | Escherichia coli |
| Ack | AAB18301.1 | 1491790 | Clostridium acetobutylicum |
| Ack | AAA72042.1 | 349834 | Methanosarcina thermophila |
| purT | AAC74919.1 | 1788155 | Escherichia coli |
| buk1 | NP_349675 | 15896326 | Clostridium acetobutylicum |
| buk2 | Q97II1 | 20137415 | Clostridium acetobutylicum |
| ackA | NP_461279.1 | 16765664 | Salmonella typhimurium |
| ACK1 | XP_001694505.1 | 159472745 | Chlamydomonas reinhardtii |
| ACK2 | XP_001691682.1 | 159466992 | Chlamydomonas reinhardtii |

The formation of acetyl-CoA from acety-lphosphate can be catalyzed by phosphotransacetylase (EC 2.3.1.8) (FIG. 5D). The pta gene from E. coli encodes an enzyme that reversibly converts acetyl-CoA into acetyl-phosphate (Suzuki, T., Biochim. Biophys. Acta 191:559-569 (969)). Additional acetyltransferase enzymes have been characterized in Bacillus subtilis (Rado and Hoch, Biochim. Biophys. Acta 321:114-125 (1973), Clostridium kluyveri (Stadtman, E., Methods Enzymol. 1:5896-599 (1955), and Thermotoga maritima (Bock et al., J. Bacteriol. 181:1861-1867 (1999)). This reaction can also be catalyzed by some phosphotranbutyrylase enzymes (EC 2.3.1.19), including the ptb gene products from Clostridium acetobutylicum (Wiesenborn et al., App. Environ. Microbiol. 55:317-322 (1989); Walter et al., Gene 134:107-111 (1993)). Additional ptb genes are found in butyrate-producing bacterium L2-50 (Louis et al., J. Bacteriol. 186:2099-2106 (2004) and Bacillus megaterium (Vazquez et al., Curr. Microbiol. 42:345-349 (2001). Homologs to the E. coli pta gene exist in several other organisms including Salmonella enterica and Chlamydomonas reinhardtii.

TABLE 28

| Protein | GenBank ID | GI Number | Organism |
|---|---|---|---|
| Pta | NP_416800.1 | 71152910 | Escherichia coli |
| Pta | P39646 | 730415 | Bacillus subtilis |
| Pta | A5N801 | 146346896 | Clostridium kluyveri |
| Pta | Q9X0L4 | 6685776 | Thermotoga maritime |
| Ptb | NP_349676 | 34540484 | Clostridium acetobutylicum |
| Ptb | AAR19757.1 | 38425288 | butyrate-producing bacterium L2-50 |
| Ptb | CAC07932.1 | 10046659 | Bacillus megaterium |
| Pta | NP_461280.1 | 16765665 | Salmonella enterica subsp. enterica |
| | | | serovar Typhimurium str. LT2 |
| PAT2 | XP_001694504.1 | 159472743 | Chlamydomonas reinhardtii |
| PAT1 | XP_001691787.1 | 159467202 | Chlamydomonas reinhardtii |

Pyruvate decarboxylase (PDC) is a key enzyme in alcoholic fermentation, catalyzing the decarboxylation of pyruvate to acetaldehyde. The PDC1 enzyme from Saccharomyces cerevisiae has been extensively studied (Killenberg-Jabs et al., Eur. J. Biochem. 268:1698-1704 (2001); Li et al., Biochemistry. 38:10004-10012 (1999); ter Schure et al., Appl. Environ. Microbiol. 64:1303-1307 (1998)). Other well-characterized PDC enzymes are found in Zymomonas mobilus (Siegert et al., Protein Eng Des Sel 18:345-357 (2005)), Acetobacter pasteurians (Chandra et al., 176:443-451 (2001)) and Kluyveromyces lactis (Krieger et al., 269:3256-3263 (2002)). The PDC1 and PDC5 enzymes of Saccharomyces cerevisiae are subject to positive transcriptional regulation by PDC2 (Hohmann et al, Mol Gen Genet 241:657-66 (1993)). Pyruvate decarboxylase activity is also possessed by a protein encoded by CTRG_03826 (GI:255729208) in Candida tropicalis, PDC1 (GI number: 1226007) in Kluyveromyces lactis, YALI0D10131g (GI:50550349) in Yarrowia lipolytica, PAS_chr3_0188 (GI:254570575) in Pichia pastoris, pyruvate decarboxylase (GI: GI:159883897) in Schizosaccharomyces pombe, ANI_1_1024084 (GI:145241548), ANI_1_796114 (GI:317034487), ANI_1_936024 (GI:317026934) and ANI_1_2276014 (GI:317025935) in Aspergillus niger.

TABLE 29

| | | GI | |
|---|---|---|---|
| Protein | GenBank ID | Number | Organism |
| pdc | P06672.1 | 118391 | Zymomonas mobilis |
| pdc1 | P06169 | 30923172 | Saccharomyces cerevisiae |
| Pdc2 | NP_010366.1 | 6320286 | Saccharomyces cerevisiae |
| Pdc5 | NP_013235.1 | 6323163 | Saccharomyces cerevisiae |
| CTRG_03826 | XP_002549529 | 255729208 | Candida tropicalis, |
| CU329670.1: | CAA90807 | 159883897 | Schizosaccharomyces |
| 585597.587312 | | | pombe |
| YALI0D10131g | XP_502647 | 50550349 | Yarrowia lipolytica |
| PAS_chr3_0188 | XP_002492397 | 254570575 | Pichia pastoris |
| pdc | Q8L388 | 20385191 | Acetobacter pasteurians |
| pdc1 | Q12629 | 52788279 | Kluyveromyces lactis |
| ANI_1_1024084 | XP_001393420 | 145241548 | Aspergillus niger |
| ANI_1_796114 | XP_001399817 | 317026934 | Aspergillus niger |
| ANI_1_936024 | XP_001396467 | 317034487 | Aspergillus niger |
| ANI_1_2276014 | XP_001388598 | 317025935 | Aspergillus niger |

Aldehyde dehydrogenase enzymes in EC class 1.2.1 catalyze the oxidation of acetaldehyde to acetate (FIG. 5F). Exemplary genes encoding this activity were described above. The oxidation of acetaldehyde to acetate can also be catalyzed by an aldehyde oxidase with acetaldehyde oxidase activity. Such enzymes can convert acetaldehyde, water and O₂ to acetate and hydrogen peroxide. Exemplary aldehyde oxidase enzymes that have been shown to catalyze this transformation can be found in Bos taurus and Mus musculus (Garattini et al., Cell Mol Life Sci 65:1019-48 (2008); Cabre et al., Biochem Soc Trans 15:882-3 (1987)). Additional aldehyde oxidase gene candidates include the two flavin- and molybdenum-containing aldehyde oxidases of Zea mays, encoded by zmAO-1 and zmAO-2 (Sekimoto et al., J Biol Chem 272:15280-85 (1997)).

TABLE 30

| Gene | GenBank Accession No. | GI No. | Organism |
|---|---|---|---|
| zmAO-1 | NP_001105308.1 | 162458742 | Zea mays |
| zmAO-2 | BAA23227.1 | 2589164 | Zea mays |
| Aox1 | O54754.2 | 20978408 | Mus musculus |
| XDH | DAA24801.1 | 296482686 | Bos taurus |

Pyruvate oxidase (acetyl-phosphate forming) can catalyze the conversion of pyruvate, oxygen and phosphate to acetyl-phosphate and hydrogen peroxide (FIG. 5G). This type of pyruvate oxidase is soluble and requires the cofactors thiamin diphosphate and flavin adenine dinucleotide (FAD). Acetyl-phosphate forming pyruvate oxidase enzymes can be found in lactic acid bacteria Lactobacillus delbrueckii and Lactobacillus plantarum (Lorquet et al., J Bacteriol 186:3749-3759 (2004); Hager et al., Fed Proc 13:734-38 (1954)). A crystal structure of the L. plantarum enzyme has been solved (Muller et al., (1994)). In Streptococcus sanguinis and Streptococcus pneumonia, acetyl-phosphate forming pyruvate oxidase enzymes are encoded by the spxB gene (Spellerberg et al., Mol Micro 19:803-13 (1996); Ramos-Montanez et al., Mol Micro 67:729-46 (2008)). The SpxR was shown to positively regulate the transcription of spxB in S. pneumoniae (Ramos-Montanez et al., supra). A similar regulator in S. sanguinis was identified by sequence homology. Introduction or modification of catalase activity can reduce accumulation of the hydrogen peroxide product.

TABLE 31

| Gene | GenBank Accession No. | GI No. | Organism |
|---|---|---|---|
| poxB | NP_786788.1 | 28379896 | Lactobacillus plantarum |
| spxB | L39074.1 | 1161269 | Streptococcus pneumoniae |
| Spd_0969 | YP_816445.1 | 116517139 | Streptococcus pneumoniae |
| (spxR) | | | |
| spxB | ZP_07887723.1 | 315612812 | Streptococcus sanguinis |
| spxR | ZP_07887944.1 GI: | 315613033 | Streptococcus sanguinis |

The pyruvate dehydrogenase (PDH) complex can catalyze the conversion of pyruvate to acetyl-CoA (FIG. 5H). The E. coli PDH complex is encoded by the genes aceEF and lpdA. Enzyme engineering efforts have improved the E. coli PDH enzyme activity under anaerobic conditions (Kim et al., J. Bacteriol. 190:3851-3858 (2008); Kim et al., Appl. Environ. Microbiol. 73:1766-1771 (2007); Zhou et al., Biotechnol. Lett. 30:335-342 (2008)). In contrast to the E. coli PDH, the B. subtilis complex is active and required for growth under anaerobic conditions (Nakano et al., 179:6749-6755 (1997)). The Klebsiella pneumoniae PDH, characterized during growth on glycerol, is also active under anaerobic conditions (Menzel et al., 56:135-142 (1997)). Crystal structures of the enzyme complex from bovine kidney (Zhou et al., 98:14802-14807 (2001)) and the E2 catalytic domain from Azotobacter vinelandii are available (Mattevi et al., Science. 255:1544-1550 (1992)). Some mammalian PDH enzymes complexes can react on alternate substrates such as 2-oxobutanoate. Comparative kinetics of Rattus norvegicus PDH and BCKAD indicate that BCKAD has higher activity on 2-oxobutanoate as a substrate (Paxton et al., Biochem. J. 234:295-303 (1986)). The S. cerevisiae complex canconsist of an E2 (LAT1) core that binds E1 (PDA1, PDB1), E3 (LPD1), and Protein X (PDX1) components (Pronk et al., Yeast 12:1607-1633 (1996)).

TABLE 32

| Gene | Accession No. | GI Number | Organism |
|---|---|---|---|
| aceE | NP_414656.1 | 16128107 | Escherichia coli |
| aceF | NP_414657.1 | 16128108 | Escherichia coli |
| lpd | NP_414658.1 | 16128109 | Escherichia coli |
| pdhA | P21881.1 | 3123238 | Bacillus subtilis |
| pdhB | P21882.1 | 129068 | Bacillus subtilis |
| pdhC | P21883.2 | 129054 | Bacillus subtilis |
| pdhD | P21880.1 | 118672 | Bacillus subtilis |
| aceE | YP_001333808.1 | 152968699 | Klebsiella pneumonia |
| aceF | YP_001333809.1 | 152968700 | Klebsiella pneumonia |
| lpdA | YP_001333810.1 | 152968701 | Klebsiella pneumonia |
| Pdha1 | NP_001004072.2 | 124430510 | Rattus norvegicus |
| Pdha2 | NP_446446.1 | 16758900 | Rattus norvegicus |
| Dlat | NP_112287.1 | 78365255 | Rattus norvegicus |
| Dld | NP_955417.1 | 40786469 | Rattus norvegicus |
| LAT1 | NP_014328 | 6324258 | Saccharomyces cerevisiae |
| PDA1 | NP_011105 | 37362644 | Saccharomyces cerevisiae |
| PDB1 | NP_009780 | 6319698 | Saccharomyces cerevisiae |
| LPD1 | NP_116635 | 14318501 | Saccharomyces cerevisiae |
| PDX1 | NP_011709 | 6321632 | Saccharomyces cerevisiae |

As an alternative to the large multienzyme PDH complexes described above, some organisms utilize enzymes in the 2-ketoacid oxidoreductase family (OFOR) to catalyze acylating oxidative decarboxylation of 2-keto-acids. Unlike the dehydrogenase complexes, these enzymes contain iron-sulfur clusters, utilize different cofactors, and use ferredoxin or flavodixin as electron acceptors in lieu of NAD(P)H. Pyruvate ferredoxin oxidoreductase (PFOR) can catalyze the oxidation of pyruvate to form acetyl-CoA (FIG. 5H). The PFOR from Desulfovibrio africanus has been cloned and expressed in E. coli resulting in an active recombinant enzyme that was stable for several days in the presence of oxygen (Pieulle et al., J Bacteriol. 179:5684-5692 (1997)). Oxygen stability is relatively uncommon in PFORs and is believed to be conferred by a 60 residue extension in the polypeptide chain of the D. africanus enzyme. The M. thermoacetica PFOR is also well characterized (Menon et al., Biochemistry 36:8484-8494 (1997)) and was even shown to have high activity in the direction of pyruvate synthesis during autotrophic growth (Furdui et al., J Biol Chem. 275:28494-28499 (2000)). Further, E. coli possesses an uncharacterized open reading frame, ydbK, that encodes a protein that is 51% identical to the M. thermoacetica PFOR. Evidence for pyruvate oxidoreductase activity in E. coli has been described (Blaschkowski et al., Eur. J Biochem. 123:563-569 (1982)). Several additional PFOR enzymes are described in Ragsdale, Chem. Rev. 103:2333-2346 (2003). Finally, flavodoxin reductases (e.g., fqrB from Helicobacter pylori or Campylobacter jejuni (St Maurice et al., J. Bacteriol. 189:4764-4773 (2007))) or Rnf-type proteins (Seedorf et al., Proc. Natl. Acad. Sci. US.A. 105:2128-2133 (2008); Herrmann et al., J. Bacteriol. 190:784-791 (2008)) provide a means to generate NADH or NADPH from the reduced ferredoxin generated by PFOR. These proteins are identified below.

TABLE 33

| Protein | GenBank ID | GI Number | Organism |
|---|---|---|---|
| Por | CAA70873.1 | 1770208 | Desulfovibrio africanus |
| Por | YP_428946.1 | 83588937 | Moorella thermoacetica |
| ydbK | NP_415896.1 | 16129339 | Escherichia coli |
| fqrB | NP_207955.1 | 15645778 | Helicobacter pylori |
| fqrB | YP_001482096.1 | 157414840 | Campylobacter jejuni |
| RnfC | EDK33306.1 | 146346770 | Clostridium kluyveri |
| RnfD | EDK33307.1 | 146346771 | Clostridium kluyveri |
| RnfG | EDK33308.1 | 146346772 | Clostridium kluyveri |
| RnfE | EDK33309.1 | 146346773 | Clostridium kluyveri |
| RnfA | EDK33310.1 | 146346774 | Clostridium kluyveri |
| RnfB | EDK33311.1 | 146346775 | Clostridium kluyveri |

Pyruvate formate-lyase (PFL, EC 2.3.1.54) (FIG. 5H), encoded by pflB in E. coli, can convert pyruvate into acetyl-CoA and formate. The activity of PFL can be enhanced by an activating enzyme encoded by pflA (Knappe et al., Proc. Natl. Acad. Sci U.S.A 81:1332-1335 (1984); Wong et al., Biochemistry 32:14102-14110 (1993)). Keto-acid formate-lyase (EC 2.3.1.-), also known as 2-ketobutyrate formate-lyase (KFL) and pyruvate formate-lyase 4, is the gene product of tdcE in E. coli. This enzyme catalyzes the conversion of 2-ketobutyrate to propionyl-CoA and formate during anaerobic threonine degradation, and can also substitute for pyruvate formate-lyase in anaerobic catabolism (Simanshu et al., J Biosci. 32:1195-1206 (2007)). The enzyme is oxygen-sensitive and, like PflB, can require post-translational modification by PFL-AE to activate a glycyl radical in the active site (Hesslinger et al., Mol. Microbiol 27:477-492 (1998)). A pyruvate formate-lyase from Archaeglubus fulgidus encoded by pflD has been cloned, expressed in E. coli and characterized (Lehtio et al., Protein Eng Des Sel 17:545-552 (2004)). The crystal structures of the A. fulgidus and E. coli enzymes have been resolved (Lehtio et al., J Mol. Biol. 357:221-235 (2006); Leppanen et al., Structure. 7:733-744 (1999)). Additional PFL and PFL-AE candidates are found in Lactococcus lactis (Melchiorsen et al., Appl Microbiol Biotechnol 58:338-344 (2002)), and Streptococcus mutans (Takahashi-Abbe et al., Oral. Microbiol Immunol. 18:293-297 (2003)), Chlamydomonas reinhardtii (Hemschemeier et al., Eukaryot. Cell 7:518-526 (2008b); Atteia et al., J. Biol. Chem. 281:9909-9918 (2006)) and Clostridium pasteurianum (Weidner et al., J Bacteriol. 178:2440-2444 (1996)).

TABLE 34

| Protein | GenBank ID | GI Number | Organism |
|---|---|---|---|
| pflB | NP_415423 | 16128870 | Escherichia coli |
| pflA | NP_415422.1 | 16128869 | Escherichia coli |
| tdcE | AAT48170.1 | 48994926 | Escherichia coli |
| pflD | NP_070278.1 | 11499044 | Archaeglubus fulgidus |
| pfl | CAA03993 | 2407931 | Lactococcus lactis |
| pfl | BAA09085 | 1129082 | Streptococcus mutans |
| PFL1 | XP_001689719.1 | 159462978 | Chlamydomonas reinhardtii |
| pflA1 | XP_001700657.1 | 159485246 | Chlamydomonas reinhardtii |
| pfl | Q46266.1 | 2500058 | Clostridium pasteurianum |
| act | CAA63749.1 | 1072362 | Clostridium pasteurianum |

The NAD(P)⁺ dependent oxidation of acetaldehyde to acetyl-CoA (FIG. 5I) can be catalyzed by an acylating acetaldehyde dehydrogenase (EC 1.2.1.10). Acylating acetaldehyde dehydrogenase enzymes of E. coli are encoded by adhE, eutE, and mhpF (Ferrandez et al, J Bacteriol 179:2573-81 (1997)). The Pseudomonas sp. CF600 enzyme, encoded by dmpF, participates in meta-cleavage pathways and forms a complex with 4-hydroxy-2-oxovalerate aldolase (Shingler et al, J Bacteriol 174:711-24 (1992)). Solventogenic organisms such as Clostridium acetobutylicum encode bifunctional enzymes with alcohol dehydrogenase and acetaldehyde dehydrogenase activities. The bifunctional C. acetobutylicum enzymes are encoded by bdh I and adhE2 (Walter, et al., J. Bacteriol. 174:7149-7158 (1992); Fontaine et al., J. Bacteriol. 184:821-830 (2002)). Yet another candidate for acylating acetaldehyde dehydrogenase is the ald gene from Clostridium beijerinckii (Toth, Appl. Environ. Microbiol. 65:4973-4980 (1999). This gene is very similar to the eutE acetaldehyde dehydrogenase genes of Salmonella typhimurium and E. coli (Toth, Appl. Environ. Microbiol. 65:4973-4980 (1999).

TABLE 35

| Protein | GenBank ID | GI Number | Organism |
|---|---|---|---|
| adhE | NP_415757.1 | 16129202 | Escherichia coli |
| mhpF | NP_414885.1 | 16128336 | Escherichia coli |
| dmpF | CAA43226.1 | 45683 | Pseudomonas sp. CF600 |
| adhE2 | AAK09379.1 | 12958626 | Clostridium acetobutylicum |
| bdh I | NP_349892.1 | 15896543 | Clostridium acetobutylicum |
| Ald | AAT66436 | 49473535 | Clostridium beijerinckii |
| eutE | NP_416950 | 16130380 | Escherichia coli |
| eutE | AAA80209 | 687645 | Salmonella typhimurium |

Threonine aldolase (EC 4.1.2.5) catalyzes the cleavage of threonine to glycine and acetaldehyde (FIG. 5J). The Saccharomyces cerevisiae and Candida albicans enzymes are encoded by GLY1 (Liu et al, Eur J Biochem 245:289-93 (1997); McNeil et al, Yeast 16:167-75 (2000)). The ltaE and glyA gene products of E. coli also encode enzymes with this activity (Liu et al, Eur J Biochem 255:220-6 (1998)).

TABLE 36

| Protein | GenBank ID | GI Number | Organism |
|---|---|---|---|
| GLY1 | NP_010868.1 | 6320789 | Saccharomyces cerevisiae |
| GLY1 | AAB64198.1 | 2282060 | Candida albicans |
| ltaE | AAC73957.1 | 1787095 | Escherichia coli |
| glyA | AAC75604.1 | 1788902 | Escherichia coli |

### Example III

### Pathways for Increasing Cytosolic Acetyl-CoA from Mitochondrial and Peroxisomal Acetyl-CoA by Carnitine-Mediated Translocation

This example describes pathways for the carnitine-mediated translocation of acetyl-CoA from mitochondria and peroxisomes to the cytosol of a eukaryotic cell.

Acetyl-CoA is a key metabolic intermediate of biosynthetic and degradation pathways that take place in different cellular compartments. For example, during growth on sugars, the majority of acetyl-CoA is generated in the mitochondria, where it feeds into the TCA cycle. During growth on fatty acid substrates such as oleate, acetyl-CoA is formed in peroxisomes where the beta-oxidation degradation reactions take place. A majority of acetyl-CoA is produced in the cytosol during growth on two-carbon substrates such as ethanol or acetate. The transport of acetyl-CoA or acetyl units among cellular compartments is essential for enabling growth on different substrates.

One approach for increasing cytosolic acetyl-CoA is to modify the transport of acetyl-CoA or acetyl units among cellular compartments. Several mechanisms for transporting acetyl-CoA or acetyl units between cellular compartments are known in the art. For example, many eukaryotic organisms transport acetyl units using the carrier molecule carnitine (van Roermund et al., EMBO J 14:3480-86 (1995)). Acetyl-carnitine shuttles between cellular compartments have been characterized in yeasts such as Candida albicans (Strijbis et al, J Biol Chem 285:24335-46 (2010)). In these shuttles, the acetyl moiety of acetyl-CoA is reversibly transferred to carnitine by acetylcarnitine transferase enzymes. Acetylcarnitine can then be transported across the membrane by acetylcarnitine/carnitine translocase enzymes. After translocation, the acetyl-CoA can be regenerated by acetylcarnitine transferase.

Exemplary acetylcarnitine translocation pathways are depicted in FIG. 6. In one pathway, mitochondrial acetyl-CoA is converted to acetylcarnitine by a mitochondrial carnitine acetyltransferase (step A). Mitochondrial acetylcarnitine can then be translocated across the mitochondrial membrane into the cytosol by a mitochondrial acetylcarnitine translocase (step D). A cytosolic acetylcarnitine transferase regenerates acetyl-CoA (step C). Peroxisomal acetyl-CoA is converted to acetylcarnitine by a peroxisomal acetylcarnitine transferase (step B). Peroxisomal acetylcarnitine can then be translocated across the peroxisomal membrane into the cytosol by a peroxisomal acetylcarnitine translocase (step E), and then converted to cytosolic acetyl-CoA by a cytosolic acetylcarnitine transferase (step C).

While some yeast organisms such as Candida albicans synthesize carnitine de novo, others organisms such as Saccharomyces cerevisiae do not (van Roermund et al., EMBO J 18:5843-52 (1999)). Organisms unable to synthesize carnitine de novo can be supplied carnitine exogenously or can be engineered to express a one or more carnitine biosynthetic pathway enzymes, in addition to the acetyltransferases and translocases required for shuttling acetyl-CoA from cellular compartments to the cytoplasm. Carnitine biosynthetic pathways are known in the art. In Candida albicans, for example, carnitine is synthesized from trimethyl-L-lysine in four enzymatic steps (Strijbis et al., FASEB J 23:2349-59 (2009)).

Enzyme candidates for carnitine shuttle proteins and the carnitine biosynthetic pathway are described in further detail in below.

Carnitine acetyltransferase (CAT, EC 2.3.1.7) reversibly links acetyl units from acetyl-CoA to the carrier molecule, carnitine. Candida albicans encodes three CAT isozymes: Cat2, Yat1 and Yat2 (Strijbis et al., J Biol Chem 285:24335-46 (2010)). Cat2 is expressed in both the mitochondrion and the peroxisomes, whereas Yat1 and Yat2 are cytosolic. The Cat2 transcript contains two start codons that are regulated under different carbon source conditions. The longer transcript contains a mitochondrial targeting sequence whereas the shorter transcript is targeted to peroxisomes. Cat2 of Saccharomyces cerevisiae and AcuJ of Aspergillus nidulans employ similar mechanisms of dual localization (Elgersma et al., EMBO J 14:3472-9 (1995); Hynes et al., Euk Cell 10:547-55 (2011)). The cytosolic CAT of A. nidulans is encoded by facC. Other exemplary CAT enzymes are found in Rattus norvegicus and Homo sapiens (Cordente et al., Biochem 45:6133-41 (2006)). Exemplary carnitine acyltransferase enzymes (EC 2.3.1.21) are the Cpt1 and Cpt2 gene products of Rattus norvegicus (de Vries et al., Biochem 36:5285-92 (1997)).

TABLE 37

| Protein | Accession # | GI number | Organism |
|---|---|---|---|
| Cat2 | AAN31660.1 | 23394954 | Candida albicans |
| Yat1 | AAN31659.1 | 23394952 | Candida albicans |
| Yat2 | XP_711005.1 | 68490355 | Candida albicans |
| Cat2 | CAA88327.1 | 683665 | Saccharomyces cerevisiae |
| Yat1 | AAC09495.1 | 456138 | Saccharomyces cerevisiae |
| Yat2 | NP_010941.1 | 6320862 | Saccharomyces cerevisiae |
| AcuJ | CBF69795.1 | 259479509 | Aspergillus nidulans |
| FacC | AAC82487.1 | 2511761 | Aspergillus nidulans |
| Crat | AAH83616.1 | 53733439 | Rattus norvegicus |
| Crat | P43155.5 | 215274265 | Homo sapiens |
| Cpt1 | AAB48046.1 | 1850590 | Rattus norvegicus |
| Cpt2 | AAB02339.1 | 1374784 | Rattus norvegicus |

Carnitine-acetylcarnitine translocases can catalyze the bidirectional transport of carnitine and carnitine-fatty acid complexes. The Cact gene product provides a mechanism of transport across the mitochondrial membrane (Ramsay et al Biochim Biophys Acta 1546:21-42 (2001)). A similar protein has been studied in humans (Sekoguchi et al., J Biol Chem 278:38796-38802 (2003)). The Saccharomyces cerevisiae mitochondrial carnitine carrier is Crc1 (van Roermund et al., supra; Palmieri et al., Biochimica et Biophys Acta 1757:1249-62 (2006)). The human carnitine translocase was able to complement a Crc1-deficient strain of S. cerevisiae (van Roermund et al., supra). Two additional carnitine translocases found in Drosophila melanogaster and Caenorhabditis elegans were also able to complement Crc1-deficient yeast (Oey et al., Mol Genet Metab 85:121-24 (2005)). Four mitochondrial carnitine/acetylcarnitine carriers were identified in Trypanosoma brucei based on sequence homology to the yeast and human transporters (Colasante et al., Mol Biochem Parasit 167:104-117 (2009)). The carnitine transporter of Candida albicans was also identified by sequence homology. An additional mitochondrial carnitine transporter is the acuH gene product of Aspergillus nidulans, which is exclusively localized to the mitochondrial membrane (Lucas et al., FEMS Microbiol Lett 201:193-8 (2006)).

TABLE 38

| Protein | Accession # | GI number | Organism |
|---|---|---|---|
| Cact | P97521.1 | 2497984 | Rattus norvegicus |
| Cadl | NP_001034444.1 | 86198310 | Homo sapiens |
| CaO19.2851 | XP_715782.1 | 68480576 | Candida albicans |
| Crc1p | NP_014743.1 | 6324674 | Saccharomyces cerevisiae |
| Dif-1 | CAA88283.1 | 829102 | Caenorhabditis elegans |
| colt | CAA73099.1 | 1944534 | Drosophila melanogaster |
| Tb11.02.2960 | EAN79492.1 | 70833990 | Trypanosoma brucei |
| Tb11.03.0870 | EAN79007.1 | 70833505 | Trypanosoma brucei |
| Tb11.01.5040 | EAN80288.1 | 70834786 | Trypanosoma brucei |
| Tb927.8.5810 | AAX69329.1 | 62175181 | Trypanosoma brucei |
| acuH | CAB44434.1 | 5019305 | Aspergillus nidulans |

Transport of carnitine and acetylcarnitine across the peroxisomal membrane has not been well-characterized. Specific peroxisomal acetylcarnitine carrier proteins in yeasts have not been identified to date. It is possible that mitochonidrial carnitine translocases also function in the peroxisomal transport of carnitine and acetylcarnitine. Alternately, the peroxisomal membrane can be permeable to carnitine and acetylcarnitine. Experimental evidence suggests that the OCTN3 protein of Mus musculus is a peroxisomal carnitine/acylcarnitine transferase.

Yet another possibility is that acetyl-CoA or acetyl-carnitine is transported across the peroxisomal or mitochondrial membranes by an acetyl-CoA transporter such as the Pxa1 and Pxa2 ABC transporter of Saccharomyces cerevisiae or the ALDP ABC transporter of Homo sapiens (van Roermund et al., FASEB J 22:4201-8 (2008)). Pxa1 and Pxa2 form a heterodimeric complex in the peroxisomal membrane and transport long-chain acyl-CoA esters (Verleur et al., Eur J Biochem 249: 657-61 (1997)). The mutant phenotype of a pxa1/pxa2 deficient yeast can be rescued by heterologous expression of ALDP, which was shown to transport a range of acyl-CoA substrates van Roermund et al., FASEB J 22:4201-8 (2008)).

TABLE 39

| Protein | Accession # | GI number | Organism |
|---|---|---|---|
| OCTAT3 | BAA78343.1 | 4996131 | Mus musculus |
| Pxa1 | AAC49009.1 | 619668 | Saccharomyces cerevisiae |
| Pxa2 | AAB51597.1 | 1931633 | Saccharomyces cerevisiae |
| ALDP | NP_000024.2 | 7262393 | Homo sapiens |

The four step carnitine biosynthetic pathway of Candida albicans was recently characterized. The pathway precursor, trimethyllysine (TML), is produced during protein degradation. TML dioxygenase (CaO13.4316) hydroxylates TML to form 3-hydroxy-6-N-trimethyllysine. A pyridoxal-5′-phosphate dependent aldolase (CaO19.6305) then cleaves HTML into 4-trimethylaminobutyraldehyde. The 4-trimethylaminobutyraldehyde is subsequently oxidized to 4-trimethylaminobutyrate by a dehydrogenase (CaO19.6306). In the final step, 4-trimethylaminobutyrate is hydroxylated to form carnitine by the gene product of CaO19.7131. Flux through the carnitine biosynthesis pathway is limited by the availability of the pathway substrate and very low levels of carnitine seem to be sufficient for normal carnitine shuttle activity (Strejbis et al., IUBMB Life 62:357-62 (2010)).

TABLE 40

| Protein | Accession # | GI number | Organism |
|---|---|---|---|
| CaO19.4316 | XP_720623.1 | 68470755 | Candida albicans |
| CaO19.6305 | XP_711090.1 | 68490151 | Candida albicans |
| CaO19.6306 | XP_711091.1 | 68490153 | Candida albicans |
| CaO19.7131 | XP_715182.1 | 68481628 | Candida albicans |

Organisms unable to synthesize carnitine de novo can uptake carnitine from the growth medium. Uptake of carnitine can be achieved by expression of a carnitine transporter such as Agp2 of S. cerevisiae (van Roermund et al., supra).

TABLE 41

| Protein | Accession # | GI number | Organism |
|---|---|---|---|
| Agp2 | NP_009690.1 | 6319608 | Saccharomyces cerevisiae |

### Example IV

### Pathways for Producing 1,3-Butanediol from Acetyl-CoA

1,3-BDO production can be achieved by several alternative pathways as described in FIG. 4. All pathways first convert two molecules of acetyl-CoA into one molecule of acetoacetyl-CoA employing a thiolase. Acetoacetyl-CoA thiolase converts two molecules of acetyl-CoA into one molecule each of acetoacetyl-CoA and CoA (step A, FIG. 4). Exemplary acetoacetyl-CoA thiolase enzymes include the gene products of atoB from E. coli (Martin et al., Nat. Biotechnol. 21:796-802 (2003), thlA and thlB from C. acetobutylicum (Hanai et al., Appl. Environ. Microbiol. 73:7814-7818 (2007); Winzer et al., J. Mol. Microbiol. Biotechnol. 2:531-541 (2000), and ERG10 from S. cerevisiae (Hiser et al., J. Biol. Chem. 269:31383-31389 (1994). The acetoacetyl-CoA thiolase from Zoogloea ramigera is irreversible in the biosynthetic direction and a crystal structure is available (Merilainen et al, Biochem 48: 11011-25 (2009)).

TABLE 42

| Protein | GenBank ID | GI number | Organism |
|---|---|---|---|
| AtoB | NP_416728 | 16130161 | Escherichia coli |
| ThlA | NP_349476.1 | 15896127 | Clostridium acetobutylicum |
| ThlB | NP_149242.1 | 15004782 | Clostridium acetobutylicum |
| ERG10 | NP_015297 | 6325229 | Saccharomyces cerevisiae |
| phbA | P07097.4 | 135759 | Zoogloea ramigera |

Acetoacetyl-CoA reductase (step H, FIG. 4) catalyzing the reduction of acetoacetyl-CoA to 3-hydroxybutyryl-CoA participates in the acetyl-CoA fermentation pathway to butyrate in several species of Clostridia and has been studied in detail (Jones and Woods, Microbiol. Rev. 50:484-524 (1986)). The enzyme from Clostridium acetobutylicum, encoded by hbd, has been cloned and functionally expressed in E. coli (Youngleson et al., J Bacteriol. 171:6800-6807 (1989)). Additionally, subunits of two fatty acid oxidation complexes in E. coli, encoded by fadB and fadJ, function as 3-hydroxyacyl-CoA dehydrogenases (Binstockand Schulz, Methods Enzymol. 71 Pt C:403-411 (1981)). Yet other gene candidates demonstrated to reduce acetoacetyl-CoA to 3-hydroxybutyryl-CoA are phbB from Zoogloea ramigera (Ploux et al., Eur. Biochem. 174:177-182 (1988) and phaB from Rhodobacter sphaeroides (Alber et al., Mol. Microbiol. 61:297-309 (2006). The former gene candidate is NADPH-dependent, its nucleotide sequence has been determined (Peoples and Sinskey, Mol. Microbiol. 3:349-357 (1989) and the gene has been expressed in E. coli. Substrate specificity studies on the gene led to the conclusion that it could accept 3-oxopropionyl-CoA as a substrate besides acetoacetyl-CoA (Ploux et al., Eur. J Biochem. 174:177-182 (1988)). Additional gene candidates include Hbd1 (C-terminal domain) and Hbd2 (N-terminal domain) in Clostridium kluyveri (Hillmer and Gottschalk, Biochim. Biophys. Acta 3334:12-23 (1974)) and HSD17B10 in Bos taurus (Wakil et al., J Biol. Chem. 207:631-638 (1954)).

TABLE 43

| Protein | Genbank ID | GI number | Organism |
|---|---|---|---|
| fadB | P21177.2 | 119811 | Escherichia coli |
| fadJ | P77399.1 | 3334437 | Escherichia coli |
| Hbd2 | EDK34807.1 | 146348271 | Clostridium kluyveri |
| Hbd1 | EDK32512.1 | 146345976 | Clostridium kluyveri |
| Hbd | P52041.2 | | Clostridium acetobutylicum |
| HSD17B10 | O02691.3 | 3183024 | Bos Taurus |
| phbB | P23238.1 | 130017 | Zoogloea ramigera |
| phaB | YP_353825.1 | 77464321 | Rhodobacter sphaeroides |

A number of similar enzymes have been found in other species of Clostridia and in Metallosphaera sedula (Berg et al., Science 318:1782-1786 (2007).

TABLE 44

| Protein | GenBank ID | GI number | Organism |
|---|---|---|---|
| Hbd | NP_349314.1 | NP_349314.1 | Clostridium |
| | | | acetobutylicum |
| Hbd | AAM14586.1 | AAM14586.1 | Clostridium |
| | | | beijerinckii |
| Msed_1423 | YP_001191505 | YP_001191505 | Metallosphaera |
| | | | sedula |
| Msed_0399 | YP_001190500 | YP_001190500 | Metallosphaera |
| | | | sedula |
| Msed_0389 | YP_001190490 | YP_001190490 | Metallosphaera |
| | | | sedula |
| Msed_1993 | YP_001192057 | YP_001192057 | Metallosphaera |
| | | | sedula |

Several acyl-CoA dehydrogenases are capable of reducing an acyl-CoA to its corresponding aldehyde (Steps E, I, FIG. 4). Exemplary genes that encode such enzymes include the Acinetobacter calcoaceticus acr1 encoding a fatty acyl-CoA reductase (Reiser and Somerville, J. Bacteriol. 179:2969-2975 (1997), the Acinetobacter sp. M-1 fatty acyl-CoA reductase (Ishige et al., Appl. Environ. Microbiol. 68:1192-1195 (2002), and a CoA- and NADP-dependent succinate semialdehyde dehydrogenase encoded by the sucD gene in Clostridium kluyveri (Sohling and Gottschalk, J. Bacteriol. 178:871-880 (1996); Sohling and Gottschalk, J. Bacteriol. 1778:871-880 (1996)). SucD of P. gingivalis is another succinate semialdehyde dehydrogenase (Takahashi et al., J. Bacteriol. 182:4704-4710 (2000). The enzyme acylating acetaldehyde dehydrogenase in Pseudomonas sp, encoded by bphG, is yet another candidate as it has been demonstrated to oxidize and acylate acetaldehyde, propionaldehyde, butyraldehyde, isobutyraldehyde and formaldehyde (Powlowski et al., J. Bacteriol. 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya et al., J. Gen. Appl. Microbiol. 18:45-55 (1972); Koo et al., Biotechnol. Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol. Biochem. 71:58-68 (2007)). Additional aldehyde dehydrogenase enzyme candidates are found in Desulfatibacillum alkenivorans, Citrobacter koseri, Salmonella enterica, Lactobacillus brevis and Bacillus selenitireducens.

TABLE 45

| Protein | GenBank ID | GI number | Organism |
|---|---|---|---|
| acr1 | YP_047869.1 | 50086355 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| Bld | AAP42563.1 | 31075383 | Clostridium saccharoperbutylacetonicum |
| Ald | ACL06658.1 | 218764192 | Desulfatibacillum alkenivorans AK-01 |
| Ald | YP_001452373 | 157145054 | Citrobacter koseri ATCC BAA-895 |
| pduP | NP_460996.1 | 16765381 | Salmonella enterica Typhimurium |
| pduP | ABJ64680.1 | 116099531 | Lactobacillus brevis ATCC 367 |
| BselDRAFT_1651 | ZP_02169447 | 163762382 | Bacillus selenitireducens MLS10 |

An additional enzyme type that converts an acyl-CoA to its corresponding aldehyde is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archaeal bacteria (Berg et al., Science 318:1782-1786 (2007); Thauer, Science 318:1732-1733 (2007)). The enzyme utilizes NADPH as a cofactor and has been characterized in Metallosphaera and Sulfolobus spp (Alber et al., J. Bacteriol. 188:8551-8559 (2006); Hugler et al., J. Bacteriol. 184:2404-2410 (2002)). The enzyme is encoded by Msed_0709 in Metallosphaera sedula (Alber et al., supra (2006); Berg et al., Science 318:1782-1786 (2007)). A gene encoding a malonyl-CoA reductase from Sulfolobus tokodaii was cloned and heterologously expressed in E. coli (Alber et al., J. Bacteriol. 188:8551-8559 (2006)). This enzyme has also been shown to catalyze the conversion of methylmalonyl-CoA to its corresponding aldehyde (WO 2007/141208 (2007)). Although the aldehyde dehydrogenase functionality of these enzymes is similar to the bifunctional dehydrogenase from Chloroflexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius and have been listed below. Yet another candidate for CoA-acylating aldehyde dehydrogenase is the ald gene from Clostridium beijerinckii (Toth et al., Appl. Environ. Microbiol. 65:4973-4980 (1999). This enzyme has been reported to reduce acetyl-CoA and butyryl-CoA to their corresponding aldehydes. This gene is very similar to eutE that encodes acetaldehyde dehydrogenase of Salmonella typhimurium and E. coli (Toth et al., supra).

TABLE 46

| Protein | GenBank ID | GI number | Organism |
|---|---|---|---|
| Msed_0709 | YP_001190808.1 | 146303492 | Metallosphaera sedula |
| Mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus |
| | | | acidocaldarius |
| Ald | AAT66436 | 9473535 | Clostridium beijerinckii |
| eutE | AAA80209 | 687645 | Salmonella typhimurium |
| eutE | P77445 | 2498347 | Escherichia coli |

Exemplary genes encoding enzymes that catalyze the conversion of an aldehyde to alcohol (i.e., alcohol dehydrogenase or equivalently aldehyde reductase) (steps C and G of FIG. 4) include alrA encoding a medium-chain alcohol dehydrogenase for C2-C14 (Tani et al., Appl. Environ. Microbiol., 66:5231-5235 (2000)), ADH2 from Saccharomyces cerevisiae (Atsumi et al., Nature, 451:86-89 (2008)), yqhD from E. coli which has preference for molecules longer than C3 (Sulzenbacher et al., J. of Molecular Biology, 342:489-502 (2004)), and bdh I and bdh II from C. acetobutylicum which converts butyraldehyde into butanol (Walter et al., J. of Bacteriology, 174:7149-7158 (1992)). The gene product of yqhD catalyzes the reduction of acetaldehyde, malondialdehyde, propionaldehyde, butyraldehyde, and acrolein using NADPH as the cofactor (Perez et al., J. Biol. Chem., 283:7346-7353 (2008)). The adhA gene product from Zymomonas mobilis has been demonstrated to have activity on a number of aldehydes including formaldehyde, acetaldehyde, propionaldehyde, butyraldehyde, and acrolein (Kinoshita et al., Appl. Microbiol. Biotechnol, 22:249-254 (1985)). Additional aldehyde reductase candidates are encoded by bdh in C. saccharoperbutylacetonicum and Cbei_1722, Cbei_2181 and Cbei_2421 in C. beijerinckii.

TABLE 47

| Protein | GenBank ID | GI number | Organism |
|---|---|---|---|
| alrA | BAB12273.1 | 9967138 | Acinetobacter sp. strain M-1 |
| ADH2 | NP_014032.1 | 6323961 | Saccharomyces cerevisiae |
| yqhD | NP_417484.1 | 16130909 | Escherichia coli |
| bdh I | NP_349892.1 | 15896543 | Clostridium acetobutylicum |
| bdh II | NP_349891.1 | 15896542 | Clostridium acetobutylicum |
| adhA | YP_162971.1 | 56552132 | Zymomonas mobilis |
| bdh | BAF45463.1 | 124221917 | Clostridium saccharoperbutylacetonicum |
| Cbei_1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |

Enzymes exhibiting 4-hydroxybutyraldehyde reductase activity (EC 1.1.1.61) also fall into this category. Such enzymes have been characterized in Ralstonia eutropha (Bravo et al., J. Forensic Sci., 49:379-387 (2004)), Clostridium kluyveri (Wolff et al., Protein Expr. Purif., 6:206-212 (1995)) and Arabidopsis thaliana (Breitkreuz et al., J. Biol. Chem., 278:41552-41556 (2003)). Yet another gene is the alcohol dehydrogenase; adhI from Geobacillus thermoglucosidasius (Jeon et al., J. Biotechnol., 135:127-133 (2008)).

TABLE 48

| Protein | GenBank ID | GI number | Organism |
|---|---|---|---|
| 4hbd | YP_726053.1 | 113867564 | Ralstonia eutropha H16 |
| 4hbd | L21902.1 | 146348486 | Clostridium kluyveri DSM 555 |
| 4hbd | Q94B07 | 75249805 | Arabidopsis thaliana |
| adhI | AAR91477.1 | 40795502 | Geobacillus |
| | | | thermoglucosidasius M10EXG |

Another exemplary enzyme is 3-hydroxyisobutyrate dehydrogenase which catalyzes the reversible oxidation of 3-hydroxyisobutyrate to methylmalonate semialdehyde. This enzyme participates in valine, leucine and isoleucine degradation and has been identified in bacteria, eukaryotes, and mammals. The enzyme encoded by P84067 from Thermus thermophilus HB8 has been structurally characterized (Lokanath et al., J. Mol. Biol., 352:905-917 (2005)). The reversibility of the human 3-hydroxyisobutyrate dehydrogenase was demonstrated using isotopically-labeled substrate (Manning et al., Biochem J., 231:481-484 (1985)). Additional genes encoding this enzyme include 3hidh in Homo sapiens (Hawes et al., Methods Enzymol, 324:218-228 (2000)) and Oryctolagus cuniculus (Hawes et al., supra; Chowdhury et al., Biosci. Biotechnol Biochem., 60:2043-2047 (1996)), mmsB in Pseudomonas aeruginosa and Pseudomonas putida (Liao et al., US patent 20050221466), and dhat in Pseudomonas putida (Aberhart et al., J. Chem. Soc., 6:1404-1406 (1979); Chowdhury et al., supra; Chowdhury et al., Biosci. Biotechnol Biochem., 67:438-441 (2003)).

TABLE 49

| Protein | GenBank ID | GI number | Organism |
|---|---|---|---|
| P84067 | P84067 | 75345323 | Thermus thermophilus |
| 3hidh | P31937.2 | 12643395 | Homo sapiens |
| 3hidh | P32185.1 | 416872 | Oryctolagus cuniculus |
| mmsB | P28811.1 | 127211 | Pseudomonas aeruginosa |
| mmsB | NP_746775.1 | 26991350 | Pseudomonas putida |
| dhat | Q59477.1 | 2842618 | Pseudomonas putida |

Exemplary two-step oxidoreductases that convert an acyl-CoA to alcohol (e.g., steps B and J of FIG. 4) include those that transform substrates such as acetyl-CoA to ethanol (e.g., adhE from E. coli (Kessler et al., FEBS Lett. 281:59-63 (1991)) and butyryl-CoA to butanol (e.g. adhE2 from C. acetobutylicum (Fontaine et al., J. Bacteriol. 184:821-830 (2002)). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya et al., J. Gen. Appl. Microbiol. 18:43-55 (1972); Koo et al., Biotechnol. Lett. 27:505-510 (2005)).

TABLE 50

| Protein | GenBank ID | GI number | Organism |
|---|---|---|---|
| adhE | NP_415757.1 | 16129202 | Escherichia coli |
| adhE2 | AAK09379.1 | 12958626 | Clostridium acetobutylicum |
| adhE | AAV66076.1 | 55818563 | Leuconostoc mesenteroides |

Another exemplary enzyme can convert malonyl-CoA to 3-HP. An NADPH-dependent enzyme with this activity has characterized in Chloroflexus aurantiacus where it participates in the 3-hydroxypropionate cycle (Hugler et al., J. Bacteriol. 184:2404-2410 (2002); Strauss and Fuchs, Eur. J. Biochem. 215:633-643 (1993). This enzyme, with a mass of 300 kDa, is highly substrate-specific and shows little sequence similarity to other known oxidoreductases (Hugler, supra (2002)). No enzymes in other organisms have been shown to catalyze this specific reaction; however there is bioinformatic evidence that other organisms can have similar pathways (Klatt et al., Environ. Microbiol. 9:2067-2078 (2007)). Enzyme candidates in other organisms including Roseiflexus castenholzii, Erythrobacter sp. NAP1 and marine gamma proteobacterium HTCC2080 can be inferred by sequence similarity.

TABLE 51

| Protein | GenBank ID | GI number | Organism |
|---|---|---|---|
| Rcas_2929 | YP_001433009.1 | 156742880 | Roseiflexus |
| | | | castenholzii |
| NAP1_02720 | ZP_01039179.1 | 85708113 | Erythrobacter |
| | | | sp. NAP1 |
| MGP2080_00535 | ZP_01626393.1 | 119504313 | marine gamma |
| | | | proteobacterium |
| | | | HTCC2080 |

Longer chain acyl-CoA molecules can be reduced by enzymes such as the jojoba (Simmondsia chinensis) FAR which encodes an alcohol-forming fatty acyl-CoA reductase. Its overexpression in E. coli resulted in FAR activity and the accumulation of fatty alcohol (Metz et al., Plant Physiol. 122:635-644 (2000)).

TABLE 52

| Protein | GenBank ID | GI number | Organism |
|---|---|---|---|
| FAR | AAD38039.1 | 5020215 | Simmondsia chinensis |

There exist several exemplary alcohol dehydrogenases that convert a ketone to a hydroxyl functional group (e.g., steps D, F and O of FIG. 4). Two such enzymes from E. coli are encoded by malate dehydrogenase (mdh) and lactate dehydrogenase (ldhA). In addition, lactate dehydrogenase from Ralstonia eutropha has been shown to demonstrate high activities on substrates of various chain lengths such as lactate, 2-oxobutyrate, 2-oxopentanoate and 2-oxoglutarate (Steinbuchel and Schlegel, Eur. J. Biochem. 130:329-334 (1983)). Conversion of the oxo functionality to the hydroxyl group can also be catalyzed by 2-keto 1,3-BDO reductase, an enzyme reported to be found in rat and in human placenta (Suda et al., Arch. Biochem. Biophys. 176:610-620 (1976); Suda et al., Biochem. Biophys. Res. Commun. 77:586-591 (1977)). An additional candidate for these steps is the mitochondrial 3-hydroxybutyrate dehydrogenase (bdh) from the human heart which has been cloned and characterized (Marks et al., J. Biol. Chem. 267:15459-15463 (1992)).

TABLE 53

| Protein | GenBank ID | GI number | Organism |
|---|---|---|---|
| Mdh | AAC76268.1 | 1789632 | Escherichia coli |
| ldhA | NP_415898.1 | 16129341 | Escherichia coli |
| Ldh | YP_725182.1 | 113866693 | Ralstonia eutropha |
| Bdh | AAA58352.1 | 177198 | Homo sapiens |

Additional exemplary enzymes can be found in Rhodococcus ruber (Kosjek et al., Biotechnol Bioeng. 86:55-62 (2004)) and Pyrococcus furiosus (van der et al., Eur. J. Biochem. 268:3062-3068 (2001)). For example, secondary alcohol dehydrogenase enzymes capable of this transformation include adh from C. beijerinckii (Hanai et al., Appl Environ Microbiol 73:7814-7818 (2007); Jojima et al., Appl Microbiol Biotechnol 77:1219-1224 (2008)) and adh from Thermoanaerobacter brockii (Hanai et al., Appl Environ Microbiol 73:7814-7818 (2007); Peretz et al., Anaerobe 3:259-270 (1997)). The cloning of the bdhA gene from Rhizobium (Sinorhizobium) Meliloti into E. coli conferred the ability to utilize 3-hydroxybutyrate as a carbon source (Aneja and Charles, J. Bacteriol. 181(3):849-857 (1999)). Additional candidates can be found in Pseudomonas fragi (Ito et al., J. Mol. Biol. 355(4) 722-733 (2006)) and Ralstonia pickettii (Takanashi et al., Antonie van Leeuwenoek, 95(3):249-262 (2009)). Information related to these proteins and genes is shown below.

TABLE 54

| Protein | GenBank ID | GI number | Organism |
|---|---|---|---|
| Sadh | CAD36475 | 21615553 | Rhodococcus rubber |
| AdhA | AAC25556 | 3288810 | Pyrococcus furiosus |
| Adh | P14941.1 | 113443 | Thermoanaerobobacter |
| | | | brockii |
| Adh | AAA23199.2 | 60592974 | Clostridium beijerinckii |
| BdhA | NP_437676.1 | 16264884 | Rhizobium (Sinorhizobium) |
| | | | Meliloti |
| PRK13394 | BAD86668.1 | 57506672 | Pseudomonas fragi |
| Bdh1 | BAE72684.1 | 84570594 | Ralstonia pickettii |
| Bdh2 | BAE72685.1 | 84570596 | Ralstonia pickettii |
| Bdh3 | BAF91602.1 | 158937170 | Ralstonia pickettii |

Acetoacetyl-CoA:acetyl-CoA transferase (i.e., step K, FIG. 4) naturally converts acetoacetyl-CoA and acetate to acetoacetate and acetyl-CoA. This enzyme can also accept 3-hydroxybutyryl-CoA as a substrate or could be engineered to do so (i.e., step M, FIG. 4). Exemplary enzymes include the gene products of atoAD from E. coli (Hanai et al., Appl Environ Microbiol 73:7814-7818 (2007)), ctfAB from C. acetobutylicum (Jojima et al., Appl Microbiol Biotechnol 77:1219-1224 (2008)), and ctfAB from Clostridium saccharoperbutylacetonicum (Kosaka et al., Biosci. Biotechnol Biochem. 71:58-68 (2007)). Information related to these proteins and genes is shown below.

TABLE 55

| Protein | GenBank ID | GI number | Organism |
|---|---|---|---|
| AtoA | P76459.1 | 2492994 | Escherichia coli |
| AtoD | P76458.1 | 2492990 | Escherichia coli |
| CtfA | NP_149326.1 | 15004866 | Clostridium acetobutylicum |
| CtfB | NP_149327.1 | 15004867 | Clostridium acetobutylicum |
| CtfA | AAP42564.1 | 31075384 | Clostridium |
| | | | saccharoperbutylacetonicum |
| CtfB | AAP42565.1 | 31075385 | Clostridium |
| | | | saccharoperbutylacetonicum |

Succinyl-CoA:3-ketoacid-CoA transferase naturally converts succinate to succinyl-CoA while converting a 3-ketoacyl-CoA to a 3-ketoacid. Exemplary succinyl-CoA:3:ketoacid-CoA transferases are present in Helicobacter pylori (Corthesy-Theulaz et al., J. Biol. Chem. 272:25659-25667 (1997)), Bacillus subtilis (Stols et al., Protein. Expr. Purif. 53:396-403 (2007)), and Homo sapiens (Fukao et al., Genomics 68:144-151 (2000); Tanaka et al., Mol. Hum. Reprod. 8:16-23 (2002)). Information related to these proteins and genes is shown below.

TABLE 56

| Protein | GenBank ID | GI number | Organism |
|---|---|---|---|
| HPAG1_0676 | YP_627417 | 108563101 | Helicobacter pylori |
| HPAG1_0677 | YP_627418 | 108563102 | Helicobacter pylori |
| ScoA | NP_391778 | 16080950 | Bacillus subtilis |
| ScoB | NP_391777 | 16080949 | Bacillus subtilis |
| OXCT1 | NP_000427 | 4557817 | Homo sapiens |
| OXCT2 | NP_071403 | 11545841 | Homo sapiens |

Additional suitable acetoacetyl-CoA and 3-hydroxybutyryl-CoA transferases are encoded by the gene products of cat1, cat2, and cat3 of Clostridium kluyveri. These enzymes have been shown to exhibit succinyl-CoA, 4-hydroxybutyryl-CoA, and butyryl-CoA acetyltransferase activity, respectively (Seedorf et al., Proc. Natl. Acad. Sci. USA 105:2128-2133 (2008); Sohling and Gottschalk, J Bacteriol 178:871-880 (1996)). Similar CoA transferase activities are also present in Trichomonas vaginalis (van Grinsven et al., J. Biol. Chem. 283:1411-1418 (2008)) and Trypanosoma brucei (Riviere et al., J Biol. Chem. 279:45337-45346 (2004)). Yet another transferase capable of the desired conversions is butyryl-CoA:acetoacetate CoA-transferase. Exemplary enzymes can be found in Fusobacterium nucleatum (Barker et al., J. Bacteriol. 152(1):201-7 (1982)), Clostridium SB4 (Barker et al., J Biol. Chem. 253(4):1219-25 (1978)), and Clostridium acetobutylicum (Wiesenborn et al., Appl. Environ. Microbiol. 55(2):323-9 (1989)). Although specific gene sequences were not provided for butyryl-CoA:acetoacetate CoA-transferase in these references, the genes FN0272 and FN0273 have been annotated as a butyrate-acetoacetate CoA-transferase (Kapatral et al., J. Bact. 184(7) 2005-2018 (2002)). Homologs in Fusobacterium nucleatum such as FN1857 and FN1856 also likely have the desired acetoacetyl-CoA transferase activity. FN1857 and FN1856 are located adjacent to many other genes involved in lysine fermentation and are thus very likely to encode an acetoacetate:butyrate CoA transferase (Kreimeyer, et al., J Biol. Chem. 282 (10) 7191-7197 (2007)). Additional candidates from Porphyrmonas gingivalis and Thermoanaerobacter tengcongensis can be identified in a similar fashion (Kreimeyer, et al., J Biol. Chem. 282 (10) 7191-7197 (2007)). Information related to these proteins and genes is shown below.

TABLE 57

| Protein | GenBank ID | GI number | Organism |
|---|---|---|---|
| Cat1 | P38946.1 | 729048 | Clostridium kluyveri |
| Cat2 | P38942.2 | 1705614 | Clostridium kluyveri |
| Cat3 | EDK35586.1 | 146349050 | Clostridium kluyveri |
| TVAG_395550 | XP_001330176 | 123975034 | Trichomonas vaginalis G3 |
| Tb11.02.0290 | XP_828352 | 71754875 | Trypanosoma brucei |
| FN0272 | NP_603179.1 | 19703617 | Fusobacterium nucleatum |
| FN0273 | NP_603180.1 | 19703618 | Fusobacterium nucleatum |
| FN1857 | NP_602657.1 | 19705162 | Fusobacterium nucleatum |
| FN1856 | NP_602656.1 | 19705161 | Fusobacterium nucleatum |
| PG1066 | NP_905281.1 | 34540802 | Porphyromonas gingivalis W83 |
| PG1075 | NP_905290.1 | 34540811 | Porphyromonas gingivalis W83 |
| TTE0720 | NP_622378.1 | 20807207 | Thermoanaerobacter |
| | | | tengcongensis MB4 |
| TTE0721 | NP_622379.1 | 20807208 | Thermoanaerobacter |
| | | | tengcongensis MB4 |

Acetoacetyl-CoA can be hydrolyzed to acetoacetate by acetoacetyl-CoA hydrolase (step K, FIG. 4). Similarly, 3-hydroxybutyryl-CoA can be hydrolyzed to 3-hydroxybutyate by 3-hydroxybutyryl-CoA hydrolase (step M, FIG. 4). Many CoA hydrolases (EC 3.1.2.1) have broad substrate specificity and are suitable enzymes for these transformations either naturally or following enzyme engineering. Though the sequences were not reported, several acetoacetyl-CoA hydrolases were identified in the cytosol and mitochondrion of the rat liver (Aragon and Lowenstein, J. Biol. Chem. 258(8):4725-4733 (1983)). Additionally, an enzyme from Rattus norvegicus brain (Robinson et al., Biochem. Biophys. Res. Commun. 71:959-965 (1976)) can react with butyryl-CoA, hexanoyl-CoA and malonyl-CoA. The acot12 enzyme from the rat liver was shown to hydrolyze C2 to C6 acyl-CoA molecules (Suematsu et al., Eur. J. Biochem. 268:2700-2709 (2001)). Though its sequence has not been reported, the enzyme from the mitochondrion of the pea leaf showed activity on acetyl-CoA, propionyl-CoA, butyryl-CoA, palmitoyl-CoA, oleoyl-CoA, succinyl-CoA, and crotonyl-CoA (Zeiher and Randall, Plant. Physiol. 94:20-27 (1990)). Additionally, a glutaconate CoA-transferase from Acidaminococcus fermentans was transformed by site-directed mutagenesis into an acyl-CoA hydrolase with activity on glutaryl-CoA, acetyl-CoA and 3-butenoyl-CoA (Mack and Buckel, FEBS Lett. 405:209-212 (1997)). This indicates that the enzymes encoding succinyl-CoA:3-ketoacid-CoA transferases and acetoacetyl-CoA:acetyl-CoA transferases can also be used as hydrolases with certain mutations to change their function. The acetyl-CoA hydrolase, ACH1, from S. cerevisiae represents another candidate hydrolase (Buu et al., J. Biol. Chem. 278:17203-17209 (2003)). Information related to these proteins and genes is shown below.

TABLE 58

| Protein | GenBank ID | GI number | Organism |
|---|---|---|---|
| Acot12 | NP_570103.1 | 18543355 | Rattus norvegicus |
| GctA | CAA57199 | 559392 | Acidaminococcus fermentans |
| GctB | CAA57200 | 559393 | Acidaminococcus fermentans |
| ACH1 | NP_009538 | 6319456 | Saccharomyces cerevisiae |

Another hydrolase is the human dicarboxylic acid thioesterase, acot8, which exhibits activity on glutaryl-CoA, adipyl-CoA, suberyl-CoA, sebacyl-CoA, and dodecanedioyl-CoA (Westin et al., J. Biol. Chem. 280:38125-38132 (2005)) and the closest E. coli homolog, tesB, which can also hydrolyze a broad range of CoA thioesters (Naggert et al., J. Biol. Chem. 266:11044-11050 (1991)) including 3-hydroxybutyryl-CoA (Tseng et al., Appl. Environ. Microbiol. 75(10):3137-3145 (2009)). A similar enzyme has also been characterized in the rat liver (Deana, Biochem. Int. 26:767-773 (1992)). Other potential E. coli thioester hydrolases include the gene products of tesA (Bonner and Bloch, J. Biol. Chem. 247:3123-3133 (1972)), ybgC (Kuznetsova et al., FEMS Microbiol. Rev. 29:263-279 (2005); Zhuang et al., FEBS Lett. 516:161-163 (2002)), paaI (Song et al., J. Biol. Chem. 281:11028-11038 (2006)), and ybdB (Leduc et al., J. Bacteriol. 189:7112-7126 (2007)). Information related to these proteins and genes is shown below.

TABLE 59

| Protein | GenBank ID | GI number | Organism |
|---|---|---|---|
| Acot8 | CAA15502 | 3191970 | Homo sapiens |
| TesB | NP_414986 | 16128437 | Escherichia coli |
| Acot8 | NP_570112 | 51036669 | Rattus norvegicus |
| TesA | NP_415027 | 16128478 | Escherichia coli |
| YbgC | NP_415264 | 16128711 | Escherichia coli |
| PaaI | NP_415914 | 16129357 | Escherichia coli |
| YbdB | NP_415129 | 16128580 | Escherichia coli |

Additional hydrolase enzymes include 3-hydroxyisobutyryl-CoA hydrolase which has been described to efficiently catalyze the conversion of 3-hydroxyisobutyryl-CoA to 3-hydroxyisobutyrate during valine degradation (Shimomura et al., J. Biol. Chem. 269:14248-14253 (1994)). Genes encoding this enzyme include hibch of Rattus norvegicus (Shimomura et al., supra (1994); Shimomura et al., Methods Enzymol. 324:229-240 (2000)) and Homo sapiens (Shimomura et al., supra (1994). Candidate genes by sequence homology include hibch of Saccharomyces cerevisiae and BC 2292 of Bacillus cereus. BC_2292 was shown to demonstrate 3-hydroxybutyryl-CoA hydrolase activity and function as part of a pathway for 3-hydroxybutyrate synthesis when engineered into Escherichia coli (Lee et al., Appl. Microbiol. Biotechnol. 79:633-641 (2008)). Information related to these proteins and genes is shown below.

TABLE 60

| Protein | GenBank ID | GI number | Organism |
|---|---|---|---|
| Hibch | Q5XIE6.2 | 146324906 | Rattus norvegicus |
| Hibch | Q6NVY1.2 | 146324905 | Homo sapiens |
| Hibch | P28817.2 | 2506374 | Saccharomyces cerevisiae |
| BC_2292 | AP09256 | 29895975 | Bacillus cereus ATCC 14579 |

An alternative method for removing the CoA moiety from acetoacetyl-CoA or 3-hydroxybutyryl-CoA (steps K and M of FIG. 4) is to apply a pair of enzymes such as a phosphate-transferring acyltransferase and a kinase to impart acetoacetyl-CoA or 3-hydroxybutyryl-CoA synthetase activity. This activity enables the net hydrolysis of the CoA-ester of either molecule with the simultaneous generation of ATP. For example, the butyrate kinase (buk)/phosphotransbutyrylase (ptb) system from Clostridium acetobutylicum has been successfully applied to remove the CoA group from 3-hydroxybutyryl-CoA when functioning as part of a pathway for 3-hydroxybutyrate synthesis (Tseng et al., Appl. Environ. Microbiol. 75(10):3137-3145 (2009)). Specifically, the ptb gene from C. acetobutylicum encodes an enzyme that can convert an acyl-CoA into an acyl-phosphate (Walter et al. Gene 134(1): p. 107-11 (1993)); Huang et al. J Mol Microbiol Biotechnol 2(1): p. 33-38 (2000). Additional ptb genes can be found in butyrate-producing bacterium L2-50 (Louis et al. J. Bacteriol. 186:2099-2106 (2004)) and Bacillus megaterium (Vazquez et al. Curr. Microbiol 42:345-349 (2001)). Additional exemplary phosphate-transferring acyltransferases include phosphotransacetylase, encoded by pta. The pta gene from E. coli encodes an enzyme that can convert acetyl-CoA into acetyl-phosphate, and vice versa (Suzuki, T. Biochim. Biophys. Acta 191:559-569 (1969)). This enzyme can also utilize propionyl-CoA instead of acetyl-CoA forming propionate in the process (Hesslinger et al. Mol. Microbiol 27:477-492 (1998)). Information related to these proteins and genes is shown below.

TABLE 61

| Protein | GenBank ID | GI number | Organism |
|---|---|---|---|
| Pta | NP_416800.1 | 16130232 | Escherichia coli |
| Ptb | NP_349676 | 15896327 | Clostridium acetobutylicum |
| Ptb | AAR19757.1 | 38425288 | butyrate-producing bacterium |
| | | | L2-50 |
| Ptb | CAC07932.1 | 10046659 | Bacillus megaterium |

Exemplary kinases include the E. coli acetate kinase, encoded by ackA (Skarstedt and Silverstein J. Biol. Chem. 251:6775-6783 (1976)), the C. acetobutylicum butyrate kinases, encoded by buk1 and buk2 ((Walter et al. Gene 134(1):107-111 (1993); Huang et al. J Mol Microbiol Biotechnol 2(1):33-38 (2000)), and the E. coli gamma-glutamyl kinase, encoded by proB (Smith et al. J. Bacteriol. 157:545-551 (1984)). These enzymes phosphorylate acetate, butyrate, and glutamate, respectively. The ackA gene product from E. coli also phosphorylates propionate (Hesslinger et al. Mol. Microbiol 27:477-492 (1998)). Information related to these proteins and genes is shown below.

TABLE 62

| Protein | GenBank ID | GI number | Organism |
|---|---|---|---|
| AckA | NP_416799.1 | 16130231 | Escherichia coli |
| Buk1 | NP_349675 | 15896326 | Clostridium acetobutylicum |
| Buk2 | Q97II1 | 20137415 | Clostridium acetobutylicum |
| ProB | NP_414777.1 | 16128228 | Escherichia coli |

The hydrolysis of acetoacetyl-CoA or 3-hydroxybutyryl-CoA can alternatively be carried out by a single enzyme or enzyme complex that exhibits acetoacetyl-CoA or 3-hydroxybutyryl-CoA synthetase activity (steps K and M, FIG. 4). This activity enables the net hydrolysis of the CoA-ester of either molecule, and in some cases, results in the simultaneous generation of ATP. For example, the product of the LSC1 and LSC2 genes of S. cerevisiae and the sucC and sucD genes of E. coli naturally form a succinyl-CoA synthetase complex that catalyzes the formation of succinyl-CoA from succinate with the concomitant consumption of one ATP, a reaction which is reversible in vivo (Gruys et al., U.S. Pat. No. 5,958,745, filed Sep. 28, 1999). Information related to these proteins and genes is shown below.

TABLE 63

| Protein | GenBank ID | GI number | Organism |
|---|---|---|---|
| SucC | NP_415256.1 | 16128703 | Escherichia coli |
| SucD | AAC73823.1 | 1786949 | Escherichia coli |
| LSC1 | NP_014785 | 6324716 | Saccharomyces cerevisiae |
| LSC2 | NP_011760 | 6321683 | Saccharomyces cerevisiae |

Additional exemplary CoA-ligases include the rat dicarboxylate-CoA ligase for which the sequence is yet uncharacterized (Vamecq et al., Biochemical J. 230:683-693 (1985)), either of the two characterized phenylacetate-CoA ligases from P. chrysogenum (Lamas-Maceiras et al., Biochem. J. 395:147-155 (2005); Wang et al., Biochem Biophy Res Commun 360(2):453-458 (2007)), the phenylacetate-CoA ligase from Pseudomonas putida (Martinez-Blanco et al., J. Biol. Chem. 265:7084-7090 (1990)), and the 6-carboxyhexanoate-CoA ligase from Bacillus subtilis (Bower et al., J. Bacteriol. 178(14):4122-4130 (1996)). Additional candidate enzymes are acetoacetyl-CoA synthetases from Mus musculus (Hasegawa et al., Biochim. Biophys. Acta 1779:414-419 (2008)) and Homo sapiens (Ohgami et al., Biochem. Pharmacol. 65:989-994 (2003)), which naturally catalyze the ATP-dependant conversion of acetoacetate into acetoacetyl-CoA. 4-Hydroxybutyryl-CoA synthetase activity has been demonstrated in Metallosphaera sedula (Berg et al., Science 318:1782-1786 (2007)). This function has been tentatively assigned to the Msed_1422 gene. Information related to these proteins and genes is shown below.

TABLE 64

| Protein | GenBank ID | GI number | Organism |
|---|---|---|---|
| Phl | CAJ15517.1 | 77019264 | Penicillium chrysogenum |
| PhlB | ABS19624.1 | 152002983 | Penicillium chrysogenum |
| PaaF | AAC24333.2 | 22711873 | Pseudomonas putida |
| BioW | NP_390902.2 | 50812281 | Bacillus subtilis |
| AACS | NP_084486.1 | 21313520 | Mus musculus |
| AACS | NP_076417.2 | 31982927 | Homo sapiens |
| Msed_1422 | YP_001191504 | 146304188 | Metallosphaera sedula |

ADP-forming acetyl-CoA synthetase (ACD, EC 6.2.1.13) is another candidate enzyme that can couple the conversion of acyl-CoA esters to their corresponding acids with the concurrent synthesis of ATP (steps K and M, FIG. 4). Several enzymes with broad substrate specificities have been described in the literature. ACD I from Archaeoglobus fulgidus, encoded by AF1211, was shown to operate on a variety of linear and branched-chain substrates including acetyl-CoA, propionyl-CoA, butyryl-CoA, acetate, propionate, butyrate, isobutyryate, isovalerate, succinate, fumarate, phenylacetate, indoleacetate (Musfeldt et al., J. Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al., supra (2004)). The enzymes from A. fulgidus, H. marismortui and P. aerophilum have all been cloned, functionally expressed, and characterized in E. coli (Musfeldt et al., supra; Brasen et al., supra (2004)). Information related to these proteins and genes is shown below.

TABLE 65

| Protein | GenBank ID | GI number | Organism |
|---|---|---|---|
| AF1211 | NP_070039.1 | 11498810 | Archaeoglobus fulgidus DSM |
| | | | 4304 |
| scs | YP_135572.1 | 55377722 | Haloarcula marismortui ATCC |
| | | | 43049 |
| PAE3250 | NP_560604.1 | 18313937 | Pyrobaculum aerophilum str. |
| | | | IM2 |

The conversion of 3-hydroxybutyrate to 3-hydroxybutyraldehyde can be carried out by a 3-hydroxybutyrate reductase (step N, FIG. 4). Similarly, the conversion of acetoacetate to acetoacetaldehyde can be carried out by an acetoacetate reductase (step L, FIG. 4). A suitable enzyme for these transformations is the aryl-aldehyde dehydrogenase, or equivalently a carboxylic acid reductase, from Nocardia iowensis. Carboxylic acid reductase catalyzes the magnesium, ATP and NADPH-dependent reduction of carboxylic acids to their corresponding aldehydes (Venkitasubramanian et al., J. Biol. Chem. 282:478-485 (2007)). This enzyme, encoded by car, was cloned and functionally expressed in E. coli (Venkitasubramanian et al., J. Biol. Chem. 282:478-485 (2007)). Expression of the npt gene product improved activity of the enzyme via post-transcriptional modification. The npt gene encodes a specific phosphopantetheine transferase (PPTase) that converts the inactive apo-enzyme to the active holo-enzyme. The natural substrate of this enzyme is vanillic acid, and the enzyme exhibits broad acceptance of aromatic and aliphatic substrates (Venkitasubramanian et al., in Biocatalysis in the Pharmaceutical and Biotechnology Industires, ed. R. N. Patel, Chapter 15, pp. 425-440, CRC Press LLC, Boca Raton, Fla. (2006)). Information related to these proteins and genes is shown below.

TABLE 66

| Protein | GenBank ID | GI number | Organism |
|---|---|---|---|
| Car | AAR91681.1 | 40796035 | Nocardia iowensis (sp. NRRL |
| | | | 5646) |
| Npt | ABI83656.1 | 114848891 | Nocardia iowensis (sp. NRRL |
| | | | 5646) |

Additional car and npt genes can be identified based on sequence homology.

TABLE 67

| Protein | GenBank ID | GI number | Organism |
|---|---|---|---|
| fadD9 | YP_978699.1 | 121638475 | Mycobacterium bovis BCG |
| BCS_2812c | YP_978898.1 | 121638674 | Mycobacterium bovis BCG |
| nfa20150 | YP_118225.1 | 54023983 | Nocardia farcinica IFM |
| | | | 10152 |
| nfa40540 | YP_120266.1 | 54026024 | Nocardia farcinica IFM |
| | | | 10152 |
| SGR_6790 | YP_001828302.1 | 182440583 | Streptomyces griseus subsp. |
| | | | griseus NBRC 13350 |
| SGR_665 | YP_001822177.1 | 182434458 | Streptomyces griseus subsp. |
| | | | griseus NBRC 13350 |
| MSMEG_2956 | YP_887275.1 | 118473501 | Mycobacterium smegmatis |
| | | | MC2 155 |
| MSMEG_5739 | YP_889972.1 | 118469671 | Mycobacterium smegmatis |
| | | | MC2 155 |
| MSMEG_2648 | YP_886985.1 | 118471293 | Mycobacterium smegmatis |
| | | | MC2 155 |
| MAP1040c | NP_959974.1 | 41407138 | Mycobacterium avium subsp. |
| | | | paratuberculosis K-10 |
| MAP2899c | NP_961833.1 | 41408997 | Mycobacterium avium subsp. |
| | | | paratuberculosis K-10 |
| MMAR_2117 | YP_001850422.1 | 183982131 | Mycobacterium marinum M |
| MMAR_2936 | YP_001851230.1 | 183982939 | Mycobacterium marinum M |
| MMAR_1916 | YP_001850220.1 | 183981929 | Mycobacterium marinum M |
| TpauDRAFT_33060 | ZP_04027864.1 | 227980601 | Tsukamurella paurometabola |
| | | | DSM 20162 |
| TpauDRAFT_20920 | ZP_04026660.1 | 227979396 | Tsukamurella paurometabola |
| | | | DSM 20162 |
| CPCC7001_1320 | ZP_05045132.1 | 254431429 | Cyanobium PCC7001 |
| DDBDRAFT_0187729 | XP_636931.1 | 66806417 | Dictyostelium discoideum |
| | | | AX4 |

An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J. Antibiot. 60(6):380-387 (2007)). Co-expression of griC and griD with SGR_665, an enzyme similar in sequence to the Nocardia iowensis npt, can be beneficial. Information related to these proteins and genes is shown below.

TABLE 68

| Protein | GenBank ID | GI number | Organism |
|---|---|---|---|
| griC | YP_001825755.1 | 182438036 | Streptomyces griseus subsp. |
| | | | griseus NBRC 13350 |
| grid | YP_001825756.1 | 182438037 | Streptomyces griseus subsp. |
| | | | griseus NBRC 13350 |

An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J. Biol. Chem. 278:8250-8256 (2003)). The gene encoding the P. chrysogenum PPTase has not been identified to date. Information related to these proteins and genes is shown below.

TABLE 69

| Protein | GenBank ID | GI number | Organism |
|---|---|---|---|
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Pencillium chrysogenum |

Any of these CAR or CAR-like enzymes can exhibit 3-hydroxybutyrate or acetoacetate reductase activity or can be engineered to do so.

Alternatively, the acetoacetyl-CoA depicted in the 1.3-BDO pathway(s) of FIG. 4 can be synthesized from acetyl-CoA and malonyl-CoA by acetoacetyl-CoA synthase, for example, as depicted in FIG. 7 (steps E and F) or FIG. 9, wherein acetyl-CoA is converted to malonyl-CoA by acetyl-CoA carboxylase, and acetoacetyl-CoA is synthesized from acetyl-CoA and malonyl-CoA by acetoacetyl-CoA synthase.

Acetoacetyl-CoA can also be synthesized from acetyl-CoA and malonyl-CoA by acetoacetyl-CoA synthase (EC 2.3.1.194). This enzyme (FhsA) has been characterized in the soil bacterium Streptomyces sp. CL190 where it participates in mevalonate biosynthesis (Okamura et al, PNAS USA 107:11265-70 (2010)). As this enzyme catalyzes an essentially irreversible reaction, it is particularly useful for metabolic engineering applications for overproducing metabolites, fuels or chemicals derived from acetoacetyl-CoA. For example, the enzyme has been heterologously expressed in organisms that biosynthesize butanol (Lan et al, PNAS USA (2012)) and poly-(3-hydroxybutyrate) (Matsumoto et al, Biosci Biotech Biochem, 75:364-366 (2011). Other relevant products of interest include 1,4-butanediol and isopropanol. Other acetoacetyl-CoA synthase genes can be identified by sequence homology to fhsA.

TABLE 70

| Protein | GenBank ID | GI Number | Organism |
|---|---|---|---|
| fhsA | BAJ83474.1 | 325302227 | Streptomyces |
| | | | sp CL190 |
| AB183750.1: | BAD86806.1 | 57753876 | Streptomyces |
| 11991 . . . 12971 | | | sp. KO-3988 |
| epzT | ADQ43379.1 | 312190954 | Streptomyces |
| | | | cinnamonensis |
| ppzT | CAX48662.1 | 238623523 | Streptomyces |
| | | | anulatus |
| O3I_22085 | ZP_09840373.1 | 378817444 | Nocardia |
| | | | brasiliensis |

### Example V

### Insertion of Nucleic Acid Sequences and Genes in S. cerevisiae

This Example describes methods for the insertion of nucleic acid sequences into S. cerevisiae. Increased production of cytosolic acetyl-CoA can be accomplished by inserting nucleic acid sequences encoding genes described in Example I. Conversion of cytosolic acetyl-CoA to 1,3-BDO can be accomplished by inserting nucleic acid sequences encoding genes described in Example II.

Nucleic acid sequences and genes can be inserted into and expressed in S. cerevisiae using several methods. Some insertion methods are plasmid-based, whereas others allow for the incorporation of the gene into the chromosome (Guthrie and Fink, Guide to Yeast Genetics and Molecular and Cell Biology, Part B, Volume 350, Academic Press (2002); Guthrie and Fink, Guide to Yeast Genetics and Molecular and Cell Biology, Part C, Volume 351, Academic Press (2002)). High copy number plasmids using auxotrophic (e.g., URA3, TRP1, HIS3, LEU2) or antibiotic selectable markers (e.g., ZeoR or KanR) can be used, often with strong, constitutive promoters such as PGK1 or ACT1 and a transcription terminator-polyadenylation region such as those from CYC1 or AOX. Many examples are available, including pVV214 (a 2 micron plasmid with URA3 selectable marker) and pVV200 (2 micron plasmid with TRP1 selectable marker) (Van et al., Yeast 20:739-746 (2003)). Alternatively, relatively low copy plasmids can be used, including pRS313 and pRS315 (Sikorski and Hieter, Genetics 122:19-27 (1989) both of which require that a promoter (e.g., PGK1 or ACT1) and a terminator (e.g., CYC1, AOX) are added.

The integration of genes into the chromosome requires an integrative promoter-based expression vector, for example, a construct that includes a promoter, the gene of interest, a terminator, and a selectable marker with a promoter, flanked by FRT sites, loxP sites, or direct repeats enabling the removal and recycling of the resistance marker. The method entails the synthesis and amplification of the gene of interest with suitable primers, followed by the digestion of the gene at a unique restriction site, such as that created by the EcoRI and XhoI enzymes (Vellanki et al., Biotechnol Lett. 29:313-318 (2007)). The gene of interest is inserted at the EcoRI and XhoI sites into a suitable expression vector, downstream of the promoter. The gene insertion is verified by PCR and DNA sequence analysis. The recombinant plasmid is then linearized and integrated at a desired site into the chromosomal DNA of S. cerevisiae using an appropriate transformation method. The cells are plated on the YPD medium with the appropriate selection marker (e.g., kanamycin) and incubated for 2-3 days. The transformants are analyzed for the requisite gene insert by colony PCR.

To remove the antibiotic marker from a construct flanked by loxP sites, a plasmid containing the Cre recombinase is introduced. Cre recombinase promotes the excision of sequences flanked by loxP sites. (Gueldener et al., Nucleic Acids Res. 30:e23 (2002)). The resulting strain is cured of the Cre plasmid by successive culturing on media without any antibiotic present. The final strain has a markerless gene deletion, and thus the same method can be used to introduce multiple insertions in the same strain. Alternatively, the FLP-FRT system can be used in an analogous manner. This system involves the recombination of sequences between short Flipase Recognition Target (FRT) sites by the Flipase recombination enzyme (FLP) derived from the 2μ plasmid of the yeast Saccharomyces cerevisiae (Sadowski, P. D., Prog. Nucleic. Acid. Res. Mol. Biol. 51:53-91 (1995); Zhu and Sadowski J. Biol. Chem. 270:23044-23054 (1995)). Similarly, gene deletion methodologies can be carried out as described in refs. Baudin et al., Nucleic. Acids Res. 21:3329-3330 (1993); Brachmann et al., Yeast 14:115-132 (1998); Giaever et al., Nature 418:387-391 (2002); Longtine et al., Yeast 14:953-961 (1998) Winzeler et al., Science 285:901-906 (1999).

### Example VI

### Insertion of Nucleic Acid Sequences and Genes in S. cerevisiae

This Example describes the insertion of genes into S. cerevisiae for the production of 1,3-BDO.

Strain construction: Saccharomyces cerevisiae haploid strain BY4741 (MATa his3Δ1 leu2Δ0 met15Δ0 ura3Δ0) with pdc5 replaced with the Kanamycin resistance gene, pdc5::kanr (clone ID 4091) from the Saccharomyces Genome Deletion Project can be further manipulated by a double crossover event using homologous recombination to replace the TRP1 gene with URA3. The resulting strain can be grown on 5-FOA plates to “URA blast” the strain, thereby selecting for clones that had ura3 mutations. A clone from this plate can be expanded. The strain with the final genotype BY4741 (MATa his3Δ1 leu2Δ0 met15Δ0 ura3Δ0 trp1::ura3 pdc5::kanr) can be used for 1,3-BDO heterologous pathway expression. The strain can be grown on synthetic defined media which contains Yeast Nitrogen Base (1.7 g/L), ammonium sulfate (5 g/L) and a complete supplement mixture (CSM) of amino acids minus -His, -Leu, -Trp, -Ura, -dextrose can also be added (Sunrise Science Products, Inc. San Diego, Calif. catalog #1788-100). An appropriate carbon source is either 0.2% glucose or 0.2% sucrose plus 2% galactose.

To construct the 1,3-BDO pathway in S. cerevisiae, genes can be identified, cloned, sequenced and expressed from expression vectors. Genes and accession numbers are described in Example I. 1,3-BDO pathway genes can be cloned into pESC vectors pESC-HIS, pESC-LEU, pESC-TRP, and pESC-URA (Stratagene, cat #217455). These are shuttle vectors that can replicate in either E. coli or S. cerevisiae. They have dual galactose (GAL1, GAL10) divergent promoters that are inhibited in the presence of dextrose (glucose) but provide inducible expression in the presence of galactose sugar. The acetoacetyl-CoA thiolase and acetoacetyl-CoA reductase can be cloned into pESC-His; 3-hydroxybutyryl-CoA reductase and 3-hydroxybutyraldehyde reductase can be cloned into pESC-Leu, and pyruvate formate lyases subunits A and B can be cloned into pESC-Ura.

All enzyme assays can be performed from cells which had first expressed the appropriate gene(s). Cells can be spun down, lysed in a bead beater with glass beads, and cell debris removed by centrifugation to generate crude extracts.

Substrate can be added to cell extracts and assayed for activity. Acetoacetyl-CoA thiolase activity can be determined by adding acetyl-CoA to extracts. If the reaction condensed the acetyl-CoA components, free CoA-SH will be released. The free CoA-SH forms a complex with DTNB to form DTNB-CoA, which can be detected by absorbance at 410 nm. To assay acetoacetyl-CoA reductase activity, acetoacetyl-CoA and NADH can be added to extracts. Acetoacetatyl-CoA absorbs at 304 nm and its decrease is used to monitor conversion of acetoacetyl-CoA to 3-hydroxybutyryl-CoA. 3-Hydroxybutyryl-CoA reductase and 3-hydroxybutyraldehyde reductase can be assayed by adding the appropriate substrate along with NADH to cell extracts. Decrease of NADH can then be assayed by fluorescence since NADH absorbs light with wavelength of 340 nm and radiates secondary (fluorescence) photons with a wavelength of 450 nm.

To detect pyruvate formate lyase activity in yeast, cells, extracts and reagents can be prepared anaerobically as the enzyme is known to be inhibited by oxygen. Because the DTNB-CoA reaction is inhibited by reducing agents required for the preparation of anaerobic extracts, assaying for the release of CoA-SH with DNTB can not be performed. Therefore, the product of the reaction (Acetyl-CoA) can be directly analyzed by mass spectrometry when extracts are provided with pyruvate.

Yeast cultures can be inoculated into synthetic defined media without His, Leu, Trp, Ura. Samples from 1,3-BDO production cultures can be collected by removing a majority of cells by centrifugation at 17,000 rpm for five minutes at room temperature in a microcentrifuge. Supernatants can be filtered through a 0.22 μm filter to remove trace amounts of cells and can be used directly for analysis by GC-MS.

The engineered strains will be characterized by measuring the growth rate, the substrate uptake rate, and the product/byproduct secretion rate. Cultures will be grown overnight and used as inoculum for a fresh batch culture for which measurements are taken during exponential growth. The growth rate can be determined by measuring optical density using a spectrophotometer (A600). Concentrations of glucose, 1,3-BDO, alcohols, and other organic acid byproducts in the culture supernatant can be determined by analytical methods including HPLC using an HPX-87H column (BioRad), or GC-MS, and used to calculate uptake and secretion rates. Cultures can then be brought to steady state exponential growth via sub-culturing for enzyme assays. All experiments will be performed with triplicate cultures.

### Example VII

### Utilization of Pathway Enzymes with a Preference for NADH

The production of acetyl-CoA from glucose can generate at most four reducing equivalents in the form of NADH. A straightforward and energy efficient mode of maximizing the yield of reducing equivalents is to employ the Embden-Meyerhof-Parnas glycolysis pathway (EMP pathway). In many carbohydrate utilizing organisms, one NADH molecule is generated per oxidation of each glyceraldehyde-3-phosphate molecule by means of glyceraldehyde-3-phosphate dehydrogenase. Given that two molecules of glyceraldehyde-3-phosphate are generated per molecule of glucose metabolized via the EMP pathway, two NADH molecules can be obtained from the conversion of glucose to pyruvate.

Two additional molecules of NADH can be generated from conversion of pyruvate to acetyl-CoA given that two molecules of pyruvate are generated per molecule of glucose metabolized via the EMP pathway. This would require employing any of the following enzymes or enzyme sets to convert pyruvate to acetyl-CoA:
1) NAD-dependant pyruvate dehydrogenase;
2) Pyruvate formate lyase and NAD-dependant formate dehydrogenase;
3) Pyruvate:ferredoxin oxidoreductase and NADH:ferredoxin oxidoreductase;
4) Pyruvate decarboxylase and an NAD-dependant acylating acetylaldehyde dehydrogenase;
5) Pyruvate decarboxylase, NAD-dependant acylating acetaldehyde dehydrogenase, acetate kinase, and phosphotransacetylase; and
6) Pyruvate decarboxylase, NAD-dependant acylating acetaldehyde dehydrogenase, and acetyl-CoA synthetase.

Overall, four molecules of NADH can be attained per glucose molecule metabolized. The 1,3-BDO pathway requires three reduction steps from acetyl-CoA. Therefore, it can be possible that each of these three reduction steps will utilize NADPH or NADH as the reducing agents, in turn converting these molecules to NADP or NAD, respectively. Therefore, it is desirable that all reduction steps are NADH-dependant in order to maximize the yield of 1,3-BDO. High yields of 1,3-BDO can thus be accomplished by:
1) Identifying and implementing endogenous or exogenous 1,3-BDO pathway enzymes with a stronger preference for NADH than other reducing equivalents such as NADPH,
2) Attenuating one or more endogenous 1,3-BDO pathway enzymes that contribute NADPH-dependant reduction activity,
3) Altering the cofactor specificity of endogenous or exogenous 1,3-BDO pathway enzymes so that they have a stronger preference for NADH than their natural versions, or
4) Altering the cofactor specificity of endogenous or exogenous 1,3-BDO pathway enzymes so that they have a weaker preference for NADPH than their natural versions.

The individual enzyme or protein activities from the endogenous or exogenous DNA sequences can be assayed using methods well known in the art. For example, the genes can be expressed in E. coli and the activity of their encoded proteins can be measured using cell extracts as described in Example V. Alternatively, the enzymes can be purified using standard procedures well known in the art and assayed for activity. Spectrophotometric based assays are particularly effective.

Several examples and methods of altering the cofactor specificity of enzymes are known in the art. For example, Khoury et al. (Protein Sci. 2009 October; 18(10): 2125-2138) created several xylose reductase enzymes with an increased affinity for NADH and decreased affinity for NADPH. Ehsani et al (Biotechnology and Bioengineering, Volume 104, Issue 2, pages 381-389, 1 Oct. 2009) drastically decreased activity of 2,3-butanediol dehydrogenase on NADH while increasing activity on NADPH. Machielsen et al (Engineering in Life Sciences, Volume 9, Issue 1, pages 38-44, February 2009) dramatically increased activity of alcohol dehydrogenase on NADH. Khoury et al (Protein Sci. 2009 October; 18(10): 2125-2138) list in Table I several previous examples of successfully changing the cofactor preference of over 25 other enzymes. Additional descriptions can be found in Lutz et al, Protein Engineering Handbook, Volume 1 and Volume 2, 2009, Wiley-VCH Verlag GmbH & Co. KGaA, in particular, Chapter 31: Altering Enzyme Substrate and Cofactor Specificity via Protein Engineering.

### Example VIII

### Determining Cofactor Preference of Pathway Enzymes

This example describes an experimental method for determining the cofactor preference of an enzyme.

Cofactor preference of enzymes for each of the pathway steps are determined by cloning the individual genes on a plasmid behind a constitutive or inducible promoter and transforming into a host organism such as Escherichia coli. For example, genes encoding enzymes that catalyze pathway steps from: 1) acetoacetyl-CoA to 3-hydroxybutyryl-CoA, 2) 3-hydroxybutyryl-CoA to 3-hydroxybutyraldehyde, 3) 3-hydroxybutyraldehyde to 1,3-butanediol, or 4) 3-hydroxybutyrate to 3-hydroxybutyraldehyde can be assembled onto the pZ-based expression vectors as described below.

Replacement of the Stuffer Fragment in the pZ-Based Expression Vectors.

Vector backbones were obtained from Dr. Rolf Lutz of Expressys (http://www.expressys.de/). The vectors and strains are based on the pZ Expression System developed by Lutz and Bujard (Nucleic Acids Res 25, 1203-1210 (1997)). The pZE13luc, pZA33luc, pZS*13luc and pZE22luc contain the luciferase gene as a stuffer fragment. To replace the luciferase stuffer fragment with a lacZ-alpha fragment flanked by appropriate restriction enzyme sites, the luciferase stuffer fragment is removed from each vector by digestion with EcoRI and XbaI. The lacZ-alpha fragment is PCR amplified from pUC19 with the following primers:

lacZalpha-RI
(SEQ ID NO: 1)
5′GACGAATTCGCTAGCAAGAGGAGAAGTCGACATGTCCAATTCACTGG
CCGTCGTTTTAC3′
lacZalpha 3′BB
(SEQ ID NO: 2)
5′-GACCCTAGGAAGCTTTCTAGAGTCGACCTATGCGGCATCAGAGCAG
A-3′

This generates a fragment with a 5′ end of EcoRI site, NheI site, a Ribosomal Binding Site, a SalI site and the start codon. On the 3′ end of the fragment are the stop codon, XbaI, HindIII, and AvrII sites. The PCR product is digested with EcoRI and AvrII and ligated into the base vectors digested with EcoRI and XbaI (XbaI and AvrII have compatible ends and generate a non-site). Because NheI and XbaI restriction enzyme sites generate compatible ends that can be ligated together (but generate a site after ligation that is not digested by either enzyme), the genes cloned into the vectors can be “Biobricked” together (http://openwetware.org/wiki/Synthetic_Biology:BioBricks). Briefly, this method enables joining an unlimited number of genes into the vector using the same 2 restriction sites (as long as the sites do not appear internal to the genes), because the sites between the genes are destroyed after each addition. These vectors can be subsequently modified using the Phusion® Site-Directed Mutagenesis Kit (NEB, Ipswich, Mass., USA) to insert the spacer sequence AATTAA between the EcoRI and NheI sites. This eliminates a putative stem loop structure in the RNA that bound the RBS and start codon.

All vectors have the pZ designation followed by letters and numbers indicating the origin of replication, antibiotic resistance marker and promoter/regulatory unit. The origin of replication is the second letter and is denoted by E for ColE1, A for p15A and S for pSC101 (as well as a lower copy number version of pSC101 designated S*)—based origins. The first number represents the antibiotic resistance marker (1 for Ampicillin, 2 for Kanamycin, 3 for Chloramphenicol). The final number defines the promoter that regulated the gene of interest (1 for PLtetO-1, 2 for PLlacO-1 and 3 for PA1lacO-1). For the work discussed here we employed three base vectors, pZS*13S, pZA33S and pZE13S, modified for the biobricks insertions as discussed above.

Plasmids containing genes encoding pathway enzymes can then transformed into host strains containing lacIQ, which allow inducible expression by addition of isopropyl β-D-1-thiogalactopyranoside (IPTG). Activities of the heterologous enzymes are tested in in vitro assays, using strain E. coli MG1655 lacIQ as the host for the plasmid constructs containing the pathway genes. Cells can be grown aerobically in LB media (Difco) containing the appropriate antibiotics for each construct, and induced by addition of IPTG at 1 mM when the optical density (OD600) reached approximately 0.5. Cells can be harvested after 6 hours, and enzyme assays conducted as discussed below.

In Vitro Enzyme Assays. To obtain crude extracts for activity assays, cells can be harvested by centrifugation at 4,500 rpm (Beckman-Coulter, Allegera X-15R) for 10 min. The pellets are resuspended in 0.3 mL BugBuster (Novagen) reagent with benzonase and lysozyme, and lysis proceeds for about 15 minutes at room temperature with gentle shaking. Cell-free lysate is obtained by centrifugation at 14,000 rpm (Eppendorf centrifuge 5402) for 30 min at 4° C. Cell protein in the sample is determined using the method of Bradford et al., Anal. Biochem. 72:248-254 (1976), and specific enzyme assays conducted as described below. Activities are reported in Units/mg protein, where a unit of activity is defined as the amount of enzyme required to convert 1 micromol of substrate in 1 minute at room temperature.

Pathway steps can be assayed in the reductive direction using a procedure adapted from several literature sources (Durre et al., FEMS Microbiol. Rev. 17:251-262 (1995); Palosaari and Rogers, Bacteriol. 170:2971-2976 (1988) and Welch et al., Arch. Biochem. Biophys. 273:309-318 (1989). The oxidation of NADH or NADPH can be followed by reading absorbance at 340 nM every four seconds for a total of 240 seconds at room temperature. The reductive assays can be performed in 100 mM MOPS (adjusted to pH 7.5 with KOH), 0.4 mM NADH or 0.4 mM NADPH, and from 1 to 50 μmol of cell extract. For carboxylic acid reductase-like enzymes, ATP can also be added at saturating concentrations. The reaction can be started by adding the following reagents: 100 μmol of 100 mM acetoacetyl-CoA, 3-hydroxybutyryl-CoA, 3-hydroxybutyrate, or 3-hydroxybutyraldehyde. The spectrophotometer is quickly blanked and then the kinetic read is started. The resulting slope of the reduction in absorbance at 340 nM per minute, along with the molar extinction coefficient of NAD(P)H at 340 nM (6000) and the protein concentration of the extract, can be used to determine the specific activity.

### Example IX

### Methods for Increasing NADPH Availability

In some cases, it can be advantageous to employ pathway enzymes that have activity using NADPH as the reducing agent. For example, NADPH-dependant pathway enzymes can be highly specific for pathway intermediates such as acetoacetyl-CoA, 3-hydroxybutyryl-CoA, 3-hydroxybutyrate, or 3-hydroxybutyraldehyde or can possess favorable kinetic properties using NADPH as a substrate. If one or more pathway steps is NADPH dependant, several alternative approaches to increase NADPH availability can be employed. These include:
1) Increasing flux relative to wild-type through the oxidative branch of the pentose phosphate pathway comprising glucose-6-phosphate dehydrogenase, 6-phosphogluconolactonase, and 6-phosphogluconate dehydrogenase (decarboxylating). This will generate 2 NADPH molecules per glucose-6-phosphate metabolized. However, the decarboxylation step will reduce the maximum theoretical yield of 1,3-butanediol.
2) Increasing flux relative to wild-type through the Entner Doudoroff pathway comprising glucose-6-phosphate dehydrogenase, 6-phosphogluconolactonase, phosphogluconate dehydratase, and 2-keto-3-deoxygluconate 6-phosphate aldolase.
3) Introducing a soluble transhydrogenase to convert NADH to NADPH.
4) Introducing a membrane-bound transhydrogenase to convert NADH to NADPH.
5) Employing an NADP-dependant glyceraldehyde-3-phosphate dehydrogenase.
6) Employing any of the following enzymes or enzyme sets to convert pyruvate to acetyl-CoA
   a) NADP-dependant pyruvate dehydrogenase;
   b) Pyruvate formate lyase and NADP-dependant formate dehydrogenase;
   c) Pyruvate:ferredoxin oxidoreductase and NADPH:ferredoxin oxidoreductase;
   d) Pyruvate decarboxylase and an NADP-dependant acylating acetylaldehyde dehydrogenase;
   e) Pyruvate decarboxylase, NADP-dependant acetaldehyde dehydrogenase, acetate kinase, and phosphotransacetylase; and
   f) Pyruvate decarboxylase, NADP-dependant acetaldehyde dehydrogenase, and acetyl-CoA synthetase; and optionally attenuating NAD-dependant versions of these enzymes.
7) Altering the cofactor specificity of a native glyceraldehyde-3-phosphate dehydrogenase, pyruvate dehydrogenase, formate dehydrogenase, or acylating acetylaldehyde dehydrogenase to have a stronger preference for NADPH than their natural versions.
8) Altering the cofactor specificity of a native glyceraldehyde-3-phosphate dehydrogenase, pyruvate dehydrogenase, formate dehydrogenase, or acylating acetylaldehyde dehydrogenase to have a weaker preference for NADH than their natural versions.

The individual enzyme or protein activities from the endogenous or exogenous DNA sequences can be assayed using methods well known in the art. For example, the genes can be expressed in E. coli and the activity of their encoded proteins can be measured using cell extracts as described in the previous example. Alternatively, the enzymes can be purified using standard procedures well known in the art and assayed for activity. Spectrophotometric based assays are particularly effective.

Several examples and methods of altering the cofactor specificity of enzymes are known in the art. For example, Khoury et al (Protein Sci. 2009 October; 18(10): 2125-2138) created several xylose reductase enzymes with an increased affinity for NADH and decreased affinity for NADPH. Ehsani et al (Biotechnology and Bioengineering, Volume 104, Issue 2, pages 381-389, 1 Oct. 2009) drastically decreased activity of 2,3-butanediol dehydrogenase on NADH while increasing activity on NADPH. Machielsen et al (Engineering in Life Sciences, Volume 9, Issue 1, pages 38-44, February 2009) dramatically increased activity of alcohol dehydrogenase on NADH. Khoury et al (Protein Sci. 2009 October; 18(10): 2125-2138) list in Table I several previous examples of successfully changing the cofactor preference of over 25 other enzymes. Additional descriptions can be found in Lutz et al, Protein Engineering Handbook, Volume 1 and Volume 2, 2009, Wiley-VCH Verlag GmbH & Co. KGaA, in particular, Chapter 31: Altering Enzyme Substrate and Cofactor Specificity via Protein Engineering.

Enzyme candidates for these steps are provided below.

TABLE 70
Glucose-6-phosphate dehydrogenase

| Protein | GenBank ID | GI Number | Organism |
|---|---|---|---|
| ZWF1 | NP_014158.1 | 6324088 | Saccharomyces |
| | | | cerevisiae S288c |
| ZWF1 | XP_504275.1 | 50553728 | Yarrowia lipolytica |
| Zwf | XP_002548953.1 | 255728055 | Candida tropicalis |
| | | | MYA-3404 |
| Zwf | XP_001400342.1 | 145233939 | Aspergillus niger |
| | | | CBS 513.88 |
| KLLA0D19855g | XP_453944.1 | 50307901 | Kluyveromyces |
| | | | lactis NRRL |
| | | | Y-1140 |

TABLE 71
6-Phosphogluconolactonase

| Protein | GenBank ID | GI Number | Organism |
|---|---|---|---|
| SOL3 | NP_012033.2 | 82795254 | Saccharomyces |
| | | | cerevisiae S288c |
| SOL4 | NP_011764.1 | 6321687 | Saccharomyces |
| | | | cerevisiae S288c |
| YALI0E11671g | XP_503830.1 | 50552840 | Yarrowia lipolytica |
| YALI0C19085g | XP_501998.1 | 50549055 | Yarrowia lipolytica |
| ANI_1_656014 | XP_001388941.1 | 145229265 | Aspergillus niger |
| | | | CBS 513.88 |
| CTRG_00665 | XP_002545884.1 | 255721899 | Candida tropicalis |
| | | | MYA-3404 |
| CTRG_02095 | XP_002547788.1 | 255725718 | Candida tropicalis |
| | | | MYA-3404 |
| KLLA0A05390g | XP_451238.1 | 50302605 | Kluyveromyces |
| | | | lactis NRRL |
| | | | Y-1140 |
| KLLA0C08415g | XP_452574.1 | 50305231 | Kluyveromyces |
| | | | lactis NRRL |
| | | | Y-1140 |

TABLE 72
6-Phosphogluconate dehydrogenase (decarboxylating)

| Protein | GenBank ID | GI Number | Organism |
|---|---|---|---|
| GND1 | NP_012053.1 | 6321977 | Saccharomyces |
| | | | cerevisiae S288c |
| GND2 | NP_011772.1 | 6321695 | Saccharomyces |
| | | | cerevisiae S288c |
| ANI_1_282094 | XP_001394208.2 | 317032184 | Aspergillus niger |
| | | | CBS 513.88 |
| ANI_1_2126094 | XP_001394596.2 | 317032939 | Aspergillus niger |
| | | | CBS 513.88 |
| YALI0B15598g | XP_500938.1 | 50546937 | Yarrowia lipolytica |
| CTRG_03660 | XP_002549363.1 | 255728875 | Candida tropicalis |
| | | | MYA-3404 |
| KLLA0A09339g | XP_451408.1 | 50302941 | Kluyveromyces |
| | | | lactis NRRL |
| | | | Y-1140 |

TABLE 73
Phosphogluconate dehydratase

| Protein | GenBank ID | GI Number | Organism |
|---|---|---|---|
| Edd | AAC74921.1 | 1788157 | Escherichia coli |
| | | | K-12 MG1655 |
| Edd | AAG29866.1 | 11095426 | Zymomonas mobilis |
| | | | subsp. mobilis ZM4 |
| Edd | YP_350103.1 | 77460596 | Pseudomonas |
| | | | fluorescens Pf0-1 |
| ANI_1_2126094 | XP_001394596.2 | 317032939 | Aspergillus niger |
| | | | CBS 513.88 |
| YALI0B15598g | XP_500938.1 | 50546937 | Yarrowia lipolytica |
| CTRG_03660 | XP_002549363.1 | 255728875 | Candida tropicalis |
| | | | MYA-3404 |
| KLLA0A09339g | XP_451408.1 | 50302941 | Kluyveromyces |
| | | | lactis NRRL |
| | | | Y-1140 |

TABLE 74
2-Keto-3-deoxygluconate 6-phosphate aldolase

| Protein | GenBank ID | GI Number | Organism |
|---|---|---|---|
| Eda | NP_416364.1 | 16129803 | Escherichia coli K-12 MG1655 |
| Eda | Q00384.2 | 59802878 | Zymomonas mobilis subsp. |
| | | | mobilis ZM4 |
| Eda | ABA76098.1 | 77384585 | Pseudomonas fluorescens Pf0-1 |

TABLE 75
Soluble transhydrogenase

| Protein | GenBank ID | GI Number | Organism |
|---|---|---|---|
| SthA | NP_418397.2 | 90111670 | Escherichia coli K-12 |
| | | | MG1655 |
| SthA | YP_002798658.1 | 226943585 | Azotobacter vinelandii DJ |
| SthA | O05139.3 | 11135075 | Pseudomonas fluorescens |

TABLE 76
Membrane-bound transhydrogenase

| Protein | GenBank ID | GI Number | Organism |
|---|---|---|---|
| ANI_1_29100 | XP_001400109.2 | 317027842 | Aspergillus niger CBS |
| | | | 513.88 |
| Pc21g18800 | XP_002568871.1 | 226943585 | 255956237 Penicillium |
| | | | chrysogenum |
| | | | Wisconsin 54-1255 |
| SthA | O05139.3 | 11135075 | Pseudomonas |
| | | | fluorescens |
| NCU01140 | XP_961047.2 | 164426165 | Neurospora crassa |
| | | | OR74A |

TABLE 77
NADP-dependant glyceraldehyde-3-phosphate dehydrogenase

| Protein | GenBank ID | GI Number | Organism |
|---|---|---|---|
| gapN | AAA91091.1 | 642667 | Streptococcus mutans |
| NP-GAPDH | AEC07555.1 | 330252461 | Arabidopsis thaliana |
| GAPN | AAM77679.2 | 82469904 | Triticum aestivum |
| gapN | CAI56300.1 | 87298962 | Clostridium |
| | | | acetobutylicum |
| NADP-GAPDH | 2D2I_A | 112490271 | Synechococcus |
| | | | elongatus PCC 7942 |
| NADP-GAPDH | CAA62619.1 | 4741714 | Synechococcus |
| | | | elongatus PCC 7942 |
| GDP1 | XP_455496.1 | 50310947 | Kluyveromyces lactis |
| | | | NRRL Y-1140 |
| HP1346 | NP_208138.1 | 15645959 | Helicobacter pylori |
| | | | 26695 |

TABLE 78
NAD-dependant glyceraldehyde-3-phosphate dehydrogenase

| Protein | GenBank ID | GI Number | Organism |
|---|---|---|---|
| TDH1 | NP_012483.1 | 6322409 | Saccharomyces |
| | | | cerevisiae s288c |
| TDH2 | NP_012542.1 | 6322468 | Saccharomyces |
| | | | cerevisiae s288c |
| TDH3 | NP_011708.1 | 632163 | Saccharomyces |
| | | | cerevisiae s288c |
| KLLA0A11858g | XP_451516.1 | 50303157 | Kluyveromyces |
| | | | lactis NRRL |
| | | | Y-1140 |
| KLLA0F20988g | XP_456022.1 | 50311981 | Kluyveromyces |
| | | | lactis NRRL |
| | | | Y-1140 |
| ANI_1_256144 | XP_001397496.1 | 145251966 | Aspergillus niger |
| | | | CBS 513.88 |
| YALI0C06369g | XP_501515.1 | 50548091 | Yarrowia lipolytica |
| CTRG_05666 | XP_002551368.1 | 255732890 | Candida tropicalis |
| | | | MYA-3404 |

TABLE 79
NADP-dependant pyruvate dehydrogenase

| Protein | GenBank ID | GI Number | Organism |
|---|---|---|---|
| PNO | Q94IN5.1 | 33112418 | Euglena |
| | | | gracilis |
| cgd4_690 | XP_625673.1 | 66356990 | Crypto- |
| | | | sporidium |
| | | | parvum Iowa II |
| TPP_PFOR_PNO | XP_002765111.11 | 294867463 | Perkinsus |
| | | | marinus ATCC |
| | | | 50983 |
| aceE | NP_414656.1 | 50303157 | Escherichia |
| | | | coli K-12 |
| | | | MG1655 |
| aceF | NP_414657.1 | 6128108 | Escherichia |
| | | | coli K-12 |
| | | | MG1655 |

Mutated LpdA from E. coli K-12 MG1655 described in Biochemistry, 1993, 32 (11), pp 2737-2740:

(SEQ ID NO: 3)
MSTEIKTQVVVLGAGPAGYSAAFRCADLGLETVIVERYNTLGGVCLNVG
CIPSKALLHVAKVIEEAKALAEHGIVFGEPKTDIDKIRTWKEKVINQLT
GGLAGMAKGRKVKVVNGLGKFTGANTLEVEGENGKTVINFDNAIIAAGS
RPIQLPFIPHEDPRIWDSTDALELKEVPERLLVMGGGIIGLEMGTVYHA
LGSQIDVVVRKHQVIRAADKDIVKVFTKRISKKFNLMLETKVTAVEAKE
DGIYVTMEGKKAPAEPQRYDAVLVAIGRVPNGKNLDAGKAGVEVDDRGF
IRVDKQLRTNVPHIFAIGDIVGQPMLAHKGVHEGHVAAEVIAGKKHYFD
PKVIPSIAYTEPEVAWVGLTEKEAKEKGISYETATFPWAASGRAIASDC
ADGMTKLIFDKESHRVIGGAIVGTNGGELLGEIGLAIEMGCDAEDIALT
IHAHPTLHESVGLAAEVFEGSITDLPNPKAKKK

Mutated LpdA from E. coli K-12 MG1655 described in Biochemistry, 1993, 32 (11), pp 2737-2740:

(SEQ ID NO: 4)
MSTEIKTQVVVLGAGPAGYSAAFRCADLGLETVIVERYNTLGGVCLNVG
CIPSKALLHVAKVIEEAKALAEHGIVFGEPKTDIDKIRTWKEKVINQLT
GGLAGMAKGRKVKVVNGLGKFTGANTLEVEGENGKTVINFDNAIIAAGS
RPIQLPFIPHEDPRIWDSTDALELKEVPERLLVMGGGIIALEMATVYHA
LGSQIDVVVRKHQVIRAADKDIVKVFTKRISKKFNLMLETKVTAVEAKE
DGIYVTMEGKKAPAEPQRYDAVLVAIGRVPNGKNLDAGKAGVEVDDRGF
IRVDKQLRTNVPHIFAIGDIVGQPMLAHKGVHEGHVAAEVIAGKKHYFD
PKVIPSIAYTEPEVAWVGLTEKEAKEKGISYETATFPWAASGRAIASDC
ADGMTKLIFDKESHRVIGGAIVGTNGGELLGEIGLAIEMGCDAEDIALT
IHAHPTLHESVGLAAEVFEGSITDLPNPKAKKK

TABLE 80
NADP-dependant formate dehydrogenase

| Protein | GenBank ID | GI Number | Organism |
|---|---|---|---|
| fdh | ACF35003. | 194220249 | Burkholderia stabilis |
| fdh | ABC20599.2 | 146386149 | Moorella thermoacetica ATCC |
| | | | 39073 |

Mutant Candida bodinii enzyme described in Journal of Molecular Catalysis B: Enzymatic, Volume 61, Issues 3-4, December 2009, Pages 157-161:

(SEQ ID NO: 5)
MKIVLVLYDAGKHAADEEKLYGCTENKLGIANWLKDQGHELITTSDKEG
ETSELDKHIPDADIIITTPFHPAYITKERLDKAKNLKLVVVAGVGSDHI
DLDYINQTGKKISVLEVTGSNVVSVAEHVVMTMLVLVRNFVPAHEQIIN
HDWEVAAIAKDAYDIEGKTIATIGAGRIGYRVLERLLPFNPKELLYYQR
QALPKEAEEKVGARRVENIEELVAQADIVTVNAPLHAGTKGLINKELLS
KFKKGAWLVNTARGAICVAEDVAAALESGQLRGYGGDVWFPQPAPKDHP
WRDMRNKYGAGNAMTPHYSGTTLDAQTRYAEGTKNILESFFTGKFDYRP
QDIILLNGEYVTKAYGKHDKK

Mutant Candida bodinii enzyme described in Journal of Molecular Catalysis B: Enzymatic, Volume 61, Issues 3-4, December 2009, Pages 157-161:

(SEQ ID NO: 6)
MKIVLVLYDAGKHAADEEKLYGCTENKLGIANWLKDQGHELITTSDKEG
ETSELDKHIPDADIIITTPFHPAYITKERLDKAKNLKLVVVAGVGSDHI
DLDYINQTGKKISVLEVTGSNVVSVAEHVVMTMLVLVRNFVPAHEQIIN
HDWEVAAIAKDAYDIEGKTIATIGAGRIGYRVLERLLPFNPKELLYYSP
QALPKEAEEKVGARRVENIEELVAQADIVTVNAPLHAGTKGLINKELLS
KFKKGAWLVNTARGAICVAEDVAAALESGQLRGYGGDVWFPQPAPKDHP
WRDMRNKYGAGNAMTPHYSGTTLDAQTRYAEGTKNILESFFTGKFDYRP
QDIILLNGEYVTKAYGKHDKK

Mutant Saccharomyces cerevisiae enzyme described in Biochem J. 2002 November 1:367(Pt. 3):841-847:

(SEQ ID NO: 7)
MSKGKVLLVLYEGGKHAEEQEKLLGCIENELGIRNFIEEQGYELVTTID
KDPEPTSTVDRELKDAEIVITTPFFPAYISRNRIAEAPNLKLCVTAGVG
SDHVDLEAANERKITVTEVTGSNVVSVAEHVMATILVLIRNYNGGHQQA
INGEWDIAGVAKNEYDLEDKIISTVGAGRIGYRVLERLVAFNPKKLLYY
ARQELPAEAINRLNEASKLFNGRGDIVQRVEKLEDMVAQSDVVTINCPL
HKDSRGLFNKKLISHMKDGAYLVNTARGAICVAEDVAEAVKSGKLAGYG
GDVWDKQPAPKDHPWRTMDNKDHVGNAMTVHISGTSLDAQKRYAQGVKN
ILNSYFSKKFDYRPQDIIVQNGSYATRAYGQKK.

TABLE 81
NADPH: ferredoxin oxidoreductase

| Protein | GenBank ID | GI Number | Organism |
|---|---|---|---|
| petH | YP_171276.1 | 56750575 | Synechococcus elongatus |
| | | | PCC 6301 |
| fpr | NP_457968.1 | 16762351 | Salmonella enterica |
| fnr1 | XP_001697352.1 | 159478523 | Chlamydomonas reinhardtii |
| rfhr1 | NP_567293.1 | 18412939 | Arabidopsis thaliana |
| aceF | NP_414657.1 | 6128108 | Escherichia coli K-12 |
| | | | MG1655 |

TABLE 82
NADP-dependant acylating acetylaldehyde dehydrogenase

| Protein | GenBank ID | GI Number | Organism |
|---|---|---|---|
| adhB | AAB06720.1 | 1513071 | Thermo- |
| | | | anaerobacter |
| | | | pseudethanolicus |
| | | | ATCC 33223 |
| TheetDRAFT_0840 | ZP_08211603. | 326390041 | Thermo- |
| | | | anaerobacter |
| | | | ethanolicus JW |
| | | | 200 |
| Cbei_3832 | YP_001310903.1 | 150018649 | Clostridium |
| | | | beijerinckii |
| | | | NCIMB 8052 |
| Cbei_4054 | YP_001311120.1 | 150018866 | Clostridium |
| | | | beijerinckii |
| | | | NCIMB 8052 |
| Cbei_4045 | YP_001311111.1 | 150018857 | Clostridium |
| | | | beijerinckii |
| | | | NCIMB 8052 |

Exemplary genes encoding pyruvate dehydrogenase, pyruvate:ferredoxin oxidoreductase, pyruvate formate lyase, pyruvate decarboxylase, acetate kinase, phosphotransacetylase and acetyl-CoA synthetase are described above in Example II.

Genes encoding enzymes that can facilitate the transport of 1,3-butanediol include glycerol facilitator protein homologs such as those provided below.

### Example X

### Engineering Saccharomyces Cerevisiae for Chemical Production

Eukaryotic hosts have several advantages over prokaryotic systems. They are able to support post-translational modifications and host membrane-anchored and organelle-specific enzymes. Genes in eukaryotes typically have introns, which can impact the timing of gene expression and protein structure.

An exemplary eukaryotic organism well suited for industrial chemical production is Saccharomyces cerevisiae. This organism is well characterized, genetically tractable and industrially robust. Genes can be readily inserted, deleted, replaced, overexpressed or underexpressed using methods known in the art. Some methods are plasmid-based whereas others allow for the incorporation of the gene into the chromosome (Guthrie and Fink. Guide to Yeast Genetics and Molecular and Cell Biology, Part B, Volume 350, Academic Press (2002); Guthrie and Fink, Guide to Yeast Genetics and Molecular and Cell Biology, Part C, Volume 351, Academic Press (2002)).

Plasmid-mediated gene expression is enabled by yeast episomal plasmids (YEps).

YEps allow for high levels of expression; however they are not very stable and they require cultivation in selective media. They also have a high maintenance cost to the host metabolism. High copy number plasmids using auxotrophic (e.g., URA3, TRP1, HIS3, LEU2) or antibiotic selectable markers (e.g., Zeo® or Kan®) can be used, often with strong, constitutive promoters such as PGK1 or ACT1 and a transcription terminator-polyadenylation region such as those from CYC1 or AOX. Many examples are available for one well-versed in the art. These include pVV214 (a 2 micron plasmid with URA3 selectable marker) and pVV200 (2 micron plasmid with TRP1 selectable marker) (Van et al., Yeast 20:739-746 (2003)). Alternatively, relatively low copy plasmids can be used. Again, many examples are available for one well-versed in the art. These include pRS313 and pRS315 (Sikorski and Hieter, Genetics 122:19-27 (1989) both of which require that a promoter (e.g., PGK1 or ACT1) and a terminator (e.g., CYC1, AOX) are added.

For industrial applications, chromosomal overexpression of genes is preferable to plasmid-mediated overexpression. Tools for inserting genes into eukaryotic organisms such as S. cerevisiae are known in the art. Particularly useful tools include yeast integrative plasmids (YIps), yeast artificial chromosomes (YACS) and gene targeting/homologous recombination. Note that these tools can also be used to insert, delete, replace, underexpress or otherwise alter the genome of the host.

Yeast integrative plasmids (YIps) utilize the native yeast homologous recombination system to efficiently integrate DNA into the chromosome. These plasmids do not contain an origin of replication and can therefore only be maintained after chromosomal integration. An exemplary construct includes a promoter, the gene of interest, a terminator, and a selectable marker with a promoter, flanked by FRT sites, loxP sites, or direct repeats enabling the removal and recycling of the resistance marker. The method entails the synthesis and amplification of the gene of interest with suitable primers, followed by the digestion of the gene at a unique restriction site, such as that created by the EcoRI and XhoI enzymes (Vellanki et al., Biotechnol Lett. 29:313-318 (2007)). The gene of interest is inserted at the EcoRI and XhoI sites into a suitable expression vector, downstream of the promoter. The gene insertion is verified by PCR and DNA sequence analysis. The recombinant plasmid is then linearized and integrated at a desired site into the chromosomal DNA of S. cerevisiae using an appropriate transformation method. The cells are plated on the YPD medium with an appropriate selection marker and incubated for 2-3 days. The transformants are analyzed for the requisite gene insert by colony PCR. To remove the antibiotic marker from a construct flanked by loxP sites, a plasmid containing the Cre recombinase is introduced. Cre recombinase promotes the excision of sequences flanked by loxP sites. (Gueldener et al., Nucleic Acids Res 30:e23 (2002)). The resulting strain is cured of the Cre plasmid by successive culturing on media without any antibiotic present. The final strain has a markerless gene deletion, and thus the same method can be used to introduce multiple insertions in the same strain. Alternatively, the FLP-FRT system can be used in an analogous manner. This system involves the recombination of sequences between short Flipase Recognition Target (FRT) sites by the Flipase recombination enzyme (FLP) derived from the 2μ plasmid of the yeast Saccharomyces cerevisiae (Sadowski, P. D., Prog. Nucleic. Acid. Res. Mol. Biol. 51:53-91 (1995); Zhu and Sadowski J. Biol. Chem. 270:23044-23054 (1995)). Similarly, gene deletion methodologies will be carried out as described in refs. Baudin et al. Nucleic. Acids Res. 21:3329-3330 (1993); Brachmann et al., Yeast 14:115-132 (1998); Giaever et al., Nature 418:387-391 (2002); Longtine et al., Yeast 14:953-961 (1998) Winzeler et al., Science 285:901-906 (1999).

Another powerful approach for manipulating the yeast chromosome is gene targeting. This approach takes advantage of the fact that double stranded DNA breaks in yeast are repaired by homologous recombination. Linear DNA fragments flanked by targeting sequences can thus be efficiently integrated into the yeast genome using the native homologous recombination machinery. In addition to the application of inserting genes, gene targeting approaches are useful for genomic DNA manipulations such as deleting genes, introducing mutations in a gene, its promoter or other regulatory elements, or adding a tag to a gene.

Yeast artificial chromosomes (YACs) are artificial chromosomes useful for pathway construction and assembly. YACs enable the expression of large sequences of DNA (100-3000 kB) containing multiple genes. The use of YACs was recently applied to engineer flavenoid biosynthesis in yeast (Naesby et al, Microb Cell Fact 8:49-56 (2009)). In this approach, YACs were used to rapidly test randomly assembled pathway genes to find the best combination.

The expression level of a gene can be modulated by altering the sequence of a gene and/or its regulatory regions. Such gene regulatory regions include, for example, promoters, enhancers, introns, and terminators. Functional disruption of negative regulatory elements such as repressors and/or silencers also can be employed to enhance gene expression. RNA based tools can also be employed to regulate gene expression. Such tools include RNA aptamers, riboswitches, antisense RNA, ribozymes and riboswitches.

For altering a gene's expression by its promoter, libraries of constitutive and inducible promoters of varying strengths are available. Strong constitutive promoters include pTEF1, pADH1 and promoters derived from glycolytic pathway genes. The pGAL promoters are well-studied inducible promoters activated by galactose and repressed by glucose. Another commonly used inducible promoter is the copper inducible promoter pCUP1 (Farhi et al, Met Eng 13:474-81 (2011)). Further variation of promoter strengths can be introduced by mutagenesis or shuffling methods. For example, error prone PCR can be applied to generate synthetic promoter libraries as shown by Alper and colleagues (Alper et al, PNAS 102:12678-83 (2005)). Promoter strength can be characterized by reporter proteins such as beta-galactosidase, fluorescent proteins and luciferase.

The placement of an inserted gene in the genome can alter its expression level. For example, overexpression of an integrated gene can be achieved by integrating the gene into repeating DNA elements such as ribosomal DNA or long terminal repeats.

For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Genetic modifications can also be made to enhance polypeptide synthesis. For example, translation efficiency is enhanced by substituting ribosome binding sites with an optimal or consensus sequence and/or altering the sequence of a gene to add or remove secondary structures. The rate of translation can also be increased by substituting one coding sequence with another to better match the codon preference of the host.

### Example XI

### Exemplary Genes for 1,3-BDO Export

1,3-butanediol must exit the production organism in order to be recovered and/or dehydrated to butadiene. Genes encoding enzymes that can facilitate the transport of 1,3-butanediol include glycerol facilitator protein homologs such as those provided below. Multidrug resistance transporters that export butanol, including OmrA, LmrA and homologs (see, e.g., Burd and Bhattacharyya, US Patent Application 20090176288) are also suitable transporters for 1,3-butanediol.

TABLE 83

| Protein | GenBank ID | GI number | Organism |
|---|---|---|---|
| glpF | NP_418362.1 | 16131765 | Escherichia coli |
| YFL054C | NP_116601.1 | 14318465 | Saccharomyces |
| | | | cerevisiae |
| YLL043W | NP_013057.1 | 6322985 | Saccharomyces |
| | | | cerevisiae |
| KLLA0E00617g | XP_453974.1 | 50307951 | Kluyveromyces |
| | | | lactis |
| ANI_1_1314144 | XP_001397337.2 | 317036426 | Aspergillus niger |
| ANI_1_3222024 | XP_001400456.1 | 145234170 | Aspergillus niger |
| ANI_1_710114 | XP_001396373.2 | 317034445 | Aspergillus niger |
| YALI0E05665p | XP_503595.1 | 50552370 | Yarrowia lipolytica |
| YALI0F00462p | XP_504820.1 | 50554823 | Yarrowia lipolytica |
| OmrA | ZP_01543718 | 118586261 | Oenococcus oeni |
| LmrA | AAB49750 | 1890649 | Lactococcus lactis |

### Example XII

### Pathways for Producing Acetyl-CoA from Pep and Pyruvate

FIG. 10 shows numerous pathways for converting PEP and pyruvate to acetyl-CoA, acetoacetyl-CoA, and further to products derived from acetoacetyl-CoA such as 1,3-butanediol. Enzymes candidates for the reactions shown in FIG. 10 are described below.

TABLE 84

| | | |
|---|---|---|
| 1.1.n.a | Oxidoreductase (alcohol to oxo) | M |
| 1.1.1.d | Malic enzyme | L |
| 1.2.1.a | Oxidoreductase (aldehyde to acid) | J |
| 1.2.1.b | Oxidoreductase (acyl-CoA to aldehyde) | G |
| 1.2.1.f | Oxidoreductase (decarboxylating acyl-CoA to | C |
| | aldehyde) | |
| 2.7.2.a | Kinase | N |
| 2.8.3.a | CoA transferase | K |
| 3.1.3.a | Phosphatase | N |
| 4.1.1.a | Decarboxylase | A, B, D |
| 6.2.1.a | CoA synthetase | K |
| 6.4.1.a | Carboxylase | D, H |

Enzyme candidates for several enzymes in FIG. 10 have been described elsewhere in the text. These include acetoacetyl-CoA synthase (Table 70), acetoacetyl-CoA thiolase (Table 42), malonyl-CoA reductase (also called malonate semialdehyde dehydrogenase (acylating) (Tables 35, 46), malate dehydrogenase (Tables 7 and 23).

1.1.n.a

Malate dehydrogenase or oxidoreductase catalyzes the oxidation of malate to oxaloacetate. Different carriers can act as electron acceptors for enzymes in this class. Malate dehydrogenase enzymes utilize NADP or NAD as electron acceptors. Malate dehydrogenase (Step M) enzyme candidates are described above in example 1 (Table 7, 23). Malate:quinone oxidoreductase enzymes (EC 1.1.5.4) are membrane-associated and utilize quinones, flavoproteins or vitamin K as electron acceptors. Malate:quinone oxidoreductase enzymes of E. coli, Helicobacter pylori and Pseudomonas syringae are encoded by mqo (Kather et al, J Bacteriol 182:3204-9 (2000); Mellgren et al., J Bacteriol 191:3132-42 (2009)). The Cgl2001 gene of C. gluamicum also encodes an MQO enzyme (Mitsuhashi et al, Biosci Biotechnol Biochem 70:2803-6 (2006)).

TABLE 85

| Protein | GenBank ID | GI Number | Organism |
|---|---|---|---|
| mqo | NP_416714.1 | 16130147 | Escherichia coli |
| mqo | NP_206886.1 | 15644716 | Helicobacter pylori |
| mqo | NP_790970.1 | 28868351 | Pseudomonas syringae |
| Cgl2001 | NP_601207.1 | 19553205 | Corynebacterium glutamicum |

1.1.1.d

Malic enzyme (malate dehydrogenase) catalyzes the reversible oxidative carboxylation of pyruvate to malate. E. coli encodes two malic enzymes, MaeA and MaeB (Takeo, J. Biochem. 66:379-387 (1969)). Although malic enzyme is typically assumed to operate in the direction of pyruvate formation from malate, the NAD-dependent enzyme, encoded by maeA, has been demonstrated to operate in the carbon-fixing direction (Stols and Donnelly, Appl. Environ. Microbiol. 63(7) 2695-2701 (1997)). A similar observation was made upon overexpressing the malic enzyme from Ascaris suum in E. coli (Stols et al., Appl. Biochem. Biotechnol. 63-65(1), 153-158 (1997)). The second E. coli malic enzyme, encoded by maeB, is NADP-dependent and also decarboxylates oxaloacetate and other alpha-keto acids (Iwakura et al., J. Biochem. 85(5):1355-65 (1979)). Another suitable enzyme candidate is me1 from Zea mays (Furumoto et al, Plant Cell Physiol 41:1200-1209 (2000)).

TABLE 86

| Protein | GenBank ID | GI Number | Organism |
|---|---|---|---|
| maeA | NP_415996 | 90111281 | Escherichia coli |
| maeB | NP_416958 | 16130388 | Escherichia coli |
| NAD-ME | P27443 | 126732 | Ascaris suum |
| Me1 | P16243.1 | 126737 | Zea mays |

1.2.1.a

The oxidation of malonate semialdehyde to malonate is catalyzed by malonate semialdehyde dehydrogenase (EC 1.2.1.15). This enzyme was characterized in Pseudomonas aeruginosa (Nakamura et al, Biochim Biophys Acta 50:147-52 (1961)). The NADP and NAD-dependent succinate semialdehyde dehydrogenase enzymes of Euglena gracilas accept malonate semialdehyde as substrates (Tokunaga et al, Biochem Biophys Act 429:55-62 (1976)). Genes encoding these enzymes has not been identified to date. Aldehyde dehydrogenase enzymes from eukoryotic organisms such as S. cerevisiae, C. albicans, Y. lipolytica and A. niger typically have broad substrate specificity and are suitable candidates. These enzymes and other acid forming aldehyde dehydrogenase and aldehyde oxidase enzymes are described earlier and listed in Tables 9 and 30. Additional MSA dehydrogenase enzyme candidates include NAD(P)+-dependent aldehyde dehydrogenase enzymes (EC 1.2.1.3). Two aldehyde dehydrogenases found in human liver, ALDH-1 and ALDH-2, have broad substrate ranges for a variety of aliphatic, aromatic and polycyclic aldehydes (Klyosov, Biochemistry 35:4457-4467 (1996a)). Active ALDH-2 has been efficiently expressed in E. coli using the GroEL proteins as chaperonins (Lee et al., Biochem. Biophys. Res. Commun. 298:216-224 (2002)). The rat mitochondrial aldehyde dehydrogenase also has a broad substrate range (Siew et al., Arch. Biochem. Biophys. 176:638-649 (1976)). The E. coli genes astD and aldH encode NAD+-dependent aldehyde dehydrogenases. AstD is active on succinic semialdehyde (Kuznetsova et al., FEMS Microbiol Rev 29:263-279 (2005)) and aldH is active on a broad range of aromatic and aliphatic substrates (Jo et al, Appl Microbiol Biotechnol 81:51-60 (2008)).

TABLE 87

| Gene | GenBank Accession No. | GI No. | Organism |
|---|---|---|---|
| astD | P76217.1 | 3913108 | Escherichia coli |
| aldH | AAC74382.1 | 1787558 | Escherichia coli |
| ALDH-2 | P05091.2 | 118504 | Homo sapiens |
| ALDH-2 | NP_115792.1 | 14192933 | Rattus norvegicus |

1.2.1.f

Malonate semialdehyde dehydrogenase (acetylating) (EC 1.2.1.18) catalyzes the oxidative decarboxylation of malonate semialdehyde to acetyl-CoA. Exemplary enzymes are encoded by ddcC of Halomonas sp. HTNK1 (Todd et al, Environ Microbiol 12:237-43 (2010)) and IolA of Lactobacillus casei (Yebra et al, AEM 73:3850-8 (2007)). The DdcC enzyme has homologs in A. niger and C. albicans, shown in the table below. The malonate semialdehyde dehydrogenase enzyme in Rattus norvegicus, Mmsdh, also converts malonate semialdehyde to acetyl-CoA (U.S. Pat. No. 8,048,624). A malonate semialdehyde dehydrogenase (acetylating) enzyme has also been characterized in Pseudomonas fluorescens, although the gene has not been identified to date (Hayaishi et al, J Biol Chem 236:781-90 (1961)). Methylmalonate semialdehyde dehydrogenase (acetylating) enzymes (EC 1.2.1.27) are also suitable candidates, as several enzymes in this class accept malonate semialdehyde as a substrate including Msdh of Bacillus subtilis (Stines-Chaumeil et al, Biochem J 395:107-15 (2006)) and the methylmalonate semialdehyde dehydrogenase of R. norvegicus (Kedishvii et al, Methods Enzymol 324:207-18 (2000)).

TABLE 88

| Protein | GenBank ID | GI Number | Organism |
|---|---|---|---|
| ddcC | ACV84070.1 | 258618587 | Halomonas sp. |
| | | | HTNK1 |
| ANI_1_1120014 | XP_001389265.1 | 145229913 | Aspergillus niger |
| ALD6 | XP_710976.1 | 68490403 | Candida albicans |
| YALI0C01859g | XP_501343.1 | 50547747 | Yarrowia lipolytica |
| mmsA_1 | YP_257876.1 | 70734236 | Pseudomonas |
| | | | fluorescens |
| mmsA_2 | YP_257884.1 | 70734244 | Pseudomonas |
| | | | fluorescens |
| PA0130 | NP_248820.1 | 15595328 | Pseudomonas |
| | | | aeruginosa |
| Mmsdh | Q02253.1 | 400269 | Rattus norvegicus |
| msdh | NP_391855.1 | 16081027 | Bacillus subtilis |
| IolA | ABP57762.1 | 145309085 | Lactobacillus casei |

2.7.2.a

Pyruvate kinase (Step 10N), also known as phosphoenolpyruvate synthase (EC 2.7.9.2), converts pyruvate and ATP to PEP and AMP. This enzyme is encoded by the PYK1 (Burke et al., J. Biol. Chem. 258:2193-2201 (1983)) and PYK2 (Boles et al., J. Bacteriol. 179:2987-2993 (1997)) genes in S. cerevisiae. In E. coli, this activity is catalyzed by the gene products of pykF and pykA. Selected homologs of the S. cerevisiae enzymes are also shown in the table below.

TABLE 89

| Protein | GenBank ID | GI Number | Organism |
|---|---|---|---|
| PYK1 | NP_009362 | 6319279 | Saccharomyces |
| | | | cerevisiae |
| PYK2 | NP_014992 | 6324923 | Saccharomyces |
| | | | cerevisiae |
| pykF | NP_416191.1 | 16129632 | Escherichia coli |
| pykA | NP_416368.1 | 16129807 | Escherichia coli |
| KLLA0F23397g | XP_456122.1 | 50312181 | Kluyveromyces |
| | | | lactis |
| CaO19.3575 | XP_714934.1 | 68482353 | Candida albicans |
| CaO19.11059 | XP_714997.1 | 68482226 | Candida albicans |
| YALI0F09185p | XP_505195 | 210075987 | Yarrowia lipolytica |
| ANI_1_1126064 | XP_001391973 | 145238652 | Apergillus niger |

2.8.3.a

Activation of malonate to malonyl-CoA is catalyzed by a CoA transferase in EC class 2.8.3.a. Malonyl-CoA:acetate CoA transferase (EC 2.8.3.3) enzymes have been characterized in Pseudomonas species including Pseudomonas fluorescens and Pseudomonas putida (Takamura et al, Biochem Int 3:483-91 (1981); Hayaishi et al, J Biol Chem 215:125-36 (1955)). Genes associated with these enzymes have not been identified to date. A mitochondrial CoA transferase found in Rattus norvegicus liver also catalyzes this reaction and is able to utilize a range of CoA donors and acceptors (Deana et al, Biochem Int 26:767-73 (1992)). Several CoA transferase enzymes described above can also be applied to catalyze step K of FIG. 10. These enzymes include acetyl-CoA transferase (Table 26), 3-HB CoA transferase (Table 8), acetoacetyl-CoA transferase (table 55), SCOT (table 56) and other CoA transferases (table 57).

3.1.3.a

Phosphoenolpyruvate phosphatase (EC 3.1.3.60, Step 10N) catalyzes the hydrolysis of PEP to pyruvate and phosphate. Numerous phosphatase enzymes catalyze this activity, including alkaline phosphatase (EC 3.1.3.1), acid phosphatase (EC 3.1.3.2), phosphoglycerate phosphatase (EC 3.1.3.20) and PEP phosphatase (EC 3.1.3.60). PEP phosphatase enzymes have been characterized in plants such as Vignia radiate, Bruguiera sexangula and Brassica nigra. The phytase from Aspergillus fumigates, the acid phosphatase from Homo sapiens and the alkaline phosphatase of E. coli also catalyze the hydrolysis of PEP to pyruvate (Brugger et al, Appl Microbiol Biotech 63:383-9 (2004); Hayman et al, Biochem J 261:601-9 (1989); et al, The Enzymes 3^{rd} Ed. 4:373-415 (1971))). Similar enzymes have been characterized in Campylobacter jejuni (van Mourik et al., Microbiol. 154:584-92 (2008)), Saccharomyces cerevisiae (Oshima et al., Gene 179:171-7 (1996)) and Staphylococcus aureus (Shah and Blobel, J. Bacteriol. 94:780-1 (1967)). Enzyme engineering and/or removal of targeting sequences may be required for alkaline phosphatase enzymes to function in the cytoplasm.

TABLE 90

| Protein | GenBank ID | GI Number | Organism |
|---|---|---|---|
| phyA | O00092.1 | 41017447 | Aspergillus fumigatus |
| Acp5 | P13686.3 | 56757583 | Homo sapiens |
| phoA | NP_414917.2 | 49176017 | Escherichia coli |
| phoX | ZP_01072054.1 | 86153851 | Campylobacter jejuni |
| PHO8 | AAA34871.1 | 172164 | Saccharomyces |
| | | | cerevisiae |
| SaurJH1_2706 | YP_001317815.1 | 150395140 | Staphylococcus aureus |

4.1.1.a

Several reactions in FIG. 10 are catalyzed by decarboxylase enzymes in EC class 4.1.1, including oxaloacetate decarboxylase (Step B), malonyl-CoA decarboxylase (step D) and pyruvate carboxylase or carboxykinase (step A).

Carboxylation of phosphoenolpyruvate to oxaloacetate is catalyzed by phosphoenolpyruvate carboxylase (EC 4.1.1.31). Exemplary PEP carboxylase enzymes are encoded by ppc in E. coli (Kai et al., Arch. Biochem. Biophys. 414:170-179 (2003), ppcA in Methylobacterium extorquens AM1 (Arps et al., J. Bacteriol. 175:3776-3783 (1993), and ppc in Corynebacterium glutamicum (Eikmanns et al., Mol. Gen. Genet. 218:330-339 (1989).

TABLE 91

| Protein | GenBank ID | GI Number | Organism |
|---|---|---|---|
| Ppc | NP_418391 | 16131794 | Escherichia coli |
| ppcA | AAB58883 | 28572162 | Methylobacterium extorquens |
| Ppc | ABB53270 | 80973080 | Corynebacterium glutamicum |

An alternative enzyme for carboxylating phosphoenolpyruvate to oxaloacetate is PEP carboxykinase (EC 4.1.1.32, 4.1.1.49), which simultaneously forms an ATP or GTP. In most organisms PEP carboxykinase serves a gluconeogenic function and converts oxaloacetate to PEP at the expense of one ATP. S. cerevisiae is one such organism whose native PEP carboxykinase, PCK1, serves a gluconeogenic role (Valdes-Hevia et al., FEBS Lett. 258:313-316 (1989). E. coli is another such organism, as the role of PEP carboxykinase in producing oxaloacetate is believed to be minor when compared to PEP carboxylase (Kim et al., Appl. Environ. Microbiol. 70:1238-1241 (2004)). Nevertheless, activity of the native E. coli PEP carboxykinase from PEP towards oxaloacetate has been recently demonstrated in ppc mutants of E. coli K-12 (Kwon et al., Microbiol. Biotechnol. 16:1448-1452 (2006)). These strains exhibited no growth defects and had increased succinate production at high NaHCO₃ concentrations. Mutant strains of E. coli can adopt Pck as the dominant CO₂-fixing enzyme following adaptive evolution (Zhang et al. 2009). In some organisms, particularly rumen bacteria, PEP carboxykinase is quite efficient in producing oxaloacetate from PEP and generating ATP. Examples of PEP carboxykinase genes that have been cloned into E. coli include those from Mannheimia succiniciproducens (Lee et al., Biotechnol. Bioprocess Eng. 7:95-99 (2002)), Anaerobiospirillum succiniciproducens (Laivenieks et al., Appl. Environ. Microbiol. 63:2273-2280 (1997), and Actinobacillus succinogenes (Kim et al. supra). The PEP carboxykinase enzyme encoded by Haemophilus influenza is effective at forming oxaloacetate from PEP. Another suitable candidate is the PEPCK enzyme from Megathyrsus maximus, which has a low Km for CO₂, a substrate thought to be rate-limiting in the E. coli enzyme (Chen et al., Plant Physiol 128:160-164 (2002); Cotelesage et al., Int. J Biochem. Cell Biol. 39:1204-1210 (2007)). The kinetics of the GTP-dependent pepck gene product from Cupriavidus necator favor oxaloacetate formation (U.S. Pat. No. 8,048,624 and Lea et al, Amino Acids 20:225-41 (2001)).

TABLE 92

| Protein | GenBank ID | GI Number | Organism |
|---|---|---|---|
| PCK1 | NP_013023 | 6322950 | Saccharomyces |
| | | | cerevisiae |
| pck | NP_417862.1 | 16131280 | Escherichia coli |
| pckA | YP_089485.1 | 52426348 | Mannheimia |
| | | | succiniciproducens |
| pckA | O09460.1 | 3122621 | Anaerobiospirillum |
| | | | succiniciproducens |
| pckA | Q6W6X5 | 75440571 | Actinobacillus |
| | | | succinogenes |
| pckA | P43923.1 | 1172573 | Haemophilus |
| | | | influenza |
| AF532733.1: | AAQ10076.1 | 33329363 | Megathyrsus |
| 1 . . . 1929 | | | maximus |
| pepck | YP_728135.1 | 113869646 | Cupriavidus necator |

Oxaloacetate decarboxylase catalyzes the decarboxylation of oxaloacetate to malonate semialdehyde. Enzymes catalyzing this reaction include kgd of Mycobacterium tuberculosis (GenBank ID: O50463.4, GI: 160395583). Enzymes evolved from kgd with improved activity and/or substrate specificity for oxaloacetate have also been described (U.S. Pat. No. 8,048,624). Additional enzymes useful for catalyzing this reaction include keto-acid decarboxylases shown in the table below.

TABLE 93

| EC number | Name |
|---|---|
| 4.1.1.1 | Pyruvate decarboxylase |
| 4.1.1.7 | Benzoylformate decarboxylase |
| 4.1.1.40 | Hydroxypyruvate decarboxylase |
| 4.1.1.43 | Ketophenylpyruvate decarboxylase |
| 4.1.1.71 | Alpha-ketoglutarate decarboxylase |
| 4.1.1.72 | Branched chain keto-acid decarboxylase |
| 4.1.1.74 | Indolepyruvate decarboxylase |
| 4.1.1.75 | 2-Ketoarginine decarboxylase |
| 4.1.1.79 | Sulfopyruvate decarboxylase |
| 4.1.1.80 | Hydroxyphenylpyruvate decarboxylase |
| 4.1.1.82 | Phosphonopyruvate decarboxylase |

The decarboxylation of keto-acids is catalyzed by a variety of enzymes with varied substrate specificities, including pyruvate decarboxylase (EC 4.1.1.1), benzoylformate decarboxylase (EC 4.1.1.7), alpha-ketoglutarate decarboxylase and branched-chain alpha-ketoacid decarboxylase. Pyruvate decarboxylase (PDC), also termed keto-acid decarboxylase, is a key enzyme in alcoholic fermentation, catalyzing the decarboxylation of pyruvate to acetaldehyde. The PDC1 enzyme from Saccharomyces cerevisiae has a broad substrate range for aliphatic 2-keto acids including 2-ketobutyrate, 2-ketovalerate, 3-hydroxypyruvate and 2-phenylpyruvate (22). This enzyme has been extensively studied, engineered for altered activity, and functionally expressed in E. coli (Killenberg-Jabs et al., Eur. J. Biochem. 268:1698-1704 (2001); Li et al., Biochemistry. 38:10004-10012 (1999); ter Schure et al., Appl. Environ. Microbiol. 64:1303-1307 (1998)). The PDC from Zymomonas mobilus, encoded by pdc, also has a broad substrate range and has been a subject of directed engineering studies to alter the affinity for different substrates (Siegert et al., Protein Eng Des Sel 18:345-357 (2005)). The crystal structure of this enzyme is available (Killenberg-Jabs et al., Eur. J. Biochem. 268:1698-1704 (2001)). Other well-characterized PDC candidates include the enzymes from Acetobacter pasteurians (Chandra et al., 176:443-451 (2001)) and Kluyveromyces lactis (Krieger et al., 269:3256-3263 (2002)).

TABLE 94

| Protein | GenBank ID | GI Number | Organism |
|---|---|---|---|
| pdc | P06672.1 | 118391 | Zymomonas mobilis |
| pdc1 | P06169 | 30923172 | Saccharomyces cerevisiae |
| pdc | Q8L388 | 20385191 | Acetobacter pasteurians |
| pdc1 | Q12629 | 52788279 | Kluyveromyces lactis |

Like PDC, benzoylformate decarboxylase (EC 4.1.1.7) has a broad substrate range and has been the target of enzyme engineering studies. The enzyme from Pseudomonas putida has been extensively studied and crystal structures of this enzyme are available (Polovnikova et al., 42:1820-1830 (2003); Hasson et al., 37:9918-9930 (1998)). Site-directed mutagenesis of two residues in the active site of the Pseudomonas putida enzyme altered the affinity (Km) of naturally and non-naturally occurring substrates (Siegert et al., Protein Eng Des Sel 18:345-357 (2005)). The properties of this enzyme have been further modified by directed engineering (Lingen et al., Chembiochem. 4:721-726 (2003); Lingen et al., Protein Eng 15:585-593 (2002)). The enzyme from Pseudomonas aeruginosa, encoded by mdlC, has also been characterized experimentally (Barrowman et al., 34:57-60 (1986)). Additional gene candidates from Pseudomonas stutzeri, Pseudomonas fluorescens and other organisms can be inferred by sequence homology or identified using a growth selection system developed in Pseudomonas putida (Henning et al., Appl. Environ. Microbiol. 72:7510-7517 (2006)).

TABLE 95

| Protein | GenBank ID | GI Number | Organism |
|---|---|---|---|
| mdlC | P20906.2 | 3915757 | Pseudomonas putida |
| mdlC | Q9HUR2.1 | 81539678 | Pseudomonas aeruginosa |
| dpgB | ABN80423.1 | 126202187 | Pseudomonas stutzeri |
| ilvB-1 | YP_260581.1 | 70730840 | Pseudomonas flourescens |

A third enzyme capable of decarboxylating 2-oxoacids is alpha-ketoglutarate decarboxylase (KGD, EC 4.1.1.71). The substrate range of this class of enzymes has not been studied to date. An exemplary KDC is encoded by kad in Mycobacterium tuberculosis (Tian et al., PNAS 102:10670-10675 (2005)). KDC enzyme activity has also been detected in several species of rhizobia including Bradyrhizobium japonicum and Mesorhizobium loti (Green et al., J Bacteriol 182:2838-2844 (2000)). Although the KDC-encoding gene(s) have not been isolated in these organisms, the genome sequences are available and several genes in each genome are annotated as putative KDCs. A KDC from Euglena gracilis has also been characterized but the gene associated with this activity has not been identified to date (Shigeoka et al., Arch. Biochem. Biophys. 288:22-28 (1991)). The first twenty amino acids starting from the N-terminus were sequenced MTYKAPVKDVKFLLDKVFKV (SEQ ID NO:8) (Shigeoka and Nakano, Arch. Biochem. Biophys. 288:22-28 (1991)). The gene could be identified by testing candidate genes containing this N-terminal sequence for KDC activity. A novel class of AKG decarboxylase enzymes has recently been identified in cyanobacteria such as Synechococcus sp. PCC 7002 and homologs (Zhang and Bryant, Science 334:1551-3 (2011)).

TABLE 96

| Protein | GenBank ID | GI Number | Organism |
|---|---|---|---|
| kgd | O50463.4 | 160395583 | Mycobacterium tuberculosis |
| kgd | NP_767092.1 | 27375563 | Bradyrhizobium japonicum |
| | | | USDA110 |
| kgd | NP_105204.1 | 13473636 | Mesorhizobium loti |
| ilvB | ACB00744.1 | 169887030 | Synechococcus sp. PCC 7002 |

A fourth candidate enzyme for catalyzing this reaction is branched chain alpha-ketoacid decarboxylase (BCKA). This class of enzyme has been shown to act on a variety of compounds varying in chain length from 3 to 6 carbons (Oku et al., J Biol Chem. 263:18386-18396 (1988); Smit et al., Appl Environ Microbiol 71:303-311 (2005)). The enzyme in Lactococcus lactis has been characterized on a variety of branched and linear substrates including 2-oxobutanoate, 2-oxohexanoate, 2-oxopentanoate, 3-methyl-2-oxobutanoate, 4-methyl-2-oxobutanoate and isocaproate (Smit et al., Appl Environ Microbiol 71:303-311 (2005)). The enzyme has been structurally characterized (Berg et al., Science. 318:1782-1786 (2007)). Sequence alignments between the Lactococcus lactis enzyme and the pyruvate decarboxylase of Zymomonas mobilus indicate that the catalytic and substrate recognition residues are nearly identical (Siegert et al., Protein Eng Des Sel 18:345-357 (2005)), so this enzyme would be a promising candidate for directed engineering. Several ketoacid decarboxylases of Saccharomyces cerevisiae catalyze the decarboxylation of branched substrates, including ARO10, PDC6, PDC5, PDC1 and THI3 (Dickenson et al, J Biol Chem 275:10937-42 (2000)). Yet another BCKAD enzyme is encoded by rv0853c of Mycobacterium tuberculosis (Werther et al, J Biol Chem 283:5344-54 (2008)). This enzyme is subject to allosteric activation by alpha-ketoacid substrates. Decarboxylation of alpha-ketoglutarate by a BCKA was detected in Bacillus subtilis; however, this activity was low (5%) relative to activity on other branched-chain substrates (Oku and Kaneda, J Biol Chem. 263:18386-18396 (1988)) and the gene encoding this enzyme has not been identified to date. Additional BCKA gene candidates can be identified by homology to the Lactococcus lactis protein sequence. Many of the high-scoring BLASTp hits to this enzyme are annotated as indolepyruvate decarboxylases (EC 4.1.1.74). Indolepyruvate decarboxylase (IPDA) is an enzyme that catalyzes the decarboxylation of indolepyruvate to indoleacetaldehyde in plants and plant bacteria. Recombinant branched chain alpha-keto acid decarboxylase enzymes derived from the E1 subunits of the mitochondrial branched-chain keto acid dehydrogenase complex from Homo sapiens and Bos taurus have been cloned and functionally expressed in E. coli (Davie et al., J. Biol. Chem. 267:16601-16606 (1992); Wynn et al., J. Biol. Chem. 267:12400-12403 (1992); Wynn et al., J. Biol. Chem. 267:1881-1887 (1992)). In these studies, the authors found that co-expression of chaperonins GroEL and GroES enhanced the specific activity of the decarboxylase by 500-fold (Wynn et al., J. Biol. Chem. 267:12400-12403 (1992)). These enzymes are composed of two alpha and two beta subunits.

TABLE 97

| Protein | GenBank ID | GI Number | Organism |
|---|---|---|---|
| kdcA | AAS49166.1 | 44921617 | Lactococcus lactis |
| PDC6 | NP_010366.1 | 6320286 | Saccharomyces cerevisiae |
| PDC5 | NP_013235.1 | 6323163 | Saccharomyces cerevisiae |
| PDC1 | P06169 | 30923172 | Saccharomyces cerevisiae |
| ARO10 | NP_010668.1 | 6320588 | Saccharomyces cerevisiae |
| THI3 | NP_010203.1 | 6320123 | Saccharomyces cerevisiae |
| rv0853c | O53865.1 | 81343167 | Mycobacterium tuberculosis |
| BCKDHB | NP_898871.1 | 34101272 | Homo sapiens |
| BCKDHA | NP_000700.1 | 11386135 | Homo sapiens |
| BCKDHB | P21839 | 115502434 | Bos taurus |
| BCKDHA | P11178 | 129030 | Bos taurus |

3-Phosphonopyruvate decarboxylase (EC 4.1.1.82) catalyzes the decarboxylation of 3-phosphonopyruvate to 2-phosphonoacetaldehyde. Exemplary phosphonopyruvate decarboxylase enzymes are encoded by dhpF of Streptomyces luridus, ppd of Streptomyces viridochromogenes, fom2 of Streptomyces wedmorensis and bcpC of Streptomyces hygroscopius (Circello et al, Chem Biol 17:402-11 (2010); Blodgett et al, FEMS Microbiol Lett 163:149-57 (2005); Hidaka et al, Mol Gen Genet 249:274-80 (1995); Nakashita et al, Biochim Biophys Acta 1490:159-62 (2000)). The Bacteroides fragilis enzyme, encoded by aepY, also decarboxylates pyruvate and sulfopyruvate (Zhang et al, J Biol Chem 278:41302-8 (2003)).

TABLE 98

| Protein | GenBank ID | GI Number | Organism |
|---|---|---|---|
| dhpF | ACZ13457.1 | 268628095 | Streptomyces luridus |
| Ppd | CAJ14045.1 | 68697716 | Streptomyces viridochromogenes |
| Fom2 | BAA32496.1 | 1061008 | Streptomyces wedmorensis |
| aepY | AAG26466.1 | 11023509 | Bacteroides fragilis |

Many oxaloacetate decarboxylase enzymes such as the eda gene product in E. coli (EC 4.1.1.3), act on the terminal acid of oxaloacetate to form pyruvate. Because decarboxylation at the 3-keto acid position competes with the malonate semialdehyde forming decarboxylation at the 2-keto-acid position, this enzyme activity can be knocked out in a host strain with a pathway proceeding through a malonate semilaldehyde intermediate.

Malonyl-CoA decarboxylase (EC 4.1.1.9) catalyzes the decarboxylation of malonyl-CoA to acetyl-CoA. Enzymes have been characterized in Rhizobium leguminosarum and Acinetobacter calcoaceticus (An et al, Eur J Biochem 257: 395-402 (1998); Koo et al, Eur Biochem 266:683-90 (1999)). Similar enzymes have been characterized in Streptomyces erythreus (Hunaiti et al, Arch Biochem Biophys 229:426-39 (1984)). A recombinant human malonyl-CoA decarboxylase was overexpressed in E. coli (Zhou et al, Prot Expr Pur 34:261-9 (2004)). Methylmalonyl-CoA decarboxylase enzymes that decarboxylate malonyl-CoA are also suitable candidates. For example, the Veillonella parvula enzyme accepts malonyl-CoA as a substrate (Hilpert et al, Nature 296:584-5 (1982)). The E. coli enzyme is encoded by ygfG (Benning et al., Biochemistry. 39:4630-4639 (2000); Haller et al., Biochemistry. 39:4622-4629 (2000)). The stereo specificity of the E. coli enzyme was not reported, but the enzyme in Propionigenium modestum (Bott et al., Eur. J. Biochem. 250:590-599 (1997)) and Veillonella parvula (Huder et al., J. Biol. Chem. 268:24564-24571 (1993)) catalyzes the decarboxylation of the (S)-stereoisomer of methylmalonyl-CoA (Hoffmann et al., FEBS. Lett. 220:121-125 (1987)). The enzymes from P. modestum and V. parvula are comprised of multiple subunits that not only decarboxylate (S)-methylmalonyl-CoA, but also create a pump that transports sodium ions across the cell membrane as a means to generate energy.

TABLE 99

| Protein | GenBank ID | GI Number | Organism |
|---|---|---|---|
| YgfG | NP_417394 | 90111512 | Escherichia coli |
| matA | Q9ZIP6 | 75424899 | Rhizobium leguminosarum |
| mdcD | AAB97628.1 | 2804622 | Acinetobacter clacoaceticus |
| mdcE | AAF20287.1 | 6642782 | Acinetobacter clacoaceticus |
| mdcA | AAB97627.1 | 2804621 | Acinetobacter clacoaceticus |
| mdcC | AAB97630.1 | 2804624 | Acinetobacter clacoaceticus |
| mcd | NP_036345.2 | 110349750 | Homo sapiens |
| mmdA | CAA05137 | 2706398 | Propionigenium modestum |
| mmdD | CAA05138 | 2706399 | Propionigenium modestum |
| mmdC | CAA05139 | 2706400 | Propionigenium modestum |
| mmdB | CAA05140 | 2706401 | Propionigenium modestum |
| mmdA | CAA80872 | 415915 | Veillonella parvula |
| mmdC | CAA80873 | 415916 | Veillonella parvula |
| mmdE | CAA80874 | 415917 | Veillonella parvula |
| mmdD | CAA80875 | 415918 | Veillonella parvula |
| mmdB | CAA80876 | 415919 | Veillonella parvula |

6.2.1.a

Activation of malonate to malonyl-CoA is catalyzed by a CoA synthetase in EC class 6.2.1.a. CoA synthetase enzymes that catalyze this reaction have not been described in the literature to date. Several CoA synthetase enzymes described above can also be applied to catalyze step K of FIG. 10. These enzymes include acetyl-CoA synthetase (Table 16, 25) and ADP forming CoA synthetases (Table 17).

6.4.1.a

Pyruvate carboxylase (EC 6.4.1.1) converts pyruvate to oxaloacetate at the cost of one ATP (step H). Exemplary pyruvate carboxylase enzymes are encoded by PYC1 (Walker et al., Biochem. Biophys. Res. Commun. 176:1210-1217 (1991) and PYC2 (Walker et al., supra) in Saccharomyces cerevisiae, and pyc in Mycobacterium smegmatis (Mukhopadhyay and Purwantini, Biochim. Biophys. Acta 1475:191-206 (2000)).

TABLE 100

| Protein | GenBank ID | GI Number | Organism |
|---|---|---|---|
| PYC1 | NP_011453 | 6321376 | Saccharomyces cerevisiae |
| PYC2 | NP_009777 | 6319695 | Saccharomyces cerevisiae |
| Pyc | YP_890857.1 | 118470447 | Mycobacterium smegmatis |

Acetyl-CoA carboxylase (EC 6.4.1.2) catalyzes the ATP-dependent carboxylation of acetyl-CoA to malonyl-CoA. This enzyme is biotin dependent and is the first reaction of fatty acid biosynthesis initiation in several organisms. Exemplary enzymes are encoded by accABCD of E. coli (Davis et al, J Biol Chem 275:28593-8 (2000)), ACC1 of Saccharomyces cerevisiae and homologs (Sumper et al, Methods Enzym 71:34-7 (1981)).

TABLE 101

| Protein | GenBank ID | GI Number | Organism |
|---|---|---|---|
| ACC1 | CAA96294.1 | 1302498 | Saccharomyces |
| | | | cerevisiae |
| KLLA0F06072g | XP_455355.1 | 50310667 | Kluyveromyces |
| | | | lactis |
| ACC1 | XP_718624.1 | 68474502 | Candida albicans |
| YALI0C11407p | XP_501721.1 | 50548503 | Yarrowia lipolytica |
| ANI_1_1724104 | XP_001395476.1 | 145246454 | Aspergillus niger |
| accA | AAC73296.1 | 1786382 | Escherichia coli |
| accB | AAC76287.1 | 1789653 | Escherichia coli |
| accC | AAC76288.1 | 1789654 | Escherichia coli |
| accD | AAC75376.1 | 1788655 | Escherichia coli |

### 5. SEQUENCE LISTING

The present specification is being filed with a computer readable form (CRF) copy of the Sequence Listing. The CRF entitled 12956-404_SEQLIST.txt, which was created on Oct. 10, 2016 and is 18,816 bytes in size, is identical to the paper copy of the Sequence Listing and is incorporated herein by reference in its entirety.

Throughout this application various publications have been referenced. The disclosures of these publications in their entireties, including GenBank and GI number publications, are hereby incorporated by reference in this application in order to more fully describe the state of the art to which this invention pertains.

Although the invention has been described with reference to the examples and embodiments provided above, it should be understood that various modifications can be made without departing from the spirit of the invention provided herein.

1.-45. (canceled)

46. A non-naturally occurring microbial organism comprising a 1,3-BDO pathway, wherein said organism comprises at least one endogenous and/or exogenous nucleic acid encoding a 1,3-BDO pathway enzyme expressed in a sufficient amount to produce 1,3-BDO, and:
(1) wherein the organism:
i. has lower or no enzymatic activity that converts acetoacetyl-CoA to acetoacetate as compared to a wild-type version of the organism;
ii. comprises a disruption in an endogenous nucleic acid encoding an enzyme that converts acetoacetyl-CoA to acetoacetate;
iii. expresses an attenuated acetoacetyl-CoA hydrolase or transferase; or
iv. comprises a disruption in an endogenous nucleic acid encoding an acetoacetyl-CoA hydrolase or transferase;
(2) wherein the organism:
i. has lower or no enzymatic activity that converts 3-hydroxybutyryl-CoA to 3-hydroxybutyrate as compared to a wild-type version of the organism;
ii. comprises a disruption in an endogenous nucleic acid encoding an enzyme that converts 3-hydroxybutyryl-CoA to 3-hydroxybutyrate;
iii. expresses an attenuated 3-hydroxybutyryl-CoA hydrolase or transferase; or
iv. comprises a disruption in an endogenous nucleic acid encoding a 3-hydroxybutyryl-CoA hydrolase or transferase;
(3) wherein the organism:
i. has lower or no enzymatic activity that converts 3-hydroxybutyraldehyde to 3-hydroxybutyrate as compared to a wild-type version of the organism;
ii. comprises a disruption in an endogenous nucleic acid encoding an enzyme that converts 3-hydroxybutyraldehyde to 3-hydroxybutyrate;
iii. expresses an attenuated 3-hydroxybutyraldehyde dehydrogenase; or
iv. comprises a disruption in an endogenous nucleic acid encoding a 3-hydroxybutyraldehyde dehydrogenase;
(4) wherein the organism:
i. has lower or no enzymatic activity that catalyzes 1,3-butanediol to 3-oxobutanol as compared to a wild-type version of the organism;
ii. comprises a disruption in an endogenous nucleic acid encoding an enzyme that catalyzes 1,3-butanediol to 3-oxobutanol;
iii. expresses an attenuated 1,3-butanediol dehydrogenase; or
iv. comprises a disruption in an endogenous nucleic acid encoding a 1,3-butanediol dehydrogenase;
(5) wherein the organism:
i. has lower or no enzymatic activity that converts G3P to glycerol as compared to a wild-type version of the organism;
ii. comprises a disruption in an endogenous nucleic acid encoding an enzyme that converts G3P to glycerol;
iii. expresses an attenuated G3P dehydrogenase or G3P phosphatase; or
iv. comprises a disruption in an endogenous nucleic acid encoding a G3P dehydrogenase or a G3P phosphatase;
(6) wherein the organism:
i. has lower or no enzymatic activity that converts pyruvate to acetaldehyde as compared to a wild-type version of the organism;
ii. comprises a disruption in an endogenous nucleic acid encoding an enzyme that converts pyruvate to acetaldehyde;
iii. expresses an attenuated pyruvate decarboxylase; or
iv. comprises a disruption in an endogenous nucleic acid encoding a pyruvate decarboxylase; or
(7) wherein the organism:
i. has lower or no enzymatic activity that converts acetyl-CoA to ethanol as compared to a wild-type version of the organism;
ii. comprises a disruption in an endogenous nucleic acid encoding an enzyme that converts acetyl-CoA to ethanol;
iii. expresses an attenuated ethanol dehydrogenase or acetaldehyde dehydrogenase; or
iv. comprises a disruption in an endogenous nucleic acid encoding an ethanol dehydrogenase or acetaldehyde dehydrogenase.

47. The organism of claim 46, wherein the organism:
i. has lower or no enzymatic activity that converts acetoacetyl-CoA to acetoacetate as compared to a wild-type version of the organism;
ii. comprises a disruption in an endogenous nucleic acid encoding an enzyme that converts acetoacetyl-CoA to acetoacetate;
iii. expresses an attenuated acetoacetyl-CoA hydrolase or transferase; or
iv. comprises a disruption in an endogenous nucleic acid encoding an acetoacetyl-CoA hydrolase or transferase;

48. The organism of claim 47, comprising a disruption in an endogenous nucleic acid encoding an acetoacetyl-CoA hydrolase or transferase.

49. The organism of claim 46, wherein the organism:
i. has lower or no enzymatic activity that converts 3-hydroxybutyryl-CoA to 3-hydroxybutyrate as compared to a wild-type version of the organism;
ii. comprises a disruption in an endogenous nucleic acid encoding an enzyme that converts 3-hydroxybutyryl-CoA to 3-hydroxybutyrate;
iii. expresses an attenuated 3-hydroxybutyryl-CoA hydrolase or transferase; or
iv. comprises a disruption in an endogenous nucleic acid encoding a 3-hydroxybutyryl-CoA hydrolase or transferase;

50. The organism of claim 49, comprising a disruption in an endogenous nucleic acid encoding a 3-hydroxybutyryl-CoA hydrolase or transferase.

51. The organism of claim 46, comprising a disruption in an endogenous nucleic acid encoding an acetoacetyl-CoA hydrolase or transferase and a disruption in an endogenous nucleic acid encoding a 3-hydroxybutyryl-CoA hydrolase or transferase.

52. The organism of claim 46, wherein the 1,3-BDO pathway enzyme is selected from the group consisting of 4B, 4C, 4D, 4E, 4F, 4G, 4H, 4I, 4J, 4L, 4N, 4O, 7E, and 7F; wherein 4B is an Acetoacetyl-CoA reductase (CoA-dependent, alcohol forming); 4C is a 3-oxobutyraldehyde reductase (aldehyde reducing); 4D is a 4-hydroxy-2-butanone reductase, 4E is an Acetoacetyl-CoA reductase (CoA-dependent, aldehyde forming), 4F is a 3-oxobutyraldehyde reductase (ketone reducing), 4G is a 3-hydroxybutyraldehyde reductase, 4H is an Acetoacetyl-CoA reductase (ketone reducing); 4I is a 3-hydroxybutyryl-CoA reductase (aldehyde forming); 4J is a 3-hydroxybutyryl-CoA reductase (alcohol forming); 4L is an acetoacetate reductase; 4N is a 3-hydroxybutyrate reductase; 4O is a 3-hydroxybutyrate dehydrogenase; 7E is an acetyl-CoA carboxylase; and 7F is an acetoacetyl-CoA synthase.

53. The organism of claim 46, wherein the 1,3-BDO pathway comprises a pathway selected from the group consisting of:
i. 4A, 4E, 4F and 4G;
ii. 4A, 4B and 4D;
iii. 4A, 4E, 4C and 4D;
iv. 4A, 4H and 4J;
v. 4A, 4H, 4I and 4G;
vi. 4A, 4H, 4M, 4N and 4G;
vii. 4A, 4K, 4O, 4N and 4G;
viii. 4A, 4K, 4L, 4F and 4G
ix. 7E, 7F, 4E, 4F and 4G;
x. 7E, 7F, 4B and 4D;
xi. 7E, 7F, 4E, 4C and 4D;
xii. 7E, 7F, 4H and 4J;
xiii. 7E, 7F, 4H, 4I and 4G;
xiv. 7E, 7F, 4H, 4M, 4N and 4G;
xv. 7E, 7F, 4K, 4O, 4N and 4G; and
xvi. 7E, 7F, 4K, 4L, 4F and 4G;
wherein 4A is an Acetoacetyl-CoA thiolase; 4B is an Acetoacetyl-CoA reductase (CoA-dependent, alcohol forming); 4C is a 3-oxobutyraldehyde reductase (aldehyde reducing); 4D is a 4-hydroxy-2-butanone reductase, 4E is an Acetoacetyl-CoA reductase (CoA-dependent, aldehyde forming), 4F is a 3-oxobutyraldehyde reductase (ketone reducing), 4G is a 3-hydroxybutyraldehyde reductase, 4H is an Acetoacetyl-CoA reductase (ketone reducing); 4I is a 3-hydroxybutyryl-CoA reductase (aldehyde forming); 4J is a 3-hydroxybutyryl-CoA reductase (alcohol forming); 4K is an acetoacetyl-CoA transferase, an acetoacetyl-CoA hydrolase, an acetoacetyl-CoA synthetase, or a phosphotransacetoacetylase and acetoacetate kinase; 4L is an acetoacetate reductase; 4M is a 3-hydroxybutyryl-CoA transferase, hydrolase, or synthetase; 4N is a 3-hydroxybutyrate reductase; 4O is a 3-hydroxybutyrate dehydrogenase; 7E is an acetyl-CoA carboxylase; and 7F is an acetoacetyl-CoA synthase.

54. The organism of claim 53, wherein the 1,3-BDO pathway comprises 4A, 4H, 4I and 4G.

55. The organism of claim 46, wherein the endogenous and/or exogenous nucleic acid is an endogenous nucleic acid.

56. The organism of claim 46, wherein the endogenous and/or exogenous nucleic acid is an exogenous nucleic acid.

57. The organism of claim 56, wherein said organism comprises two, three, four, five, or six exogenous nucleic acids each encoding a 1,3-BDO pathway enzyme.

58. The organism of claim 56, wherein said at least one exogenous nucleic acid is a heterologous nucleic acid.

59. The organism of claim 46, wherein said organism further comprises:
(1) a pentose phosphate pathway, wherein said organism comprises at least one endogenous and/or exogenous nucleic acid encoding a pentose phosphate pathway enzyme selected from the group consisting of glucose-6-phosphate dehydrogenase, 6-phosphogluconolactonase, and 6 phosphogluconate dehydrogenase (decarboxylating); or
(2) an Entner Doudoroff pathway, wherein said organism comprises at least one endogenous and/or exogenous nucleic acid encoding an Entner Doudoroff pathway enzyme selected from the group consisting of glucose-6-phosphate dehydrogenase, 6-phosphogluconolactonase, phosphogluconate dehydratase, and 2-keto-3-deoxygluconate 6-phosphate aldolase.

60. The organism of claim 46, wherein said organism is in a substantially anaerobic culture medium.

61. A culture medium comprising the non-naturally occurring organism of claim 46.

62. The culture medium of claim 61 further comprising 1,3-BDO.

63. A method for producing 1,3-BDO, comprising culturing the organism of claim 46 under conditions and for a sufficient period of time to produce 1,3-BDO.

64. The method of claim 63 further comprising separating 1,3-BDO from other components in the culture.

65. The method of claim 64, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, absorption chromatography, or ultrafiltration.

66. The method of claim 64, wherein the separating comprises distillation.
